SEQUENCE LISTING (4 sequences)

SEQ ID NO: 1 - protein, 21 aa, Homo sapiens
GIVEQCCTSICSLYQLENYCN

SEQ ID NO: 2 - protein, 30 aa, homo sapiens
FVNQHLCGSHLVEALYLVCGERGFFYTPKT

SEQ ID NO: 3 - protein, 22 aa, Artificial Sequence
  Generic A-chain Polypeptide
  MISC_FEATURE (1)..(1): Xaa is Gly or Lys
  MISC_FEATURE (3)..(3): Xaa is Val, Gly, or Lys
  MISC_FEATURE (5)..(5): Xaa is Gln or Lys
  MISC_FEATURE (8)..(8): Xaa is Thr, His, or Lys
  MISC_FEATURE (9)..(9): Xaa is Ser or Lys
  MISC_FEATURE (10)..(10): Xaa is Ile or Lys
  MISC_FEATURE (13)..(13): Xaa is Leu or Lys
  MISC_FEATURE (14)..(14): Xaa is Tyr or Lys
  MISC_FEATURE (15)..(15): Xaa is Gln or Lys
  MISC_FEATURE (18)..(18): Xaa is Asn or Lys
  MISC_FEATURE (21)..(21): Xaa is Asn, Gly, or Lys
  MISC_FEATURE (22)..(22): Xaa is Arg, Lys, or absent
XIXEXCCXXXCSXXXLEXYCXX

SEQ ID NO: 4 - protein, 35 aa, Artificial Sequence
  Generic B-chain polypeptide
  MISC_FEATURE (1)..(1): Xaa is Phe or Lys
  MISC_FEATURE (3)..(3): Xaa is Asn or Lys
  MISC_FEATURE (4)..(4): Xaa is Gln or Lys
  MISC_FEATURE (16)..(16): Xaa is Tyr or Lys
  MISC_FEATURE (17)..(17): Xaa is Leu or Lys
  MISC_FEATURE (25)..(25): Xaa is Phe or Lys
  MISC_FEATURE (28)..(28): Xaa is Pro or Lys
  MISC_FEATURE (29)..(29): Xaa is Lys, Pro, Arg, or absent
  MISC_FEATURE (30)..(30): Xaa is Thr or absent
  MISC_FEATURE (31)..(31): Xaa is Arg if Xaa30 is Thr, or absent
  MISC_FEATURE (32)..(32): Xaa is Pro if Xaa31 is Arg, or absent
  MISC_FEATURE (33)..(33): Xaa is Arg if Xaa32 is Pro, or absent
  MISC_FEATURE (34)..(34): Xaa is Pro if Xaa33 is Arg, or absent
  MISC_FEATURE (35)..(35): Xaa is ArgR if Xaa34 is Pro, or absent
XVXXHLCGSHLVEALXXVCGERGFXYTXXXXXXXX

(19) United States
(12) Patent Application Publication
(10) Pub. No.: US 2022/0273770 A1
(43) Pub. Date: Sep. 1, 2022

(54) GLUCOSE-RESPONSIVE INSULIN CONJUGATES

(71) Applicants: Danqing FENG, Kenilworth, NJ (US); Erin N. GUIDRY, Kenilworth, NJ (US); Pei HUO, Kenilworth, NJ (US); Andrew J. KASSICK, Wexford, PA (US); Ahmet KEKEC, Kenilworth, NJ (US); Songnian LIN, Kenilworth, NJ (US); Christopher R MOYES, Westfield, NJ (US); Dmitri A. PISSARNITSKI, Kenilworth, NJ (US); Lin YAN, Kenilworth, NJ (US); Yuping ZHU, Kenilworth, NJ (US); Merck Sharp & Dohme Corp., Rahway, NJ (US)

(72) Inventors: Danqing Feng, Green Brook, NJ (US); Erin N. Guidry, Cranford, NJ (US); Pei Huo, Millburn, NJ (US); Andrew J. Kassick, Wexford, PA (US); Ahmet Kekec, Hoboken, NJ (US); Songnian Lin, Holmdel, NJ (US); Christopher R. Moyes, Westfield, NJ (US); Dmitri A. Pissarnitski, Scotch Plains, NJ (US); Lin Yan, East Brunswick, NJ (US); Yuping Zhu, Basking Ridge, NJ (US)

(73) Assignee: Merck Sharp & Dohme Corp., Rahway, NJ (US)

(21) Appl. No.: 17/629,501

(22) PCT Filed: Jul. 23, 2020
(86) PCT No.: PCT/US2020/043168

§ 371 Date: Jan. 24, 2022

Related U.S. Application Data
(60) Provisional application No. 62/880,270, filed on Jul. 30, 2019.

(51) Int. Cl. A61K 38/28; A61K 47/54; A61P 3/10

(52) U.S. Cl. CPC A61K 38/28; A61K 47/549; A61P 3/10

(57) ABSTRACT

Glucose-responsive insulin conjugates that contain one or more trisaccharides are provided. Such insulin conjugates may display a pharmacokinetic (PK) and/or pharmacodynamic (PD) profile that is responsive to the systemic concentrations of a saccharide such as glucose or alpha-methylmannose, even when administered to a subject in need thereof in the absence of an exogenous multivalent saccharide-binding molecule.

### FIELD OF THE INVENTION

The present disclosure relates to glucose-responsive insulin conjugates that contain one or more trisaccharides. In particular aspects, the insulin conjugate that displays a pharmacokinetic (PK) and/or pharmacodynamic (PD) profile that is responsive to the systemic concentrations of a saccharide, such as glucose or alpha-methyl mannose, even when administered to a subject in need thereof in the absence of an exogenous multivalent saccharide-binding molecule.

### REFERENCE TO SEQUENCE LISTING SUBMITTED ELECTRONICALLY

The sequence listing of the present application is submitted electronically via EFS-Web as an ASCII-formatted sequence listing, with a file name of “24761WOPCT-SEQLIST-22JUN2020”, a creation date of Jun. 22, 2020, and a size of 3.32 KB. This sequence listing submitted via EFS-Web is part of the specification and is herein incorporated by reference in its entirety.

### BACKGROUND OF THE INVENTION

The majority of known “controlled-release” drug delivery systems are incapable of providing drugs to a patient at intervals and concentrations that are in direct proportion to the amount of a molecular indicator (e.g., a metabolite) present in the human body. The drugs in these systems are thus not literally “controlled,” but simply provided in a slow-release format that is independent of external or internal factors.

The treatment of diabetes mellitus with injectable insulin is a well-known and studied example in which uncontrolled, slow release of insulin is undesirable. In fact, it is apparent that the simple replacement of the hormone is not sufficient to prevent the pathological sequelae associated with this disease. Insulin replacement therapy for glycemic control in diabetic patients is often insufficient due to the inability of these exogenous insulins to function in response to varying glucose concentration. Among approaches to develop glucose-responsive insulins, conjugation of a cluster of sugars, e.g., D-mannose and L-fucose, to insulin has been reported in patent literature that potentially offer such glucose responsive insulins. The cluster of sugar moieties, acting as substrate of endogenous mannose receptor, potentially affect the pharmacokinetic properties of their corresponding insulin conjugates in a way that is sensitive to the endogenous glucose concentration, rendering these insulin conjugates low risk of hypoglycemia.

### SUMMARY OF THE INVENTION

The present disclosure relates to glucose-responsive insulin conjugates, which comprise at least one trisaccharide, and their synthesis. These insulin conjugates may display a pharmacokinetic (PK) and/or pharmacodynamic (PD) profile that is responsive to the systemic concentrations of a saccharide such as glucose or alpha-methyl mannose when administered to a subject in need thereof. In general, the conjugates comprise an insulin or insulin analog molecule covalently attached at its N-terminal amino groups of A-chain, such as ^{A1}Gly, and B-chain ^{B1}Phe, respectively, or 6-amino group of the side chain of B²⁹Lys, or any Lys residue engineered into insulin backbone, via a linker to a trisaccharide cluster of sugar moieties. Specifically, the linker-trisaccharide moieties are conjugated onto the side-chain amino group of B29 lysine or any other lysine and/or A1 and B1 amino groups of insulins or insulin analogs. Such conjugates offer a balanced binding profile against both insulin receptor and mannose receptor. These conjugates demonstrate glucose lowering in the presence of alpha-methyl mannose, a surrogate for glucose, and are potentially useful for the treatment of diabetes with lower risk of hypoglycemia.

Other embodiments, aspects and features of the present disclosure are either further described in or will be apparent from the ensuing description, examples and appended claims.

### BRIEF DESCRIPTION OF THE DRAWINGS

FIG. 1 shows plasma glucose depression curves in non-diabetic male Yucatan minipigs equipped with dual vascular access ports (n=3 per study) following i.v. injection of conjugate IOC-1 at 0.69 nmol/kg under conditions of PBS infusion or i.v. alpha methyl mannose (αMM) infusion.

FIG. 2 shows plasma glucose depression curves in non-diabetic male Yucatan minipigs equipped with dual vascular access ports (n=3 per study) following i.v. injection of conjugate IOC-2 at 0.35 nmol/kg under conditions of PBS infusion or i.v. alpha methyl mannose (αMM) infusion.

FIG. 3 shows plasma glucose depression curves in non-diabetic male Yucatan minipigs equipped with dual vascular access ports (n=3 per study) following i.v. injection of conjugate IOC-3 at 0.35 nmol/kg under conditions of PBS infusion or i.v. alpha methyl mannose (αMM) infusion.

FIG. 4 shows plasma glucose depression curves in non-diabetic male Yucatan minipigs equipped with dual vascular access ports (n=3 per study) following i.v. injection of conjugate IOC-4 at 0.35 nmol/kg under conditions of PBS infusion or i.v. alpha methyl mannose (αMM) infusion.

FIG. 5 shows plasma glucose depression curves in non-diabetic male Yucatan minipigs equipped with dual vascular access ports (n=3 per study) following i.v. injection of conjugate IOC-5 at 0.35 nmol/kg under conditions of PBS infusion or i.v. alpha methyl mannose (αMM) infusion.

FIG. 6 shows plasma glucose depression curves in non-diabetic male Yucatan minipigs equipped with dual vascular access ports (n=3 per study) following i.v. injection of conjugate IOC-6 at 0.35 nmol/kg under conditions of PBS infusion or i.v. alpha methyl mannose (αMM) infusion.

FIG. 7 shows plasma glucose depression curves in non-diabetic male Yucatan minipigs equipped with dual vascular access ports (n=3 per study) following i.v. injection of conjugate IOC-7 at 0.35 nmol/kg under conditions of PBS infusion or i.v. alpha methyl mannose (αMM) infusion.

FIG. 8 shows plasma glucose depression curves in non-diabetic male Yucatan minipigs equipped with dual vascular access ports (n=3 per study) following i.v. injection of conjugate IOC-8 at 0.17 nmol/kg under conditions of PBS infusion or i.v. alpha methyl mannose (αMM) infusion.

FIG. 9 shows plasma glucose depression curves in non-diabetic male Yucatan minipigs equipped with dual vascular access ports (n=3 per study) following i.v. injection of conjugate IOC-9 at 0.35 nmol/kg under conditions of PBS infusion or i.v. alpha methyl mannose (αMM) infusion.

FIG. 10 shows plasma glucose depression curves in non-diabetic male Yucatan minipigs equipped with dual vascular access ports (n=3 per study) following i.v. injection of conjugate IOC-11 at 0.35 nmol/kg under conditions of PBS infusion or i.v. alpha methyl mannose (αMM) infusion.

FIG. 11 shows plasma glucose depression curves in non-diabetic male Yucatan minipigs equipped with dual vascular access ports (n=3 per study) following i.v. injection of conjugate IOC-12 at 0.35 nmol/kg under conditions of PBS infusion or i.v. alpha methyl mannose (αMM) infusion.

FIG. 12 shows plasma glucose depression curves in non-diabetic male Yucatan minipigs equipped with dual vascular access ports (n=3 per study) following i.v. injection of conjugate IOC-14 at 0.35 nmol/kg under conditions of PBS infusion or i.v. alpha methyl mannose (αMM) infusion.

FIG. 13 shows plasma glucose depression curves in non-diabetic male Yucatan minipigs equipped with dual vascular access ports (n=3 per study) following i.v. injection of conjugate IOC-15 at 0.35 nmol/kg under conditions of PBS infusion or i.v. alpha methyl mannose (αMM) infusion.

FIG. 14 shows plasma glucose depression curves in non-diabetic male Yucatan minipigs equipped with dual vascular access ports (n=3 per study) following i.v. injection of conjugate IOC-17 at 0.17 nmol/kg under conditions of PBS infusion or i.v. alpha methyl mannose (αMM) infusion.

FIG. 15 shows plasma glucose depression curves in non-diabetic male Yucatan minipigs equipped with dual vascular access ports (n=3 per study) following i.v. injection of conjugate IOC-18 at 0.35 nmol/kg under conditions of PBS infusion or i.v. alpha methyl mannose (αMM) infusion.

FIG. 16 shows plasma glucose depression curves in non-diabetic male Yucatan minipigs equipped with dual vascular access ports (n=3 per study) following i.v. injection of conjugate IOC-20 at 0.17 nmol/kg under conditions of PBS infusion or i.v. alpha methyl mannose (αMM) infusion.

FIG. 17 shows plasma glucose depression curves in non-diabetic male Yucatan minipigs equipped with dual vascular access ports (n=3 per study) following i.v. injection of conjugate IOC-23 at 0.17 nmol/kg under conditions of PBS infusion or i.v. alpha methyl mannose (αMM) infusion.

FIG. 18 shows plasma glucose depression curves in non-diabetic male Yucatan minipigs equipped with dual vascular access ports (n=3 per study) following i.v. injection of conjugate IOC-24 at 0.17 nmol/kg under conditions of PBS infusion or i.v. alpha methyl mannose (αMM) infusion.

FIG. 19 shows plasma glucose depression curves in non-diabetic male Yucatan minipigs equipped with dual vascular access ports (n=3 per study) following i.v. injection of conjugate IOC-25 at 0.17 nmol/kg under conditions of PBS infusion or i.v. alpha methyl mannose (αMM) infusion.

FIG. 20 shows plasma glucose depression curves in non-diabetic male Yucatan minipigs equipped with dual vascular access ports (n=3 per study) following i.v. injection of conjugate IOC-28 at 0.35 nmol/kg under conditions of PBS infusion or i.v. alpha methyl mannose (αMM) infusion.

FIG. 21 shows plasma glucose depression curves in non-diabetic male Yucatan minipigs equipped with dual vascular access ports (n=3 per study) following i.v. injection of conjugate IOC-29 at 0.17 nmol/kg under conditions of PBS infusion or i.v. alpha methyl mannose (αMM) infusion.

FIG. 22 shows plasma glucose depression curves in non-diabetic male Yucatan minipigs equipped with dual vascular access ports (n=3 per study) following i.v. injection of conjugate IOC-30 at 0.17 nmol/kg under conditions of PBS infusion or i.v. alpha methyl mannose (αMM) infusion.

FIG. 23 shows plasma glucose depression curves in non-diabetic male Yucatan minipigs equipped with dual vascular access ports (n=3 per study) following i.v. injection of conjugate IOC-32 at 0.17 nmol/kg under conditions of PBS infusion or i.v. alpha methyl mannose (αMM) infusion.

FIG. 24 shows plasma glucose depression curves in non-diabetic male Yucatan minipigs equipped with dual vascular access ports (n=3 per study) following i.v. injection of conjugate IOC-47 at 0.17 nmol/kg under conditions of PBS infusion or i.v. alpha methyl mannose (αMM) infusion.

FIG. 25 shows plasma glucose depression curves in non-diabetic male Yucatan minipigs equipped with dual vascular access ports (n=3 per study) following i.v. injection of conjugate IOC-63 at 0.17 nmol/kg under conditions of PBS infusion or i.v. alpha methyl mannose (αMM) infusion.

FIG. 26 shows plasma glucose depression curves in non-diabetic male Yucatan minipigs equipped with dual vascular access ports (n=3 per study) following i.v. injection of conjugate IOC-65 at 0.17 nmol/kg under conditions of PBS infusion or i.v. alpha methyl mannose (αMM) infusion.

FIG. 27 shows plasma glucose depression curves in non-diabetic male Yucatan minipigs equipped with dual vascular access ports (n=3 per study) following i.v. injection of conjugate IOC-69 at 0.35 nmol/kg under conditions of PBS infusion or i.v. alpha methyl mannose (αMM) infusion.

FIG. 28 shows plasma glucose depression curves in non-diabetic male Yucatan minipigs equipped with dual vascular access ports (n=3 per study) following i.v. injection of conjugate IOC-70 at 0.35 nmol/kg under conditions of PBS infusion or i.v. alpha methyl mannose (αMM) infusion.

FIG. 29 shows plasma glucose depression curves in non-diabetic male Yucatan minipigs equipped with dual vascular access ports (n=3 per study) following i.v. injection of conjugate IOC-71 at 0.17 nmol/kg under conditions of PBS infusion or i.v. alpha methyl mannose (αMM) infusion.

FIG. 30 shows plasma glucose depression curves in non-diabetic male Yucatan minipigs equipped with dual vascular access ports (n=3 per study) following i.v. injection of conjugate IOC-73 at 0.17 nmol/kg under conditions of PBS infusion or i.v. alpha methyl mannose (αMM) infusion.

FIG. 31 shows plasma glucose depression curves in non-diabetic male Yucatan minipigs equipped with dual vascular access ports (n=3 per study) following i.v. injection of conjugate IOC-78 at 0.17 nmol/kg under conditions of PBS infusion or i.v. alpha methyl mannose (αMM) infusion.

FIG. 32 shows plasma glucose depression curves in non-diabetic male Yucatan minipigs equipped with dual vascular access ports (n=3 per study) following i.v. injection of conjugate IOC-111 at 0.17 nmol/kg under conditions of PBS infusion or i.v. alpha methyl mannose (αMM) infusion.

FIG. 33 shows plasma glucose depression curves in non-diabetic male Yucatan minipigs equipped with dual vascular access ports (n=3 per study) following i.v. injection of conjugate IOC-112 at 0.17 nmol/kg under conditions of PBS infusion or i.v. alpha methyl mannose (αMM) infusion.

FIG. 34 shows plasma glucose depression curves in non-diabetic male Yucatan minipigs equipped with dual vascular access ports (n=3 per study) following i.v. injection of conjugate IOC-115 at 0.17 nmol/kg under conditions of PBS infusion or i.v. alpha methyl mannose (αMM) infusion.

FIG. 35 shows plasma glucose depression curves in non-diabetic male Yucatan minipigs equipped with dual vascular access ports (n=3 per study) following i.v. injection of conjugate IOC-120 at 0.17 nmol/kg under conditions of PBS infusion or i.v. alpha methyl mannose (αMM) infusion.

FIG. 36 shows plasma glucose depression curves in non-diabetic male Yucatan minipigs equipped with dual vascular access ports (n=3 per study) following i.v. injection of conjugate IOC-128 at 0.17 nmol/kg under conditions of PBS infusion or i.v. alpha methyl mannose (αMM) infusion.

FIG. 37 shows plasma glucose depression curves in non-diabetic male Yucatan minipigs equipped with dual vascular access ports (n=3 per study) following i.v. injection of conjugate IOC-129 at 0.17 nmol/kg under conditions of PBS infusion or i.v. alpha methyl mannose (αMM) infusion.

### DETAILED DESCRIPTION OF THE INVENTION

### Definitions

Definitions of specific functional groups, chemical terms, and general terms used throughout the specification are described in more detail below. For purposes of this disclosure, the chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5th Edition, John Wiley & Sons, Inc., New York, 2001; Larock, Comprehensive Organic Transformations, VCH Publishers, Inc., New York, 1989; Carruthers, Some Modern Methods of Organic Synthesis, 3rd Edition, Cambridge University Press, Cambridge, 1987.

As used herein, the term “acyl,” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl; cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl, substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

As used herein, the term “aliphatic” or “aliphatic group” denotes an optionally substituted hydrocarbon moiety that may be straight-chain (i.e., unbranched), branched, or cyclic (“carbocyclic”) and may be completely saturated or may contain one or more units of unsaturation, but that is not aromatic. Unless otherwise specified, aliphatic groups contain 1 to 12 carbon atoms. In some embodiments, aliphatic groups contain 1 to 6 carbon atoms. In some embodiments, aliphatic groups contain 1 to 4 carbon atoms, and in yet other embodiments aliphatic groups contain 1 to 3 carbon atoms. Suitable aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

As used herein, the term “alkyl” refers to optionally substituted saturated, straight- or branched-chain hydrocarbon radicals derived from an aliphatic moiety containing between 1 and 6 carbon atoms by removal of a single hydrogen atom. In some embodiments, the alkyl group employed in the disclosure contains 1 to 5 carbon atoms. In another embodiment, the alkyl group employed contains 1 to 4 carbon atoms. In still other embodiments, the alkyl group contains 1 to 3 carbon atoms. In yet another embodiment, the alkyl group contains 1 or 2 carbons. Examples of alkyl radicals include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, n-heptyl, n-octyl, n-decyl, n-undecyl, dodecyl, and the like. In embodiments, the alkyl group may be substituted by replacing one or more hydrogen atoms with independently selected substituents.

As used herein, the term “alkenyl” denotes an optionally substituted monovalent group derived from a straight- or branched-chain aliphatic moiety having at least one carbon-carbon double bond by the removal of a single hydrogen atom. In particular embodiments, the alkenyl group employed in the disclosure contains 2 to 6 carbon atoms. In particular embodiments, the alkenyl group employed in the disclosure contains 2 to 5 carbon atoms. In some embodiments, the alkenyl group employed in the disclosure contains 2 to 4 carbon atoms. In another embodiment, the alkenyl group employed contains 2 or 3 carbon atoms. Alkenyl groups include, for example, ethenyl, propenyl, butenyl, 1-methyl-2-buten-1-yl, and the like. In embodiments, the alkenyl group may be substituted by replacing one or more hydrogen atoms with independently selected substituents.

As used herein, the term “alkynyl” refers to an optionally substituted monovalent group derived from a straight- or branched-chain aliphatic moiety having at least one carbon-carbon triple bond by the removal of a single hydrogen atom. In particular embodiments, the alkynyl group employed in the disclosure contains 2 to 6 carbon atoms. In particular embodiments, the alkynyl group employed in the disclosure contains 2 to 5 carbon atoms. In some embodiments, the alkynyl group employed in the disclosure contains 2 to 4 carbon atoms. In another embodiment, the alkynyl group employed contains 2 or 3 carbon atoms. Representative alkynyl groups include, but are not limited to, ethynyl, 2-propynyl (propargyl), 1-propynyl, and the like. In embodiments, the alkynyl group may be substituted by replacing one or more hydrogen atoms with independently selected substituents.

As used herein, the term “aryl” used alone or as part of a larger moiety as in “aralkyl”, “aralkoxy”, or “aryloxyalkyl”, refers to an optionally substituted monocyclic and bicyclic ring systems having a total of 5 to 10 ring members, wherein at least one ring in the system is aromatic and wherein each ring in the system contains 3 to 7 ring members. The term “aryl” may be used interchangeably with the term “aryl ring”. In particular embodiments of the present invention, “aryl” refers to an aromatic ring system that includes, but not limited to, phenyl (“Ph”), biphenyl, naphthyl, anthracyl and the like, which may bear one or more substituents.

As used herein, the term “arylalkyl” refers to an alkyl group substituted with an aryl group (e.g., an aromatic or heteroaromatic group).

As used herein, the term “carbonyl” refers to a monovalent or bivalent moiety containing a carbon-oxygen double bond. Non-limiting examples of carbonyl groups include aldehydes, ketones, carboxylic acids, ester, amide, enones, acyl halides, anhydrides, ureas, carbamates, carbonates, thioesters, lactones, lactams, hydroxamates, isocyanates, and chloroformates.

As used herein, the terms “cycloaliphatic”, “carbocycle”, or “carbocyclic”, used alone or as part of a larger moiety, refer to an optionally substituted, saturated or partially unsaturated, cyclic aliphatic monocyclic or bicyclic ring systems, as described herein, having from 3 to 10 members. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, and cyclooctadienyl. In some embodiments, the cycloalkyl has 3 to 6 carbons.

As used herein, the terms “halo” and “halogen” refer to an atom selected from fluorine (fluoro, —F), chlorine (chloro, —Cl), bromine (bromo, —Br), and iodine (iodo, —I).

As used herein, the terms “heteroaliphatic” or “heteroaliphatic group”, denote an optionally substituted hydrocarbon moiety having, in addition to carbon atoms, from 1 to 5 heteroatoms, that may be straight-chain (i.e., unbranched), branched, or cyclic (“heterocyclic”) and may be completely saturated or may contain one or more units of unsaturation, but which is not aromatic. Unless otherwise specified, heteroaliphatic groups contain 1 to 6 carbon atoms wherein lto 3 carbon atoms are optionally and independently replaced with heteroatoms selected from oxygen, nitrogen and sulfur. In some embodiments, heteroaliphatic groups contain 1 to 4 carbon atoms, wherein 1 or 2 carbon atoms are optionally and independently replaced with heteroatoms selected from oxygen, nitrogen and sulfur. In yet other embodiments, heteroaliphatic groups contain 1 to 3 carbon atoms, wherein one carbon atom is optionally and independently replaced with a heteroatom selected from oxygen, nitrogen and sulfur. Suitable heteroaliphatic groups include, but are not limited to, linear or branched, heteroalkyl, heteroalkenyl, and heteroalkynyl groups.

As used herein, the term “heteroaralkyl” refers to an alkyl group substituted by a heteroaryl, wherein the alkyl and heteroaryl portions independently are optionally substituted.

As used herein, the term “heteroaryl” used alone or as part of a larger moiety, e.g., “heteroaralkyl”, or “heteroaralkoxy”, refers to an optionally substituted group having 5 to 10 ring atoms, preferably 5, 6, or 9 ring atoms; having 6, 10, or 14 7E electrons shared in a cyclic array; and having, in addition to carbon atoms, from 1 to 5 heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, carbocyclic, or heterocyclic rings, where the radical or point of attachment is on the heteroaromatic ring. Non-limiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydro-quinolinyl, and tetrahydroisoquinolinyl. A heteroaryl group may be mono- or bicyclic. The term “heteroaryl” may be used interchangeably with the terms “heteroaryl ring”, “heteroaryl group”, or “heteroaromatic”, which are unsubstituted unless otherwise noted.

As used herein, the term “heteroatom” refers to nitrogen, oxygen, or sulfur, and includes any oxidized form of nitrogen or sulfur, and any quaternized form of a basic nitrogen. The term “nitrogen” also includes a substituted nitrogen.

As used herein, the terms “heterocycle”, “heterocyclyl”, “heterocyclic radical”, and “heterocyclic ring” are used interchangeably and refer to a stable optionally substituted 5- to 7-membered monocyclic or 7- to 10-membered bicyclic heterocyclic moiety that is either saturated or partially unsaturated, and having, in addition to carbon atoms, one or more heteroatoms, as defined above. A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, pyrrolidinyl, pyrrolidonyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or carbocyclic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “unsaturated” means that a moiety has one or more double or triple bonds.

As used herein, the term “partially unsaturated” refers to a ring moiety that includes at least one double or triple bond. The term “partially unsaturated” is intended to encompass rings having multiple sites of unsaturation but is not intended to include aryl or heteroaryl moieties, as herein defined.

As described herein, conjugates of the disclosure may contain “optionally substituted” moieties. In general, optionally substituted conjugates and moieties may be unsubstituted or substituted. The term “substituted” means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable”, as used herein, refers to compounds that are not substantially altered when subjected to conditions to allow for their production, detection, and, in particular embodiments, their recovery, purification, and use for one or more of the purposes disclosed herein.

Suitable monovalent substituents on a substitutable carbon atom of an “optionally substituted” group are independently selected from the group consisting of halogen; —(CH₂)₀₋₄R^{o}; —(CH₂)₀₋₄OR^{o}; —O—(CH₂)₀₋₄C(O)OR^{o}; —(CH₂)₀₋₄CH(OR^{o})₂; —(CH₂)₀₋₄SR^{o}; —(CH₂)₀₋₄Ph that may be substituted with R^{o}; —(CH₂)₀₋₄O(CH₂)₀₋₁Ph that may be substituted with R^{o}; —CH═CHPh that may be substituted with R^{o}; —NO₂; —CN; —N₃; —(CH₂)₀₋₄N(R^{o})₂; —(CH₂)₀₋₄N(R^{o})C(O)R^{o}; —N(R^{o}C(S)R^{o}; —(CH₂)₀₋₄N(R^{o})C(O)NR^{o}₂; —N(R^{o})C(S)NR^{o}₂; —(CH₂)₀₋₄N(R^{o})C(O)OR^{o}; —N(R^{o})N(R^{o})C(O)R^{o}; —N(R^{o})N(R^{o})C(O)NR^{o}₂; —N(R^{o})N(R^{o})C(O)OR^{o}; —(CH₂)₀₋₄C(O)R^{o}; —C(S)R^{o}; —(CH₂)₀₋₄C(O)OR^{o}; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄SR—, SC(S)SR^{o}; —(CH₂)₀₋₄SC(O)R^{o}; —(CH₂)₀₋₄C(O)NR^{o}₂; —C(S)NR^{o}₂; —C(S)SR^{o}; —SC(S)SR^{o}, —(CH₂)₀₋₄OC(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄OS(O)₂R^{o}; —S(O)₂NR^{o}₂; —(CH₂)₀₋₄S(O)R^{o}; —N(R^{o})S(O)₂NR^{o}₂; —N(R^{o})S(O)₂R^{o}; —N(OR^{o})R^{o}; —C(NH)NR^{o}₂; —P(O)₂R^{o}; —P(O)R^{o}₂; —OP(O)R^{o}₂; —OP(O)(OR^{o})₂; SiR^{o}₃; —(C₁₋₄ straight or branched)alkylene)O—N(R^{o})₂; or —(C₁₋₄ straight or branched)alkylene)C(O)O—N(R^{o})₂, wherein each R^{o}may be substituted as defined below and is independently selected from hydrogen, C₁₋₆ aliphatic, —CH₂Ph, —O(CH₂)₀₋₁Ph, or a 5- or 6-membered saturated, partially unsaturated, or aryl ring having 0 to 4 heteroatoms independently selected from nitrogen, oxygen, or sulfur, or, notwithstanding the definition above, two independent occurrences of R^{o}, taken together with their intervening atom(s), form a 3- to 12-membered saturated, partially unsaturated, or aryl mono- or bicyclic ring having 0 to 4 heteroatoms independently selected from nitrogen, oxygen, or sulfur, which may be substituted as defined below.

Suitable monovalent substituents on R^{o} (or the ring formed by taking two independent occurrences of R^{o}together with their intervening atoms), are independently selected from the group consisting of halogen, —(CH₂)₀₋₂R^{⋅}, -(haloR^{⋅}), —(CH₂)₀₋₂OH, —(CH₂)₀₋₂OR^{⋅}, —(CH₂)₀₋₂CH(OR^{⋅})₂; —O(haloR^{⋅}), —CN, —N₃, —(CH₂)₀₋₂C(O)R^{⋅}, —(CH₂)₀₋₂C(O)OH, —(CH₂)₀₋₂C(O)OR^{⋅}, —(CH₂)₀₋₂SR^{⋅}, —(CH₂)₀₋₂SH, —(CH₂)₀₋₂NH₂, —(CH₂)₀₋₂NHR^{⋅}, —(CH₂)₀₋₂NR^{⋅}₂, —NO₂, —SiR^{⋅}₃, —OSiR^{⋅}₃, —C(O)SR^{⋅}, —(C₁₋₄ straight or branched alkylene)C(O)OR^{⋅}, or —SSR^{⋅} wherein each R^{⋅} is unsubstituted or where preceded by “halo” is substituted only with one or more halogens, and is independently selected from C₁₋₄ aliphatic, —CH₂Ph, —O—(CH₂)₀₋₁Ph, or a 5- or 6-membered saturated, partially unsaturated, or aryl ring having 0 to 4 heteroatoms independently selected from nitrogen, oxygen, or sulfur. Suitable divalent substituents on a saturated carbon atom of R^{o} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic that may be substituted as defined below, or an unsubstituted 5- or 6-membered saturated, partially unsaturated, or aryl ring having 0 to 4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic that may be substituted as defined below, or an unsubstituted 5- or 6-membered saturated, partially unsaturated, or aryl ring having 0 to 4 heteroatoms independently selected from nitrogen, oxygen, or sulfur.

Suitable substituents on the aliphatic group of R* include halogen, —R^{⋅}, —(haloR^{⋅}), —OH, —OR^{⋅}, —O(haloR^{⋅}), —CN, —C(O)OH, —C(O)OR^{⋅}, —NH₂, —NHR^{⋅}, —NR^{⋅}₂, or —NO₂, wherein each R^{⋅} is unsubstituted or where preceded by “halo” is substituted only with one or more halogens, and is independently C₁₋₄ aliphatic, —CH₂Ph, —O(CH₂)₀₋₁Ph, or a 5- or 6-membered saturated, partially unsaturated, or aryl ring having 0 to 4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR⁵⁵⁴ ₂, —C(NH)NR^{†}₂, or —N(R^{†})S(O)₂R^{†}; wherein each R^{†} is independently hydrogen, C1-6 aliphatic which may be substituted as defined below, unsubstituted —OPh, or an unsubstituted 5- or 6-membered saturated, partially unsaturated, or aryl ring having 0 to 4 heteroatoms independently selected from nitrogen, oxygen, or sulfur, or, notwithstanding the definition above, two independent occurrences of R^{†}, taken together with their intervening atom(s) form an unsubstituted 3- to 12-membered saturated, partially unsaturated, or aryl mono- or bicyclic ring having 0 to 4 heteroatoms independently selected from nitrogen, oxygen, or sulfur.

Suitable substituents on the aliphatic group of R^{†} are independently selected from the group consisting of halogen, —R^{⋅}, -(haloR^{⋅}), —OH, —OR^{⋅}, —O(haloR^{⋅}), —CN, —C(O)OH, —C(O)OR^{⋅}, —NH₂, —NHR^{⋅}, —NR^{⋅}₂, or —NO₂, wherein each R^{⋅} is unsubstituted or where preceded by “halo” is substituted only with one or more halogens, and is independently C₁₋₄ aliphatic, —CH₂Ph, —O(CH₂)₀₋₁Ph, or a 5- or 6-membered saturated, partially unsaturated, or aryl ring having 0 to 4 heteroatoms independently selected from nitrogen, oxygen, or sulfur.

As used herein, the term “suitable protecting group,” refers to amino protecting groups or hydroxyl protecting groups depending on its location within the compound and includes those described in detail in Protecting Groups in Organic Synthesis, T. W. Greene and P. G. M. Wuts, 3rd edition, John Wiley & Sons, 1999.

As used herein, the term “biodegradable” refers to molecules that degrade (i.e., lose at least some of their covalent structure) under physiological or endosomal conditions. Biodegradable molecules are not necessarily hydrolytically degradable and may require enzymatic action to degrade.

As used herein, an “exogenous” molecule is one which is not present at significant levels in a patient unless administered to the patient. In particular embodiments, the patient is a mammal, e.g., a human, a dog, a cat, a rat, a minipig, etc. As used herein, a molecule is not present at significant levels in a patient if normal serum for that type of patient includes less than 0.1 mM of the molecule. In particular embodiments, normal serum for the patient may include less than 0.08 mM, less than 0.06 mM, or less than 0.04 mM of the molecule.

As used herein, “normal serum” is serum obtained by pooling approximately equal amounts of the liquid portion of coagulated whole blood from five or more non-diabetic patients. A non-diabetic human patient is a randomly selected 18- to 30-year old who presents with no diabetic symptoms at the time blood is drawn.

As used herein, a “polymer” or “polymeric structure” is a structure that includes a string of covalently bound monomers. A polymer can be made from one type of monomer or more than one type of monomer. The term “polymer” therefore encompasses copolymers, including block-copolymers in which different types of monomer are grouped separately within the overall polymer. A polymer can be linear or branched.

As used herein, a “polypeptide” is a polymer made of amino acids that are connected via peptide bonds (or amide bonds). The terms “polypeptide”, “protein”, “oligopeptide”, and “peptide” may be used interchangeably. Polypeptides may contain natural amino acids, non-natural amino acids (i.e., compounds that do not occur in nature but that can be incorporated into a polypeptide chain) and/or amino acid analogs as are known in the art. Also, one or more of the amino acid residues in a polypeptide may be modified, for example, by the addition of a chemical entity such as a carbohydrate group, a phosphate group, a farnesyl group, an isofarnesyl group, a fatty acid group, a linker for conjugation, functionalization, or other modification, etc. These modifications may include cyclization of the peptide, the incorporation of D-amino acids, etc.

As used herein, a “polysaccharide” is a large polymer made of many individual monosaccharides that are connected via glycosidic bonds. The terms “polysaccharide”, “carbohydrate”, and “oligosaccharide” may be used interchangeably. The polymer may include natural monosaccharides (e.g., arabinose, lyxose, ribose, xylose, ribulose, xylulose, allose, altrose, galactose, glucose, gulose, idose, mannose, talose, fructose, psicose, sorbose, tagatose, mannoheptulose, sedoheptulose, octolose, and sialose) and/or modified monosaccharides (e.g., 2′-fluororibose, 2′-deoxyribose, and hexose). Exemplary disaccharides include sucrose, lactose, maltose, trehalose, gentiobiose, isomaltose, kojibiose, laminaribiose, mannobiose, melibiose, nigerose, rutinose, and xylobiose.

As used herein, the term “treat” (or “treating”, “treated”, “treatment”, etc.) refers to the administration of a conjugate of the present disclosure to a subject in need thereof with the purpose to alleviate, relieve, alter, ameliorate, improve or affect a condition (e.g., diabetes), a symptom or symptoms of a condition (e.g., hyperglycemia), or the predisposition toward a condition. For example, as used herein the term “treating diabetes” will refer in general to maintaining glucose blood levels near normal levels and may include increasing or decreasing plasma glucose levels depending on a given situation.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, the term “pharmaceutically acceptable salt” refers to salts of compounds that retain the biological activity of the parent compound, and that are not biologically or otherwise undesirable. Many of the compounds disclosed herein are capable of forming acid and/or base salts by virtue of the presence of amino and/or carboxyl groups or groups similar thereto.

Pharmaceutically acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

As used herein, the terms “effective amount” or “therapeutically effective amount” refer to a nontoxic but sufficient amount of an insulin analog to provide the desired effect. For example, one desired effect would be the prevention or treatment of hyperglycemia. The amount that is “effective” will vary from subject to subject, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

As used herein, the term “parenteral” means not through the alimentary canal but by some other route such as intranasal, inhalation, subcutaneous, intramuscular, intraspinal, or intravenous.

As used herein, the term “insulin” means the active principle of the pancreas that affects the metabolism of carbohydrates in the animal body and that is of value in the treatment of diabetes mellitus. The term includes synthetic and biotechnologically derived products that are the same as, or similar to, naturally occurring insulins in structure, use, and intended effect and are of value in the treatment of diabetes mellitus.

As used herein, the term “insulin or insulin molecule” is a generic term that designates the 51 amino acid heterodimer comprising the A-chain peptide having the amino acid sequence shown in SEQ ID NO: 1 and the B-chain peptide having the amino acid sequence shown in SEQ ID NO: 2, wherein the cysteine residues a positions 6 and 11 of the A chain are linked in a disulfide bond, the cysteine residues at position 7 of the A chain and position 7 of the B chain are linked in a disulfide bond, and the cysteine residues at position 20 of the A chain and 19 of the B chain are linked in a disulfide bond.

As used herein, the terms “insulin analog” or “insulin analogue” as used herein include any heterodimer insulin analog or single-chain insulin analog that comprises one or more modifications of the native A-chain peptide and/or B-chain peptide. Modifications include but are not limited to substituting an amino acid for the native amino acid at a position selected from A1, A4, A5, A8, A9, A10, Al2, A13, A14, A15, A16, A17, A18, A19, A21, B1, B2, B3, B4, B5, B9, B10, B13, B14, B15, B16, B17, B18, B20, B21, B22, B23, B26, B27, B28, B29, B30; inserting or adding an amino acid to position A22, A23, A24, B31, B32, B33, B34, or B35; deleting any or all of the amino acids at positions Bl, B2, B3, B4, B30, or B26-30; or any combination thereof. In general, in the insulin analogs the cysteine residues a positions 6 and 11 of the A chain are linked in a disulfide bond, the cysteine residues at position 7 of the A chain and position 7 of the B chain are linked in a disulfide bond, and the cysteine residues at position 20 of the A chain and 19 of the B chain are linked in a disulfide bond. Examples of insulin analogs include but are not limited to the heterodimer and single-chain analogues disclosed in U.S. Pat. No. 8,722,620 and published International Application WO20100080606, WO2009099763, and WO2010080609, the disclosures of which are incorporated herein by reference. Examples of single-chain insulin analogues also include but are not limited to those disclosed in published International Applications WO9634882, WO95516708, WO2005054291, WO2006097521, WO2007104734, WO2007104736, WO2007104737, WO2007104738, WO2007096332, WO2009132129; U.S. Pat. Nos. 5,304,473 and 6,630,348; and Kristensen et al., BIOCHEM. J. 305: 981-986 (1995), the disclosures of which are each incorporated herein by reference.

As used herein, the term “amino acid modification” refers to a substitution of an amino acid, or the derivation of an amino acid by the addition and/or removal of chemical groups to/from the amino acid and includes substitution with any of the 20 amino acids commonly found in human proteins, as well as atypical or non-naturally occurring amino acids. Commercial sources of atypical amino acids include Sigma-Aldrich (Milwaukee, Wis.), ChemPep Inc. (Miami, Fla.), and Genzyme Pharmaceuticals (Cambridge, Mass.). Atypical amino acids may be purchased from commercial suppliers, synthesized de novo, or chemically modified or derivatized from naturally occurring amino acids.

As used herein, the term “amino acid substitution” refers to the replacement of one amino acid residue by a different amino acid residue.

As used herein, the term “conservative amino acid substitution” is defined herein as exchanges within one of the following five groups:
I. Small aliphatic, nonpolar or slightly polar residues: Ala, Ser, Thr, Pro, Gly;
II. Polar, negatively charged residues and their amides: Asp, Asn, Glu, Gln, cysteic acid and homocysteic acid;
III. Polar, positively charged residues: His, Arg, Lys; Ornithine (Orn)
IV. Large, aliphatic, nonpolar residues: Met, Leu, Ile, Val, Cys, Norleucine (NIe), homocysteine
V. Large, aromatic residues: Phe, Tyr, Trp, acetyl phenylalanine

The disclosure provides methods for controlling the pharmacokinetic (PK) and/or pharmacodynamic (PD) profiles of insulin in a manner that is responsive to the systemic concentrations of a saccharide such as glucose. The methods are based in part on the discovery, disclosed in U.S. Application Publication No. 2011/0301083, that when particular insulin conjugates are modified to include high affinity saccharide ligands such as branched trimannose, they could be made to exhibit PK/PD profiles that responded to saccharide concentration changes even in the absence of an exogenous multivalent saccharide-binding molecule.

In general, the insulin conjugates of the present invention comprise an insulin analog molecule covalently attached to at least one linker covalently attached to a ligand comprising or consisting of a trisaccharide. In particular embodiments, the ligands are capable of competing with a saccharide (e.g., glucose or alpha-methyl mannose) for binding to an endogenous saccharide-binding molecule. In particular embodiments, the ligands are capable of competing with glucose or alpha-methyl mannose for binding to Con A. In particular embodiments, the linker is non-polymeric. In particular embodiments, the conjugate may have a polydispersity index of one and a MW of less than about 20,000 Da. In particular embodiments, the conjugate is of formula (I) as defined and described herein. In particular embodiments, the conjugate is long acting (i.e., exhibits a PK profile that is more sustained than soluble recombinant human insulin (RHI)).

### Insulin Conjugates

This disclosure relates to glucose-responsive insulin conjugates, which comprise trisaccharides, and their synthesis. These insulin conjugates may display a pharmacokinetic (PK) and/or pharmacodynamic (PD) profile that is responsive to the systemic concentrations of a saccharide, such as glucose or alpha-methyl mannose, when administered to a subject in need thereof. In one aspect, the insulin conjugates that comprise an insulin analog molecule covalently attached to at least one linker comprising a trisaccharide sugar cluster, having two or more monomers or subunits linked through the amide bond.

When the insulin conjugate herein is administered to a mammal at least one pharmacokinetic or pharmacodynamic property of the conjugate is sensitive to the serum concentration of a saccharide. In particular embodiments, the PK and/or PD properties of the conjugate are sensitive to the serum concentration of an endogenous saccharide such as glucose. In particular embodiments, the PK and/or PD properties of the conjugate are sensitive to the serum concentration of an exogenous saccharide, e.g., without limitation, mannose, L-fucose, N-acetyl glucosamine and/or alpha-methyl mannose.

### PK and PD Properties

In various embodiments, the pharmacokinetic and/or pharmacodynamic behavior of the insulin conjugate herein may be modified by variations in the serum concentration of a saccharide. For example, from a pharmacokinetic (PK) perspective, the serum concentration curve may shift upward when the serum concentration of the saccharide (e.g., glucose) increases or when the serum concentration of the saccharide crosses a threshold (e.g., is higher than normal glucose levels).

In particular embodiments, the serum concentration curve of an insulin conjugate is substantially different when administered to the mammal under fasted and hyperglycemic conditions. As used herein, the term “substantially different” means that the two curves are statistically different as determined by a student t-test (p<0.05). As used herein, the term “fasted conditions” means that the serum concentration curve was obtained by combining data from five or more fasted non-diabetic individuals. In particular embodiments, a fasted non-diabetic individual is a randomly selected 18- to 30-year old human who presents with no diabetic symptoms at the time blood is drawn and who has not eaten within 12 hours of the time blood is drawn. As used herein, the term “hyperglycemic conditions” means that the serum concentration curve was obtained by combining data from five or more fasted non-diabetic individuals in which hyperglycemic conditions (glucose Cₘₐₓ at least 100 mg/dL above the mean glucose concentration observed under fasted conditions) were induced by concurrent administration of conjugate and glucose. Concurrent administration of conjugate and glucose simply requires that the glucose Cₘₐₓ occur during the period when the conjugate is present at a detectable level in the serum. For example, a glucose injection (or ingestion) could be timed to occur shortly before, at the same time or shortly after the conjugate is administered. In particular embodiments, the conjugate and glucose are administered by different routes or at different locations. For example, in particular embodiments, the conjugate is administered subcutaneously while glucose is administered orally or intravenously.

In particular embodiments, the serum Cₘₐₓ of the conjugate is higher under hyperglycemic conditions as compared to fasted conditions. Additionally or alternatively, in particular embodiments, the serum area under the curve (AUC) of the conjugate is higher under hyperglycemic conditions as compared to fasted conditions. In various embodiments, the serum elimination rate of the conjugate is slower under hyperglycemic conditions as compared to fasted conditions. In particular embodiments, the serum concentration curve of the conjugates can be fit using a two-compartment bi-exponential model with one short and one long half-life. The long half-life appears to be particularly sensitive to glucose concentration. Thus, in particular embodiments, the long half-life is longer under hyperglycemic conditions as compared to fasted conditions. In particular embodiments, the fasted conditions involve a glucose Cₘₐₓ of less than 100 mg/dL (e.g., 80 mg/dL, 70 mg/dL, 60 mg/dL, 50 mg/dL, etc.). In particular embodiments, the hyperglycemic conditions involve a glucose Cₘₐₓ in excess of 200 mg/dL (e.g., 300 mg/dL, 400 mg/dL, 500 mg/dL, 600 mg/dL, etc.). It will be appreciated that other PK parameters such as mean serum residence time (MRT), mean serum absorption time (MAT), etc. could be used instead of or in conjunction with any of the aforementioned parameters.

The normal range of glucose concentrations in humans, dogs, cats, and rats is 60 to 200 mg/dL. One skilled in the art will be able to extrapolate the following values for species with different normal ranges (e.g., the normal range of glucose concentrations in miniature pigs is 40 to 150 mg/d1). Glucose concentrations below 60 mg/dL are considered hypoglycemic. Glucose concentrations above 200 mg/dL are considered hyperglycemic. In particular embodiments, the PK properties of the conjugate may be tested using a glucose clamp method (see Examples) and the serum concentration curve of the conjugate may be substantially different when administered at glucose concentrations of 50 and 200 mg/dL, 50 and 300 mg/dL, 50 and 400 mg/dL, 50 and 500 mg/dL, 50 and 600 mg/dL, 100 and 200 mg/dL, 100 and 300 mg/dL, 100 and 400 mg/dL, 100 and 500 mg/dL, 100 and 600 mg/dL, 200 and 300 mg/dL, 200 and 400 mg/dL, 200 and 500 mg/dL, 200 and 600 mg/dL, etc. Additionally or alternatively, the serum Tₘₐₓ, serum Cₘₐₓ, mean serum residence time (MRT), mean serum absorption time (MAT) and/or serum half-life may be substantially different at the two glucose concentrations. As discussed below, in particular embodiments, 100 mg/dL and 300 mg/dL may be used as comparative glucose concentrations. It is to be understood however that the present disclosure encompasses each of these embodiments with an alternative pair of comparative glucose concentrations including, without limitation, any one of the following pairs: 50 and 200 mg/dL, 50 and 300 mg/dL, 50 and 400 mg/dL, 50 and 500 mg/dL, 50 and 600 mg/dL, 100 and 200 mg/dL, 100 and 400 mg/dL, 100 and 500 mg/dL, 100 and 600 mg/dL, 200 and 300 mg/dL , 200 and 400 mg/dL, 200 and 500 mg/dL, 200 and 600 mg/dL, etc.

Thus, in particular embodiments, the Cₘₐₓ of the conjugate is higher when administered to the mammal at the higher of the two glucose concentrations (e.g., 300 vs. 100 mg/dL glucose). In particular embodiments, the Cₘₐₓ of the conjugate is at least 50% (e.g., at least 100%, at least 200% or at least 400%) higher when administered to the mammal at the higher of the two glucose concentrations (e.g., 300 vs. 100 mg/dL glucose).

In particular embodiments, the AUC of the conjugate is higher when administered to the mammal at the higher of the two glucose concentrations (e.g., 300 vs. 100 mg/dL glucose). In particular embodiments, the AUC of the conjugate is at least 50% (e.g., at least 100%, at least 200% or at least 400%) higher when administered to the mammal at the higher of the two glucose concentrations (e.g., 300 vs. 100 mg/dL glucose).

In particular embodiments, the serum elimination rate of the insulin conjugate is slower when administered to the mammal at the higher of the two glucose concentrations (e.g., 300 vs. 100 mg/dL glucose). In particular embodiments, the serum elimination rate of the conjugate is at least 25% (e.g., at least 50%, at least 100%, at least 200%, or at least 400%) faster when administered to the mammal at the lower of the two glucose concentrations (e.g., 100 vs. 300 mg/dL glucose).

In particular embodiments, the serum concentration curve of insulin conjugates may be fit using a two-compartment bi-exponential model with one short and one long half-life. The long half-life appears to be particularly sensitive to glucose concentration. Thus, in particular embodiments, the long half-life is longer when administered to the mammal at the higher of the two glucose concentrations (e.g., 300 vs. 100 mg/dL glucose). In particular embodiments, the long half-life is at least 50% (e.g., at least 100%, at least 200% or at least 400%) longer when administered to the mammal at the higher of the two glucose concentrations (e.g., 300 vs. 100 mg/dL glucose).

In particular embodiments, the present disclosure provides a method in which the serum concentration curve of an insulin conjugate is obtained at two different glucose concentrations (e.g., 300 vs. 100 mg/dL glucose); the two curves are fit using a two-compartment bi-exponential model with one short and one long half-life; and the long half-lives obtained under the two glucose concentrations are compared. In particular embodiments, this method may be used as an assay for testing or comparing the glucose sensitivity of one or more insulin conjugates.

In particular embodiments, the present disclosure provides a method in which the serum concentration curves of a conjugated drug (e.g., an insulin conjugate of the present disclosure) and an unconjugated version of the drug (e.g., recombinant human insulin or “RHI”) are obtained under the same conditions (e.g., fasted conditions); the two curves are fit using a two-compartment bi-exponential model with one short and one long half-life; and the long half-lives obtained for the conjugated and unconjugated drug are compared. In particular embodiments, this method may be used as an assay for identifying conjugates that are cleared more rapidly than the unconjugated drug.

In particular embodiments, the serum concentration curve of an insulin conjugate is substantially the same as the serum concentration curve of an unconjugated version of the drug when administered to the mammal under hyperglycemic conditions. As used herein, the term “substantially the same” means that there is no statistical difference between the two curves as determined by a student t-test (p>0.05). In particular embodiments, the serum concentration curve of the insulin conjugate is substantially different from the serum concentration curve of an unconjugated version of the drug when administered under fasted conditions. In particular embodiments, the serum concentration curve of the insulin conjugate is substantially the same as the serum concentration curve of an unconjugated version of the drug when administered under hyperglycemic conditions and substantially different when administered under fasted conditions.

In particular embodiments, the hyperglycemic conditions involve a glucose Cₘₐₓ in excess of 200 mg/dL (e.g., 300 mg/dL, 400 mg/dL, 500 mg/dL, 600 mg/dL, etc.). In particular embodiments, the fasted conditions involve a glucose C_{max of less than} 100 mg/dL (e.g., 80 mg/dL, 70 mg/dL, 60 mg/dL, 50 mg/dL, etc.). It will be appreciated that any of the aforementioned PK parameters such as serum Tₘₐₓ, serum Cₘₐₓ, AUC, mean serum residence time (MRT), mean serum absorption time (MAT) and/or serum half-life could be compared.

From a pharmacodynamic (PD) perspective, the bioactivity of the insulin conjugate may increase when the glucose concentration increases or when the glucose concentration crosses a threshold, e.g., is higher than normal glucose levels. In particular embodiments, the bioactivity of an insulin conjugate is lower when administered under fasted conditions as compared to hyperglycemic conditions. In particular embodiments, the fasted conditions involve a glucose Cₘₐₓ of less than 100 mg/dL (e.g., 80 mg/dL, 70 mg/dL, 60 mg/dL, 50 mg/dL, etc.). In particular embodiments, the hyperglycemic conditions involve a glucose Cₘₐₓ in excess of 200 mg/dL (e.g., 300 mg/dL, 400 mg/dL, 500 mg/dL, 600 mg/dL, etc.).

In particular embodiments, the PD properties of the insulin conjugate may be tested by measuring the glucose infusion rate (GIR) required to maintain a steady glucose concentration. According to such embodiments, the bioactivity of the insulin conjugate may be substantially different when administered at glucose concentrations of 50 and 200 mg/dL, 50 and 300 mg/dL, 50 and 400 mg/dL, 50 and 500 mg/dL, 50 and 600 mg/dL, 100 and 200 mg/dL, 100 and 300 mg/dL, 100 and 400 mg/dL, 100 and 500 mg/dL, 100 and 600 mg/dL, 200 and 300 mg/dL, 200 and 400 mg/dL, 200 and 500 mg/dL, 200 and 600 mg/dL, etc. Thus, in particular embodiments, the bioactivity of the insulin conjugate is higher when administered to the mammal at the higher of the two glucose concentrations (e.g., 300 vs. 100 mg/dL glucose). In particular embodiments, the bioactivity of the conjugate is at least 25% (e.g., at least 50% or at least 100%) higher when administered to the mammal at the higher of the two glucose concentrations (e.g., 300 vs. 100 mg/dL glucose).

In particular embodiments, the PD behavior for the insulin analog can be observed by comparing the time to reach minimum plasma glucose concentration (T_{nadir}), the duration over which the blood glucose level (BGL) remains below a particular percentage of the initial value (e.g., 70% of initial value or T70% BGL), etc.

In general, it will be appreciated that any of the PK and PD characteristics discussed in this section can be determined according to any of a variety of published pharmacokinetic and pharmacodynamic methods (e.g., see Baudys et al., Bioconjugate Chem. 9:176-183, 1998 for methods suitable for subcutaneous delivery). It is also to be understood that the PK and/or PD properties may be measured in any mammal (e.g., a human, a rat, a cat, a minipig, a dog, etc.). In particular embodiments, PK and/or PD properties are measured in a human. In particular embodiments, PK and/or PD properties are measured in a rat. In particular embodiments, PK and/or PD properties are measured in a minipig. In particular embodiments, PK and/or PD properties are measured in a dog.

It will also be appreciated that while the foregoing was described in the context of glucose-responsive insulin conjugates, the same properties and assays apply to insulin conjugates that are responsive to other saccharides including exogenous saccharides, e.g., mannose, L-fucose, N-acetyl glucosamine, alpha-methyl mannose, etc. As discussed in more detail below and in the Examples, instead of comparing PK and/or PD properties under fasted and hyperglycemic conditions, the PK and/or PD properties may be compared under fasted conditions with and without administration of the exogenous saccharide. It is to be understood that conjugates can be designed that respond to different Cₘₐₓ values of a given exogenous saccharide.

This disclosure relates to glucose-responsive insulin conjugates, which comprise an insulin or insulin analog molecule covalently attached via a linker to at least one trisaccharide clusters of sugar moieties, and their synthesis. These insulin conjugates may display a pharmacokinetic (PK) and/or pharmacodynamic (PD) profile that is responsive to the systemic concentrations of a saccharide, such as glucose or alpha-methyl mannose, when administered to a subject in need thereof.

In general, the conjugates comprise an insulin or insulin analog molecule covalently attached at its ^{A1}Gly, ^{B1}Phe, and/or ^{B29}Lys amino acid or Lys on another position to one or more trisaccharide clusters of sugar moieties. In specific embodiments, the conjugates comprise an insulin or insulin analog molecule covalently attached at its ^{A1}Gly, ^{B1}Phe, and/or ^{B29}Lys amino acid or Lys on another position to one or two trisaccharide clusters of sugar moieties. Specifically, the one or more trisaccharide clusters of sugar moieties is conjugated onto the side chain amino group of B29 lysine or A1 and B1 amino groups of insulins.

In embodiments of the conjugate, the conjugate comprises an insulin or insulin analog molecule conjugated to at least one or more ligands comprising trisaccharide clusters of sugar moieties.

In embodiments of the conjugate, the conjugate comprises an insulin or insulin analog molecule conjugated to at least two ligands comprising trisaccharide clusters of sugar moieties. In a further embodiment, the conjugate comprises an insulin or insulin analog molecule conjugated to at least three ligands comprising trisaccharide clusters of sugar moieties.

In particular embodiments of the conjugate, the conjugate displays a pharmacodynamic (PD) and/or pharmacokinetic (PK) profile that is sensitive to the serum concentration of a serum saccharide when administered to a subject in need thereof in the absence of an exogenous saccharide binding molecule.

In particular embodiments of the conjugate, the serum saccharide is glucose or alpha-methyl mannose.

In particular embodiments of the conjugate, the conjugate binds an endogenous saccharide binding molecule at a serum glucose concentration of 60 mg/dL or less when administered to a subject in need thereof.

In particular embodiments of the conjugate, the endogenous saccharide binding molecule is human mannose receptor 1.

### Ligand(s)

This disclosure relates to glucose-responsive insulin conjugates that comprise trisaccharide clusters of sugar moieties, and their synthesis. These insulin conjugates may display a pharmacokinetic (PK) and/or pharmacodynamic (PD) profile that is responsive to the systemic concentrations of a saccharide, such as glucose or alpha-methyl mannose, when administered to a subject in need thereof.

In general, the insulin conjugates comprise an insulin analog molecule covalently attached to at least one linker having at least one ligand wherein the ligand comprises or consists of one or more trisaccharides. In particular embodiments, the insulin conjugates may further include one or more linear linkers, each comprising a single ligand, which comprises or consists of one or more trisaccharides. In particular embodiments, the insulin conjugates may further include one or more branched linkers that each includes at least two, three, four, five, or more ligands, where each ligand independently comprises or consists of one or more trisaccharides. When more than one ligand is present the ligands may have the same or different chemical structures.

In particular embodiments, the ligands are capable of competing with a saccharide (e.g., glucose, alpha-methylmannose, or mannose) for binding to an endogenous saccharide-binding molecule (e.g., without limitation surfactant proteins A and D or members of the selectin family). In particular embodiments, the ligands are capable of competing with a saccharide (e.g., glucose, alpha-methylmannose, or mannose) for binding to cell-surface sugar receptor (e.g., without limitation macrophage mannose receptor, glucose transporter ligands, endothelial cell sugar receptors, or hepatocyte sugar receptors). In particular embodiments, the ligands are capable of competing with glucose for binding to an endogenous glucose-binding molecule (e.g., without limitation surfactant proteins A and D or members of the selectin family). In particular embodiments, the ligands are capable of competing with glucose or alpha-methyl mannose for binding to the human macrophage mannose receptor 1 (MRC1). In particular embodiments, the ligands are capable of competing with a saccharide for binding to a non-human lectin (e.g., Con A). In particular embodiments, the ligands are capable of competing with glucose, alpha-methyl mannose, or mannose for binding to a non-human lectin (e.g., Con A). Exemplary glucose-binding lectins include calnexin, calreticulin, N-acetylglucosamine receptor, selectin, asialoglycoprotein receptor, collectin (mannose-binding lectin), mannose receptor, aggrecan, versican, pisum sativum agglutinin (PSA), vicia faba lectin, lens culinaris lectin, soybean lectin, peanut lectin, lathyrus ochrus lectin, sainfoin lectin, sophora japonica lectin, bowringia milbraedii lectin, concanavalin A (Con A), and pokeweed mitogen.

In particular embodiments, the ligand(s) may have a saccharide having the same chemical structure as glucose or may be a chemically related species of glucose, e.g., glucosamine. In various embodiments, it may be advantageous for the ligand(s) to have a different chemical structure from glucose, e.g., in order to fine-tune the glucose response of the conjugate. For example, in particular embodiments, one might use a ligand that includes glucose, mannose, L-fucose or derivatives of these (e.g., α-L-fucopyranoside, mannosamine, β-linked N-acetyl mannosamine, methylglucose, methylmannose, ethylglucose, ethylmannose, propylglucose, propylmannose, etc.) and/or higher order combinations of these (e.g., a bimannose, linear and/or branched trimannose, etc.).

In particular embodiments, the ligand(s) include(s) a trisaccharide. In some embodiments, the ligand(s) comprise a trisaccharide and one or more amine groups. In some embodiments, the ligand(s) comprise a trisaccharide and ethyl group. In particular embodiments, the trisaccharide and amine group are separated by a C₁-C₃ alkyl group. In some embodiments, the ligand is α-aminoethyl glucopyranoside (AEG). In some embodiments, the ligand is α-aminoethyl mannopyranoside (AEM). In some embodiments, the ligand is α-(1-3, 1-6) dimannopyranosyl-α-aminoethylglucopyranoside (α-AEGDM). In some embodiments, the ligand is α-(1-3, 1-6) dimannopyranosyl-β-aminoethylglucopyranoside (β-AEGDM). In some embodiments, the ligand is α-(1-3, 1-6) dimannopyranosyl-α-aminoethyl mannopyranoside (α-AETM (1-3,1-6 linkage)). In some embodiments, the ligand is α-(1-3, 1-6) dimannopyranosyl-β-aminoethyl mannopyranoside ((3-AETM (1-3,1-6 linkage)). In some embodiments, the ligand is α-(1-3, 1-4) dimannopyranosyl-α-aminoethyl mannopyranoside (α-AETM (1-3,1-4 linkage)). In some embodiments, the ligand is α-(1-3, 1-6) dimannopyranosyl α-aminoethyl (2-deoxy-2-fluoro-mannopyranoside (α-AE(2-deoxy-2-F)MDM)). In some embodiments, the ligand is α-(1-3, 1-6) dimannopyranosyl α-aminoethyl (2-deoxy-2-fluoro-glucopyranoside (α-AE(2-deoxy-2-F)GDM). In some embodiments, the ligand is α-(1-3, 1-6) dimannopyranosyl β-aminoethyl (2-deoxy-2-fluoro-glucopyranoside (β-AE(2-deoxy-2-F)GDM). In some embodiments, the ligand is α-(1-2, 1-4) dimannopyranosyl a-aminopropyl mannopyranoside (α-APTM (1-2,1-4 linkage)). In some embodiments, the ligand is α-(1-2, 1-6) dimannopyranosyl β-aminopropyl mannopyranoside (β-APTM (1-3,1-6 linkage)). In some embodiments, the ligand is α-(1-2) mannosyl α-(1-6) fucosyl α-aminopropyl mannopyranoside (α-APM(man 1-3, fucose 1-6)). In some embodiments, the ligand is α-(1-3, 1-6) difucosyl α-aminoethyl mannopyranoside (AEM(fucose 1-3, fucose 1-6)). In some embodiments, the ligand is α-(1-3, 1-6) dimannopyranosyl-α-aminoethyl-C-mannopyranoside (APTM(tetrahydropyran surrogate)). In some embodiments, the ligand is α-(1-3, 1-6) dimannopyranosyl-α-N-methyl aminoethyl mannopyranoside (N-Me AETM). In some embodiments, the ligand is α-(1-3, 1-6) dimannopyranosyl-β-aminoethyl-N-acetylglucosamine (β-AEGADM). In particular embodiments, the saccharide is of the “D” configuration and in other embodiments, the saccharide is of the “L” configuration. Below are the structures of exemplary saccharides having an amine group separated from the saccharide by an ethyl group wherein R may be hydrogen or a carbonyl group of the linker. Other exemplary ligands will be recognized by those skilled in the art.

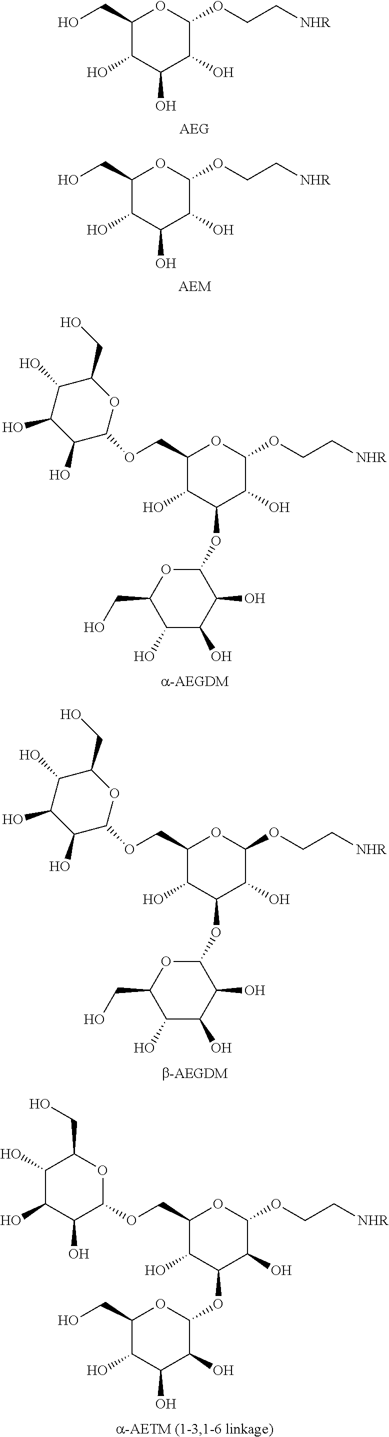

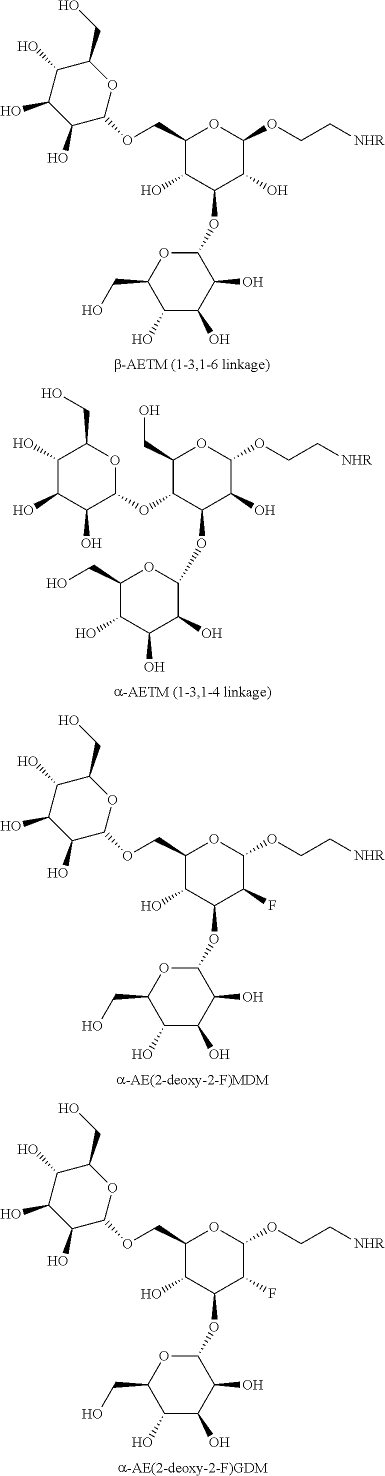

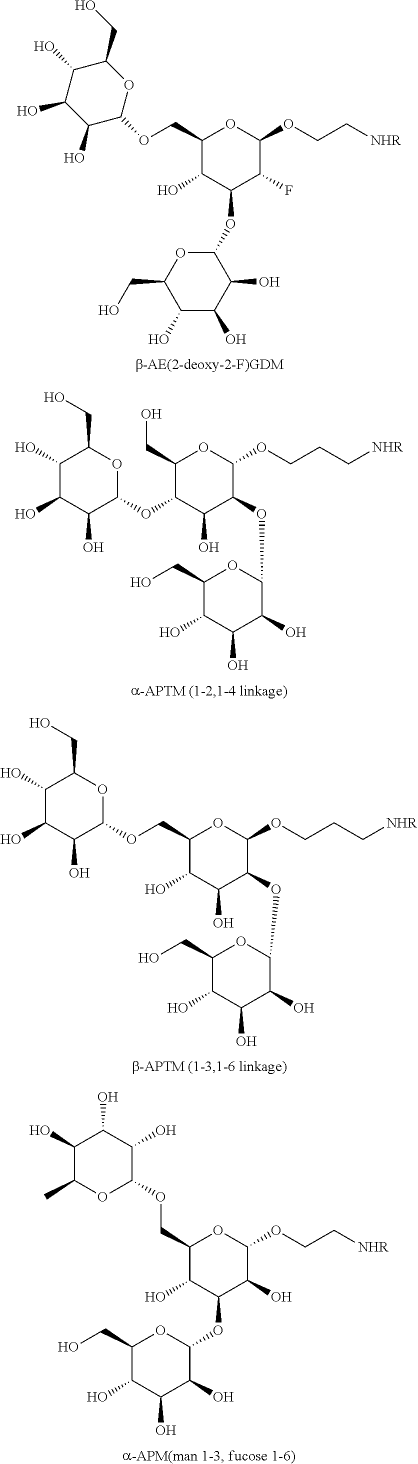

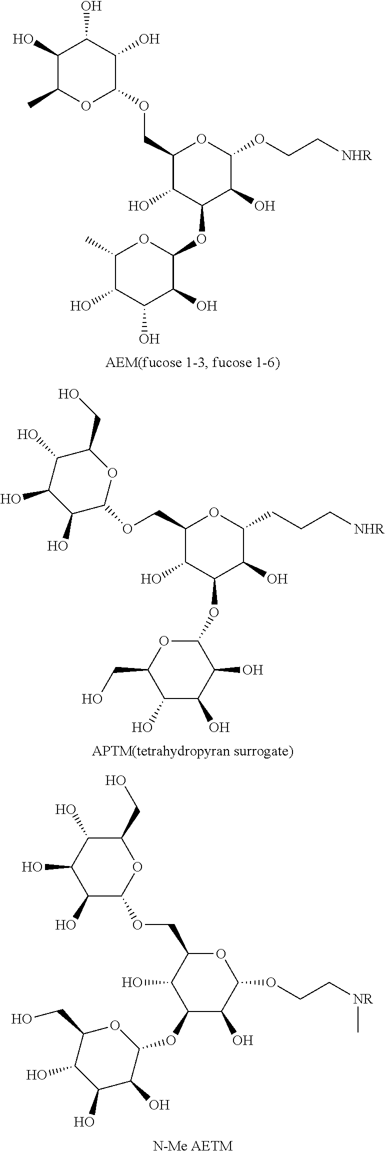

### Insulin

As used herein, the term “insulin conjugate” includes insulin conjugates comprising an insulin analog molecule wherein the insulin analog comprises an amino acid sequence that differs from the native or wild-type human insulin amino acid sequence by at least one amino acid substitution, deletion, rearrangement, or addition. The wild-type sequence of human insulin (A-chain and B-chain) is shown below.

A-Chain polypeptide:
(SEQ ID NO: 1)
GIVEQCCTSICSLYQLENYCN
B-Chain polypeptide:
(SEQ ID NO: 2)
FVNQHLCGSHLVEALYLVCGERGFFYTPKT

In particular aspects of the conjugate, the insulin analog comprises an A chain polypeptide sequence comprising a sequence of X₁I X₂E X₃CCX₄ X₅ X₆CS X₇ X₈ X₉LE X₁₀YC X₁₁X₁₂ (SEQ ID NO: 3); and a B chain polypeptide sequence comprising a sequence of X₁₃VX₁₄X₁₅HLCGSHLVEALX₁₆X₁₇VCGERGFX₁₈YTX₁₉X₂₀X₂₁X₂₂X₂₃X₂₄X₂₅X₂₆ (SEQ ID NO: 4) wherein

X₁ is glycine (G) or lysine (K);

X₂ is valine (V), glycine (G), or lysine (K);

X₃ is glutamine (Q) or lysine (K);

X₄ is threonine (T), histidine (H), or lysine (K);

X₅ is serine (S) or lysine (K);

X₆ is isoleucine (I) or lysine (K);

X₇ is leucine (L) or lysine (K);

X₈ is tyrosine (Y) or lysine (K);

X₉ is glutamine (Q) or lysine (K);

X₁₀ is asparagine (N) or lysine (K);

X₁₁ is asparagine (N), glycine (G), or lysine (K);

X₁₂ is arginine (R), lysine (K), or absent;

X₁₃ is phenylalanine (F) or lysine (K);

X₁₄ is asparagine (N) or lysine (K);

X₁₅ is glutamine (Q) or lysine (K);

X₁₆ is tyrosine (Y) or lysine (K);

X₁₇ is leucine (L) or lysine (K);

X₁₈ is phenylalanine (F) or lysine (K);

X₁₉ is proline (P) or lysine (K):

X₂₀ is lysine (K), proline (P), arginine (R), or is absent;

X₂₁ is threonine (T) or absent;

X₂₂ is arginine (R) if X₂₁ is threonine (T), or absent;

X₂₃ is proline (P) if X₂₂ is arginine (R), or absent;

X₂₄ is arginine (R) if X₂₃ is proline (P), or absent;

X₂₅ is proline (P) if X₂₄ is arginine (R), or absent; and

X₂₆ is arginine (R) if X₂₅ is proline (P), or absent, with the proviso that at least one of X₁, X₃, X₅, X₆, X₇, X₈, X₉, Xio, X₁₂, X₁₃, X₁₄, X₁₅, X₁₆, X₁₇, X₁₈, and X₁₉ is a lysine (K) and when X₁₉ is lysine (K) then X₂₀ is absent or if X₂₀ is present then at least one of X₁, X₃, X₄, X₅, X₆, X₇, X₈, X₉, X₁₀, X₁₁, X₁₂, X₁₃, X₁₄, X₁₅, X₁₆, and X₁₇ is lysine (K), or X₄ is histidine (H), or Xii is glycine (G); or at least one of X₁₂ or X₂₁ is present.

In particular aspects of the conjugate, the insulin analog is GlyA21 human insulin; GlyA3 human insulin; LysA22 human insulin; LysB3 human insulin; HisA8 human insulin; GlyA21 ArgA22 human insulin; DesB30 human insulin; LysA9 DesB30 human insulin; GlyA21 DesB30 human insulin; LysA22 DesB30 human insulin; LysB3 DesB30 human insulin; LysA1 ArgB29 DesB30 human insulin; LysA5 ArgB29 DesB30 human insulin; LysA9 ArgB29 DesB30 human insulin; LysA10 ArgB29 DesB30 human insulin; LysA13 ArgB29 DesB30 human insulin; LysA14 ArgB29 DesB30 human insulin; LysA15 ArgB29 DesB30 human insulin; LysA18 ArgB29 DesB30 human insulin; LysA22 ArgB29 DesB30 human insulin; LysA1 GlyA21 ArgB29 DesB30 human insulin; GlyA21 ArgB29 DesB30 human insulin; LysB1 ArgB29 DesB30 human insulin; LysB3 ArgB29 DesB30 human insulin; LysB4 ArgB29 DesB30 human insulin; LysB16 ArgB29 DesB30 human insulin; LysB17 ArgB29 DesB30 human insulin; LysB25 ArgB29 DesB30 human insulin; GlyA21 ArgB31 ProB32 ArgB33 ProB34 ArgB35 human insulin; or GlyA21 ArgA22 ArgB31 ProB32 ArgB33 human insulin. Herein, glycine is denoted as Gly or G; lysine is denoted as Lys or K; histidine is denoted as His or H; arginine is denoted as Arg or R; and “Des” refers to a deletion of the amino acid at the indicated position.

### Methods for Conjugating Insulin Analog Molecules are Described Below.

In particular embodiments, an insulin analog molecule is conjugated to a linker via the A1 amino acid residue. In particular embodiments, the A1 amino acid residue is glycine. It is to be understood however, that the present disclosure is not limited to N-terminal conjugation and that in particular embodiments an insulin analog molecule may be conjugated via a non-terminal A-chain amino acid residue. In particular, the present disclosure encompasses conjugation via the ε-amine group of a lysine residue present at any position in the A-chain, including at position A1. It will be appreciated that different conjugation positions on the A-chain may lead to different reductions in insulin activity.

In particular embodiments, an insulin analog molecule is conjugated to the linker via the B1 amino acid residue. In particular embodiments, the B1 amino acid residue is phenylalanine. It is to be understood however, that the present disclosure is not limited to N-terminal conjugation and that in particular embodiments an insulin analog molecule may be conjugated via a non-terminal B-chain amino acid residue. In particular, the present disclosure encompasses conjugation via the ε-amine group of a lysine residue present at any position in the B-chain, including position B1. It will be appreciated that different conjugation positions on the B-chain may lead to different reductions in insulin activity.

In particular embodiments, an insulin analog molecule is conjugated to the linker via the B29 amino acid residue. In particular embodiments the B29 amino acid residue is lysine. It is to be understood however, that the present disclosure is not limited to N-terminal conjugation and that in particular embodiments an insulin analog molecule may be conjugated via a non-terminal B-chain amino acid residue. In particular, the present disclosure encompasses conjugation via the ε-amine group of a lysine residue present at any position in the B-chain, including position B29. It will be appreciated that different conjugation positions on the B-chain may lead to different reductions in insulin activity.

In particular embodiments, an insulin analog molecule is conjugated to the linker via acylation of the ε-amine group of lysine. In particular, the present disclosure encompasses conjugation via the ε-amine group of a lysine residue present at any position on the insulin or insulin analog molecule. It will be appreciated that different conjugation positions may lead to different reductions in insulin activity.

In particular embodiments, the ligands are conjugated to more than one conjugation point on the insulin analog molecule. For example, an insulin analog molecule can be conjugated at both the A1 N-terminus and the ε-amino group of a lysine at position A5, A9, A10, A13, A14, A15, A18, A22, B1, B3, B4, B16, B17, B25, B28, or B29. In some embodiments, an insulin molecule can be conjugated at the A1 N-terminus, the B1 N-terminus, and the ε-amino group of lysine. In yet other embodiments, protecting groups are used such that conjugation takes place at the B1 and ε-amino group of lysine or B1 and A1 positions. It will be appreciated that any combination of conjugation points on an insulin molecule may be employed.

Optionally, components may be covalently bound to a linker using “click chemistry” reactions as is known in the art. These include, for example, cycloaddition reactions, nucleophilic ring-opening reactions, and additions to carbon-carbon multiple bonds (e.g., see Kolb and Sharpless, Drug Discovery Today 8: 1128-1137, 2003, and references cited therein as well as Dondoni, Chem. Asian J 2:700-708, 2007 and references cited therein). As discussed above, in various embodiments, the components may be bound to a linker via natural or chemically added pendant groups. In general, it will be appreciated that the first and second members of a pair of reactive groups (e.g., a carboxyl group and an amine group which react to produce an amide bond) can be present on either one of the components and linker (i.e., the relative location of the two members is irrelevant as long as they react to produce a conjugate).

### Insulin Conjugates

In particular embodiments, provided are insulin and insulin analog conjugates wherein the conjugate is characterized as having a ratio of EC50 or inflection point (IP, as defined below) as determined by a functional insulin receptor phosphorylation assay as opposed to the IC50 or IP as determined by a competition binding assay at the macrophage mannose receptor is about 0.5:1 to about 1:100, about 1:1 to about 1:50, about 1:1 to about 1:20, or about 1:1 to about 1:10. In further aspects, the above conjugate is characterized as having a ratio of EC50 or IP as determined by a functional insulin receptor phosphorylation assay as opposed to the IC50 or IP as determined by a competition binding assay at the macrophage mannose receptor is about 0.5:1 to about 1:100, about 1:1 to about 1:50, about 1:1 to about 1:20, or about 1:1 to about 1:10.

The term “IP” refers to the inflection point, which is a point on a curve at which the curvature or concavity changes sign from plus to minus or from minus to plus. In general, IP is usually equivalent to the EC50 or IC50.

In particular aspects, the IC50 or IP as determined by a competition binding assay at the macrophage mannose receptor may be less than about 100 nM and greater than about 0.5 nM. In particular aspects, the IC50 or IP is less than about 50 nM and greater than about 1 nM, less than about 25 nM and greater than about 1 nM, or less than about 20 nM and greater than about 1 nM. In particular aspects, the IC50 or IP as determined by a functional insulin receptor phosphorylation assay may be less than about 100 nM and greater than about 0.5 nM. In particular aspects, the IC50 or IP is less than about 50 nM and greater than about 1 nM, less than about 25 nM and greater than about 1 nM, or less than about 20 nM and greater than about 1 nM.

The instant disclosure relates to glucose-responsive insulin conjugates having general formula (I):

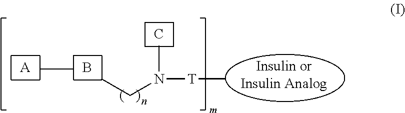

wherein

(a) the insulin or insulin analog is selected from human insulin, porcine insulin, insulin lispro, insulin aspart, insulin glulisine, insulin glargine, insulin detemir, GlyA21 human insulin, GlyA3 human insulin, LysA22 human insulin, LysB3 human insulin, HisA8 human insulin, GlyA21 ArgA22 human insulin, DesB30 human insulin, LysA9 DesB30 human insulin, GlyA21 DesB30 human insulin, LysA22 DesB30 human insulin, LysB3 DesB30 human insulin, LysA1 ArgB29 DesB30 human insulin, LysA5 ArgB29 DesB30 human insulin, LysA9 ArgB29 DesB30 human insulin, LysA10 ArgB29 DesB30 human insulin, LysA13 ArgB29 DesB30 human insulin, LysA14 ArgB29 DesB30 human insulin, LysA15 ArgB29 DesB30 human insulin, LysA18 ArgB29 DesB30 human insulin, LysA22 ArgB29 DesB30 human insulin, LysA1 GlyA21 ArgB29 DesB30 human insulin, GlyA21 ArgB29 DesB30 human insulin, LysB1 ArgB29 DesB30 human insulin, LysB3 ArgB29 DesB30 human insulin, LysB4 ArgB29 DesB30 human insulin, LysB16 ArgB29 DesB30 human insulin, LysB17 ArgB29 DesB30 human insulin, LysB25 ArgB29 DesB30 human insulin, GlyA21 ArgB31 ProB32 ArgB33 ProB34 ArgB35 human insulin, GlyA21 ArgA22 ArgB31 ProB32 ArgB33 human insulin, and insulin analogs that comprise

(i) an A chain polypeptide sequence comprising a sequence of X₁I X₂E X₃CCX₄ X₅X₆CS X₇ X₈ X₉LE X₁₀YC X₁₁X₁₂ (SEQ ID NO: 3) and

(ii) a B chain polypeptide sequence comprising a sequence of X₁₃VX₁₄X₁₅HLCGSHLVEALX₁₆X₁₇VCGERGFX₁₈YTX₁₉X₂₀X₂₁X₂₂X₂₃X₂₄X₂₅X₂₆ (SEQ ID NO: 4) wherein:

X₁ is glycine (G) or lysine (K), X₂ is valine (V), glycine (G), or lysine (K),

X₃ is glutamine (Q) or lysine (K),

X₄ is threonine (T) or histidine (H),

X₅ is serine (S) or lysine (K),

X₆ is isoleucine (I) or lysine (K),

X₇ is leucine (L) or lysine (K),

X₈ is tyrosine (Y) or lysine (K),

X₉ is glutamine (Q) or lysine (K),

X₁₀ is asparagine (N) or lysine (K),

X₁₁ is asparagine (N) or glycine (G),

X₁₂ is arginine (R), lysine (K), or absent,

X₁₃ is phenylalanine (F) or lysine (K),

X₁₄ is asparagine (N) or lysine (K),

X₁₅ is glutamine (Q) or lysine (K),

X₁₆ is tyrosine (Y) or lysine (K),

X₁₇ is leucine (L) or lysine (K),

X₁₈ is phenylalanine (F) or lysine (K),

X₁₉ is proline (P) or lysine (K),

X₂₀ is lysine (K), proline (P), or arginine (R),

X₂₁ is threonine (T) or absent,

X₂₂ is arginine (R) if X₂₁ is threonine (T), or absent,

X₂₃ is proline (P) if X₂₂ is arginine (R), or absent,

X₂₄ is arginine (R) if X₂₃ is proline (P), or absent,

X₂₅ is proline (P) if X₂₄ is arginine (R), or absent, and

X₂₆ is arginine (R) if X₂₅ is proline (P), or absent,

with the proviso that at least one of X₁, X₃, X₅, X₆, X₇, X₈, X₉, X₁₀, X₁₂, X₁₃, X₁₄, X₁₅, X₁₆, X₁₇, X₁₈, and X₁₉ is a lysine (K) and when X₁₉ is lysine (K) then X₂₀ is absent or if X₂₀ is present then at least one of X₁, X₃, X₄, X₅, X₆, X₇, X₈, X₉, X₁₀, X₁₁, X₁₂, X₁₃, X₁₄, X₁₅, X₁₆, and X₁₇ is lysine (K), or X₄ is histidine (H), or X₁₁ is glycine (G), or at least one of X₁₂ or X₂₁ is present;

(b) the linker T is covalently linked to the amino group at position A1 of the insulin or insulin analog molecule; position B1 of the insulin or insulin analog molecule; position B29 of the insulin or insulin analog molecule; or other lysine residue of the insulin or insulin analog molecule;

(c) each occurrence of [symbol] is an independently selected trisaccharide;

(d) each [symbol] is selected independently from carbon and oxygen;

(e) each [symbol] is selected independently from H and CH₃;

(f) m is the number of individual, independently selected monomeric units

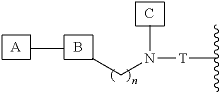

that are conjugated to the insulin or insulin analog, and is selected from 1, 2, or 3;

(g) n is the number of methylene units, and is selected from 1, 2, or 3.

In embodiments of the conjugate, the saccharides are of the “D” configuration, and in other embodiments, the saccharides are of the “L” configuration. In still further embodiments, the saccharides are independently of either the “D” configuration or the “L” configuration.

### Description of Exemplary Groups

[symbol] (Trisaccharide)

In embodiments, each occurrence of [symbol] is an independently selected trisaccharide. In particular embodiments, each [symbol] comprises a saccharide, independently selected from the group consisting of glucopyranoside, mannopyranoside, 2-deoxy-2-fluoro-glucopyranoside, and 2-deoxy-2-fluoro-mannopyranoside, which is bonded to two additional saccharides, each independently selected from mannose and fucose.

[symbol]

In embodiments, each occurrence of [symbol] is independently selected from O and CR¹₂, wherein each R¹ is selected independently from H and halogen. In particular embodiments, one or more occurrence of [symbol] is CR¹₂. In still more particular embodiments, one or more occurrence of [symbol] is CH₂.

[symbol]

In embodiments, each occurrence of [symbol] is independently selected from H and CR²₃, wherein each R² is selected independently from H and halogen. In particular embodiments, one or more occurrence of [symbol] is CR²₃. In still more particular embodiments, one or more occurrence of [symbol] is CH₃.

### T (Linker)

In particular embodiments, each occurrence of T is independently a bivalent, straight or branched, saturated or unsaturated, optionally substituted C₁₋₂₀ hydrocarbon chain wherein one or more methylene units of T are optionally and independently replaced by —O—, —S—, —N(R)—, —C(O)—, —C(O)O—, —OC(O)—, —N(R)C(O)—, —C(O)N(R)—, —S(O)—, —S(O)₂—, —N(R)SO₂—, SO₂N(R)—, a heterocyclic group, an aryl group, or a heteroaryl group, wherein R is H or C₁₋₄ alkyl. In particular embodiments, one, two, three, four, or five methylene units of T are optionally and independently replaced. In particular embodiments, T is constructed from a C₁₋₁₀, C₁₋₈, C₁₋₆, C₁₋₄, C₂₋₁₂, C₄₋₁₂, C₆₋₁₂, C₈₋₁₂, or C₁₀₋₁₂ hydrocarbon chain wherein one or more methylene units of T are optionally and independently replaced by —O—, —S—, —N(R)—, —C(O)—, C(O)O—, OC(O)—, —N(R)C(O)—, —C(O)N(R)—, —S(O)—, —S(O)₂—, —N(R)SO₂—, SO₂N(R)—, a heterocyclic group, an aryl group, or a heteroaryl group. In some embodiments, one or more methylene units of T is replaced by a heterocyclic group. In some embodiments, one or more methylene units of T is replaced by a triazole moiety. In particular embodiments, one or more methylene units of T is replaced by —C(O)—. In particular embodiments, one or more methylene units of T is replaced by —C(O)N(R)—. In particular embodiments, one or more methylene units of T is replaced by —O—.

In particular embodiments, each individual T may be selected from structure

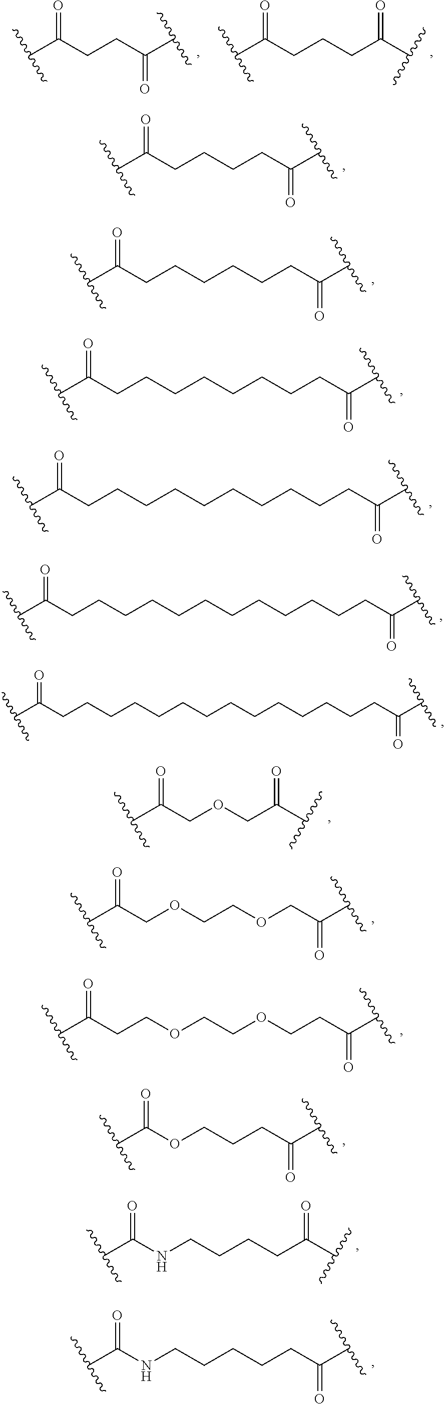

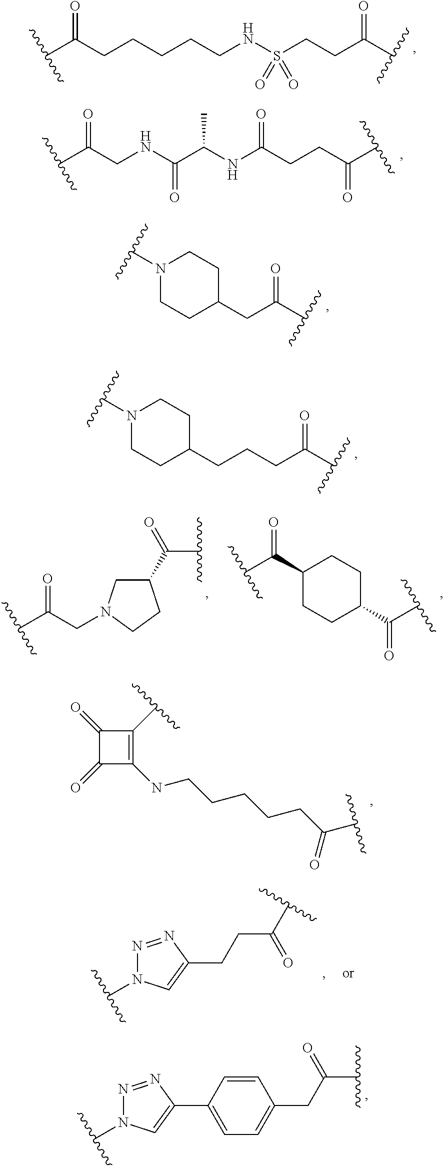

wherein the wavy line indicates the bond is linked to an atom comprising the linker.

Particular components may naturally possess more than one of the same chemically reactive moieties. In some examples, it is possible to choose the chemical reaction type and conditions to selectively react with the component at only one of those sites. For example, in the case where insulin is conjugated through reactive amines, in particular embodiments, the N-terminal α-Phe-B1 may be more desirable as a site of attachment over the N-terminal α-Gly-A1 and ε-Lys-B29 to preserve insulin bioactivity (e.g., see Mei et al., Pharm. Res. 16: 1680-1686, 1999 and references cited therein as well as Tsai et al., J. Pharm. Sci. 86: 1264-1268, 1997). In an exemplary reaction between insulin with hexadecenal (an aldehyde-terminated molecule), researchers found that mixing the two components overnight in a 1.5M pH 6.8 sodium salicylate aqueous solution containing 54% isopropanol at a ratio of 1:6 (insulin:aldehyde mol/mol) in the presence of sodium cyanoborohydride resulted in over 80% conversion to the single-substituted Phe-Bl secondary amine-conjugated product (Mei et al., Pharm. Res. 16:1680-1686, 1999). Their studies showed that the choice of solvent, pH, and insulin:aldehyde ratio all affected the selectivity and yield of the reaction. In most cases, however, achieving selectivity through choice of chemical reaction conditions is difficult. Therefore, in particular embodiments, it may be advantageous to selectively protect the component (e.g., insulin) at all sites other than the desired site for reaction, followed by a deprotection step after the material has been reacted and purified. For example, there are numerous examples of selective protection of insulin amine groups available in the literature including those that may be deprotected under slightly acidic (citraconic anhydride), and basic (methyl sulfonyl chloride or “MSC”; fluorenylmethyl oxycarbonyl chloride or “Fmoc”) conditions (e.g., see Tsai et al., J. Pharm. Sci. 86: 1264-1268, 1997; Dixon et al., Biochem. J. 109: 312-314, 1968; and Schuettler et al., D. Brandenburg Hoppe Seyler's Z. Physiol. Chem. 360: 1721, 1979). In one example, the Gly-A1 and Lys-B29 amines may be selectively protected with tert-butoxycarbonyl (BOC) groups that are then removed after conjugation by incubation for one hour at 4° C. in a 90% trifluoroacetic acid (TFA)/10% anisole solution. In one embodiment, a dry powder of insulin is dissolved in anhydrous dimethylsulfoxide (DMSO) followed by an excess of triethylamine (TEA). To this solution, approximately two equivalents of di-tert-butyl dicarbonate solution in THF are added slowly and the solution allowed to mix for 30 to 60 minutes. After reaction, the crude solution is poured in an excess of acetone followed by dropwise addition of dilute HCl to precipitate the reacted insulin. The precipitated material is centrifuged, washed with acetone and dried completely under vacuum.

The desired di-BOC protected product may be separated from unreacted insulin analog, undesired di-BOC isomers, and mono-BOC and tri-BOC byproducts using preparative reverse phase HPLC or ion exchange chromatography (e.g., see Tsai et al., J. Pharm. Sci. 86: 1264-1268, 1997). In the case of reverse phase HPLC, a solution of the crude product in 70% water/30% acetonitrile containing 0.1% TFA is loaded onto a C8 column and eluted with an increasing acetonitrile gradient. The desired di-BOC peak is collected, the acetonitrile removed and lyophilized to obtain the product.

In particular aspects of the conjugate, the insulin analog is conjugated to at least one linker selected from ML-1, ML-2, ML-3, ML-4, ML-5, ML-6, ML-7, ML-8, ML-9, ML-10, ML-11, ML-12, ML-13, ML-14, ML-15, ML-16, ML-17, ML-18, ML-19, ML-20, ML-21, ML-22, ML-23, ML-24, ML-25, ML-26, ML-27, ML-28, ML-29, ML-30, ML-31, ML-32, ML-33, ML-34, ML-35, ML-36, ML-37, ML-38, ML-39, ML-40, ML-41, ML-42, ML-43, ML-44, ML-45, ML-46, ML-47, ML-48, ML-49, ML-50, ML-51, ML-52, ML-53, ML-54, ML-55, and ML-56. Each conjugation may independently be an amide linkage between the linker and the N-terminal amino group of the A chain polypeptide or B chain polypeptide or the epsilon amino group of a lysine residue within the A chain polypeptide or B chain polypeptide.

Embodiments of this disclosure provide conjugates having the formula as set forth in Table 1 for IOC-1, 10C-2, IOC-3, IOC-4, IOC-5, IOC-6, IOC-7, IOC-8, IOC-9, IOC-10, IOC-11, IOC-12, IOC-13, IOC-14, IOC-15, IOC-16, IOC-17, IOC-18, IOC-19, IOC-20, IOC-21, IOC-22, IOC-23, IOC-24, IOC-25, IOC-26, IOC-27, IOC-28, IOC-29, IOC-30, IOC-31, IOC-32, IOC-33, IOC-34, IOC-35, IOC-36, IOC-37, IOC-38, IOC-39, IOC-41, IOC-42, IOC-43, IOC-44, IOC-45, IOC-46, IOC-47, IOC-48, IOC-49, IOC-50, IOC-51, IOC-52, IOC-53, IOC-54, IOC-55, IOC-56, IOC-57, IOC-58, IOC-59, IOC-60, IOC-61, IOC-62, IOC-63, IOC-64, IOC-65, IOC-66, IOC-67, IOC-68, IOC-69, IOC-70, IOC-71, IOC-72, IOC-73, IOC-74, IOC-75, IOC-76, IOC-77, IOC-79, IOC-80, IOC-81, IOC-82, IOC-83, IOC-84, IOC-85, IOC-86, IOC-87, IOC-88, IOC-89, IOC-90, IOC-91, IOC-92, IOC-93, IOC-94, IOC-95, IOC-96, IOC-97, IOC-98, IOC-99, IOC-100, IOC-101, IOC-102, IOC-103, IOC-104, IOC-105, IOC-106, IOC-107, IOC-108, IOC-109, IOC-110, IOC-111, IOC-112, IOC-113, IOC-114, IOC-115, IOC-116, IOC-117, IOC-118, IOC-119, IOC-120, IOC-121, IOC-122, IOC-123, IOC-124, IOC-125, IOC-126, IOC-127, IOC-128, IOC-129, IOC-130, IOC-131, IOC-132, IOC-133, IOC-134, IOC-135, IOC-136, IOC-137, IOC-138, IOC-139, IOC-140, IOC-141, IOC-142, IOC-143, IOC-144, IOC-145, IOC-146, IOC-147, and IOC-148.

| Conjugate | Compound Formula & Structure |
|---|---|
| IOC-1 | 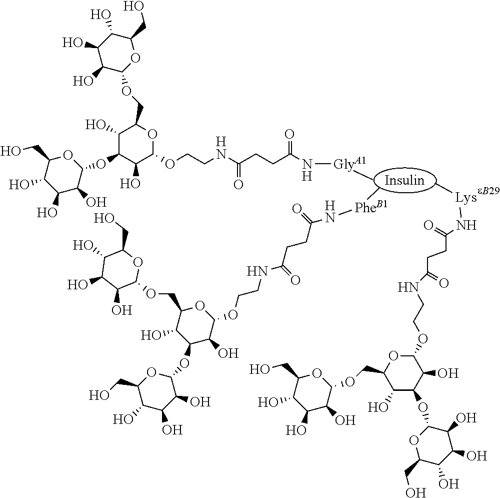 |
| IOC-2 | 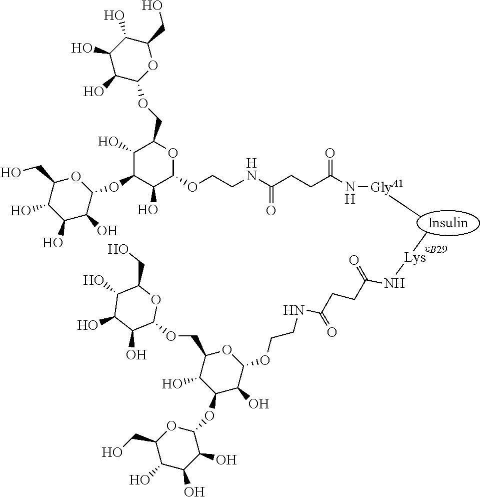 |
| IOC-3 | 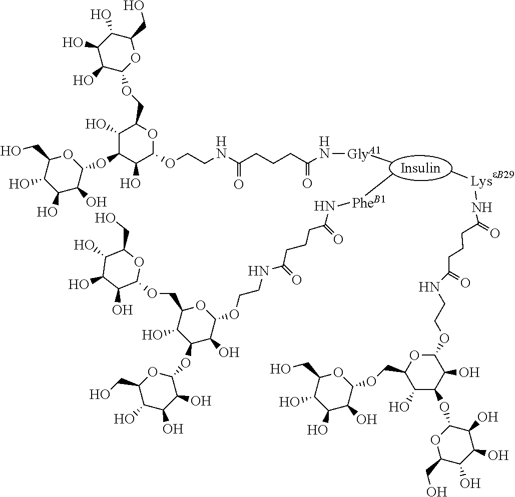 |
| IOC-4 | 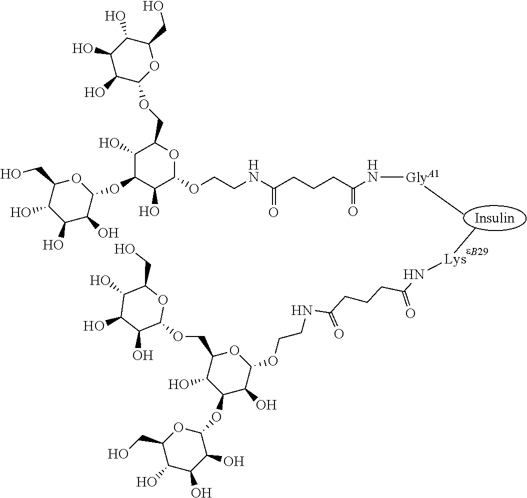 |
| IOC-5 | 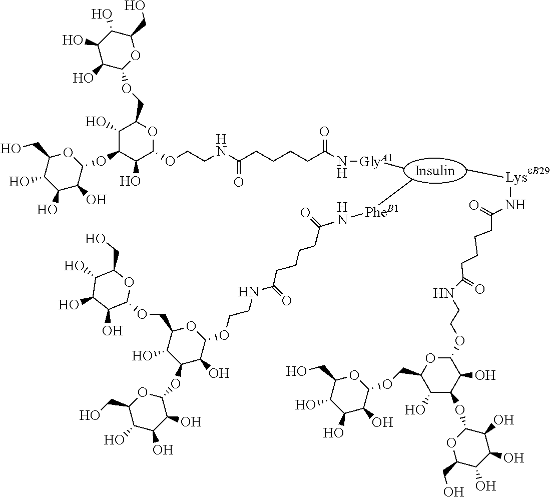 |
| IOC-6 | 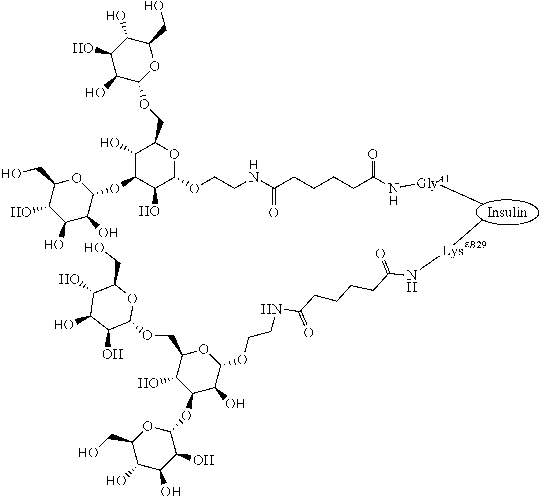 |
| IOC-7 | 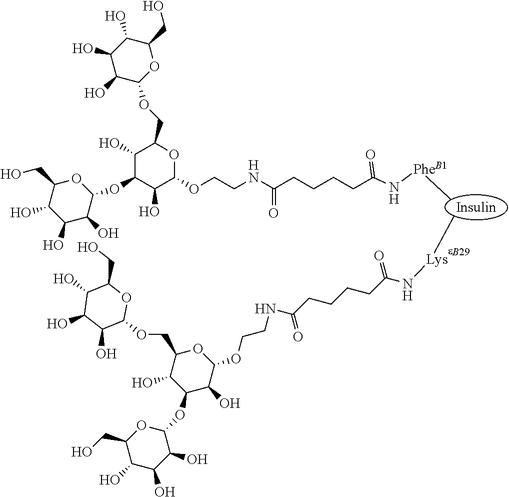 |
| IOC-8 | 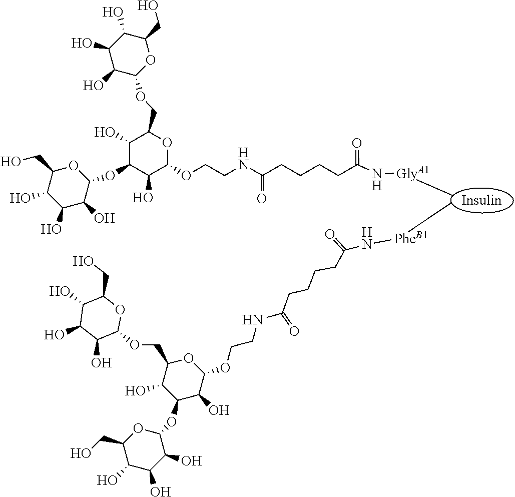 |
| IOC-9 | 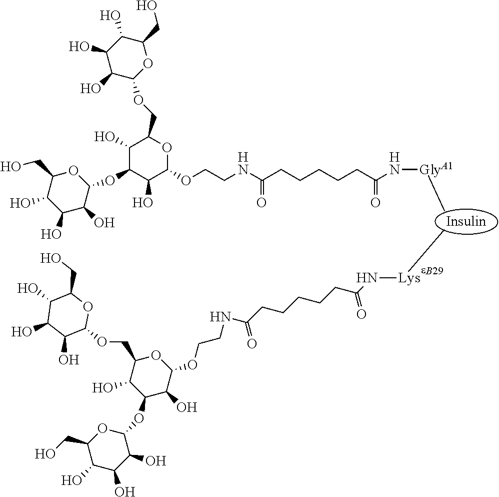 |
| IOC-10 | 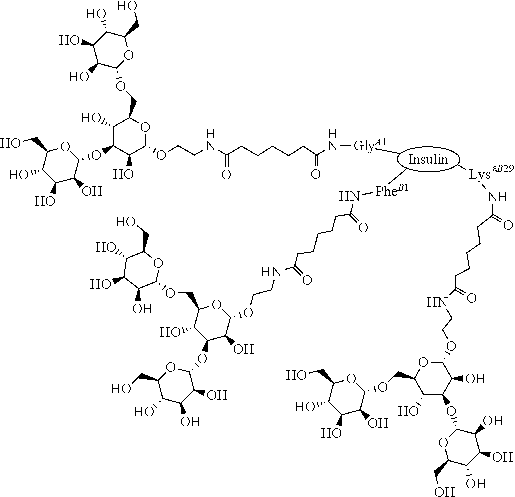 |
| IOC-11 | 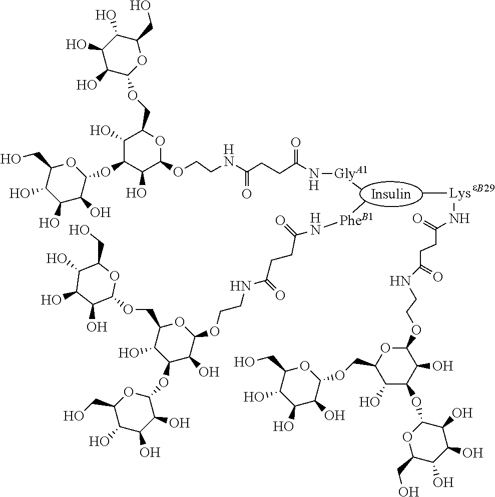 |
| IOC-12 | 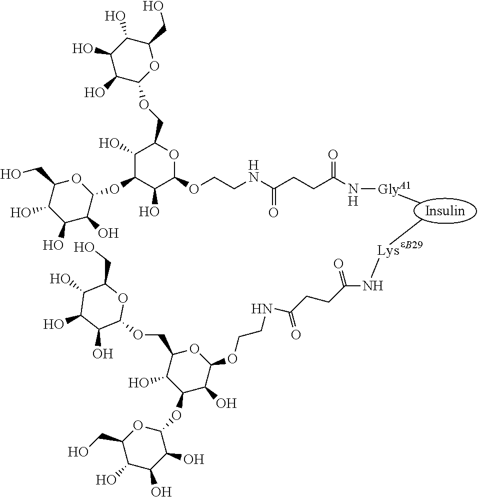 |
| IOC-13 | 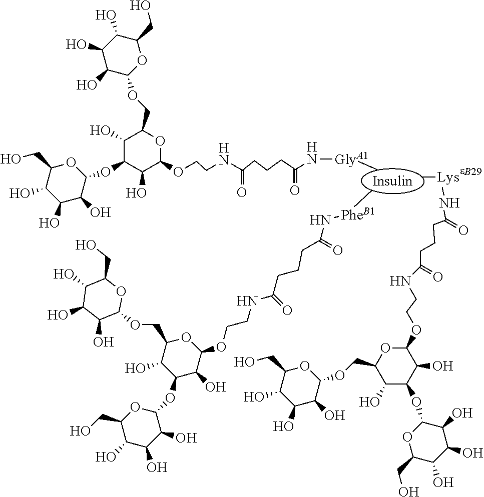 |
| IOC-14 | 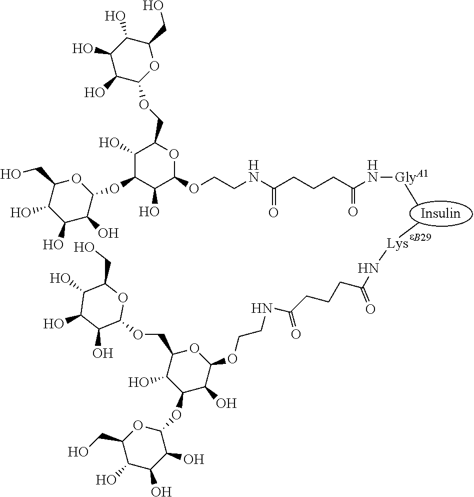 |
| IOC-15 | 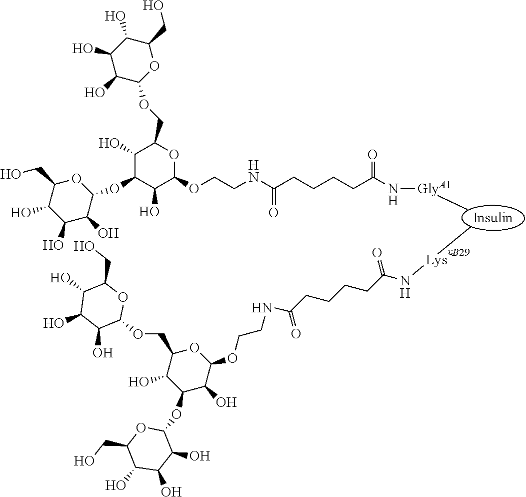 |
| IOC-16 | 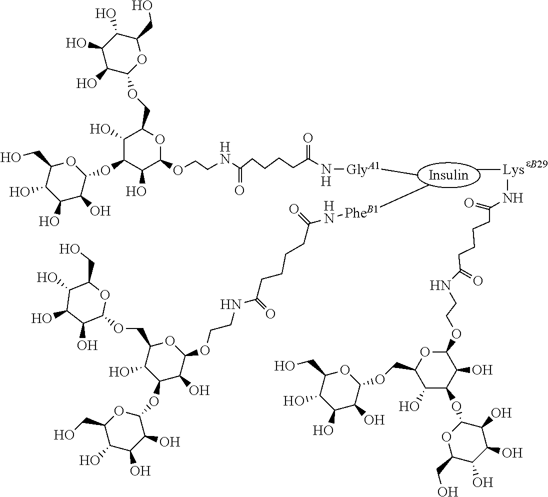 |
| IOC-17 | 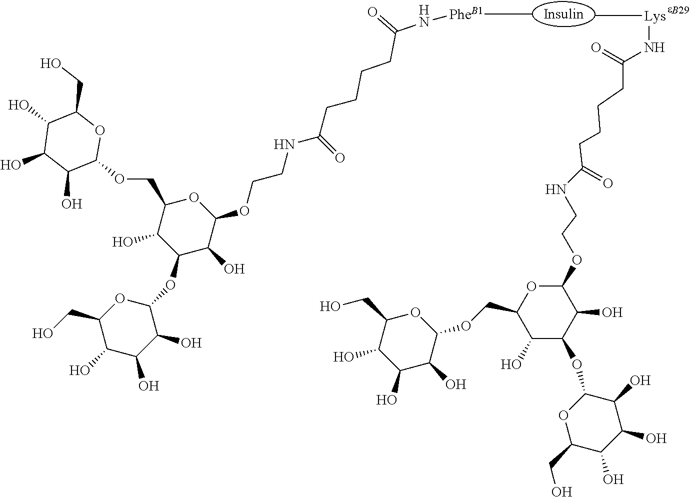 |
| IOC-18 | 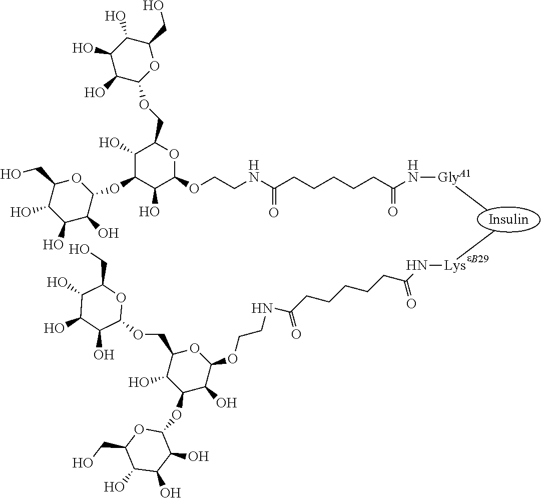 |
| IOC-19 | 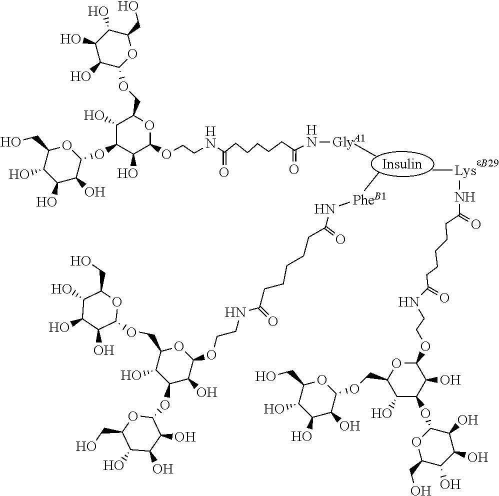 |
| IOC-20 | 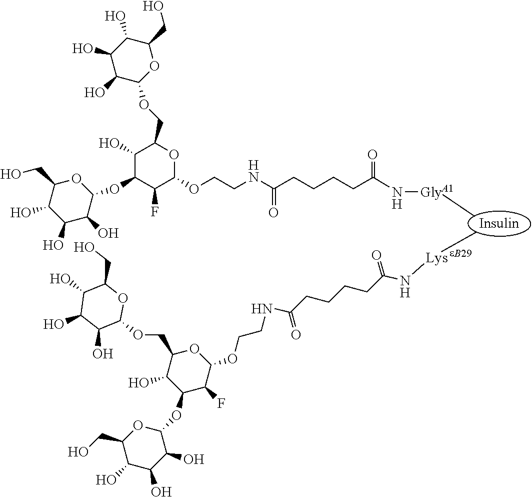 |
| IOC-21 | 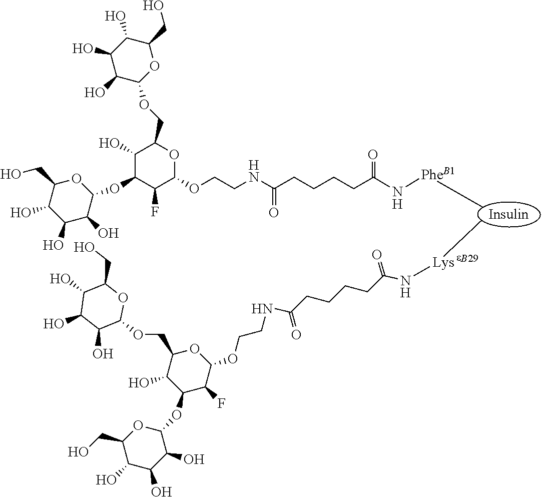 |
| IOC-22 | 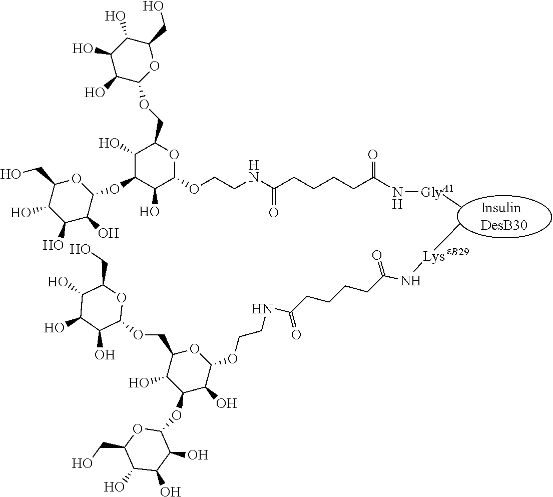 |
| IOC-23 | 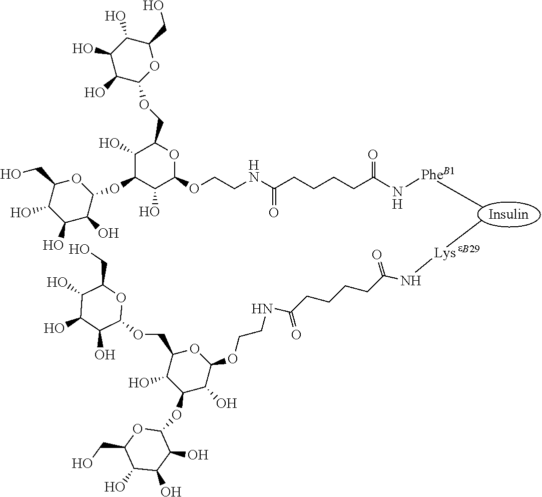 |
| IOC-24 | 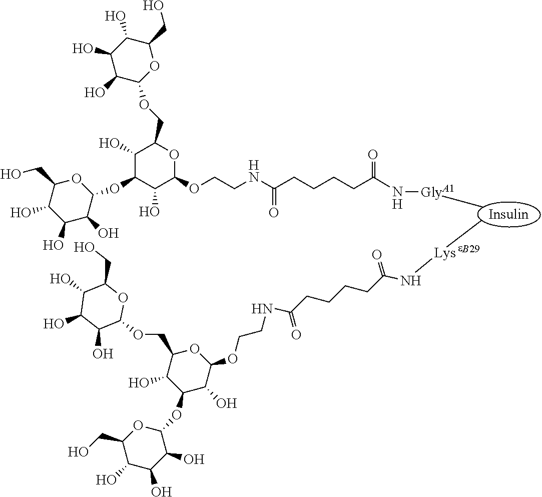 |
| IOC-25 | 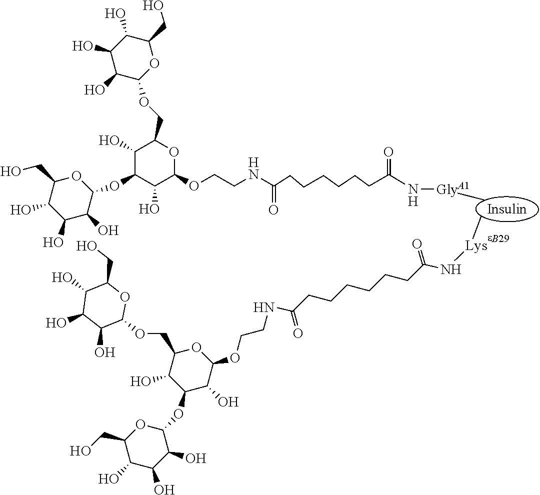 |
| IOC-26 | 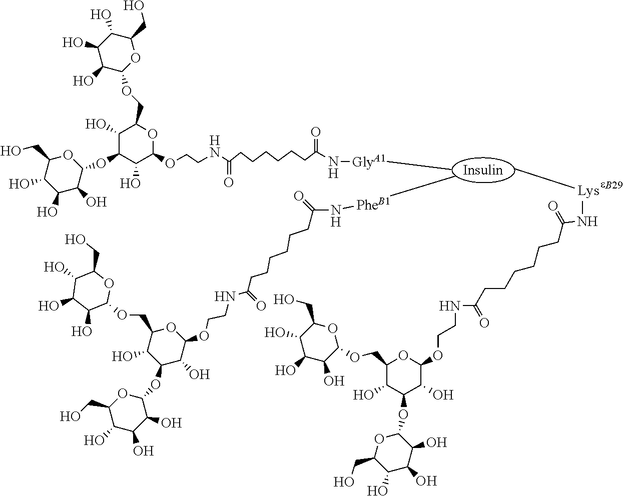 |
| IOC-27 | 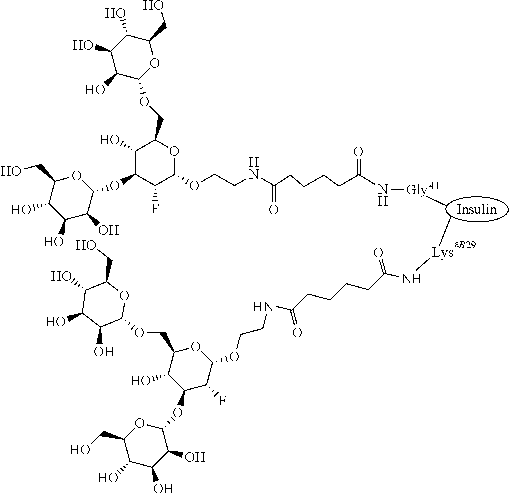 |
| IOC-28 | 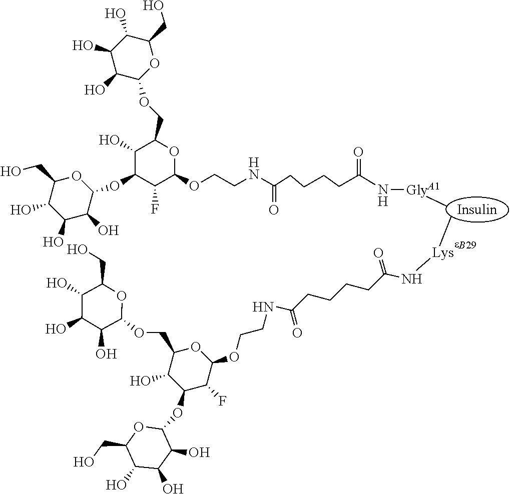 |
| IOC-29 | 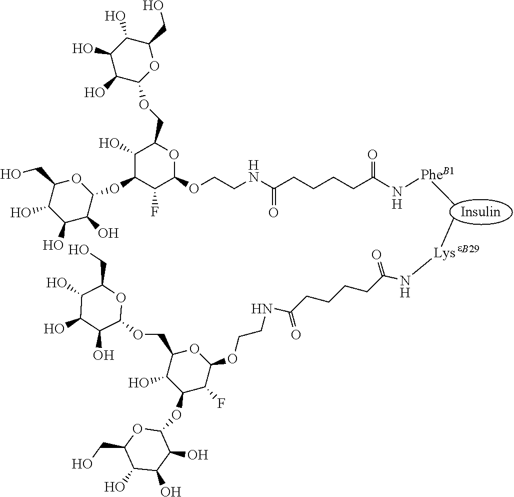 |
| IOC-30 | 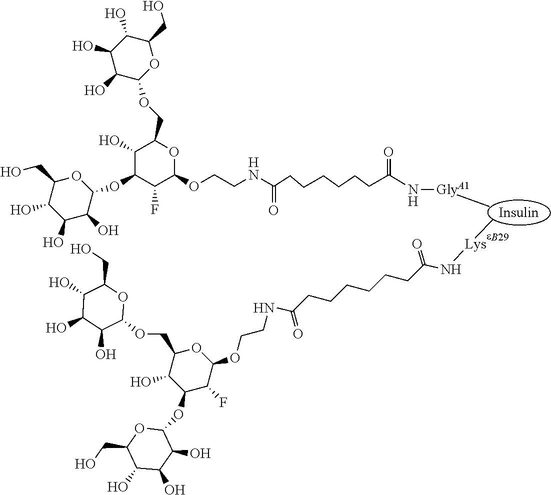 |
| IOC-31 | 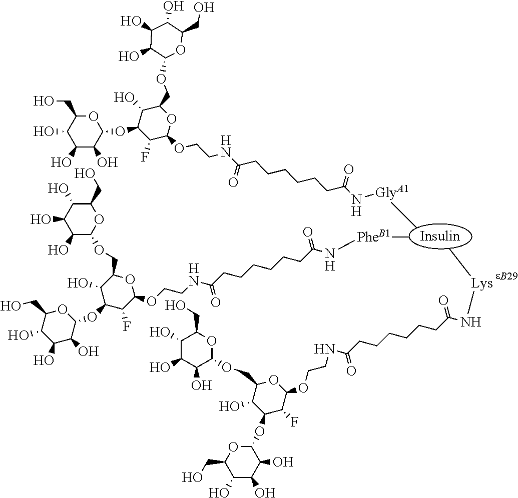 |
| IOC-32 | 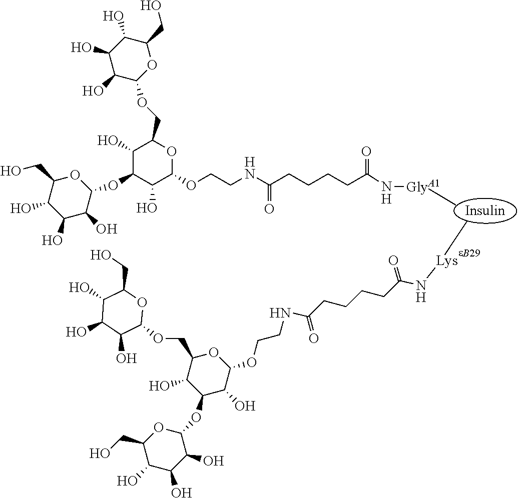 |
| IOC-33 | 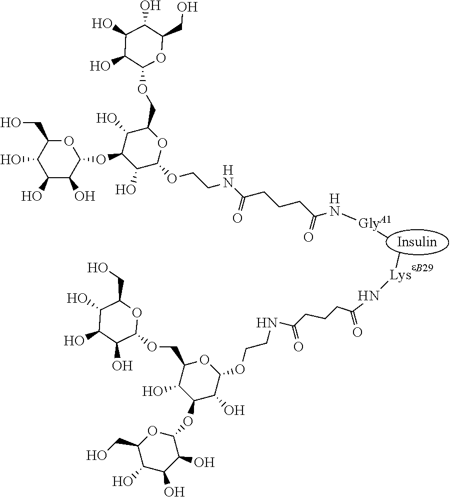 |
| IOC-34 | 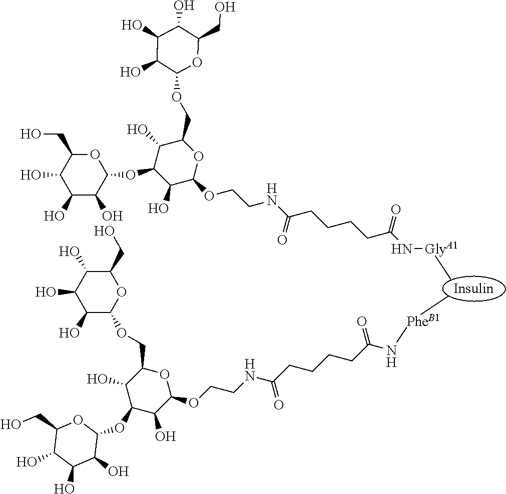 |
| IOC-35 | 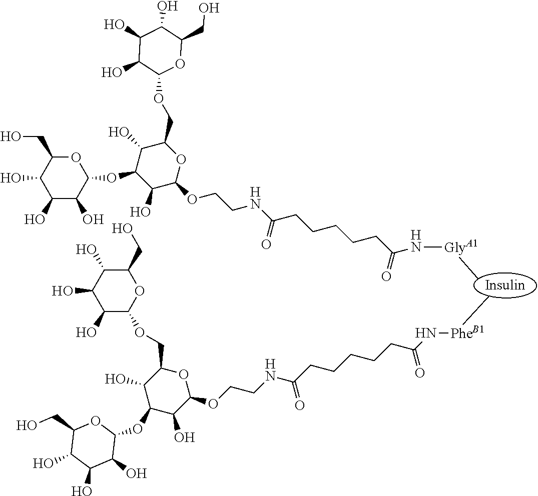 |
| IOC-36 | 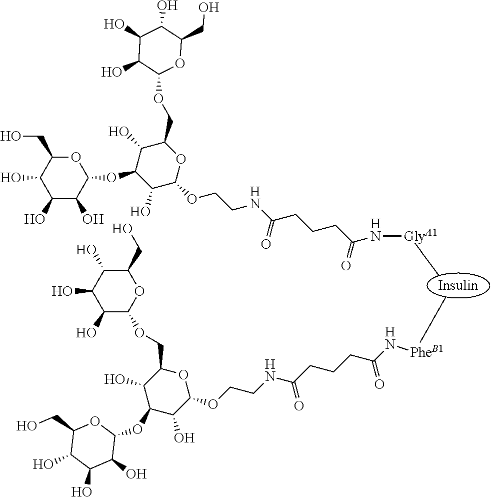 |
| IOC-37 | 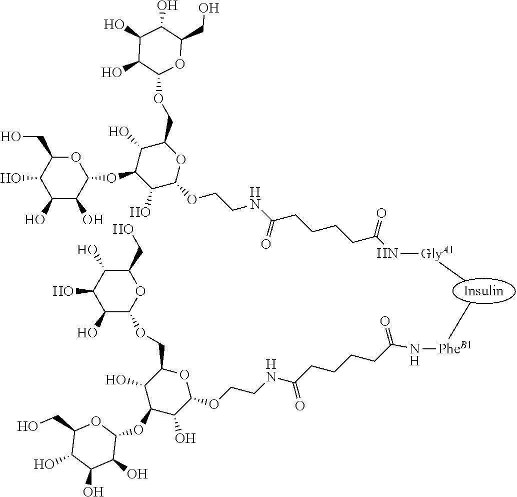 |
| IOC-38 | 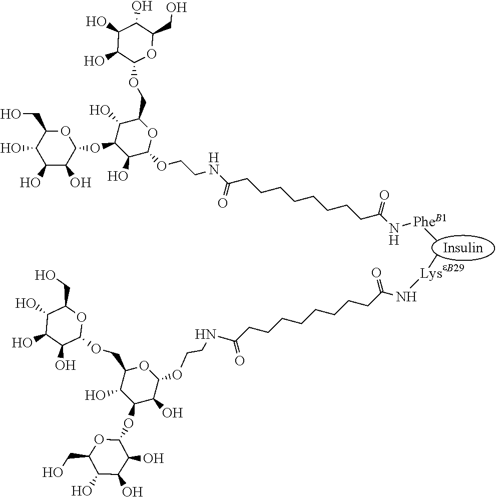 |
| IOC-39 | 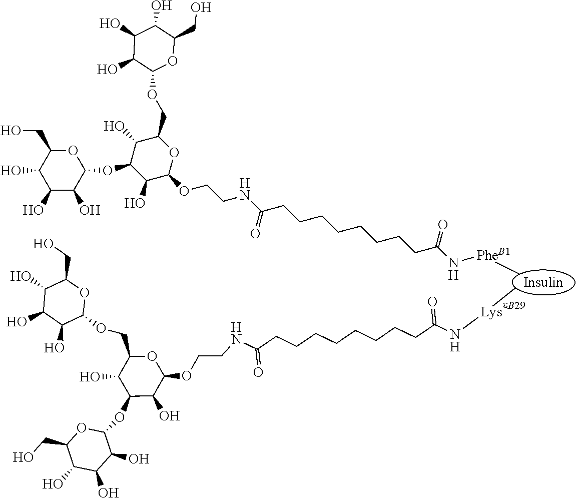 |
| IOC-41 | 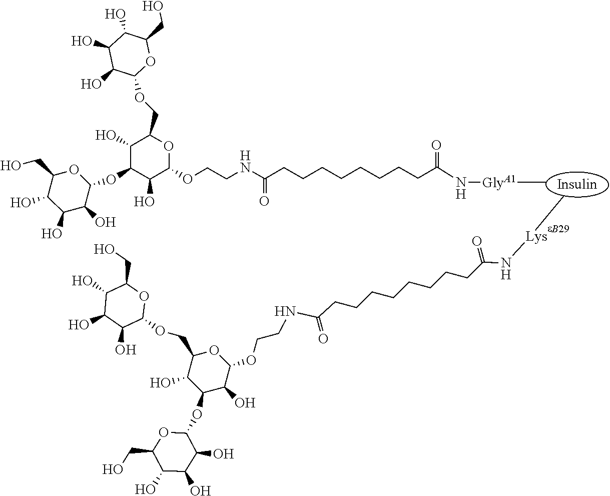 |
| IOC-42 | 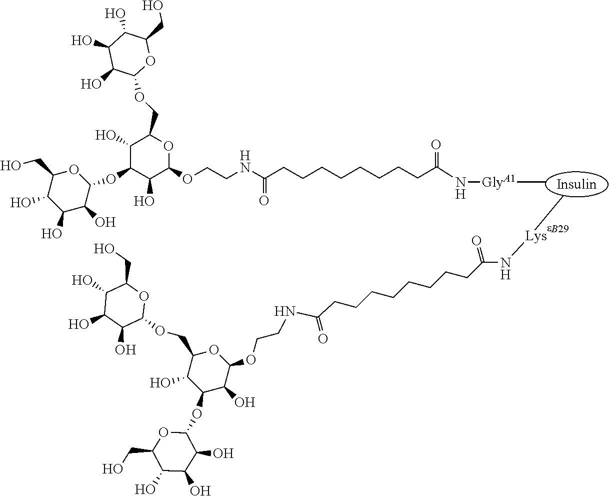 |
| IOC-43 | 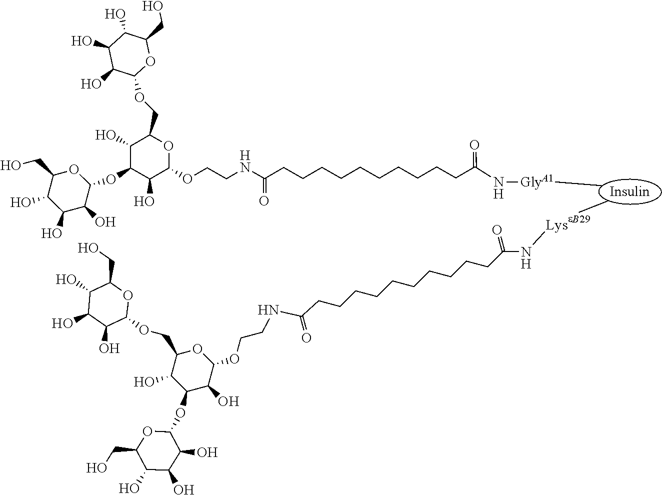 |
| IOC-44 | 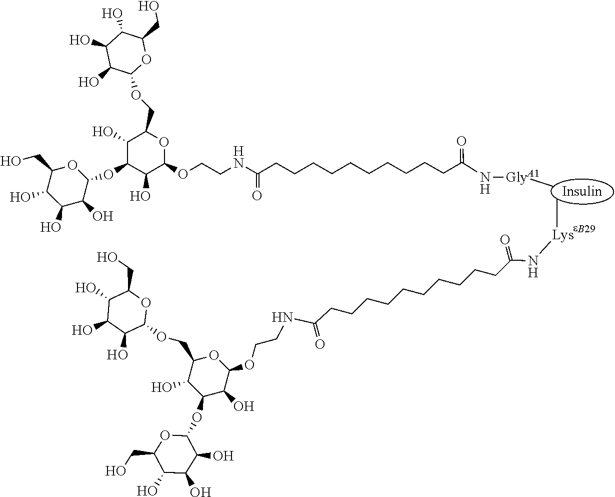 |
| IOC-45 | 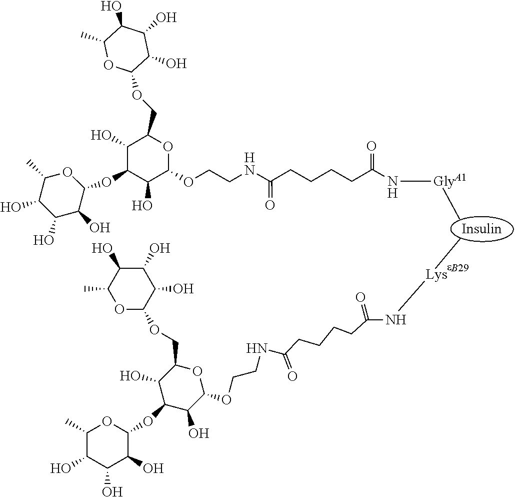 |
| IOC-46 | 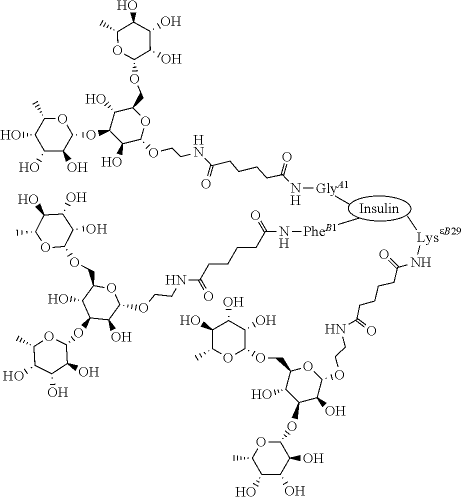 |
| IOC-47 | 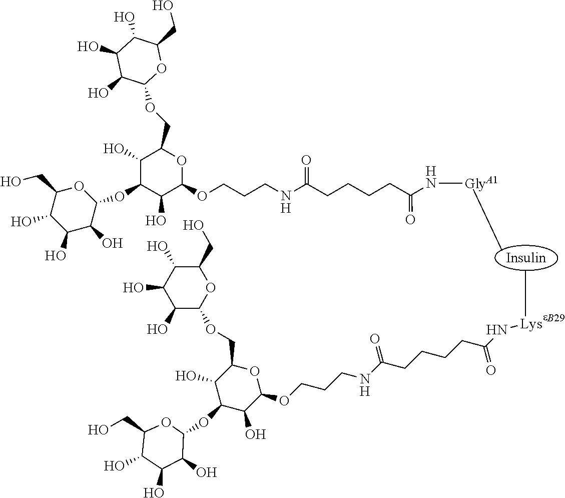 |
| IOC-48 | 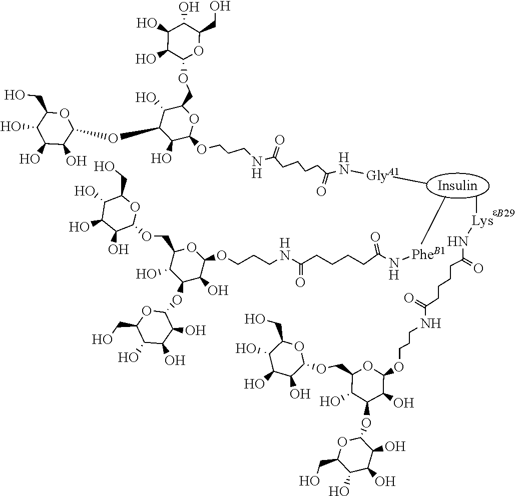 |
| IOC-49 | 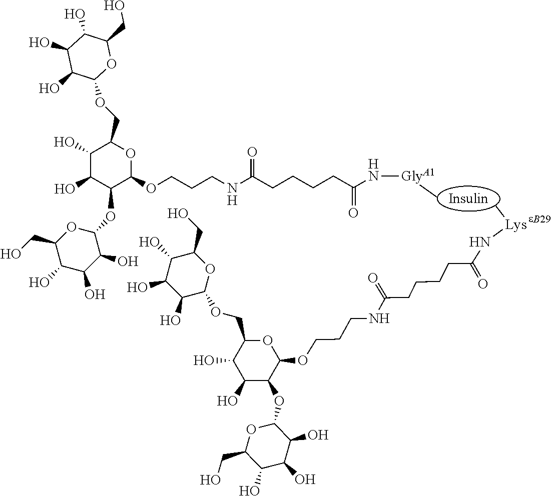 |
| IOC-50 | 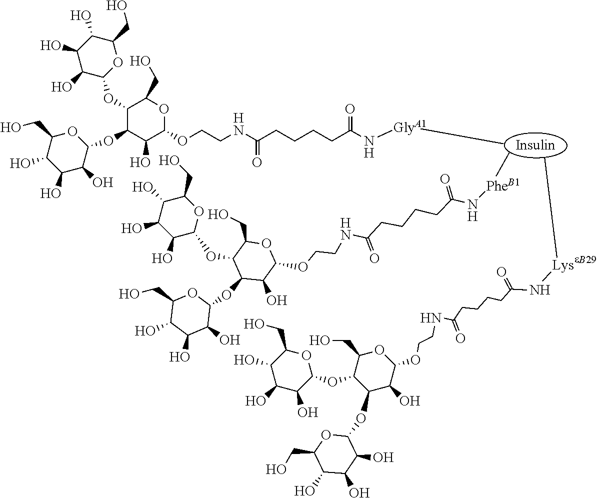 |
| IOC-51 | 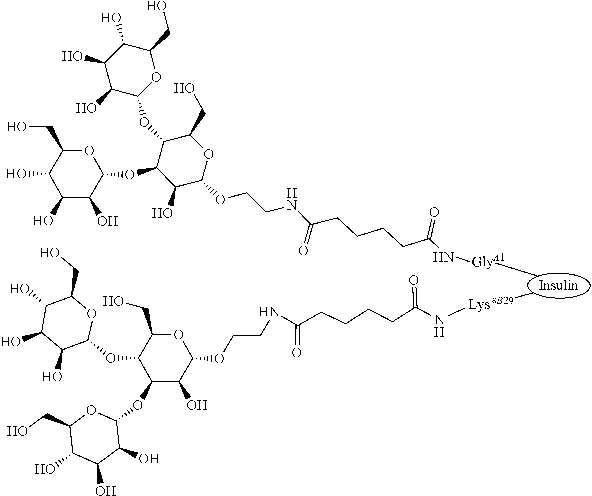 |
| IOC-52 | 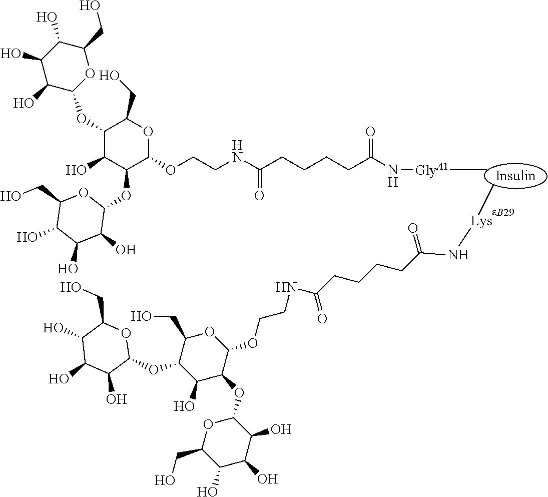 |
| IOC-53 | 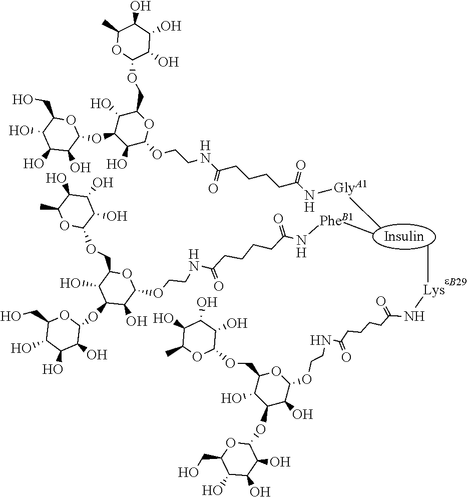 |
| IOC-54 | 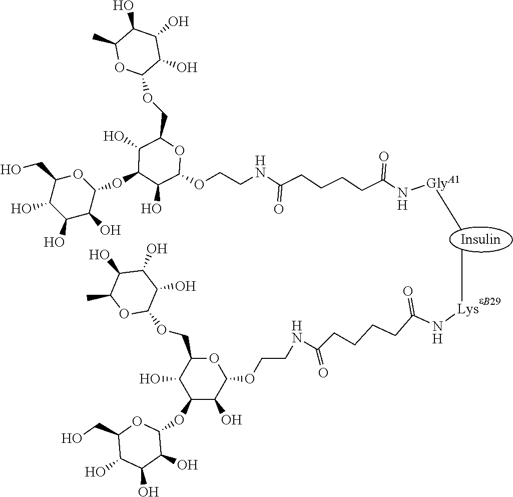 |
| IOC-55 | 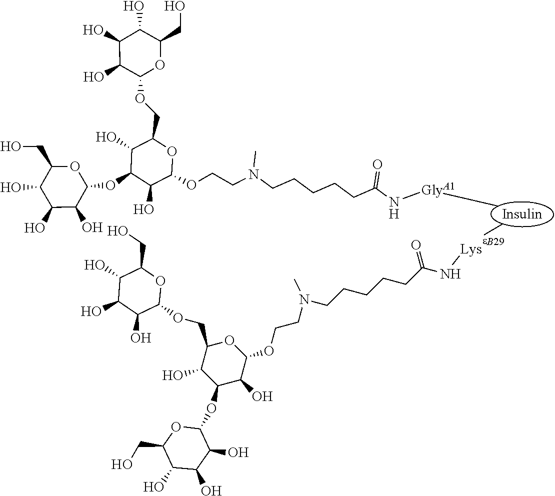 |
| IOC-56 | 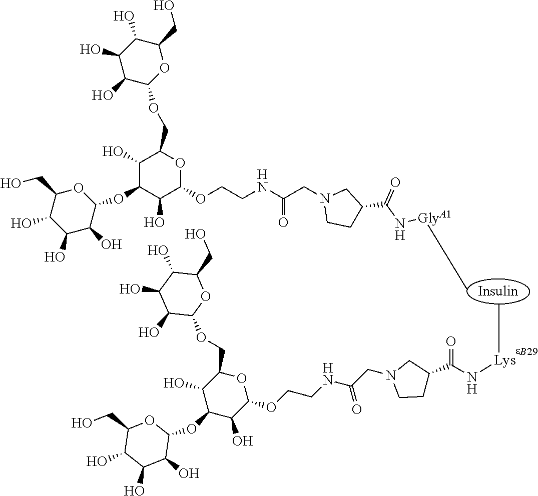 |
| IOC-57 | 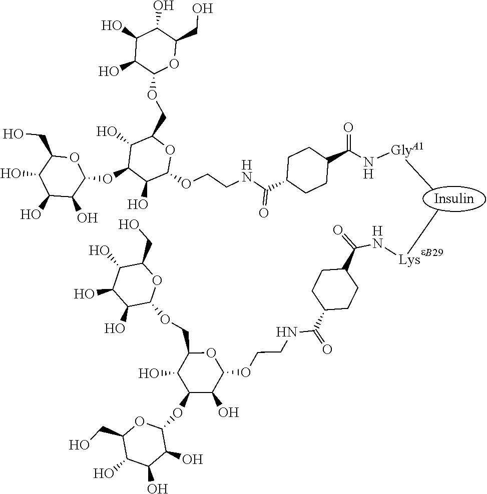 |
| IOC-58 | 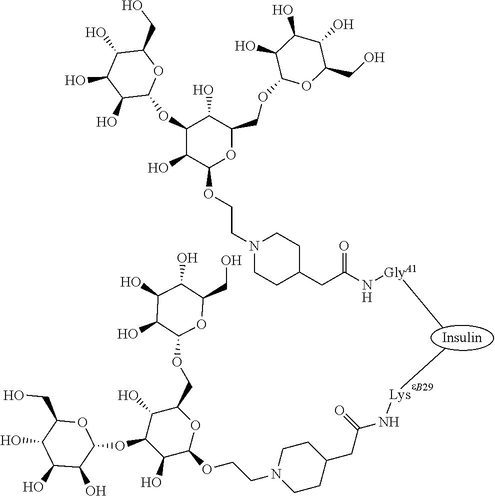 |
| IOC-59 | 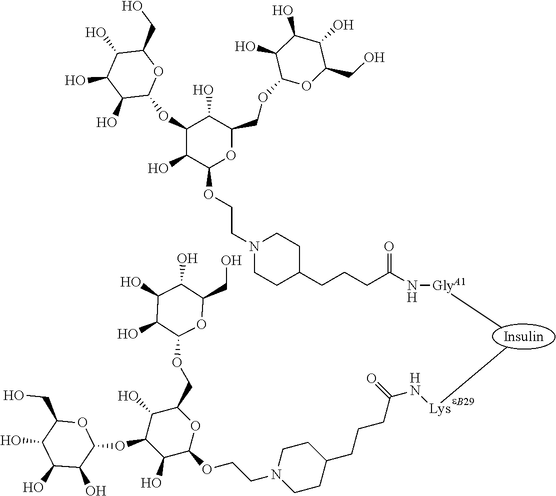 |
| IOC-60 | 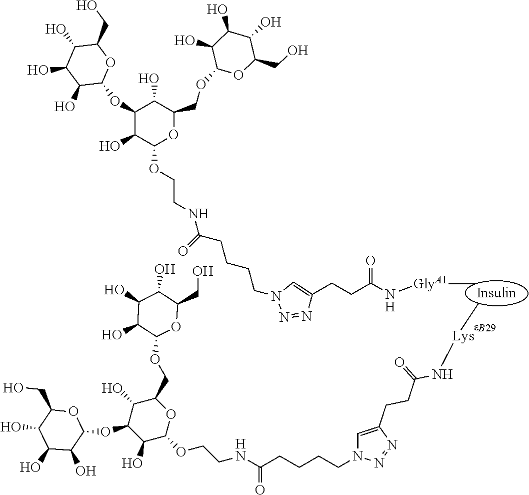 |
| IOC-61 | 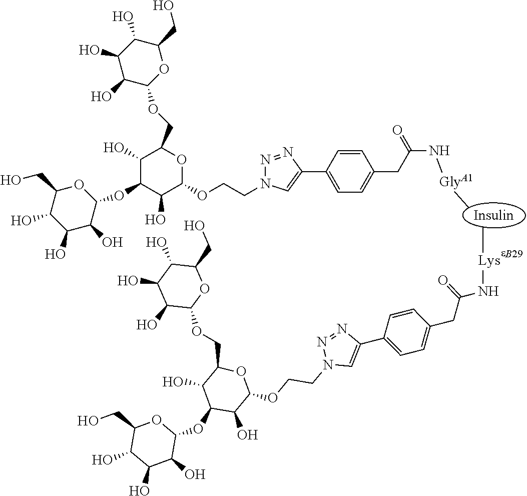 |
| IOC-62 | 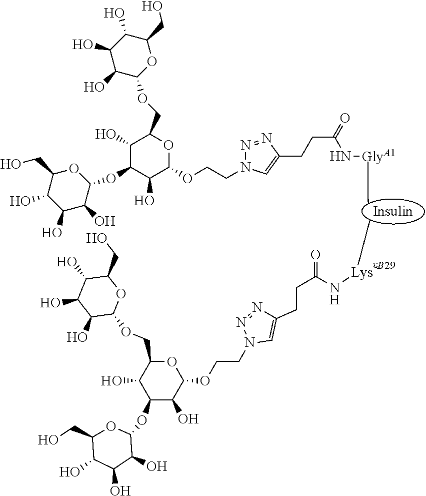 |
| IOC-63 | 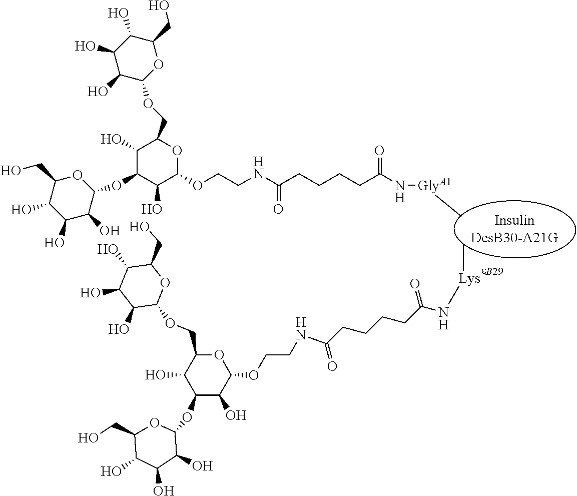 |
| IOC-64 | 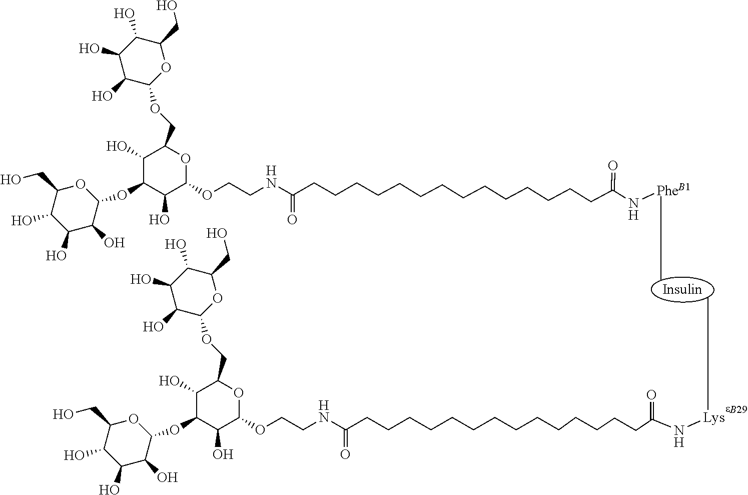 |
| IOC-65 | 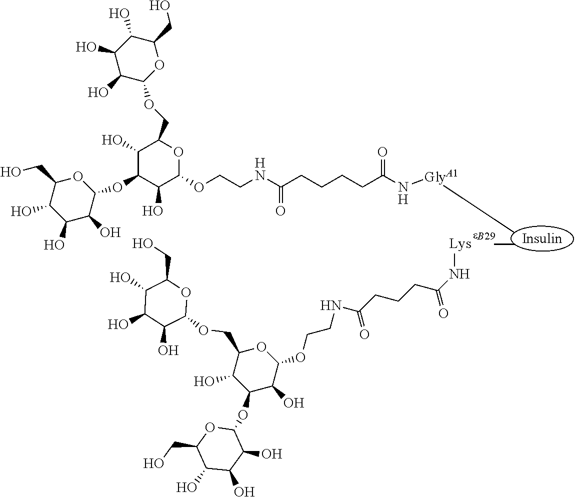 |
| IOC-66 | 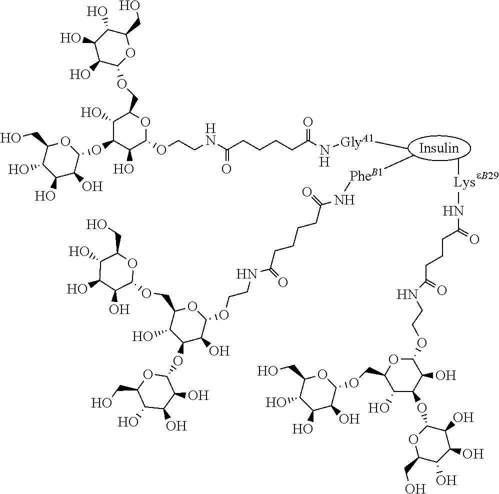 |
| IOC-67 | 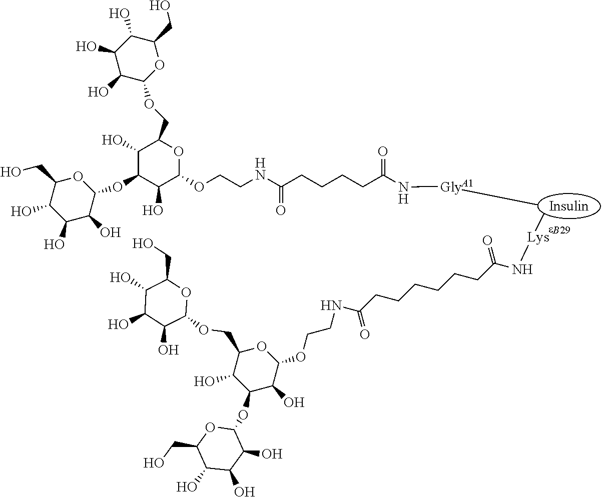 |
| IOC-68 | 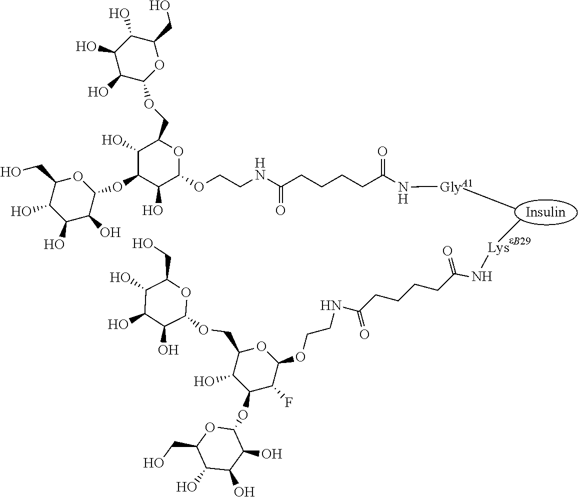 |
| IOC-69 | 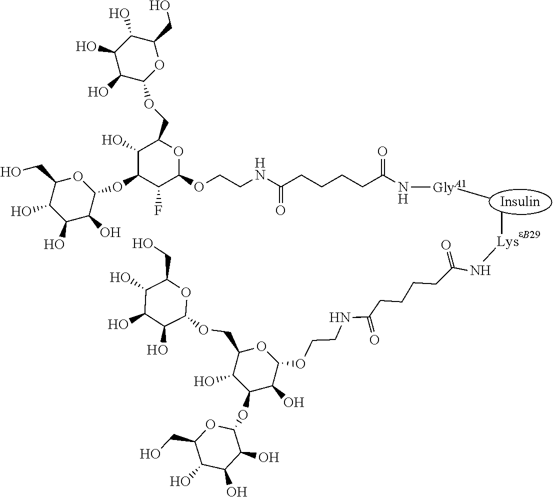 |
| IOC-70 | 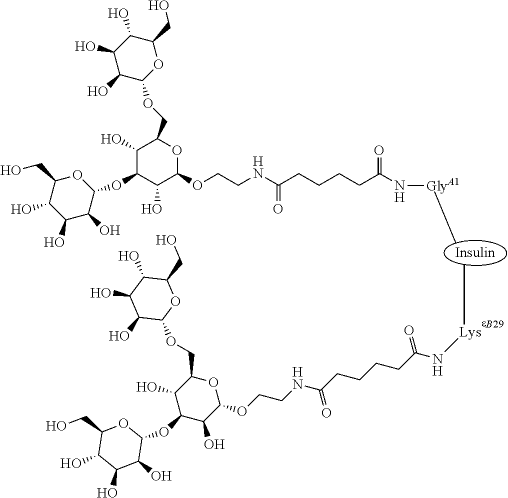 |
| IOC-71 | 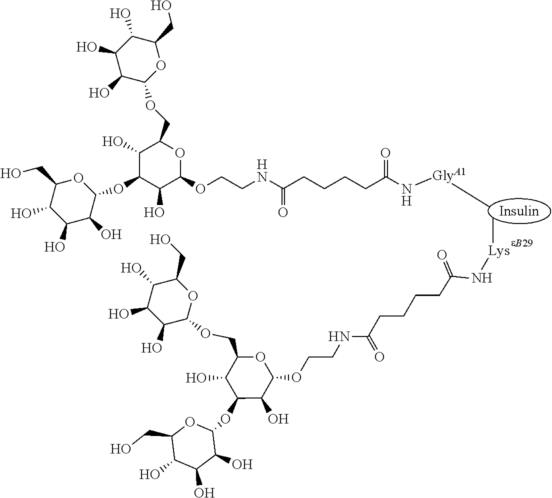 |
| IOC-72 | 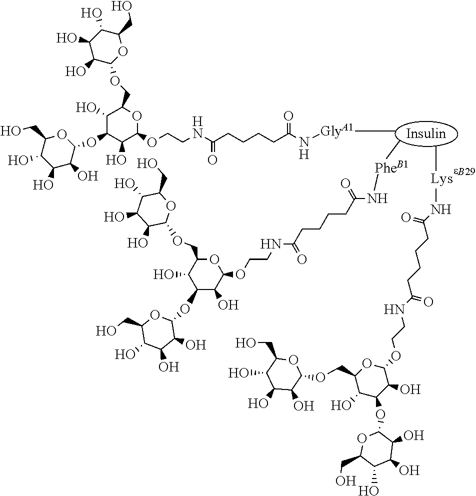 |
| IOC-73 | 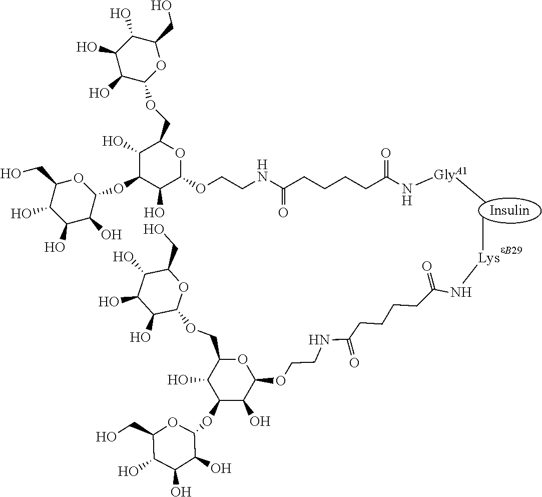 |
| IOC-74 | 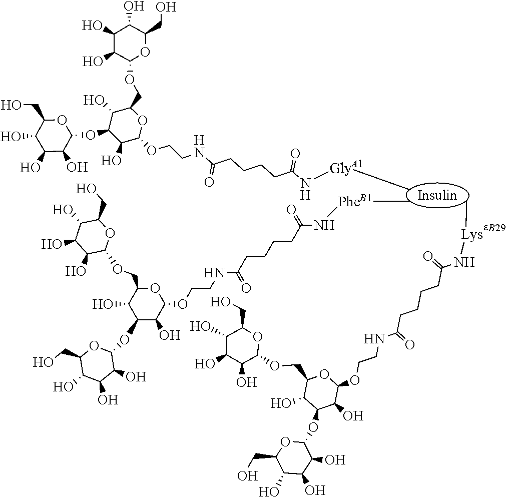 |
| IOC-75 | 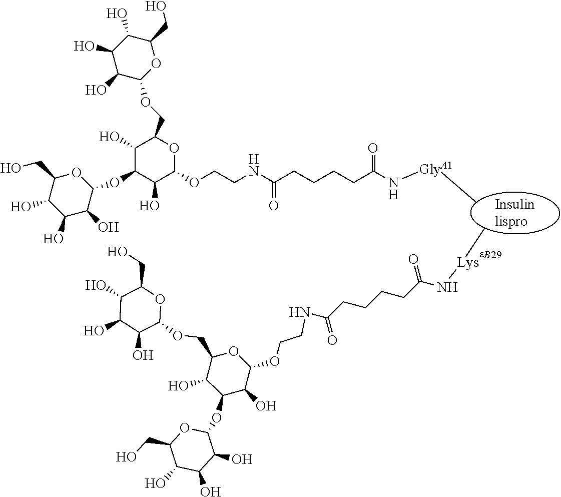 |
| IOC-76 | 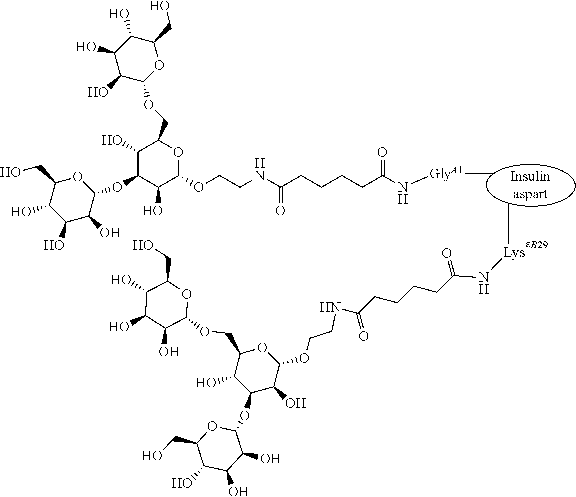 |
| IOC-77 | 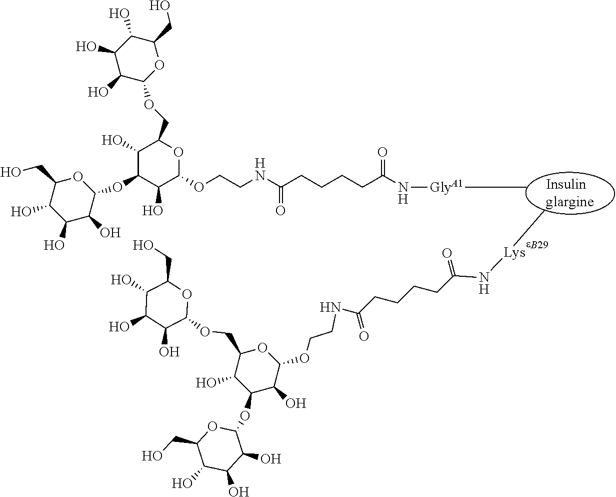 |
| IOC-79 | 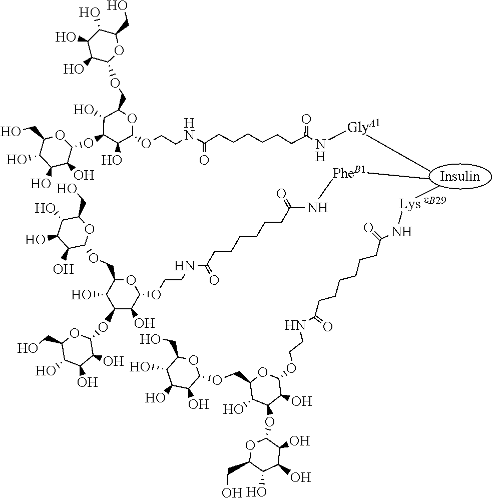 |
| IOC-80 | 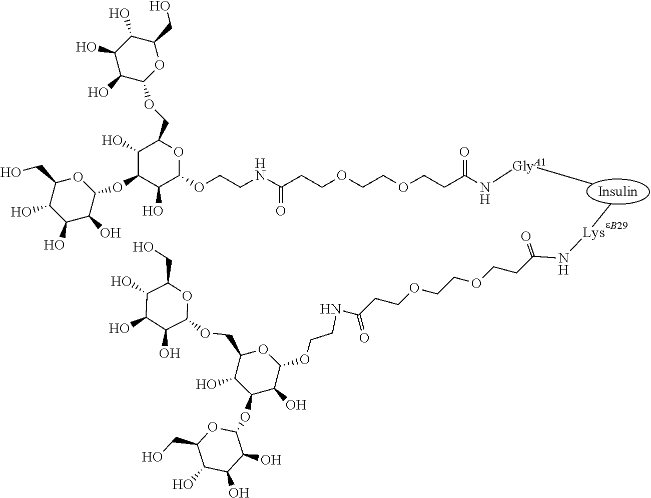 |
| IOC-81 | 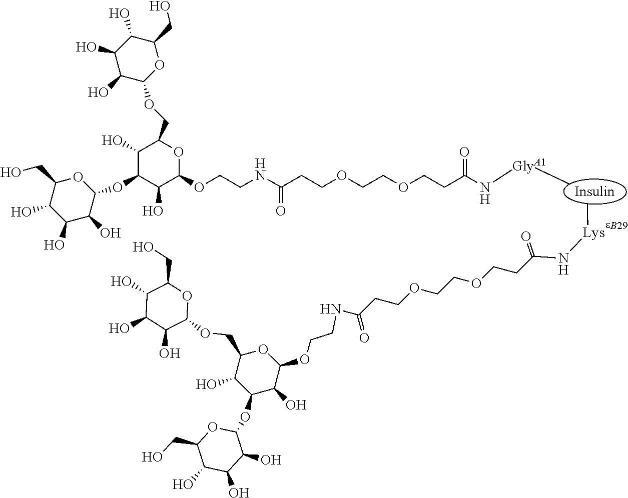 |
| IOC-82 | 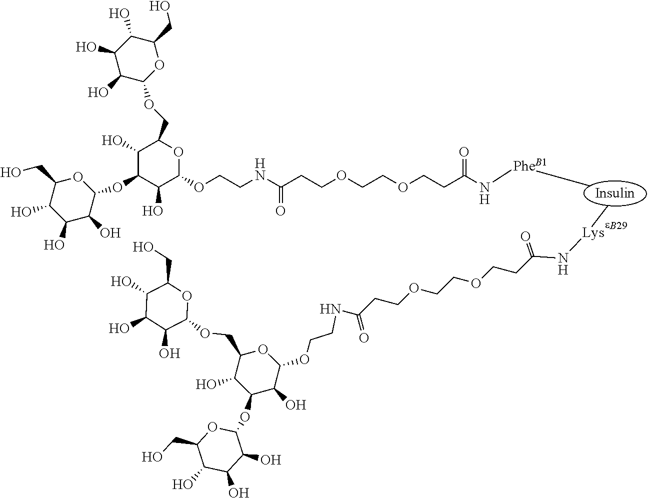 |
| IOC-83 | 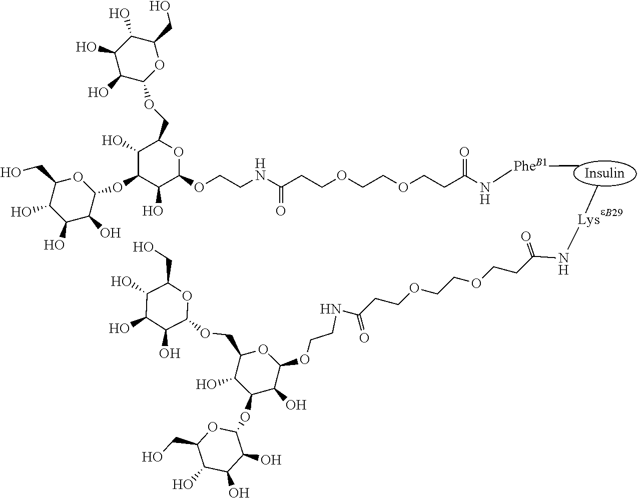 |
| IOC-84 | 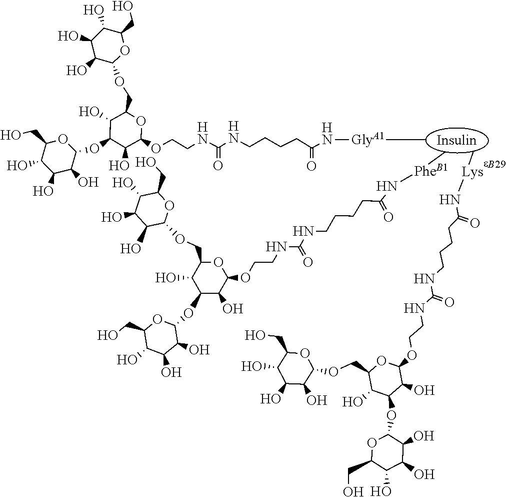 |
| IOC-85 | 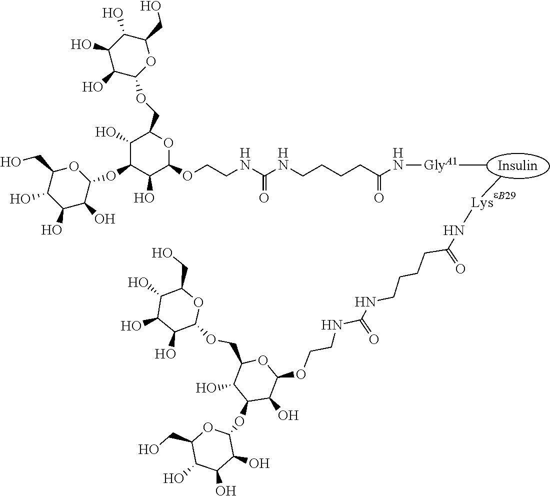 |
| IOC-86 | 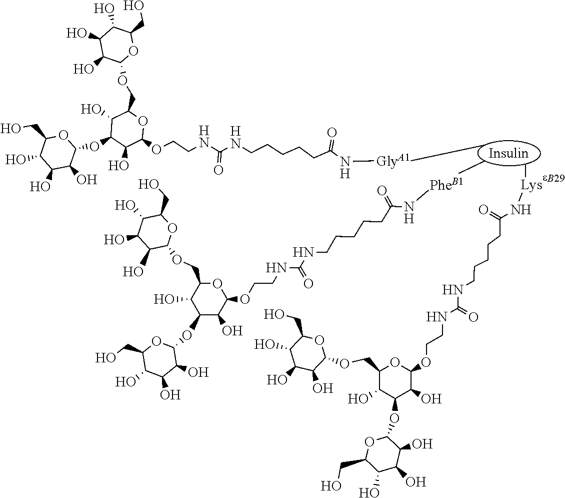 |
| IOC-87 | 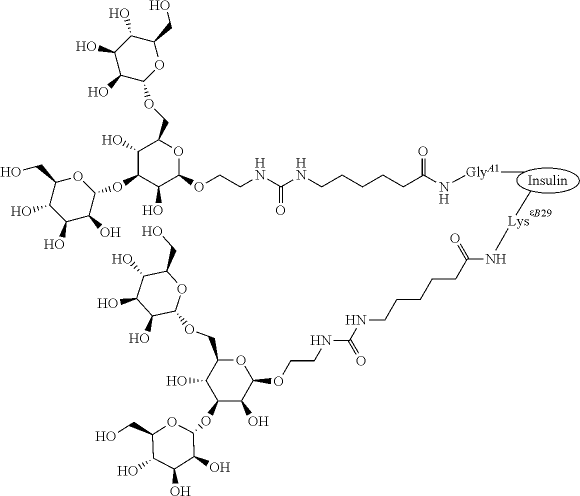 |
| IOC-88 | 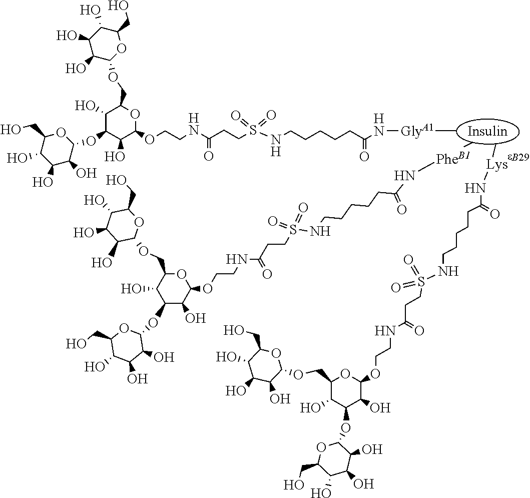 |
| IOC-89 | 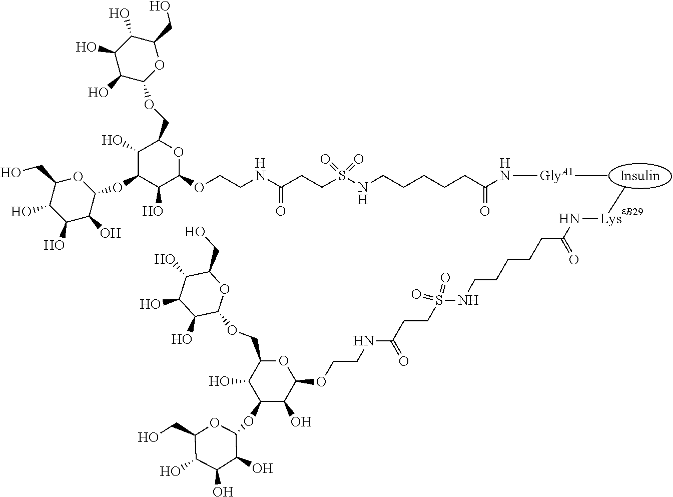 |
| IOC-90 | 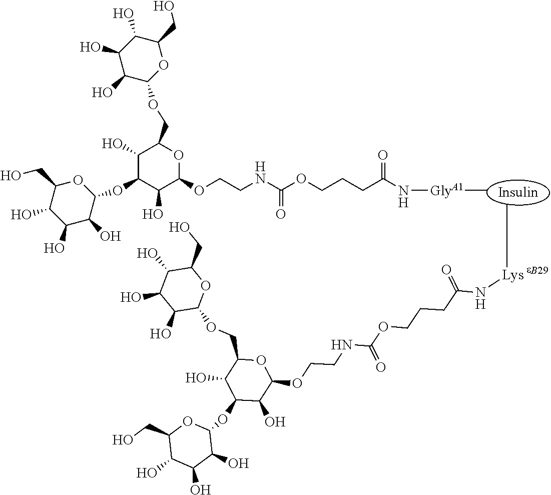 |
| IOC-91 | 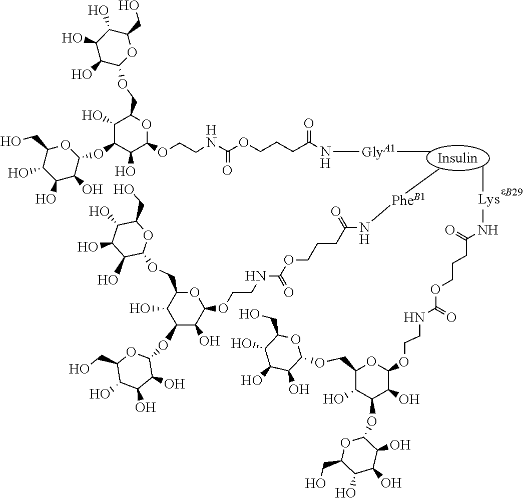 |
| IOC-92 | 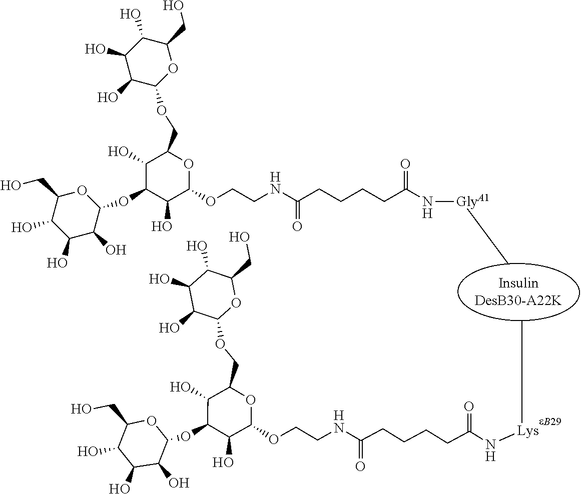 |
| IOC-93 | 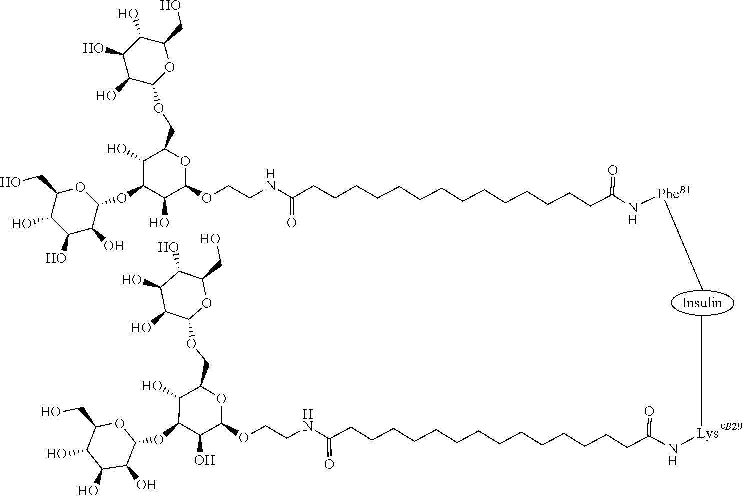 |
| IOC-94 | 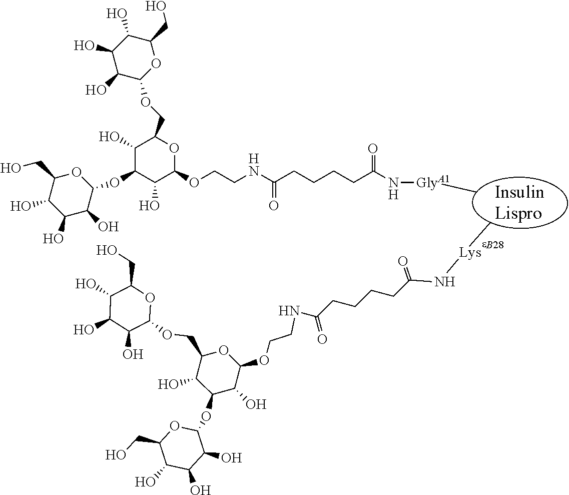 |
| IOC-95 | 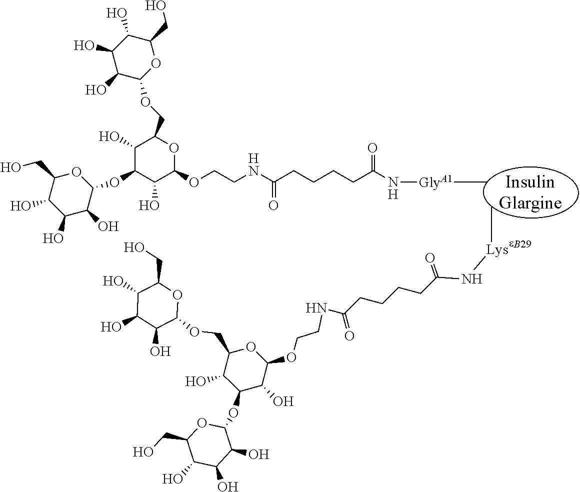 |
| IOC-96 | 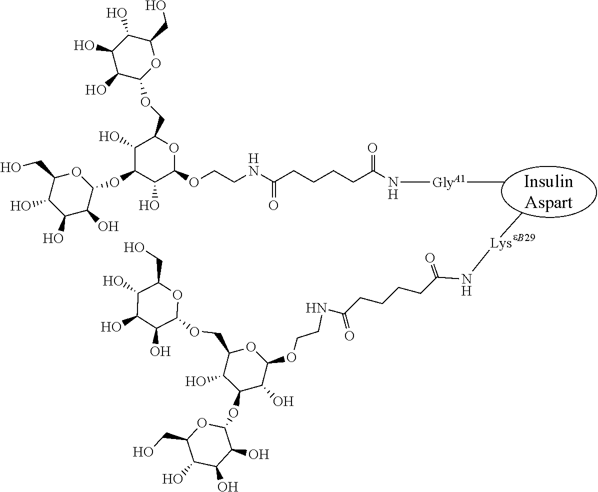 |
| IOC-97 | 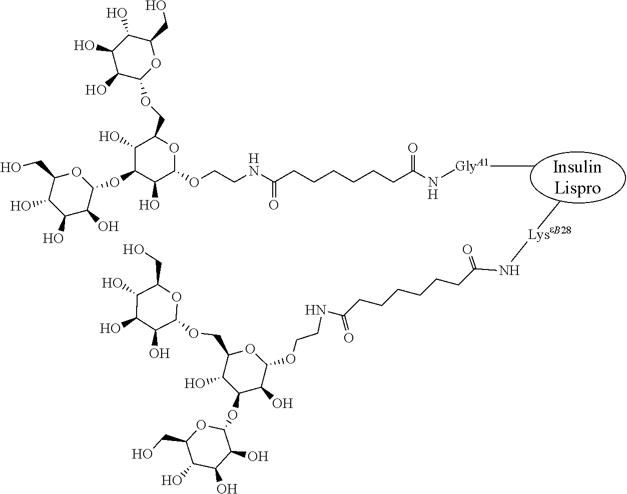 |
| IOC-98 | 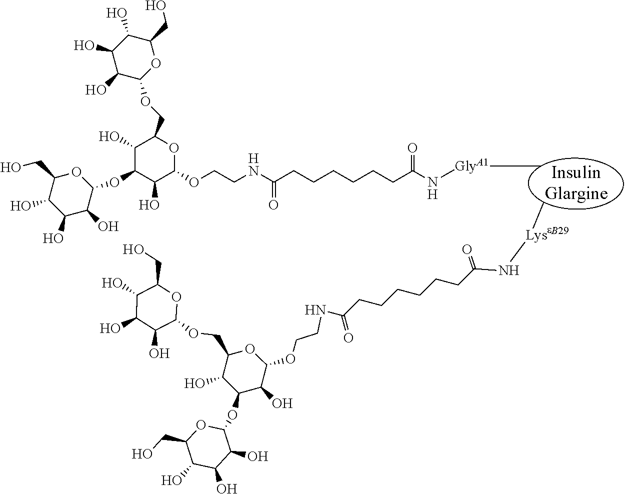 |
| IOC-99 | 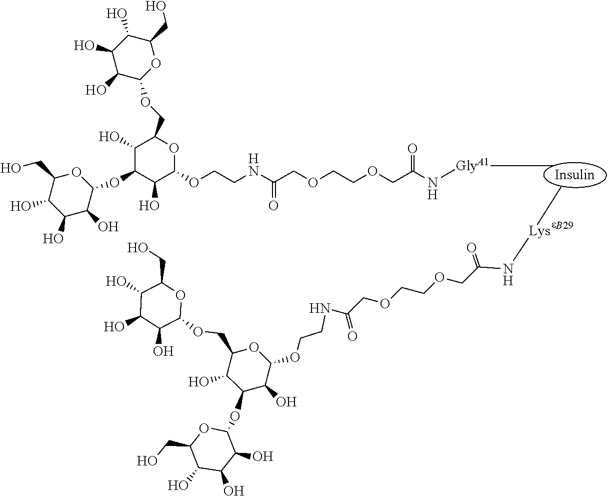 |
| IOC-100 | 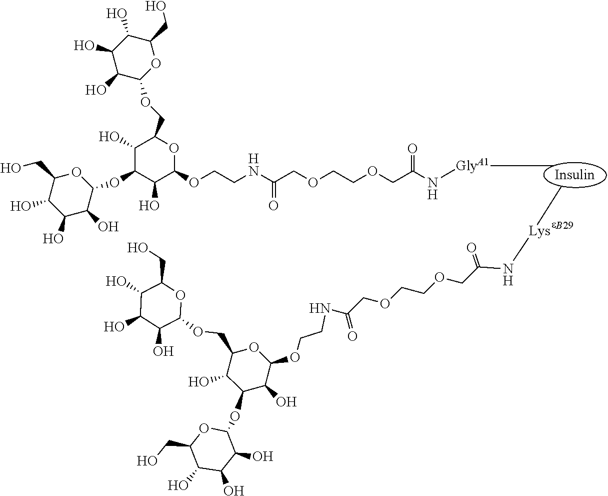 |
| IOC-101 | 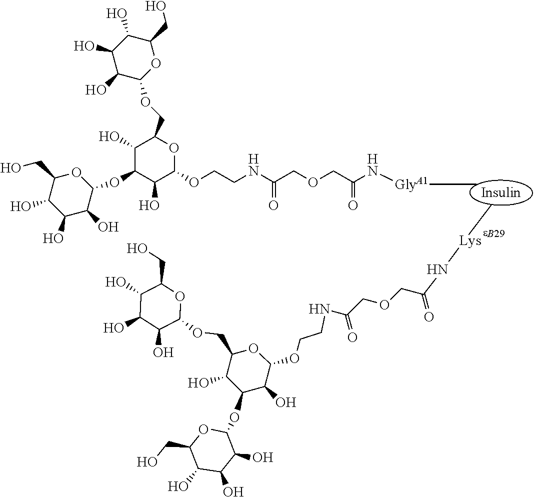 |
| IOC-102 | 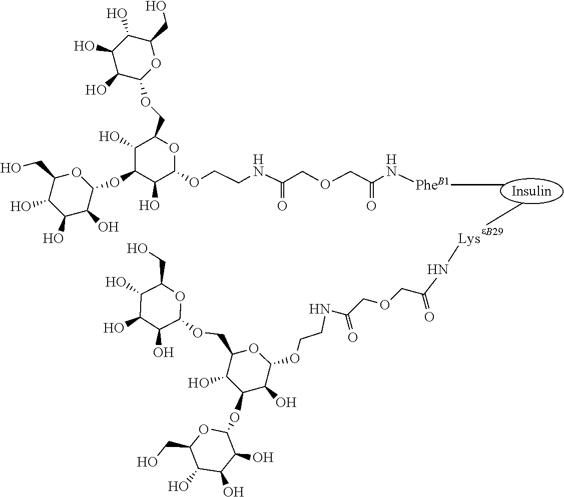 |
| IOC-103 | 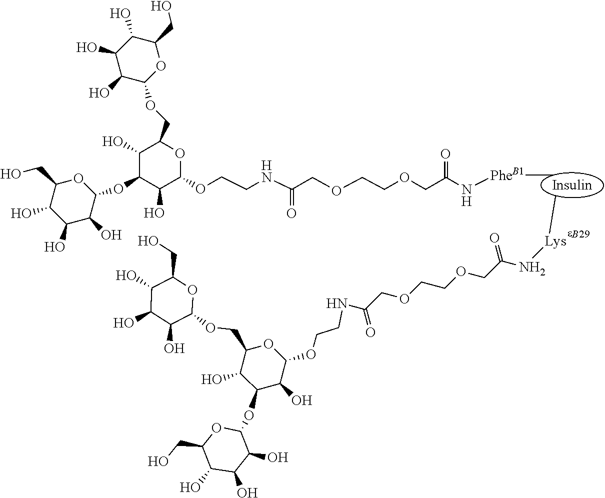 |
| IOC-104 | 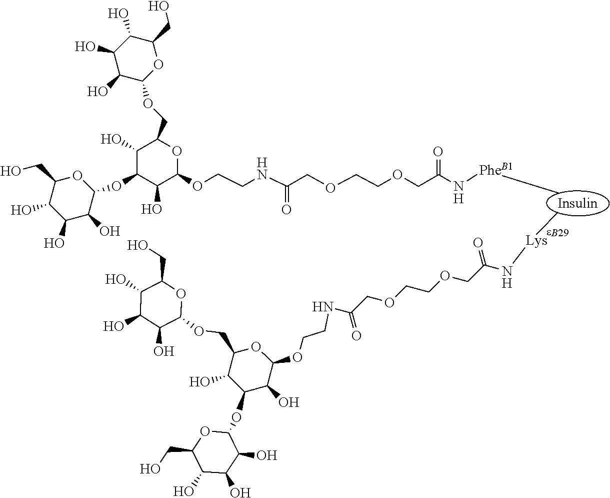 |
| IOC-105 | 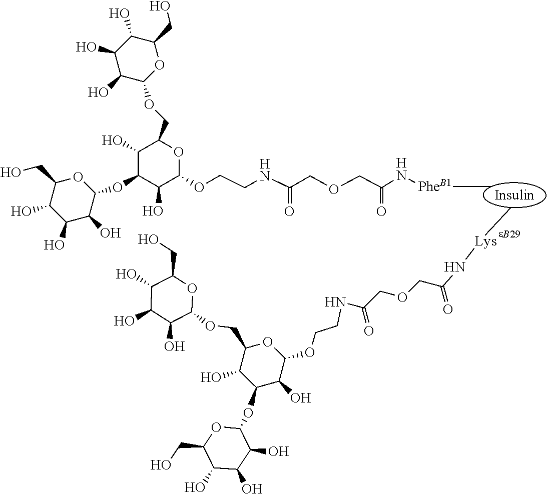 |
| IOC-106 | 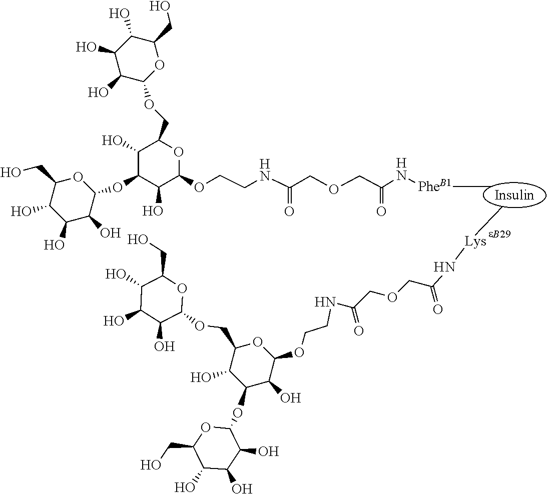 |
| IOC-107 | 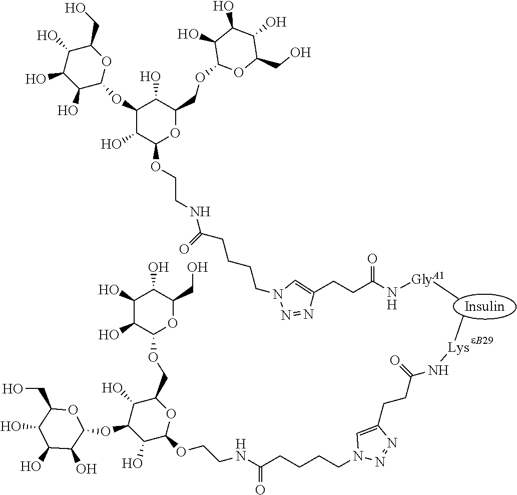 |
| IOC-108 | 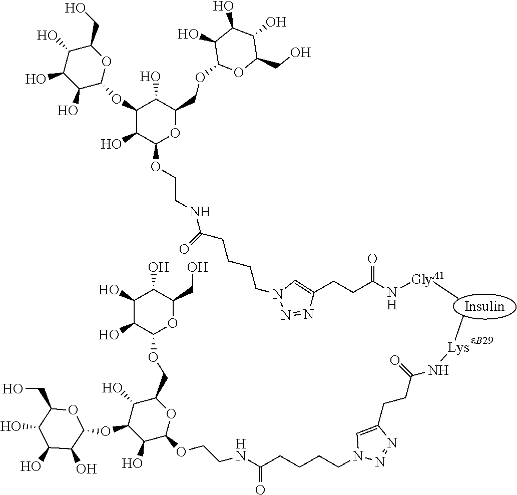 |
| IOC-109 | 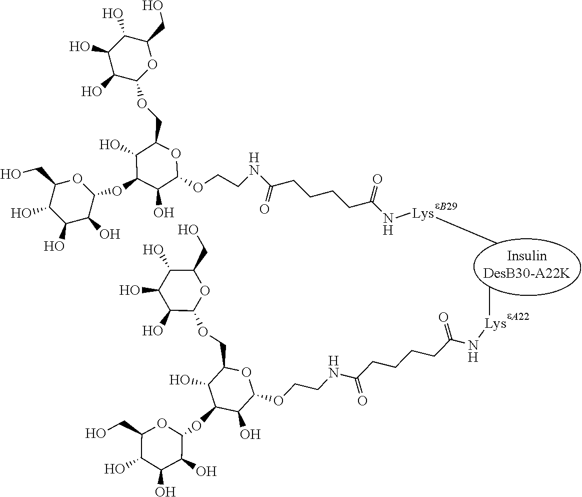 |
| IOC-110 | 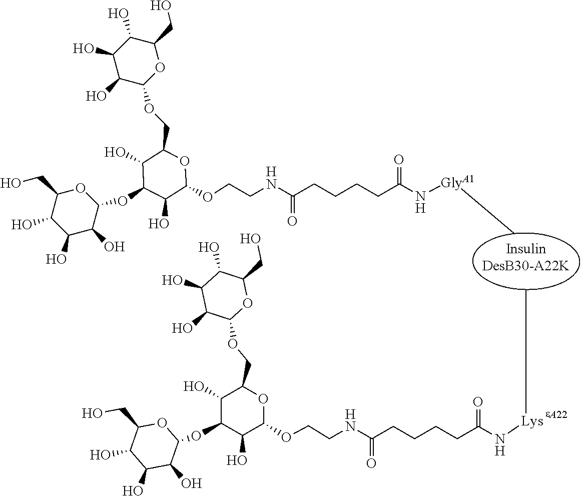 |
| IOC-111 | 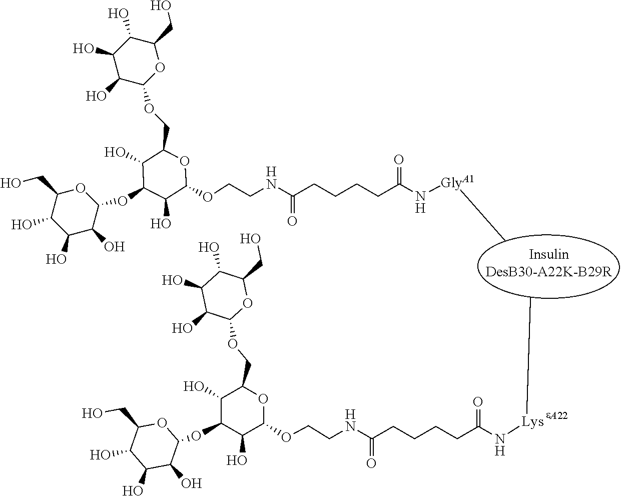 |
| IOC-112 | 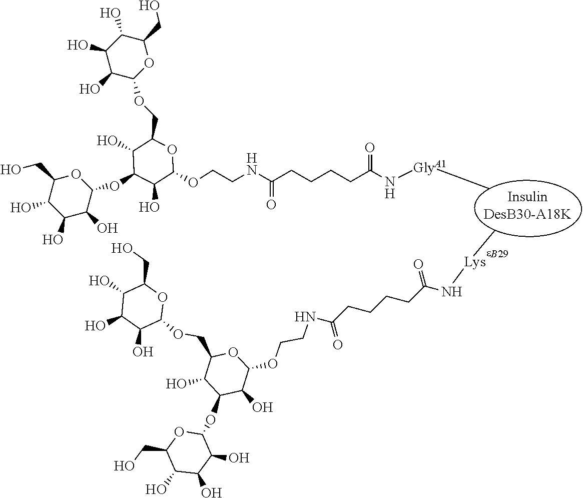 |
| IOC-113 | 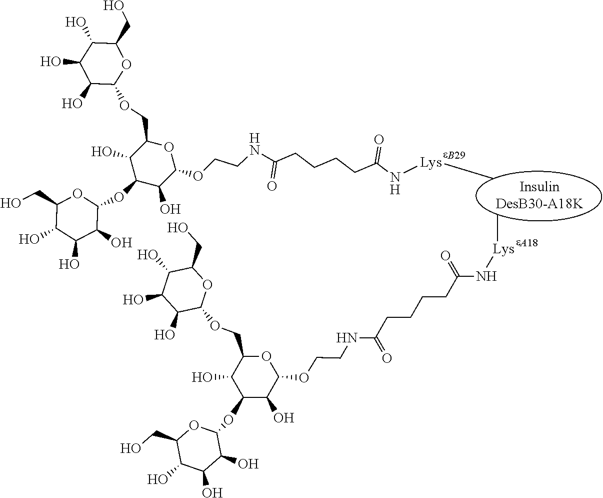 |
| IOC-114 | 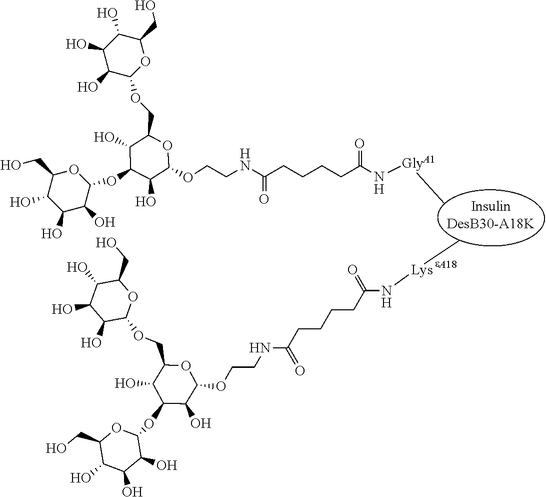 |
| IOC-115 | 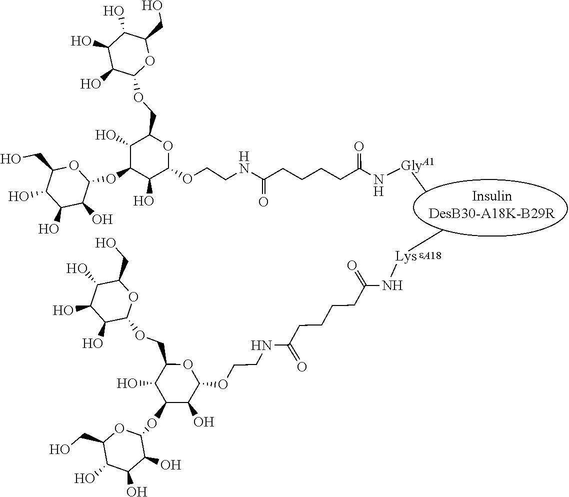 |
| IOC-116 | 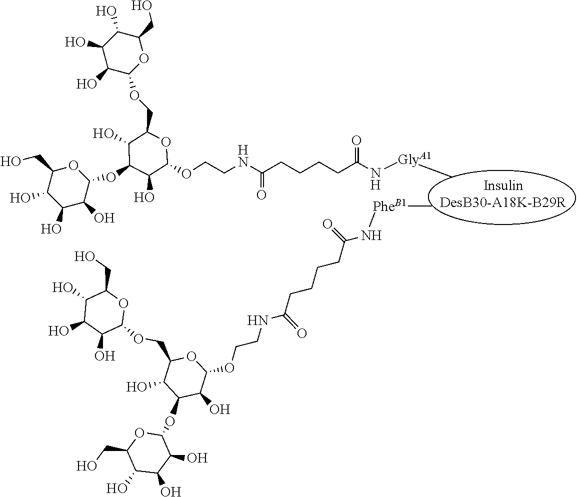 |
| IOC-117 | 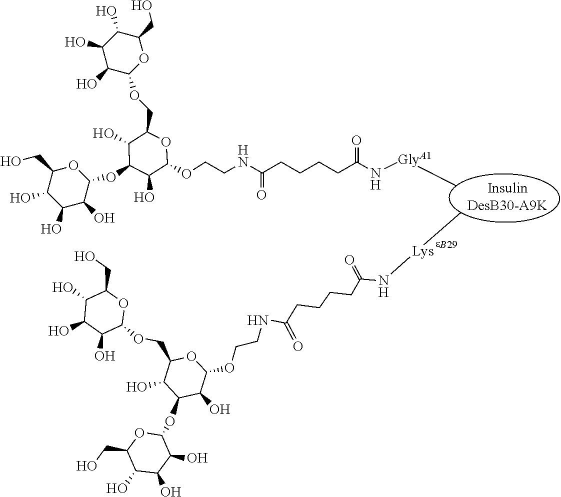 |
| IOC-118 | 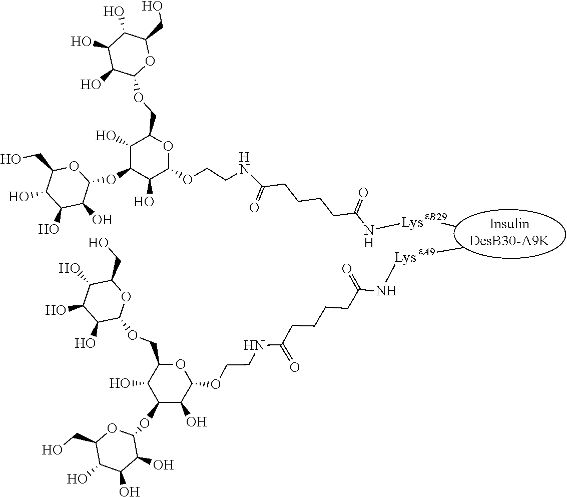 |
| IOC-119 | 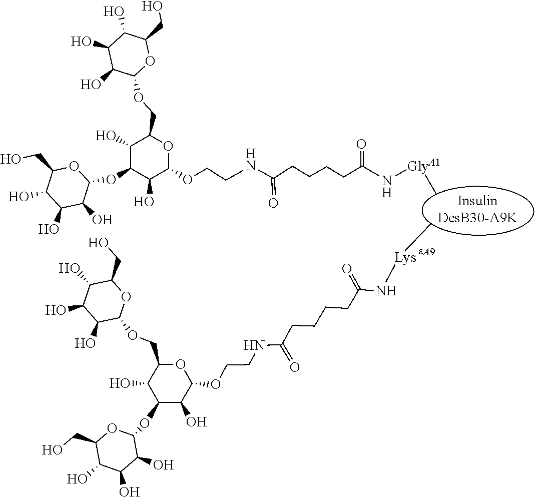 |
| IOC-120 | 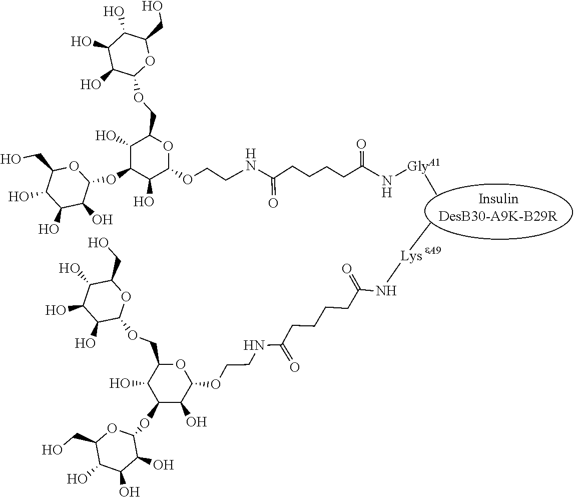 |
| IOC-121 | 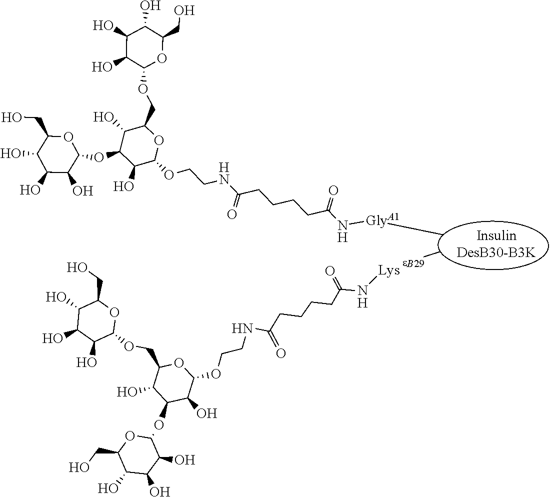 |
| IOC-122 | 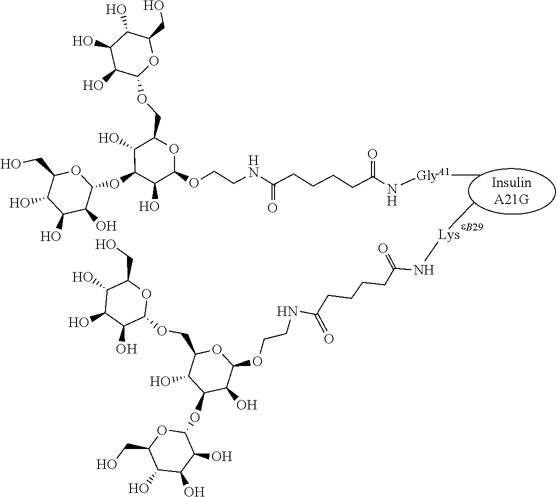 |
| IOC-123 | 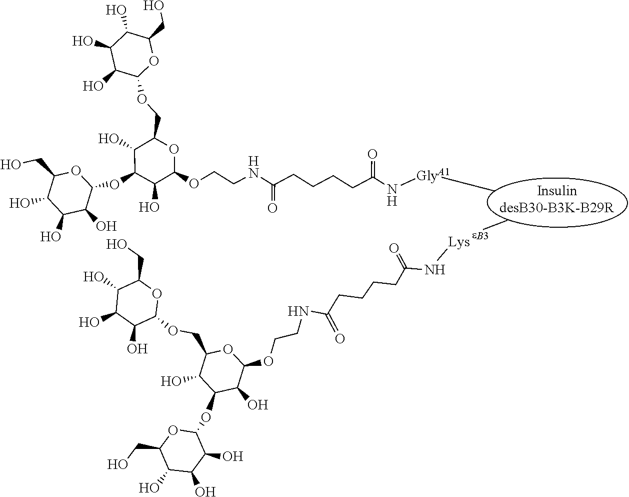 |
| IOC-124 | 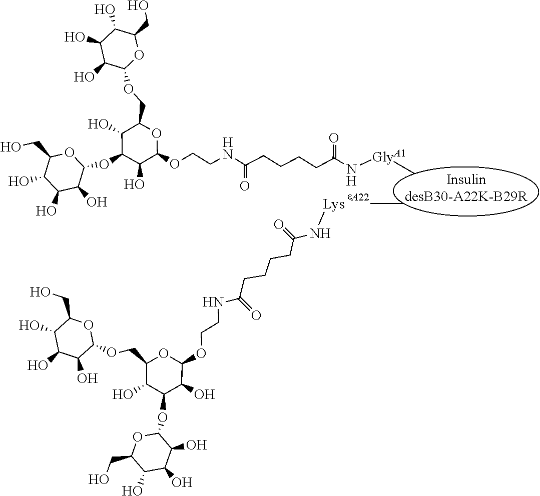 |
| IOC-125 | 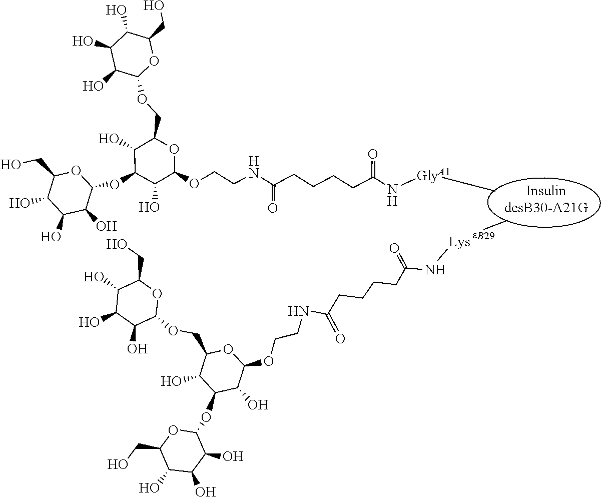 |
| IOC-126 | 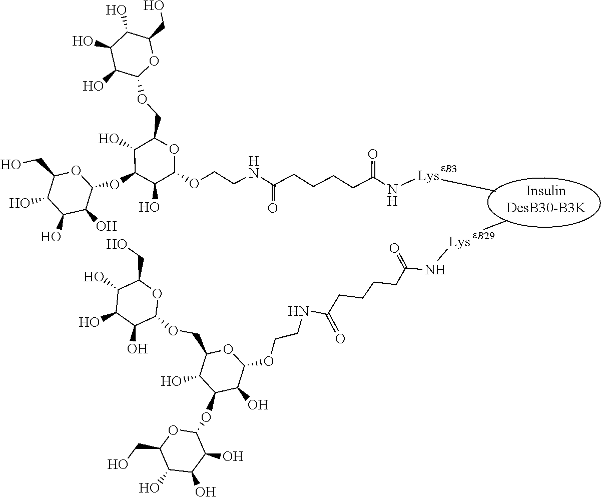 |
| IOC-127 | 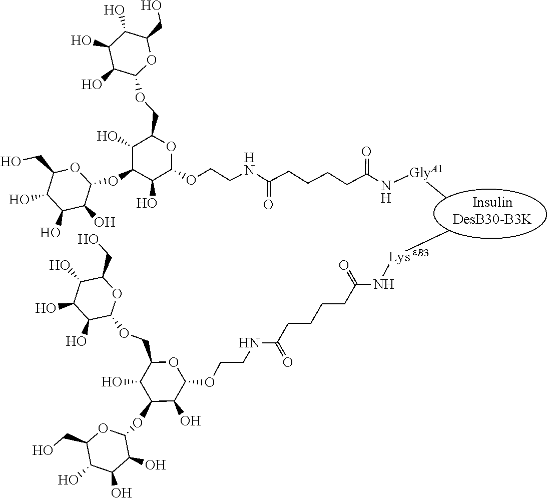 |
| IOC-128 | 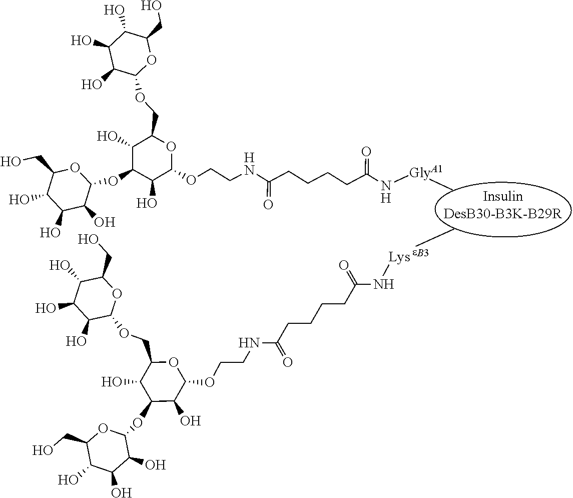 |
| IOC-129 | 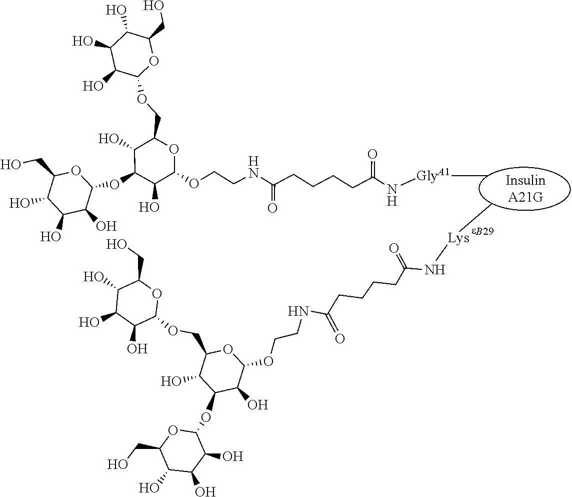 |
| IOC-130 | 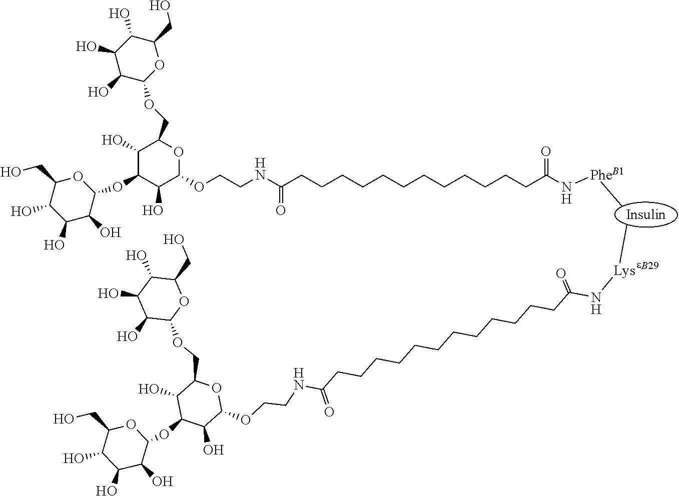 |
| IOC-131 | 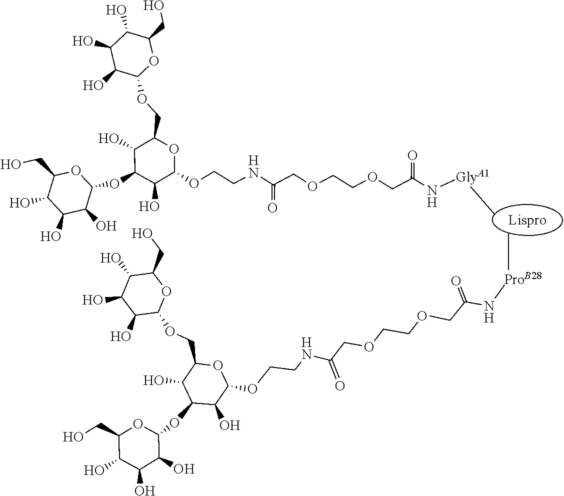 |
| IOC-132 | 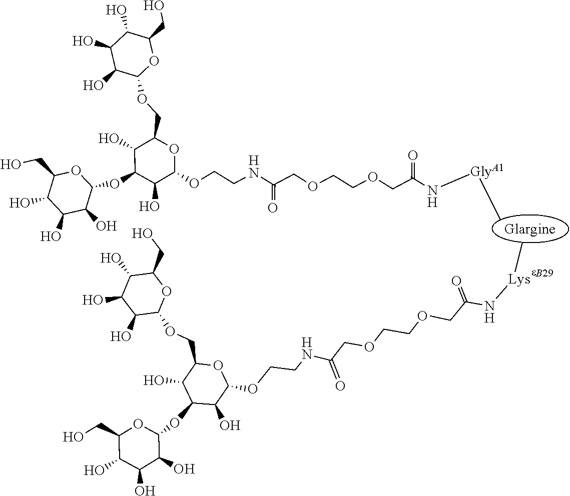 |
| IOC-133 | 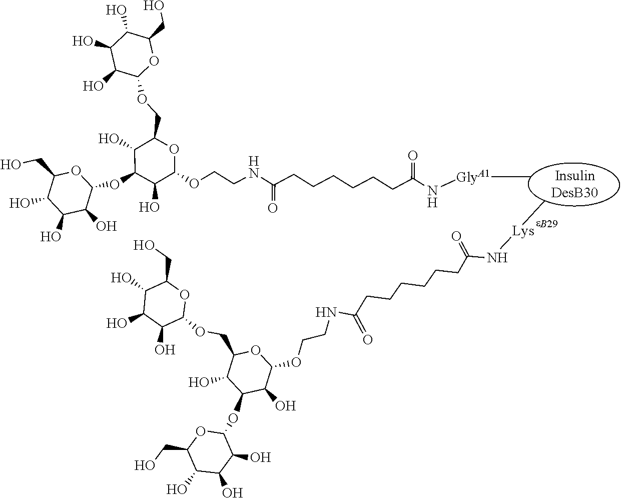 |
| IOC-134 | 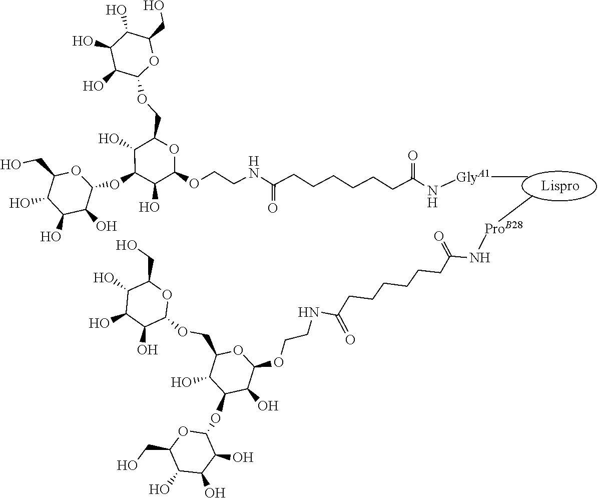 |
| IOC-135 | 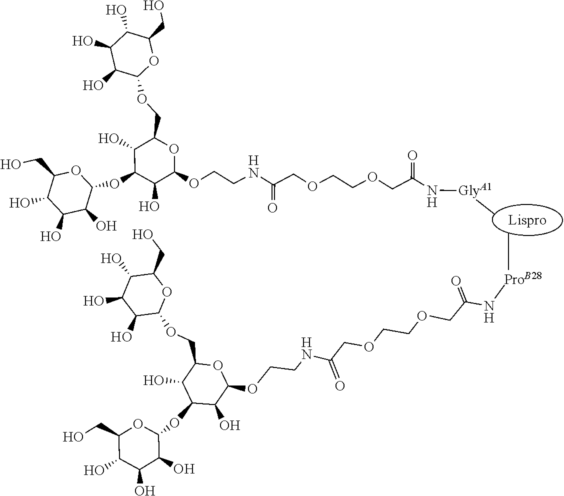 |
| IOC-136 | 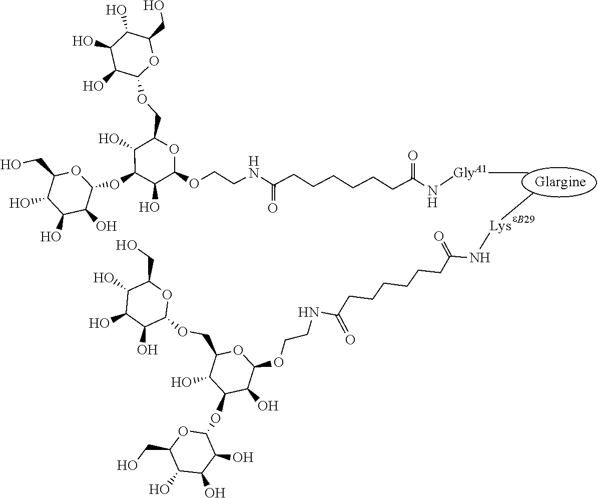 |
| IOC-137 | 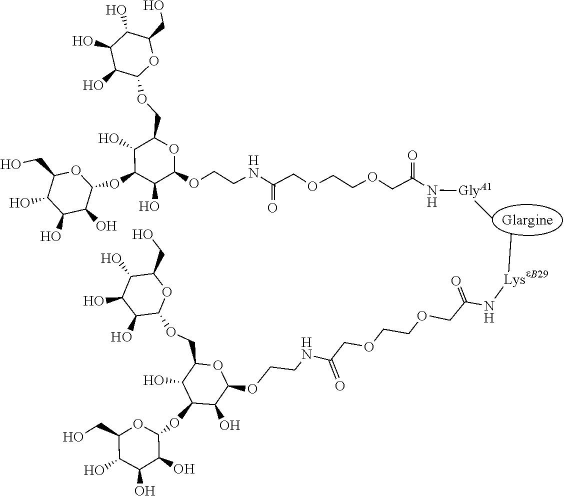 |
| IOC-138 | 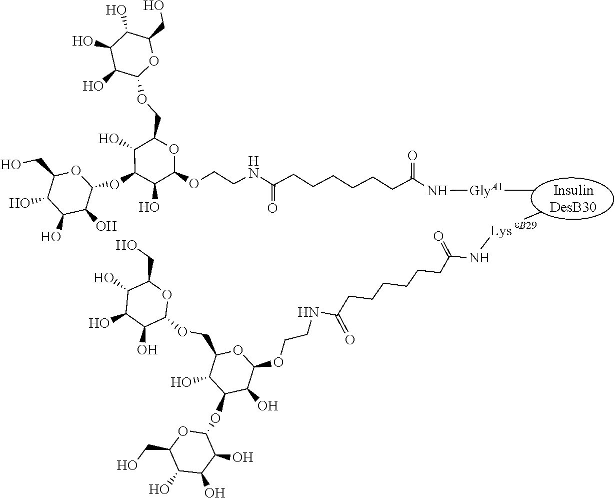 |
| IOC-139 | 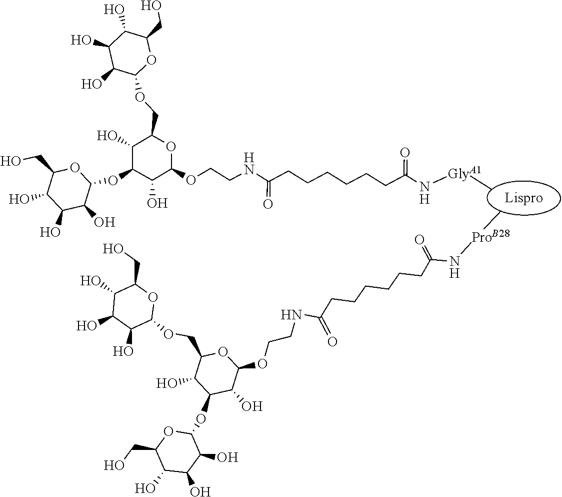 |
| IOC-140 | 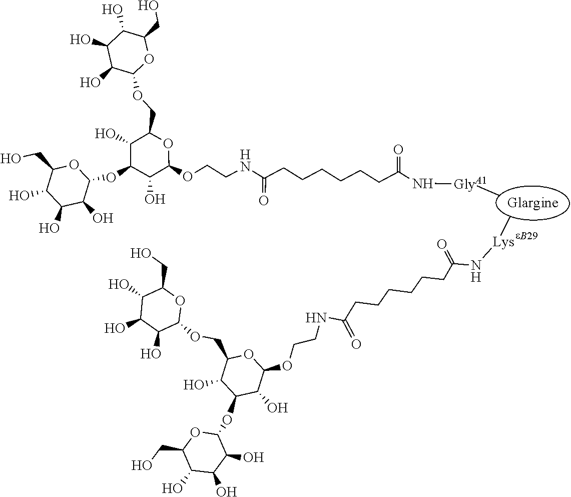 |
| IOC-141 | 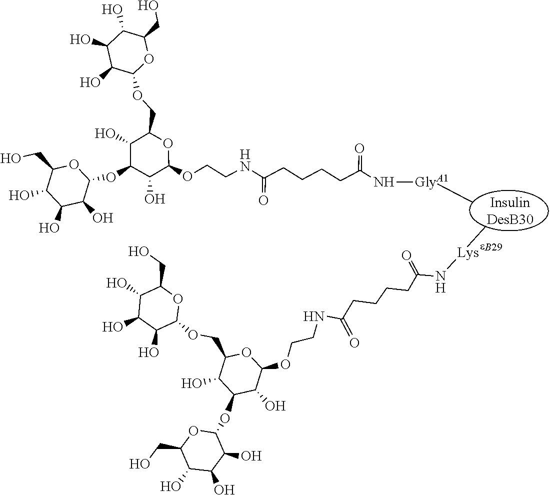 |
| IOC-142 | 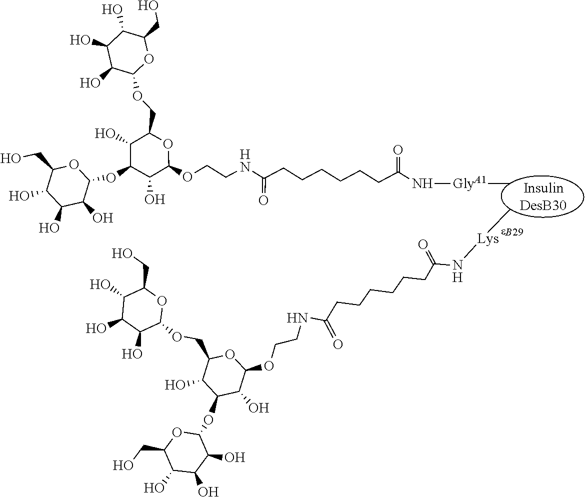 |
| IOC-143 | 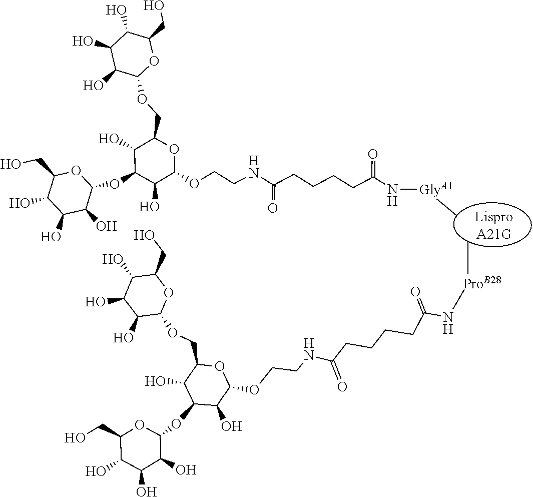 |
| IOC-144 | 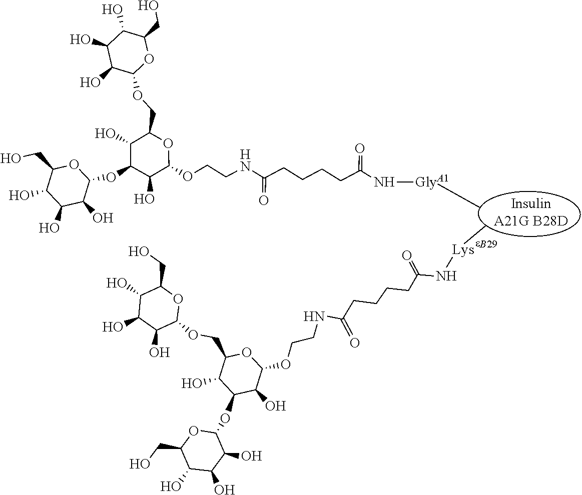 |
| IOC-145 | 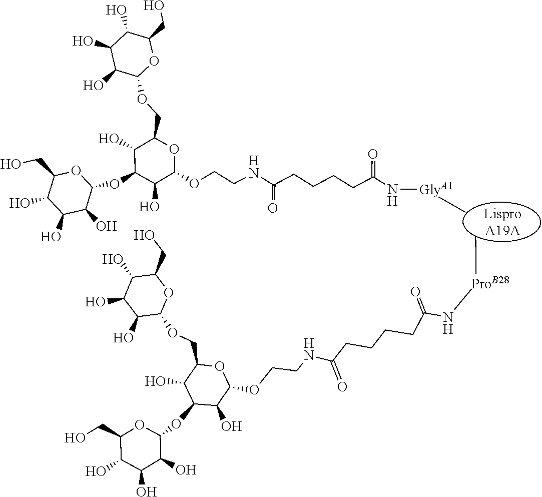 |
| IOC-146 | 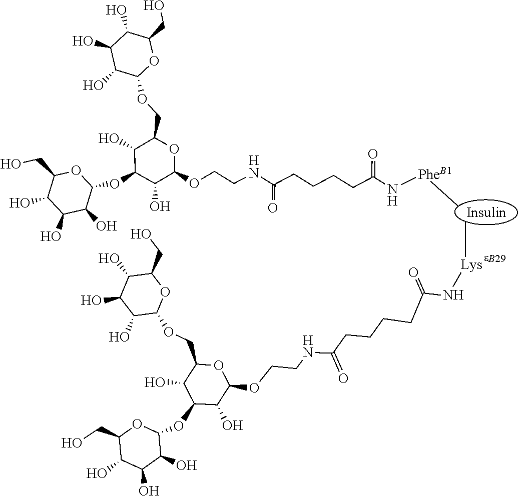 |
| IOC-147 | 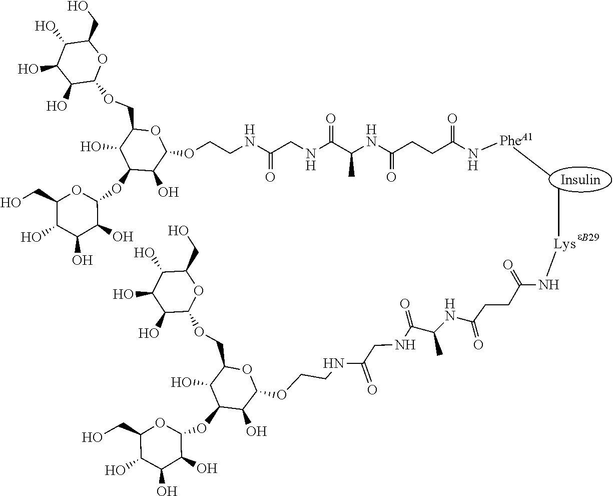 |
| IOC-148 | 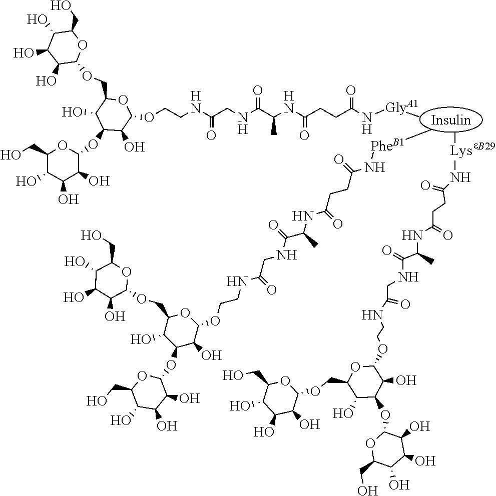 |

Additional embodiments of the disclosure provide for the use of any one of the conjugates disclosed herein for the manufacture of a medicament to treat diabetes.

Additional embodiments of the disclosure provide for the use of any one of the conjugates disclosed herein for the manufacture of a medicament to treat a Type I diabetes, Type II diabetes, gestational diabetes, impaired glucose tolerance, or prediabetes.

Additional embodiments of the disclosure provide a composition comprising of any one of the conjugates disclosed herein and a pharmaceutically acceptable carrier.

Additional embodiments of the disclosure provide for use of the composition comprising of any one of the conjugates disclosed herein and a pharmaceutically acceptable carrier for the treatment of diabetes. In particular aspects, the diabetes is Type I diabetes, Type II diabetes, or gestational diabetes.

The disclosure further provides embodiments of a method for treating a subject who has diabetes, comprising administering to the subject an effective amount of the composition comprising of any one of the conjugates disclosed herein and a pharmaceutically acceptable carrier for treating the diabetes, wherein said administering treats the diabetes. In particular aspects, the diabetes is Type I diabetes, Type II diabetes, or gestational diabetes.

The disclosure further provides embodiments of a composition comprising any one of the conjugates disclosed herein, wherein the conjugate is characterized as having a ratio of EC50 or IP as determined by a functional insulin receptor phosphorylation assay to the IC50 or IP as determined by a competition binding assay at the macrophage mannose receptor that is about 0.5:1 to about 1:100, about 1:1 to about 1:50, about 1:1 to about 1:20, or about 1:1 to about 1:10; and a pharmaceutically acceptable carrier.

The disclosure still further provides embodiments of a method for treating a subject who has diabetes, comprising administering to the subject a composition comprising any one of the conjugates disclosed herein, wherein the conjugate is characterized as having a ratio of EC50 or IP as determined by a functional insulin receptor phosphorylation assay to the IC50 or IP as determined by a competition binding assay at the macrophage mannose receptor that is about 0.5:1 to about 1:100, about 1:1 to about 1:50, about 1:1 to about 1:20, or about 1:1 to about 1:10; and a pharmaceutically acceptable carrier, wherein the administering treats the diabetes. In particular aspects, the diabetes is Type I diabetes, Type II diabetes, or gestational diabetes.

### Sustained Release Formulations

In particular embodiments, it may be advantageous to administer an insulin conjugate in a sustained fashion (i.e., in a form that exhibits an absorption profile that is more sustained than soluble recombinant human insulin). This will provide a sustained level of conjugate that can respond to fluctuations in glucose on a timescale that is more closely related to the typical glucose fluctuation timescale (i.e., hours rather than minutes). In particular embodiments, the sustained release formulation may exhibit a zero-order release of the conjugate when administered to a mammal under non-hyperglycemic conditions (i.e., fasted conditions). It will be appreciated that any formulation that provides a sustained absorption profile may be used. In particular embodiments this may be achieved by combining the conjugate with other ingredients that slow its release properties into systemic circulation.

For example, PZI (protamine zinc insulin) formulations may be used for this purpose. The present disclosure encompasses amorphous and crystalline forms of these PZI formulations.

Thus, in particular embodiments, a formulation of the present disclosure includes from about 0.05 to about 10 mg protamine/mg conjugate, for example, from about 0.2 to about 10 mg protamine/mg conjugate, e.g., about 1 to about 5 mg protamine/mg conjugate.

In particular embodiments, a formulation of the present disclosure includes from about 0.006 to about 0.5 mg zinc/mg conjugate, for example, from about 0.05 to about 0.5 mg zinc/mg conjugate, e.g., about 0.1 to about 0.25 mg zinc/mg conjugate.

In particular embodiments, a formulation of the present disclosure includes protamine and zinc in a ratio (w/w) in the range of about 100:1 to about 5:1, for example, from about 50:1 to about 5:1, e.g., about 40:1 to about 10:1. In particular embodiments, a PZI formulation of the present disclosure includes protamine and zinc in a ratio (w/w) in the range of about 20:1 to about 5:1, for example, about 20:1 to about 10:1, about 20:1 to about 15:1, about 15:1 to about 5:1, about 10:1 to about 5:1, about 10:1 to about 15:1.

One or more of the following components may be included in the PZI formulation: an antimicrobial preservative, an isotonic agent, and/or an unconjugated insulin molecule.

In particular embodiments, a formulation of the present disclosure includes an antimicrobial preservative (e.g., m-cresol, phenol, methylparaben, or propylparaben). In particular embodiments, the antimicrobial preservative is m-cresol. For example, in particular embodiments, a formulation may include from about 0.1 to about 1.0% v/v m-cresol. For example, from about 0.1 to about 0.5% v/v m-cresol, e.g., about 0.15 to about 0.35% v/v m-cresol.

In particular embodiments, a formulation of the present disclosure includes a polyol as isotonic agent (e.g., mannitol, propylene glycol or glycerol). In particular embodiments the isotonic agent is glycerol. In particular embodiments, the isotonic agent is a salt, e.g., NaCl. For example, a formulation may comprise from about 0.05 to about 0.5M NaCl, e.g., from about 0.05 to about 0.25M NaCl or from about 0.1 to about 0.2M NaCl.

In particular embodiments, a formulation of the present disclosure includes an amount of unconjugated insulin molecule. In particular embodiments, a formulation includes a molar ratio of conjugated insulin molecule to unconjugated insulin molecule in the range of about 100:1 to 1:1, e.g., about 50:1 to 2:1, or about 25:1 to 2:1.

The present disclosure also encompasses the use of standard sustained (also called extended) release formulations that are well known in the art of small molecule formulation (e.g., see Remington's Pharmaceutical Sciences, 19th ed., Mack Publishing Co., Easton, Pa., 1995). The present disclosure also encompasses the use of devices that rely on pumps or hindered diffusion to deliver a conjugate on a gradual basis. In particular embodiments, a long-acting formulation may (additionally or alternatively) be provided by using a modified insulin molecule. For example, one could use insulin glargine (LANTUS®) or insulin detemir (LEVEMIR®) instead of wild-type human insulin in preparing the conjugate. Insulin glargine is an exemplary long-acting insulin analog in which Asn at position A21 of the A-chain has been replaced by glycine and two arginine residues are at the C-terminus of the B-chain. The effect of these changes is to shift the isoelectric point, producing an insulin that is insoluble at physiological pH but is soluble at pH 4. Insulin detemir is another long-acting insulin analog in which Thr at position B30 of the B-chain has been deleted and a C14 fatty acid chain has been attached to the Lys at position B29.

### Uses of Conjugates

In another aspect, the present disclosure provides methods of using the insulin conjugates. In general, the insulin conjugates can be used to controllably provide insulin to an individual in need in response to a saccharide (e.g., glucose or an exogenous saccharide such as mannose, alpha-methyl mannose, L-fucose, etc.). The disclosure encompasses treating diabetes by administering an insulin conjugate of the present disclosure. Although the insulin conjugates can be used to treat any patient (e.g., dogs, cats, cows, horses, sheep, pigs, mice, etc.), they are preferably used in the treatment of humans. An insulin conjugate may be administered to a patient by any route. In general, the present disclosure encompasses administration by oral, intravenous, intramuscular, intra-arterial, subcutaneous, intraventricular, transdermal, rectal, intravaginal, intraperitoneal, topical (as by powders, ointments, or drops), buccal, or as an oral or nasal spray or aerosol. General considerations in the formulation and manufacture of pharmaceutical compositions for these different routes may be found, for example, in Remington's Pharmaceutical Sciences, 19th ed., Mack Publishing Co., Easton, Pa., 1995. In various embodiments, the conjugate may be administered subcutaneously, e.g., by injection. The insulin conjugate may be dissolved in a carrier for ease of delivery. For example, the carrier can be an aqueous solution including, but not limited to, sterile water, saline or buffered saline.

In general, a therapeutically effective amount of the insulin conjugate will be administered. The term “therapeutically effective amount” means a sufficient amount of the insulin conjugate to treat diabetes at a reasonable benefit/risk ratio, which involves a balancing of the efficacy and toxicity of the insulin conjugate. In various embodiments, the average daily dose of insulin is in the range of 10 to 200 U, e.g., 25 to 100 U (where 1 Unit of insulin is ˜0.04 mg). In particular embodiments, an amount of conjugate with these insulin doses is administered on a daily basis. In particular embodiments, an amount of conjugate with 5 to 10 times these insulin doses is administered on a weekly basis. In particular embodiments, an amount of conjugate with 10 to 20 times these insulin doses is administered on a bi-weekly basis. In particular embodiments, an amount of conjugate with 20 to 40 times these insulin doses is administered on a monthly basis.

In particular embodiments, a conjugate of the present disclosure may be used to treat hyperglycemia in a patient (e.g., a mammalian or human patient). In particular embodiments, the patient is diabetic. However, the present methods are not limited to treating diabetic patients. For example, in particular embodiments, a conjugate may be used to treat hyperglycemia in a patient with an infection associated with impaired glycemic control. In particular embodiments, a conjugate may be used to treat diabetes.

In particular embodiments, when an insulin conjugate or formulation of the present disclosure is administered to a patient (e.g., a mammalian patient), it induces less hypoglycemia than an unconjugated version of the insulin molecule. In particular embodiments, a formulation of the present disclosure induces a lower HbAl c value in a patient (e.g., a mammalian or human patient) than a formulation comprising an unconjugated version of the insulin molecule. In particular embodiments, the formulation leads to an HbA1c value that is at least 10% lower (e.g., at least 20% lower, at least 30% lower, at least 40% lower, at least 50% lower) than a formulation comprising an unconjugated version of the insulin molecule. In particular embodiments, the formulation leads to an HbA1c value of less than 7%, e.g., in the range of about 4 to about 6%. In particular embodiments, a formulation comprising an unconjugated version of the insulin molecule leads to an HbAl c value in excess of 7%, e.g., about 8 to about 12%.

### Exogenous Trigger

As mentioned previously, the methods, conjugates and compositions that are described herein are not limited to glucose responsive-conjugates. As demonstrated in the Examples, several exemplary insulin conjugates were also responsive to exogenous saccharides such as alpha-methyl mannose. It will therefore be appreciated that in particular embodiments an insulin conjugate may be triggered by exogenous administration of a saccharide other than glucose such as alpha-methyl mannose or any other saccharide that can alter the PK or PD properties of the conjugate.

Once a conjugate has been administered as described above (e.g., as a sustained release formulation), it can be triggered by administration of a suitable exogenous saccharide. In a particular embodiment, a triggering amount of the exogenous saccharide is administered. As used herein, a “triggering amount” of exogenous saccharide is an amount sufficient to cause a change in at least one PK and/or PD property of the conjugate (e.g., Cₘₐₓ, AUC, half-life, etc. as discussed previously). It is to be understood that any of the aforementioned methods of administration for the conjugate apply equally to the exogenous saccharide. It is also to be understood that the methods of administration for the conjugate and exogenous saccharide may be the same or different. In various embodiments, the methods of administration are different (e.g., for purposes of illustration the conjugate may be administered by subcutaneous injection on a weekly basis while the exogenous saccharide is administered orally on a daily basis). The oral administration of an exogenous saccharide is of particular value because it facilitates patient compliance. In general, it will be appreciated that the PK and PD properties of the conjugate will be related to the PK profile of the exogenous saccharide. Thus, the conjugate PK and PD properties can be tailored by controlling the PK profile of the exogenous saccharide. As is well known in the art, the PK profile of the exogenous saccharide can be tailored based on the dose, route, frequency and formulation used. For example, if a short and intense activation of the conjugate is desired then an oral immediate release formulation might be used. In contrast, if a longer less intense activation of conjugate is desired then an oral extended release formulation might be used instead. General considerations in the formulation and manufacture of immediate and extended release formulation may be found, for example, in Remington's Pharmaceutical Sciences, 19th ed., Mack Publishing Co., Easton, Pa., 1995.

It will also be appreciated that the relative frequency of administration of a conjugate of the present disclosure and an exogenous saccharide may be the same or different. In particular embodiments, the exogenous saccharide is administered more frequently than the conjugate. For example, in particular embodiment, the conjugate may be administered daily while the exogenous saccharide is administered more than once a day. In particular embodiment, the conjugate may be administered twice weekly, weekly, biweekly or monthly while the exogenous saccharide is administered daily. In particular embodiments, the conjugate is administered monthly and the exogenous saccharide is administered twice weekly, weekly, or biweekly. Other variations on these schemes will be recognized by those skilled in the art and will vary depending on the nature of the conjugate and formulation used.

The following examples are intended to promote a further understanding of the present invention.

### EXAMPLES

### General Procedures

All chemicals were purchased from commercial sources, unless otherwise noted. Reactions sensitive to moisture or air were performed under nitrogen or argon using anhydrous solvents and reagents. The progress of reactions was monitored by analytical thin layer chromatography (TLC), high performance liquid chromatography-mass spectrometry (HPLC-MS), or ultra performance liquid chromatography-mass spectrometry (UPLC-MS). TLC was performed on E. Merck TLC plates precoated with silica gel 60E-254, layer thickness 0.25 mm. The plates were visualized using 254 nm UV and/or by exposure to cerium ammonium molybdate (CAM) or p-anisaldehyde staining solutions followed by charring. High performance liquid chromatography (HPLC) was conducted on a Waters Acquity™ UPLC® using BEH C18, 1.7 μm, 1.0×50 mm column with gradient 10:90-99:1 v/v CH₃CN/H₂O+v 0.05% TFA over 2.0 min; flow rate 0.3 mL/min, UV range 215 nm (LC-MS Method A). Mass analysis was performed on a Waters Micromass® ZQ™ with electrospray ionization in positive ion detection mode and the scan range of the mass-to-charge ratio was either 170-900 or 500-1500. Ultra performance liquid chromatography (UPLC) was performed on a Waters Acquity™ UPLC® system using the following methods:

UPLC-MS Method A: Waters Acquity™ UPLC® BEH C18 1.7 μm 2.1×100 mm column with gradient 10:90-70:30 v/v CH₃CN/H₂O+v 0.1% TFA over 4.0 min and 70:30-95:5 v/v CH₃CN/H₂O+ v 0.1% TFA over 0.4 min; flow rate 0.3 mL/min, UV wavelength 200-300 nm.

UPLC-MS Method B: Waters Acquity™ UPLC® BEH C18 1.7 μm 2.1×100 mm column with gradient 60:40-100:0 v/v CH₃CN/H₂O+ v 0.1% TFA over 4.0 min and 100:0-95:5 v/v CH₃CN/H₂O+ v 0.1% TFA over 0.4 min; flow rate 0.3 mL/min, UV wavelength 200-300 nm.

UPLC-MS Method C: Waters Acquity™ UPLC® HSS T3 1.7 μm 2.1×100 mm column with gradient 0:100-40:60 v/v CH₃CN/H₂O+ v 0.05% TFA over 8.0 min and 40:60-10:90 v/v CH₃CN/H₂O+ v 0.05% TFA over 2.0 min; flow rate 0.3 mL/min, UV wavelength 200-300 nm.

UPLC-MS Method D: Waters Acquity™ UPLC® BEH C18 1.7 μm 2.1×100 mm column with gradient 0:100-60:40 v/v CH₃CN/H₂O+ v 0.1% TFA over 8.0 min and 60:40-90:10 v/v CH₃CN/H₂O+ v 0.1% TFA over 3.0 min and hold at 100:0 v/v CH₃CN/H₂O+ v 0.1% TFA for 2 min; flow rate 0.3 mL/min, UV wavelength 200-300 nm.

UPLC-MS Method E: Waters Acquity™ UPLC® BEH C8 1.7 μm 2.1×100 mm column with gradient 10:90-55:45 v/v CH₃CN/H₂O+ v 0.1% TFA over 4.2 min and 100: 0-95:5 v/v CH₃CN/H₂O+ v 0.1% TFA over 0.4 min; flow rate 0.3 mL/min, UV wavelength 200-300 nm.

UPLC-MS Method F: Waters Acquity™ UPLC® BEH C8 1.7 μm 2.1×100 mm column with gradient 10:90-90:10 v/v CH₃CN/H₂O+ v 0.1% TFA over 4.2 min and 90:10-95:5 v/v CH₃CN/H₂O+ v 0.1% TFA over 0.4 min; flow rate 0.3 mL/min, UV wavelength 200-300 nm.

UPLC-MS Method G: Waters Acquity™ UPLC® BEH300 C4 1.7 μm 2.1×100 mm column with gradient 10:90-90:10 v/v CH₃CN/H₂O+ v 0.1% TFA over 4.0 min and 90:10-95:5 v/v CH₃CN/H₂O+ v 0.1% TFA over 0.4 min; flow rate 0.3 mL/min, UV wavelength 200-300 nm.

Mass analysis was performed on a Waters Micromass® LCT Premier™ XE with electrospray ionization in positive ion detection mode and the scan range of the mass-to-charge ratio was 300-2000. The identification of the produced insulin conjugates was confirmed by comparing the theoretical molecular weight to the experimental value that was measured using UPLC-MS. For the determination of the position of sugar modification(s), specifically, insulin conjugates were subjected to dithiothreitol (DTT) treatment (for a/b chain) or endoproteinsase Glu-C digestion (with reduction and alkylation), and then the resulting peptides were analyzed by LC-MS. Based on the measured masses, the sugar positions were deduced.

Flash chromatography was performed using either a Biotage Flash Chromatography apparatus (Dyax Corp.) or a CombiFlash® Rf instrument (TELEDYNE ISCO). Normal-phase chromatography was carried out on silica gel (20-70 μm, 60 Å pore size) in pre-packed cartridges of the size noted. Concentration of organic solutions was carried out on a rotary evaporator under reduced pressure. Reverse-phase chromatography was carried out on C18-bonded silica gel (20-60 μm, 60-100 Å pore size) in pre-packed cartridges of the size noted. Preparative scale HPLC was performed on Gilson GX-281 Liquid Handler powered by Gilson 333-334 binary system using Waters Delta Pak C4 15 μm, 300 Å, 50×250 mm column or Kromasil® C8 10 μm, 100 Å, 50×250 mm column, flow rate 85 mL/min, with gradient noted. Ion exchange chromatography was carried out on Gilson 215 Liquid Handler powered by Gilson 332 binary system using PolyLC PolySULFOEthyl A 9.4×250 mm column, with gradient 5-25% Mobile Phase B in Mobile Phase A (Mobile Phase A: 0.1% (v/v) H₃PO₄/25% ACN in water, mobile phase B: 0.1% (v/v) H₃PO₄/25%ACN/0.5M NaCl in water, over 30 min, flow rate 15 mL/min). Concentration and diafiltration of aqueous solutions or HPLC fractions were carried out using Amicon Ultra-15 Centrifugal Filter Units (Millipore) with 10K MWCO, unless noted otherwise, on a Hettich Rotina 380R Benchtop Centrifuge at 3500 RPM and 4° C., or freeze-dried on a VirTis Freezemobile Freeze Dryer (SP Scientific).

¹H-NMR spectra were acquired at 500 MHz (or otherwise specified) spectrometers in deuterated solvents noted. Chemical shifts were reported in parts per million (ppm). Tetramethylsilane (TMS) or residual proton peak of deuterated solvents was used as an internal reference. Coupling constants (J) were reported in hertz (Hz).

Abbreviations: acetic acid (AcOH), acetonitrile (ACN or MeCN), aqueous (aq), tert-butoxycarbonyl protecting group (Boc), O-(7-azabenzotriazol-1-yl)-N,N,N′,N′-tetramethyl uronium hexafluorophosphate) (HATU), column volume (CV), N,N′-Dicyclohexylcarbodiimide (DCC), dichloromethane (DCM), deethyl amine (DEA), diethyl ether (ether or Et₂O), N,N-diisopropylethylamine or Fllinig's base (DIPEA), N,N-dimethylacetamide (DMA), (4-dimethyl amino)pyridine (DMAP), N,N-dimethylformamide (DMF), dimethylsulfoxide (DMSO), ethyl acetate (EtOAc), N-(3-dimethylaminopropyl)-N′-ethylcarbodiimide hydrochloride (EDC), gram(s) (g), 1-hydroxybenzotriazole hydrate (HOBt), hour(s) (h or hr), isopropyl alcohol (IPA), liquid chromatography-mass spectrometry (LC-MS), mass spectrum (ms or MS), N-methyl morpholine (NMM), microliter(s) (pL), milligram(s) (mg), milliliter(s) (mL), millimole (mmol), minute(s) (min), tert-butyl ester (OtBu), pentafluorphenol-tetramethyluronium hexafluoro phosphate (PFTU), petroleum ether (PE), silicon dioxide (SiO₂), retention time (t_{R}), room temperature (rt), saturated (sat.), sat. aq. sodium chloride solution (brine), triethylamine (TEA), trifluoroacetic acid (TFA), trifluoroacetic anhydride (TFAA), tetrahydrofuran (THF), N,N,N′,N′-tetramethyl-O—(N-succinimidyOuronium tetrafluoroborate (TSTU), trimethylsilyl trifluoromethane sulfonate (TMSOTf), 2,3,4-O-trimethyl silyl (per-TMS), trimethylsilyl iodide (TMS-I), 9-fluorenylmethyl N-succinimidyl carbonate (Fmoc-OSU), and weight (wt).

### Example 1

### 6-[(2,5-Dioxopyrrolidin-1-yl)oxy]-N-(2-{[α-D-mannopyranosyl-(1→3)-[α-D-mannopyranosyl-(1→6)]-β-D-glucopyranosyl]oxy}ethyl)-6-oxohexanamide (ML-1)

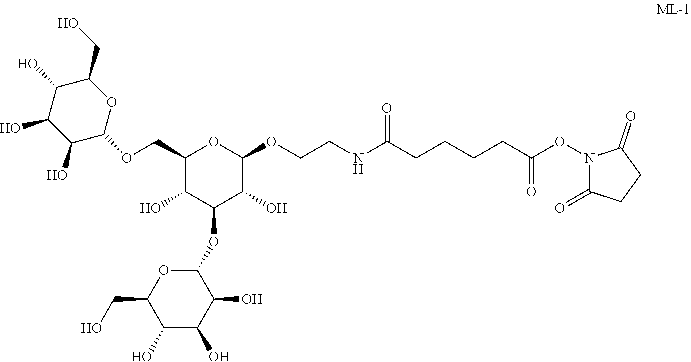

### Step 1: benzyl 6-[(2,5-dioxopyrrolidin-1-yl)oxy]-6-oxohexanoate

To a solution of 6-(benzyloxy)-6-oxohexanoic acid (3.3 g, 13.97 mmol) in DMF (50 mL) at 0° C. was added TSTU (4.3 g, 14.28 mmol) and DIPEA (2.5 mL, 14.31 mmol). After stirring at 0° C. for 1 h, the reaction mixture was partitioned between Et₂O and water. The organic layer was separated, and the aqueous layer was further extracted with Et₂O (2×150 mL). The combined organic phase was washed with brine, dried over Na₂SO₄, filtered and concentrated to afford the title compound. UPLC Method B: calculated for C₁₇H₁₉NO₆ 333.12, observed m/e: 334.10 [M+1]; t_{R}=3.75 min. ¹H NMR (CDCl₃) δ 7.40-7.30 (5H, m), 5.10 (2H, s), 2.80 (4H, s), 2.62-2.58 (2H, m), 2.41-2.37 (2H, m), 1.80-1.72 (4H, m).

### Step 2: benzyl 6-({2-[(α-D-mannopyranosyl-(1→3)-[α-D-mannopyranosyl-(1→6)]-β-D-glucopyranosyl)oxy]ethyl}amino)-6-oxohexanoate

To a solution of 2-aminoethyl α-D-mannopyranosyl-(1→3)-[α-D-mannopyranosyl-(1→6)]-β-D-glucopyranoside (52 mg, 0.095 mmol) in DMF (2 mL) at 0° C. was added benzyl 6-[(2,5-dioxopyrrolidin-1-yl)oxy]-6-oxohexanoate (36.5 mg, 0.109 mmol) and TEA (0.02 mL, 0.143 mmol). After stirring at 0° C. for 1 h, the reaction mixture was concentrated, and the residue was purified by flash chromatography on C18 reverse silica gel column, eluting with 5-60% ACN in H₂O to give the title compound. UPLC Method B: calculated for C₃₃H₅₁NO₁₉ 765.31, observed m/e=766.40 [M+1]; t_{R}=2.52 min.

### Step 3: 6-({2-[(α-D-mannopyranosyl-(1→3)-[α-D-mannopyranosyl-(1→6)]-β-D-glucopyranosyl)oxy]ethyl}amino)-6-oxohexanoic acid

A mixture of benzyl 6-({2-[(α-D-mannopyranosyl-(1→3)-[α-D-mannopyranosyl-(1→6)]-β-D-glucopyranosyl)oxy]ethyl}amino)-6-oxohexanoate (61 mg, 0.080 mmol) and Pd/C (5 mg, 4.7 μmol) in water (2 mL) was allowed to stir under H₂ at rt for 24 h. The catalyst was filtered off and washed with H₂O (3×10 mL). The filtrate was concentrated to give the title compound. UPLC Method B: calculated for C₂₆H₄₅NO₁₉ 675.26, observed m/e: 676.36 [M+1]; t_{R}=0.85 min.

### Step 4: 6-[(2,5-Dioxopyrrolidin-1-yl)oxy]-N-(2-{[α-D-mannopyranosyl-(1→3)-[α-D-mannopyranosyl-(1→6)]-β-D-glucopyranosyl]oxy}ethyl)-6-oxohexanamide

To a solution of 6-({2-[(α-D-mannopyranosyl-(1→3)-[α-D-mannopyranosyl-(1→6)]-β-D-glucopyranosyl)oxy]ethyl}amino)-6-oxohexanoic acid (53.8 mg, 0.080 mmol) in DMF (1 mL) at 0° C. was added TSTU (25 mg, 0.083 mmol) and DIPEA (0.02 mL, 0.115 mmol). After stirring at 0° C. for 1 h, the reaction was quenched by the addition of TFA (0.012 mL, 0.159 mmol). The residue was transferred dropwise, via auto pipette, to a tube containing anhydrous ACN (40 mL). The precipitate was collected through centrifugation (3000 rpm, 15 min, at 4° C.), washed with anhydrous ACN (1 mL) and dried to yield the title compound. UPLC Method B: calculated for C₃₀H₄₈N₂O₂₁ 772.27, observed m/e: 773.39 [M+1]; t_{R}=0.95 min.

### Example 2

### 6-[(2,5-Dioxopyrrolidin-1-yl)oxy]-N-(2-{[α-D-mannopyranosyl-(1→3)-[α-D-mannopyranosyl-(1→6)]-β-D-glucopyranosyl]oxy}ethyl)-6-oxo-octanamide (ML-2)

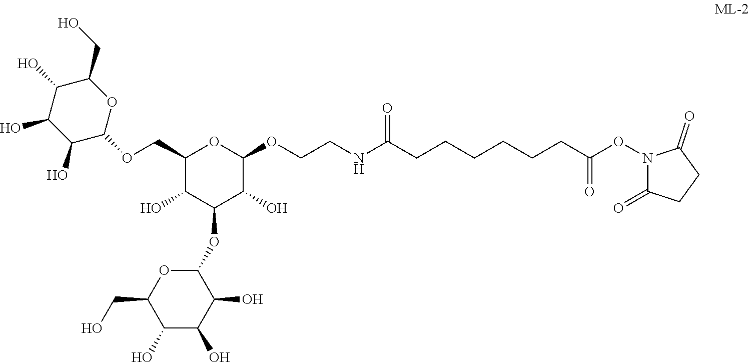

### Step 1: 8-(benzyloxy)-8-oxooctanoic acid

To a solution of octanedioic acid (4.0 g, 22.96 mmol) and p-toluenesulfonic acid (200 mg, 1.051 mmol) in toluene (12 mL) was added benzyl alcohol (2.6 mL, 25.01 mmol). The resultant mixture was heated to reflux, stirred at reflux for 5 h, cooled to rt, and concentrated under reduced pressure. The residue was purified by flash chromatography on a silica gel column (80 g), eluting with 0-100% EtOAc in hexanes to give the title compound. UPLC Method B: calculated for C₁₇H₂₄NO₄ 292.17, observed m/e: 293.1 [M+1]; t_{R}=1.22/2.0 min. ¹H NMR (CDCl₃) δ 7.38-7.32 (s; 5H); 5.11 (s; 2H); 2.35 (dt; J=9.52; 7.48 Hz; 4H); 1.61-1.66 (m; 4H); 1.33-1.35 (m; 4H).

### Step 2: benzyl 8-({2-[(α-D-mannopyranosyl-(1→3)-[α-D-mannopyranosyl-(1→6)]-β-D-glucopyranosyl)oxy]ethyl}amino)-8-oxo-octanoate

To a solution of 2-aminoethyl α-D-mannopyranosyl-(1→3)-[α-D-mannopyranosyl-(1→6)]-β-D-glucopyranoside (500 mg, 0.913 mmol) in DMF (2 mL) at rt was added 8-(benzyloxy)-8-oxooctanoic acid (260 mg, 0.984 mmol), HOBT (170 mg, 1.110 mmol), EDC (400 mg, 2.087 mmol). After stirring at rt for 24 h, the reaction mixture was concentrated, and the residue was purified by flash chromatography on C18 reverse silica gel column (50g), eluting with 5-60% ACN in H₂O to give the title compound. UPLC Method B: calculated for C₃₃H₅₅NO₁₉ 793.34, observed m/e=794.48 [M+1]; t_{R}=3.18 min. ¹H NMR (D₂O) δ 7.41 (t; J=5.75 Hz; 5H); 5.16 (d; J=1.76 Hz; 1H); 5.14 (s; 2H); 4.83 (d; J=1.76 Hz; 1H); 4.43 (d; J=8.01 Hz; 1H); 4.01 (dd; J=3.35; 1.79 Hz; 1H); 3.89-3.96 (m; 4H); 3.67-3.85 (m; 9H); 3.57-3.62 (m; 5H); 3.40-3.44 (m; 1H); 3.33-3.36 (m; 1H); 3.27-3.32 (m; 1H); 2.39 (t; J=7.27 Hz; 2H); 2.17 (t; J=7.34 Hz; 2H); 1.58 (t; J=7.21 Hz; 2H); 1.51 (t; J=6.82 Hz; 2H); 1.24 (t; J=5.11 Hz; 4H).

### Step 3: 8-({2-[(α-D-mannopyranosyl-(1→3)-[α-D-mannopyranosyl-(1→6)]-β-D-glucopyranosyl)oxy]ethyl}amino)-8-oxooctanoic acid

A mixture of benzyl 8-({2-[(α-D-mannopyranosyl-(1→3)-[α-D-mannopyranosyl-(1→6)]-β-D-glucopyranosyl)oxy]ethyl}amino)-8-oxooctanoate (340 mg, 0.428 mmol) and Pd/C (25 mg, 0.023 mmol) in water (6 mL) was allowed to stir under H₂ at rt for 24 h. The catalyst was filtered off and washed with H₂O (3×10 mL). The filtrate was concentrated to give the title compound. UPLC Method B: calculated for C₂₈H₄₉NO₀₂₁ 735.28, observed m/e: 736.40 [M+1]; t_{R}=1.09 min. ¹H NMR (D₂O) δ 5.18 (d; J=1.77 Hz; 1H); 4.86 (d; J=1.77 Hz; 1H); 4.46 (d; J=8.01 Hz; 1H); 4.03 (dd; J=3.36; 1.79 Hz; 1H); 3.89-3.99 (m; 4H); 3.69-3.85 (m; 9H); 3.60-3.64 (m; 5H); 3.45 (ddd; J=14.46; 6.75; 4.11 Hz; 1H); 3.29-3.38 (m; 2H); 2.33 (t; J=7.42 Hz; 2H); 2.23 (t; J=7.33 Hz; 2H); 1.54-1.60 (m; 4H); 1.29-1.31 (m; 4H).

### Step 4: 8-[(2,5-Dioxopyrrolidin-1-yl)oxy]-N-(2-{[α-D-mannopyranosyl-(1→3)-[β-D-mannopyranosyl-(1→6)]-α-D-glucopyranosyl]oxy}ethyl)-8-oxo-octanamide

To a solution of 8-({2-[(α-D-mannopyranosyl-(1→3)-[α-D-mannopyranosyl-(1→6)]-β-D-glucopyranosyl)oxy]ethyl}amino)-8-oxo-octanoic acid (290 mg, 0.412 mmol) in DMF (3 mL) at 0° C. was added TSTU (130 mg, 0.433 mmol) and DIPEA (0.01 mL, 0.573 mmol). After stirring at 0° C. for 1 h, the reaction was quenched by the addition of TFA (554, 0.714 mmol). The residue was transferred dropwise, via autopipette, to a tube containing anhydrous EtOAc (45 mL). The precipitate was collected through centrifugation (3000 rpm, 15 min, at 4° C.), washed with anhydrous EtOAc (1 mL) and dried to yield the title compound. UPLC Method B: calculated for C₃₄H₅₅N₂O₂₁ 828.34, observed m/e: 829.21 [M+1]; t_{R}=0.57 min. ¹H NMR (D₂O) δ 5.17 (d; J=1.72 Hz; 1H); 4.85 (d; J=1.73 Hz; 1H); 4.45 (d; J=8.01 Hz; 1H); 4.01 (dd; J=3.34; 1.79 Hz; 1H); 3.90-3.96 (m; 4H); 3.67-3.86 (m; 9H); 3.58-3.62 (m; 5H); 3.41-3.45 (m; 1H); 3.28-3.37 (m; 2H); 2.91 (s; 4H); 2.69 (t; J=7.32 Hz; 2H); 2.23 (t; J=7.30 Hz; 2H); 1.70 (p; J=7.40 Hz; 2H); 1.55-1.61 (m; 2H); 1.30-1.40 (m; 4H).

### Example 3

### 6-[(2,5-Dioxopyrrolidin-1-yl)oxy]-N-(2-{[α-D-mannopyranosyl-(1→3)-[α-D-mannopyranosyl-(1→6)]-α-D-mannopyranosyl]oxy}ethyl)-6-oxohexanamide (ML-3)

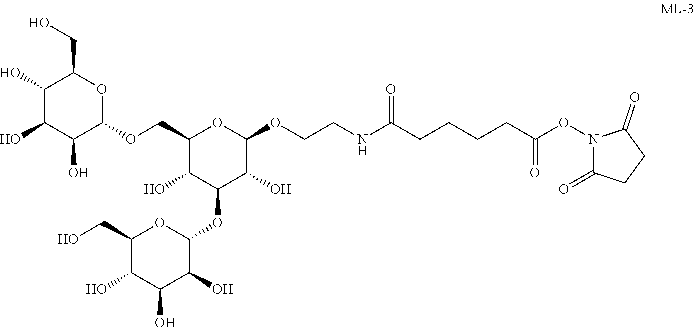 [SMILES: O=C(CCCCC(=O)ON1C(=O)CCC1=O)NCCO[C@@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@H]1O]

### Step 1: benzyl 6-[(2,5-dioxopyrrolidin-1-yl)oxy]-6-oxohexanoate

To a solution of 6-(benzyloxy)-6-oxohexanoic acid (3.3 g, 13.97 mmol) in DMF (50 mL) at 0° C. was added TSTU (4.3 g, 14.28 mmol) and DIPEA (2.5 mL, 14.31 mmol). After stirring at 0° C. for 1 h, the reaction mixture was partitioned between Et₂O and water. The organic layer was separated, and the aqueous layer was further extracted with Et₂O (2×150 mL). The combined organic phase was washed with brine, dried over Na₂SO₄, filtered and concentrated to afford the title compound. UPLC Method B: calculated for C₁₇H₁₉NO₆ 333.12, observed m/e: 334.10 [M+1]; t_{R}=3.75 min. ¹H NMR (CDCl₃) δ 7.40-7.30 (5H, m), 5.10 (2H, s), 2.80 (4H, s), 2.62-2.58 2H, m), 2.41-2.37 (2H, m), 1.80-1.72 (4H, m).

### Step 2: benzyl 6-({2-[(α-D-mannopyranosyl-(1→3)-[α-D-mannopyranosyl-(1→6)]-α-D-mannopyranosyl)oxy]ethyl}amino)-6-oxohexanoate

To a solution of 2-aminoethyl α-D-mannopyranosyl-(1→3)-[α-D-mannopyranosyl-(1→6)]-α-D-mannopyranoside (1.23 g, 2.247 mmol, WO 2010/088294 A1) in DMF (20 mL) at 0° C. was added benzyl 6-[(2,5-dioxopyrrolidin-1-yl)oxy]-6-oxohexanoate (1.02 g, 3.06 mmol) and TEA (0.5 mL, 3.59 mmol). After stirring at 0° C. for 1 h, the reaction mixture was concentrated, and the residue was purified by flash chromatography on C18 reverse silica gel column, eluting with 0-40% ACN in H₂O to give the title compound. UPLC Method B: calculated for C₃₃H₅₁NO₁₉ 765.31, observed m/e=766.26 [M+1]; t_{R}=4.04 min. ¹H NMR (D₂O) δ 7.43-7.37 (5H, m), 5.14 (2H, s), 5.07-5.06 (1H, m), 4.82-4.81 (1H, m), 4.77-4.76 (1H, m), 4.06-4.01 (2H, m), 3.96-3.92 (2H, m), 3.87-3.81 (5H, m), 3.79-3.77 (1H, m), 3.74-3.67 (5H, m), 3.65-3.60 (4H, m), 3.53-3.49 (1H, m), 3.37-3.35 (2H, m), 2.43-2.40 (2H, m), 2.22-2.19 (2H, m), 1.62-1.52 (4H, m).

### Step 3: 6-({2-[(α-D-mannopyranosyl-(1→3)-[α-D-mannopyranosyl-(1→6)]-α-D-mannopyranosyl)oxy]ethyl}amino)-6-oxohexanoic acid

A mixture of benzyl 6-({2-[(α-D-mannopyranosyl-(1→3)-[α-D-mannopyranosyl-(1→6)]-α-D-mannopyranosyl)oxy]ethyl}amino)-6-oxohexanoate (1.15 g, 1.502 mmol) and Pd/C (80 mg, 0.075 mmol) in water (10 mL) was allowed to stir under H₂ at rt for 16 h. The catalyst was filtered off and washed with H₂O (3×10 mL). The filtrate was concentrated to give the title compound. UPLC Method B: calculated for C₂₆H₄₅NO₁₉ 675.26, observed m/e: 676.21 [M+1]; t_{R}=3.50 min.

### Step 4: 6-[(2,5-Dioxopyrrolidin-1-yl)oxy]-N-(2-{[α-D-mannopyranosyl-(1→3)-[α-D-mannopyranosyl-(1→6]-β-D-mannopyranosyl]oxy}ethyl)-6-oxohexanamide

To a solution of 6-({2-[(α-D-mannopyranosyl-(1→3)-1α-D-mannopyranosyl-(1→6)1-α-D-mannopyranosyl)oxy]ethyl}amino)-6-oxohexanoic acid (1.55 g, 2.294 mmol) in DMF (22 mL) at 0° C. was added TSTU (760 mg, 2.52 mmol) and DIPEA (0.52 mL, 2.98 mmol). After stirring at 0° C. for 1 h, the reaction was quenched by the addition of TFA (371 μL, 4.82 mmol), and the resulting mixture was concentrated down to about 3 mL. The residue was transferred dropwise, via autopipette, to a tube containing anhydrous ACN (45 mL). The precipitate was collected through centrifugation (3000 rpm, 15 min, at 4° C.), washed with anhydrous ACN (1 mL) and dried to yield the title compound. UPLC Method B: calculated for C₃₀H₄₈N₂O₂₁ 772.27, observed m/e: 773.23 [M+1]; t_{R}=3.65 min. ¹H NMR (D₂O) δ 5.07-5.06 (1H, m), 4.84-4.83 (1H, m), 4.79-4.78 (1H, m), 4.06-4.01 (2H, m), 3.96-3.93 (2H, m), 3.87-3.83 (5H, m), 3.80-3.78 (1H, m), 3.75-3.69 (5H, m), 3.67-3.61 (4H, m), 3.57-3.52 (1H, m), 3.41-3.38 (2H, m), 2.91 (4H, s), 2.75-2.71 (2H, m), 2.29-2.25 (2H, m), 1.75-1.58 (4H, m).

### Example 4

### 2-[(2,5-Dioxopyrrolidin-1-yl)oxy]-N-(2-{[α-D-mannopyranosyl-(1→3)-[α-D-mannopyranosyl-(1→6)]-α-D-mannopyranosyl]oxy}ethyl)-2-oxoethoxy-acetamide (ML-4)

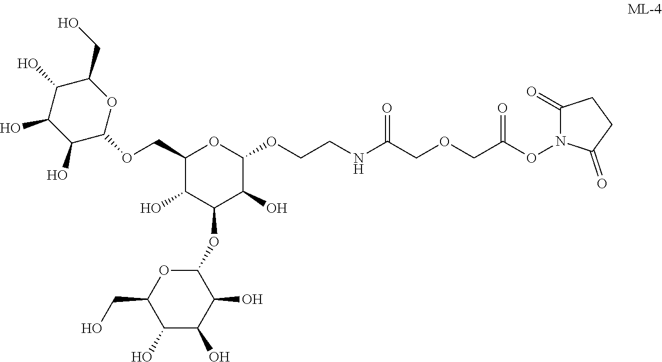

### Step 1: 2-({2-[(α-D-mannopyranosyl-(1→3)-[α-D-mannopyranosyl-(1→6)]-α-D-mannopyranosyl)oxy]ethyl}amino)-2-oxoethoxy-acetic acid

To a solution of 2-aminoethyl α-D-mannopyranosyl-(1→3)-[α-D-mannopyranosyl-(1→6)]-α-D-mannopyranoside (100 mg, 0.183 mmol, WO 2010/088294 A1) in DMF (0.8 mL) at rt was added 1,4-dioxane-2,6-dione (22 mg, 0.190 mmol). After stirring at rt for 24 h, the reaction mixture was concentrated, and the residue was purified by flash chromatography on C18 reverse silica gel column, eluting with 5-60% ACN in H₂O to give the title compound. UPLC Method B: calculated for C₂₄H₄₁NO₂₀ 663.22, observed m/e=664.35; t_{R}=0.84 min ¹H NMR (500 MHz, D₂O): δ 5.08 (d; J=1.72 Hz; 1H); 4.86 (d; J=1.73 Hz; 1H); 4.81 (d; J=1.72 Hz; 1H); 4.18-4.20 (m; 2H); 4.12 (s; 2H); 4.02-4.07 (m; 2H); 3.94-3.97 (m; 2H); 3.71-3.87 (m; 11H); 3.58-3.68 (m; 5H); 3.43-3.52 (m; 2H).

### Step 2: 2-[(2,5-Dioxopyrrolidin-1-yl)oxy]-N-(2-{[α-D-mannopyranosyl-(1→3)-[α-D-mannopyranosyl-(1→6)]-α-D-mannopyranosyl]oxy}ethyl)-2-oxoethoxy-acetamide

To a solution of 2-({2-[(α-D-mannopyranosyl-(1→3)-[α-D-mannopyranosyl-(1→6)]-α-D-mannopyranosyl)oxy]ethyl}amino)-2-oxoethoxy-acetic acid (70 mg, 0.105 mmol) in DMF (2 mL) at 0° C. was added TSTU (39 mg, 0.130 mmol) and DIPEA (18 μL, 0.103 mmol). After stirring at 0° C. for 1 h, the reaction was quenched by the addition of TFA (12 μL, 0.156 mmol). The residue was transferred dropwise, via autopipette, to a tube containing anhydrous EtOAc (45 mL). The precipitate was collected through centrifugation (3000 rpm, 15 min, at 4° C.), washed with anhydrous EtOAc (1 mL) and dried to yield the title compound. UPLC Method B: calculated for C₂₈H₄₄N₂O₂₂ 760.24, observed m/e: 761.29 [M+1]; t_{R}=1.07 min. ¹H NMR (D₂O) δ 4.99 (d; J=1.76 Hz; 1H); 4.78 (d; J=1.75 Hz; 1H); 4.73 (d; J=1.81 Hz; 1H); 4.12 (s; 2H); 4.04 (s; 2H); 3.94-3.99 (m; 2H); 3.86-3.89 (m; 2H); 3.63-3.79 (m; 11H); 3.50-3.60 (m; 5H); 3.35-3.42 (m; 2H); 2.68 (s; 4H).

### Example 5

### 2-[(2,5-Dioxopyrrolidin-1-yl)oxy]-N-(2-{[α-D-mannopyranosyl-(1→3)-[α-D-mannopyranosyl-(1→6)]-β-D-mannopyranosyl]oxy}ethyl)-2-oxoethoxy-acetamide (ML-5)

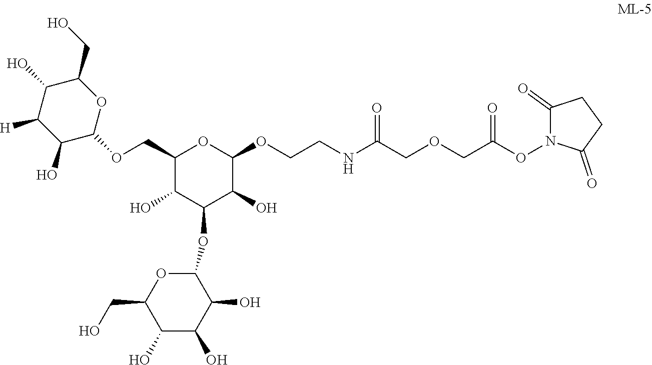

The title compound was prepared using procedures analogous to those described for ML-4 substituting 2-aminoethyl α-D-mannopyranosyl-(1→3)-[α-D-mannopyranosyl-(1→6)]-β-D-mannopyranoside for 2-aminoethyl α-D-mannopyranosyl-(1→3)-[α-D-mannopyranosyl-(1→6)]-α-D-mannopyranoside in Example 4, Step 1. UPLC Method B: calculated for C₂₈H₄₄N₂O₂₂ 760.24, observed m/e: 761.36 [M+1]; t_{R}=1.01 min.

### Example 6

### 4-[(2,5-Dioxopyrrolidin-1-yl)oxy]-N-(2-{[α-D-mannopyranosyl-(1→3)-[α-D-mannopyranosyl-(1→6)]-α-D-mannopyranosyl]oxy}ethyl)-4-oxo-butanamide (ML-6)

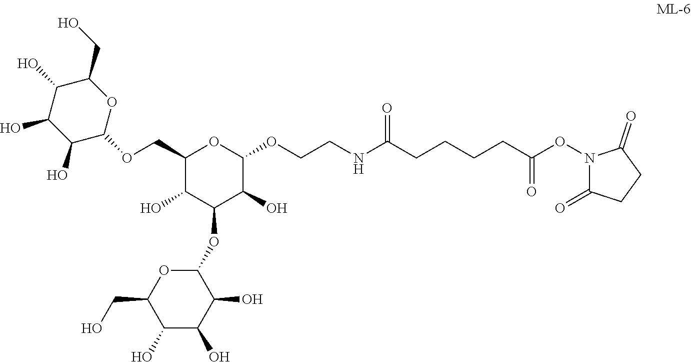

The title compound was prepared using procedures analogous to those described for ML-1 in Example 1, substituting 4-(benzyloxy)-4-oxobutanoic acid for 6-(benzyloxy)-6-oxohexanoic acid in Step 1, and substituting 2-aminoethyl α-D-mannopyranosyl-(1→3)-[α-D-mannopyranosyl-(1→6)]-α-D-mannopyranoside for 2-aminoethyl α-D-mannopyranosyl-(1→3)-[α-D-mannopyranosyl-(1→6)]-β-D-glucopyranoside in Step 2. UPLC Method B: calculated for C₂₈H₄₄N₂O₂₁ 744.24, observed m/e: 745.29 [M+1]; t_{R}=0.93 min.

### Example 7

### 4-[(2,5-Dioxopyrrolidin-1-yl)oxy]-N-(2-{[α-D-mannopyranosyl-(1→3)-[α-D-mannopyranosyl-(1→6)]-β-D-mannopyranosyl]oxy}ethyl)-4-oxo-butanamide (ML-7)

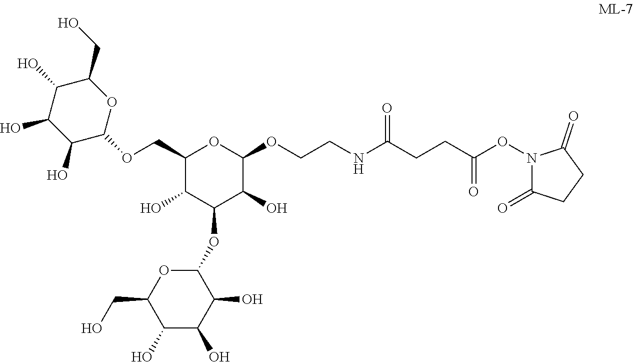

The title compound was prepared using procedures analogous to those described for ML-1 in Example 1, substituting 4-(benzyloxy)-4-oxobutanoic acid for 6-(benzyloxy)-6-oxohexanoic acid in Step 1, and substituting 2-aminoethyl α-D-mannopyranosyl-(1→3)-[α-D-mannopyranosyl-(1→6)]-β-D-mannopyranoside for 2-aminoethyl α-D-mannopyranosyl-(1→3)-[α-D-mannopyranosyl-(1→6)]-β-D-glucopyranoside in Step 2. UPLC Method B: calculated for C₂₈H₄₄N₂O₂₁ 744.24, observed m/e: 745.27 [M+1]; t_{R}=1.11 min.

### Example 8

### 5-[(2,5-Dioxopyrrolidin-1-yl)oxy]-N-(2-{[α-D-mannopyranosyl-(1→3)-[α-D-mannopyranosyl-(1→6)]-α-D-mannopyranosyl]oxy}ethyl)-5-oxo-pentanamide (ML-8)

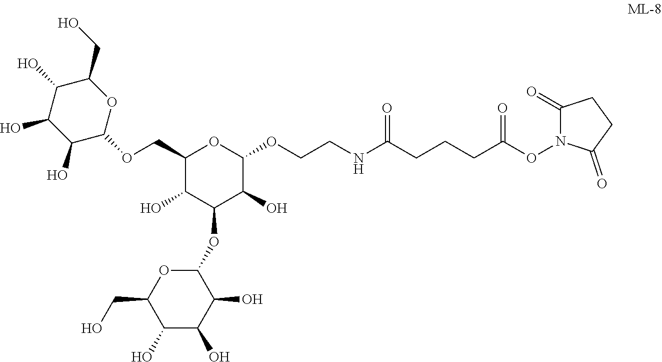

The title compound was prepared using procedures analogous to those described for ML-1 in Example 1, substituting 5-(benzyloxy)-5-oxopentanoic acid for 6-(benzyloxy)-6-oxohexanoic acid in Step 1, and substituting 2-aminoethyl α-D-mannopyranosyl-(1→3)-[α-D-mannopyranosyl-(1→6)]-α-D-mannopyranoside for 2-aminoethyl α-D-mannopyranosyl-(1→3)-[α-D-mannopyranosyl-(1→6)]-α-D-glucopyranoside in Step 2. UPLC Method B: calculated for C₂₉H₄₆N₂O₂₁ 758.26, observed m/e: 759.25 [M+1]; t_{R}=1.53 min.

### Example 9

### 5-[(2,5-Dioxopyrrolidin-1-yl)oxy]-N-(2-{[α-D-mannopyranosyl-(1→3)-[α-D-mannopyranosyl-(1→6)]-β-D-mannopyranosyl]oxy}ethyl)-5-oxo-pentanamide (ML-9)

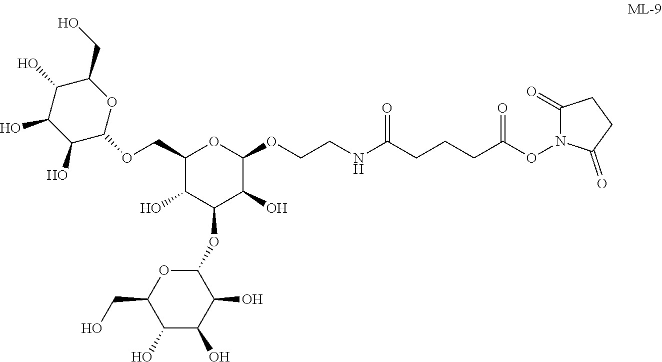

The title compound was prepared using procedures analogous to those described for ML-1 in Example 1, substituting 5-(benzyloxy)-5-oxopentanoic acid for 6-(benzyloxy)-6-oxohexanoic acid in Step 1, and substituting 2-aminoethyl α-D-mannopyranosyl-(1→3)-1α-D-mannopyranosyl-(1→6)1-β-D-mannopyranoside for 2-aminoethyl α-D-mannopyranosyl-(1→3)-[α-D-mannopyranosyl-(1→6)]-β-D-glucopyranoside in Step 2. UPLC Method B: calculated for C₂₉H₄₆N₂O₂₁ 758.26, observed m/e: 759.24 [M+1]; t_{R}=1.14 min.

### Example 10

### 5-[(2,5-Dioxopyrrolidin-1-yl)oxy]-N-(2-{[α-D-mannopyranosyl-(1→3)-[α-D-mannopyranosyl-(1→6)]-α-D-glucopyranosyl]oxy}ethyl)-5-oxo-pentanamide (ML-10)

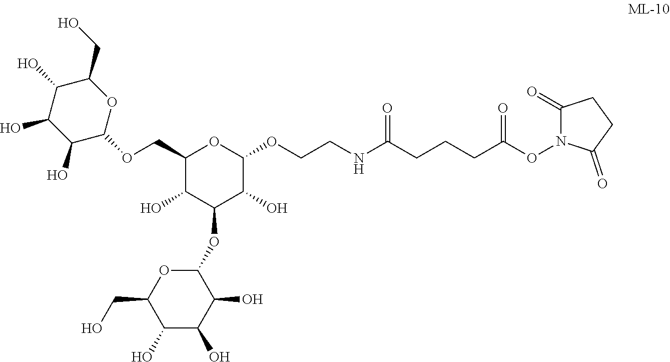

The title compound was prepared using procedures analogous to those described for ML-1 in Example 1, substituting 5-(benzyloxy)-5-oxopentanoic acid for 6-(benzyloxy)-6-oxohexanoic acid in Step 1, and substituting 2-aminoethyl α-D-mannopyranosyl-(1→3)-[α-D-mannopyranosyl-(1→6)]-α-D-glucopyranoside for 2-aminoethyl α-D-mannopyranosyl-(1→3)-[α-D-mannopyranosyl-(1→6)]-β-D-glucopyranoside in Step 2. UPLC Method B: calculated for C₂₉H₄₆N₂O₂₁ 758.26, observed m/e: 759.33 [M+1]; t_{R}=2.25 min.

### Example 11

### 6-[(2,5-Dioxopyrrolidin-1-yl)oxy]-N-(2-{[α-D-mannopyranosyl-(1→3)-[α-D-mannopyranosyl-(1→6)]-α-D-mannopyranosyl]oxy}ethyl)-6-oxo-hexanamide (ML-11)

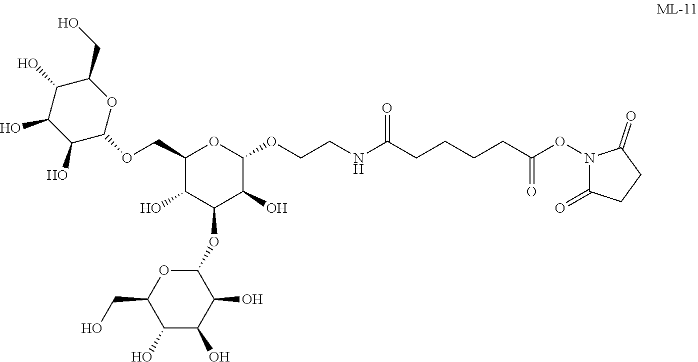

The title compound was prepared using procedures analogous to those described for ML-1 in Example 1, substituting 2-aminoethyl α-D-mannopyranosyl-(1→3)-[α-D-mannopyranosyl-(1→6)]-α-D-mannopyranoside for 2-aminoethyl α-D-mannopyranosyl-(1→3)-[α-D-mannopyranosyl-(1→6)]-β-D-glucopyranoside in Step 2. UPLC Method B: calculated for C₃₀H₄₈N₂O₂₁ 772.27, observed m/e: 773.23 [M+1]; t_{R}=0.94 min.

### Example 12

### 6-[(2,5-Dioxopyrrolidin-1-yl)oxy]-N-(2-{[α-D-mannopyranosyl-(1→3)-[α-D-mannopyranosyl-(1→6)]-β-D-mannopyranosyl]oxy}ethyl)-6-oxo-hexanamide (ML-12)

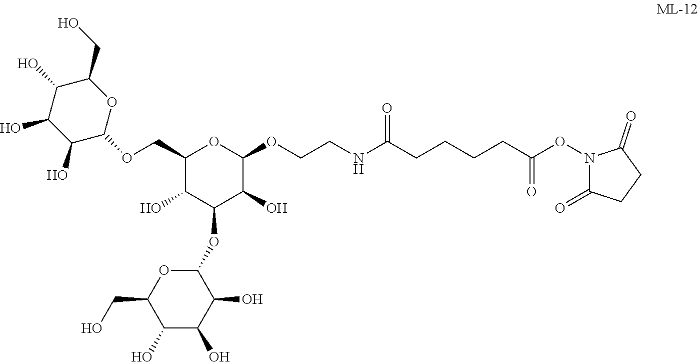

The title compound was prepared using procedures analogous to those described for ML-1 in Example 1, substituting 2-aminoethyl α-D-mannopyranosyl-(1→3)-[α-D-mannopyranosyl-(1→6)]-β-D-mannopyranoside for 2-aminoethyl α-D-mannopyranosyl-(1→3)-[α-D-mannopyranosyl-(1→6)]-β-D-glucopyranoside in Step 2. UPLC Method B: calculated for C₃₀H₄₈N₂O₂₁ 772.27, observed m/e: 773.31 [M+1]; t_{R}=1.11 min.

### Example 13

### 6-[(2,5-Dioxopyrrolidin-1-yl)oxy]-N-(2-{[α-D-mannopyranosyl-(1→3)-[α-D-mannopyranosyl-(1→6)]-2-deoxy-2-fluoro-α-D-mannopyranosyl]oxy}ethyl)-6-oxohexanamide (ML-13)

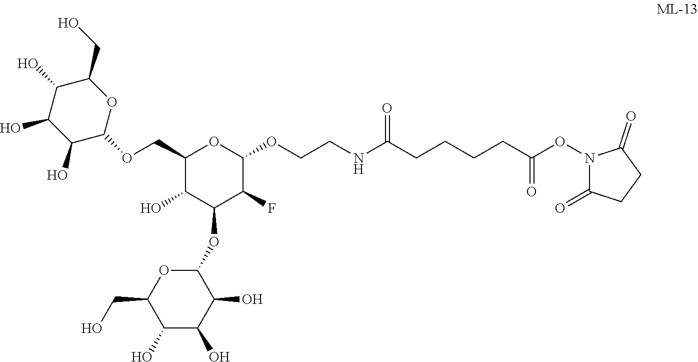

The title compound was prepared using procedures analogous to those described for ML-1 in Example 1, substituting 2-aminoethyl α-D-mannopyranosyl-(1→3)-1α-D-mannopyranosyl-(1→6)]-2-deoxy-2-fluoro-α-D-mannopyranoside for 2-aminoethyl α-D-mannopyranosyl-(1→3)-[α-D-mannopyranosyl-(1→6)]-β-D-glucopyranoside in Step 2. UPLC Method B: calculated for C₃₀H₄₇FN₂O₂₀ 774.27, observed m/e: 775.36 [M+1]; t_{R}=1.19 min.

### Example 14

### 6-[(2,5-Dioxopyrrolidin-1-yl)oxy]-N-(2-{[α-D-mannopyranosyl-(1→3)-[α-D-mannopyranosyl-(1→6)]-α-D-glucopyranosyl]oxy}ethyl)-6-oxo-hexanamide (ML-14)

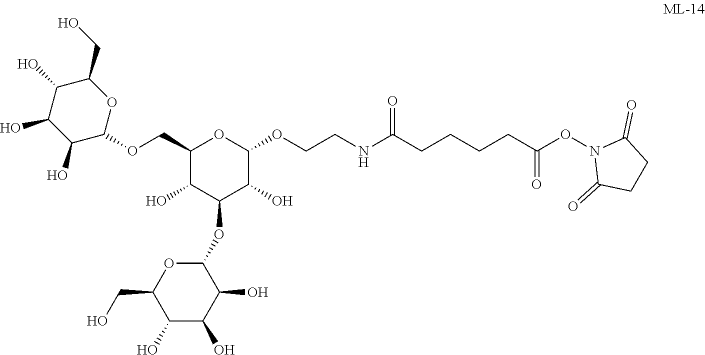

The title compound was prepared using procedures analogous to those described for ML-1 in Example 1, substituting 2-aminoethyl α-D-mannopyranosyl-(1→3)-[α-D-mannopyranosyl-(1→6)]-α-D-glucopyranoside for 2-aminoethyl α-D-mannopyranosyl-(1→3)-[α-D-mannopyranosyl-(1→6)]-β-D-glucopyranoside in Step 2. UPLC Method B: calculated for C₃₀H₄₈N₂O₂₁ 772.27, observed m/e: 773.35 [M+1]; t_{R}=1.25 min.

### Example 15

### 6-[(2,5-Dioxopyrrolidin-1-yl)oxy]-N-(2-{[α-D-mannopyranosyl-(1→3)-[α-D-mannopyranosyl-(1→6)]-2-deoxy-2-fluoro-α-D-glucopyranosyl]oxy}ethyl)-6-oxo-hexanamide (ML-15)

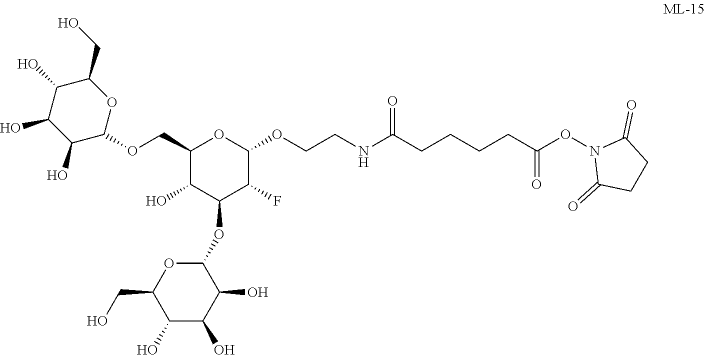

The title compound was prepared using procedures analogous to those described for ML-1 in Example 1, substituting 2-aminoethyl α-D-mannopyranosyl-(1→3)-[α-D-mannopyranosyl-(1→6)]-2-deoxy-2-fluoro-α-D-glucopyranoside for 2-aminoethyl α-D-mannopyranosyl-(1→3)-[α-D-mannopyranosyl-(1→6)]-β-D-glucopyranoside in Step 2. UPLC Method B: calculated for C₃₀H₄₇FN₂O₂₀ 774.27, observed m/e: 775.18 [M+1]; t_{R}=1.07 min.

### Example 16

### 6-[(2,5-Dioxopyrrolidin-1-yl)oxy]-N-(2-{[α-D-mannopyranosyl-(1→3)-[α-D-mannopyranosyl-(1→6)]-2-deoxy-2-fluoro-β-D-glucopyranosyl]oxy}ethyl)-6-oxo-hexanamide (ML-16)

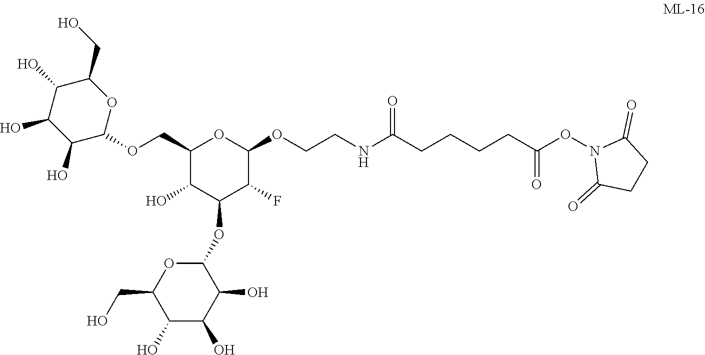

The title compound was prepared using procedures analogous to those described for ML-1 in Example 1, substituting 2-aminoethyl α-D-mannopyranosyl-(1→3)-[α-D-mannopyranosyl-(1→6)]-2-deoxy-2-fluoro-β-D-glucopyranoside for 2-aminoethyl α-D-mannopyranosyl-(1→3)-[α-D-mannopyranosyl-(1→6)]-β-D-glucopyranoside in Step 2. UPLC Method B: calculated for C₃₀H₄₇FN₂O₂₀ 774.27, observed m/e: 775.34 [M+1]; t_{R}=1.05 min.

### Example 17

### 7-[(2,5-Dioxopyrrolidin-1-yl)oxy]-N-(2-{[α-D-mannopyranosyl-(1→3)-[α-D-mannopyranosyl-(1→6)]-α-D-mannopyranosyl]oxy}ethyl)-7-oxo-heptanamide (ML-17)

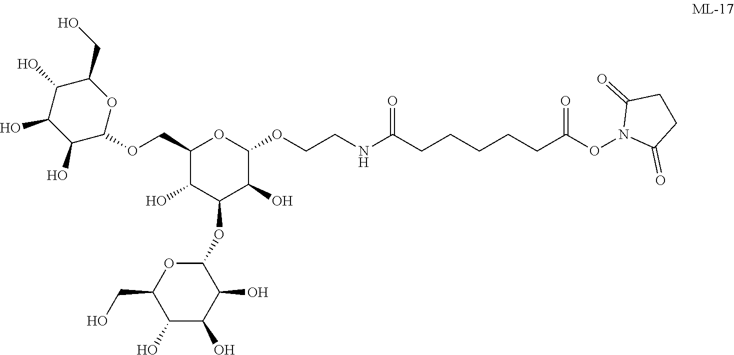

The title compound was prepared using procedures analogous to those described for ML-2 in Example 2, substituting heptanedioic acid for octanedioic acid in Step 1, and substituting 2-aminoethyl α-D-mannopyranosyl-(1→3)-[α-D-mannopyranosyl-(1→6)]-α-D-mannopyranoside for 2-aminoethyl α-D-mannopyranosyl-(1→3)-[α-D-mannopyranosyl-(1→6)]-β-D-glucopyranoside in Step 2. UPLC Method B: calculated for C₃₁H₅₀N₂O₂₁ 786.29, observed m/e: 787.28 [M+1]; t_{R}=1.10 min.

### Example 18

### 7-[(2,5-Dioxopyrrolidin-1-yl)oxy]-N-(2-{[α-D-mannopyranosyl-(1→3)-[α-D-mannopyranosyl-(1→6)]-β-D-mannopyranosyl]oxy}ethyl)-7-oxo-heptanamide (ML-18)

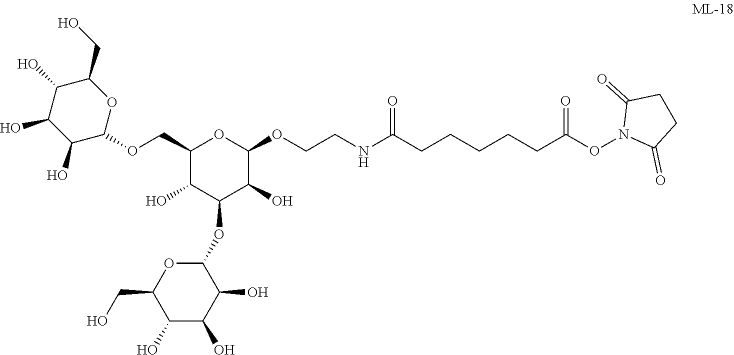

The title compound was prepared using procedures analogous to those described for ML-2 in Example 2, substituting heptanedioic acid for octanedioic acid in Step 1, and substituting 2-aminoethyl α-D-mannopyranosyl-(1→3)-[α-D-mannopyranosyl-(1→6)]-β-D-mannopyranoside for 2-aminoethyl α-D-mannopyranosyl-(1→3)-[α-D-mannopyranosyl-(1→6)]-β-D-glucopyranoside in Step 2. UPLC Method B: calculated for C₃₁H₅₀N₂O₂₁ 786.29, observed m/e: 787.29 [M+1]; t_{R}=1.11 min.

### Example 19

### 8-[(2,5-Dioxopyrrolidin-1-yl)oxy]-N-(2-{[α-D-mannopyranosyl-(1→3)-[α-D-mannopyranosyl-(1→6)]-2-deoxy-2-fluoro β-D-glucopyranosyl]oxy}ethyl)-8-oxo-octanamide (ML-19)

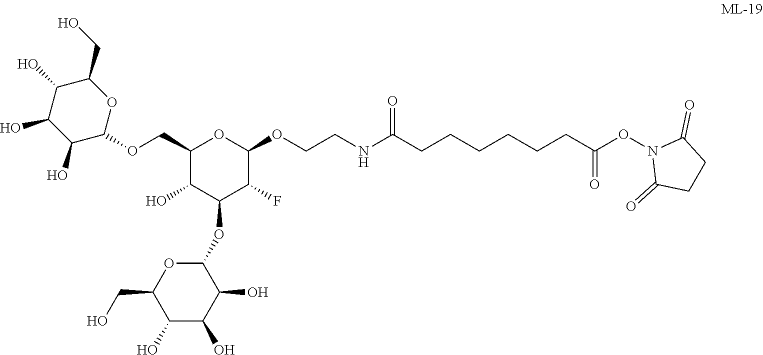

The title compound was prepared using procedures analogous to those described for ML-2 in Example 2, substituting 2-aminoethyl α-D-mannopyranosyl-(1→3)-[α-D-mannopyranosyl-(1→6)]-2-deoxy-2-fluoro β-D-glucopyranoside for 2-aminoethyl α-D-mannopyranosyl-(1→3)-[α-D-mannopyranosyl-(1→6)]-β-D-glucopyranoside in Step 2. UPLC Method B: calculated for C₃₂H₅₁FN₂O₂₀: 802.30, observed m/e: 803.34 [M+1]; t_{R}=1.41 min.

### Example 20

### 2-(2-(2-[(2,5-Dioxopyrrolidin-1-yl)oxy]-N-(2-{[α-D-mannopyranosyl-(1→3)-[α-D-mannopyranosyl-(1→6)]-α-D-mannopyranosyl]oxy}ethyl)-2-oxoethoxy)ethoxy)-acetamide (ML-20)

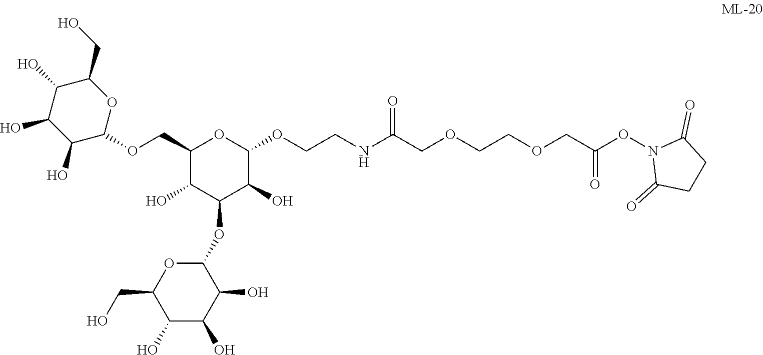

The title compound was prepared using procedures analogous to those described for ML-2 in Example 2, substituting 2,2′-(ethane-1,2-diylbis(oxy))diacetic acid for octanedioic acid in Step 1, and substituting 2-aminoethyl α-D-mannopyranosyl-(1→3)-[α-D-mannopyranosyl-(1→6)]-α-D-mannopyranoside for 2-aminoethyl α-D-mannopyranosyl-(1→3)-[α-D-mannopyranosyl-(1→6)]-β-D-glucopyranoside in Step 2. UPLC Method B: calculated for C₃₀H₄₈N₂O₂₃: 804.26, observed m/e: 805.30 [M+1]; t_{R}=0.90 min.

### Example 21

### 2-(2-(2-[(2,5-Dioxopyrrolidin-1-yl)oxy]-N-(2-{[α-D-mannopyranosyl-(1→3)-[α-D-mannopyranosyl-(1→6)]-β-D-mannopyranosyl]oxy}ethyl)-2-oxoethoxy)ethoxy)-acetamide (ML-21)

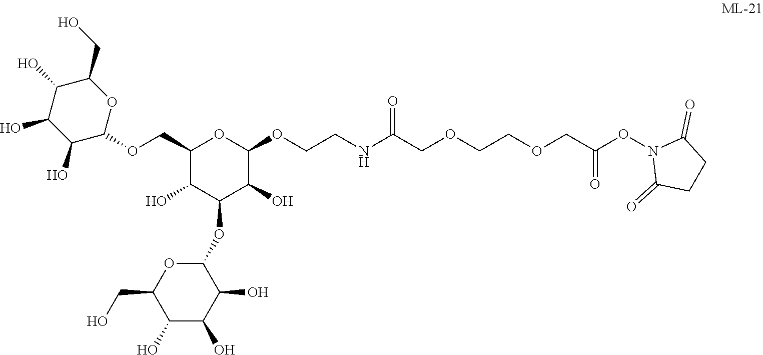

The title compound was prepared using procedures analogous to those described for ML-2 in Example 2, substituting 2,2′-(ethane-1,2-diylbis(oxy))diacetic acid for octanedioic acid in Step 1, and substituting 2-aminoethyl α-D-mannopyranosyl-(1→3)-[α-D-mannopyranosyl-(1→6)]-β-D-glucopyranoside with 2-aminoethyl α-D-mannopyranosyl-(1→3)-[α-D-mannopyranosyl-(1→6)]-β-D-mannopyranoside in Step 2. UPLC Method B: calculated for C₃₀H₄₈N₂O₂₃ 804.26, observed m/e: 805.31 [M+1]; t_{R}=0.91 min.

### Example 22

### 10-[(2,5-Dioxopyrrolidin-1-yl)oxy]-N-(2-{[α-D-mannopyranosyl-(1→3)-[α-D-mannopyranosyl-(1→6)]-α-D-mannopyranosyl]oxy}ethyl)-10-oxo-decanamide (ML-22)

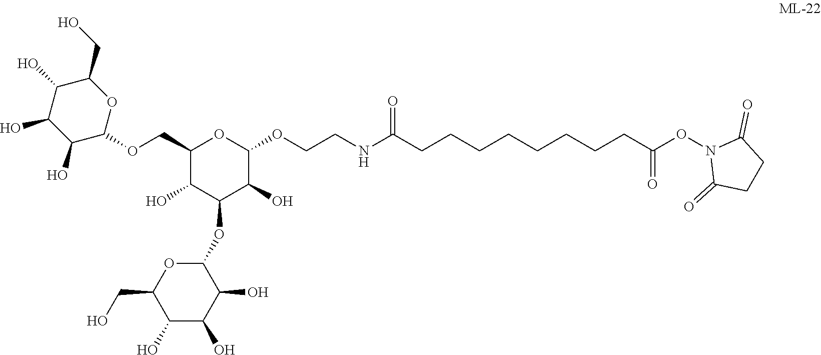

The title compound was prepared using procedures analogous to those described for ML-2 in Example 2, substituting decanedioic acid for octanedioic acid in Step 1, and substituting 2-aminoethyl α-D-mannopyranosyl-(1→3)-[α-D-mannopyranosyl-(1→6)]-α-D-mannopyranoside for 2-aminoethyl α-D-mannopyranosyl-(1→3)-[α-D-mannopyranosyl-(1→6)]-β-D-glucopyranoside in Step 2. UPLC Method B: calculated for C₃₄H₅₆N₂O₂₁ 828.34, observed m/e: 829.30 [M+1]; t_{R}=1.25 min.

### Example 23

### 10-[(2,5-Dioxopyrrolidin-1-yl)oxy]-N-(2-{[α-D-mannopyranosyl-(1→3)-[α-D-mannopyranosyl-(1→6)]-β-D-mannopyranosyl]oxy}ethyl)-10-oxo-decanamide (ML-23)

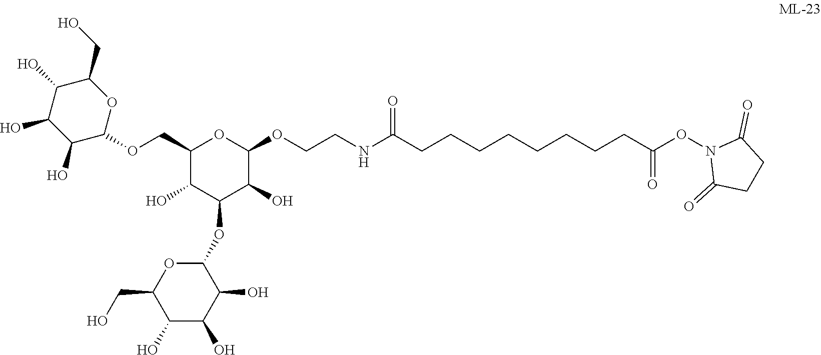

The title compound was prepared using procedures analogous to those described for ML-2 in Example 2, substituting decanedioic acid for octanedioic acid in Step 1, and substituting 2-aminoethyl α-D-mannopyranosyl-(1→3)-[α-D-mannopyranosyl-(1→6)]-β-D-glucopyranoside with 2-aminoethyl α-D-mannopyranosyl-(1→3)-[α-D-mannopyranosyl-(1→6)]-β-D-mannopyranoside in Step 2. UPLC Method B: calculated for C₃₄H₅₆N₂O₂₁ 828.34, observed m/e: 829.30 [M+1]; t_{R}=1.23 min.

### Example 24

### 3-(2-β-[(2,5-Dioxopyrrolidin-1-yl)oxy]-N-(2-{[α-D-mannopyranosyl-(1→3)-[α-D-mannopyranosyl-(1→6)]-α-D-mannopyranosyl]oxy}ethyl)-3-oxopropoxy)ethoxy)-propanamide (ML-24)

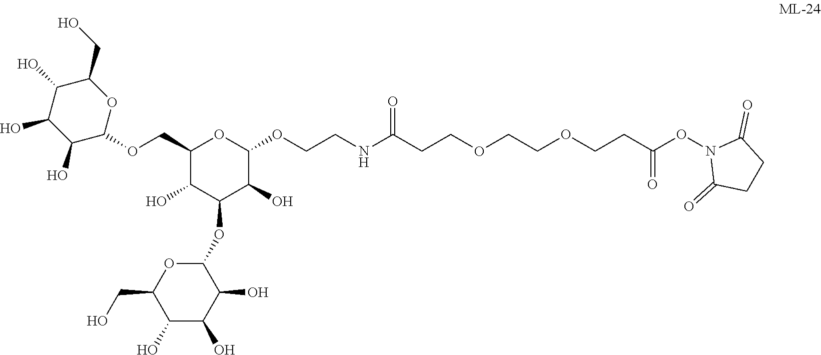

The title compound was prepared using procedures analogous to those described for ML-2 in Example 2, substituting 3,3′-(ethane-1,2-diylbis(oxy))dipropionic acid for octanedioic acid in Step 1, and substituting 2-aminoethyl α-D-mannopyranosyl-(1→3)-[α-D-mannopyranosyl-(1→6)]-α-D-mannopyranoside for 2-aminoethyl α-D-mannopyranosyl-(1→3)-[α-D-mannopyranosyl-(1→6)]-β-D-glucopyranoside in Step 2. UPLC Method B: calculated for C₃₂H₅₂N₂O₂₃ 832.30, observed m/e: 833.40 [M+1]; t_{R}=1.54 min.

### Example 25

### −(2-(3-[(2,5-Dioxopyrrolidin-1-yl)oxy]-N-(2-{[α-D-mannopyranosyl-(1→3)-[α-D-mannopyranosyl-(1→6)]-β-D-mannopyranosyl]oxy}ethyl)-3-oxopropoxy)ethoxy)-propanamide (ML-25)

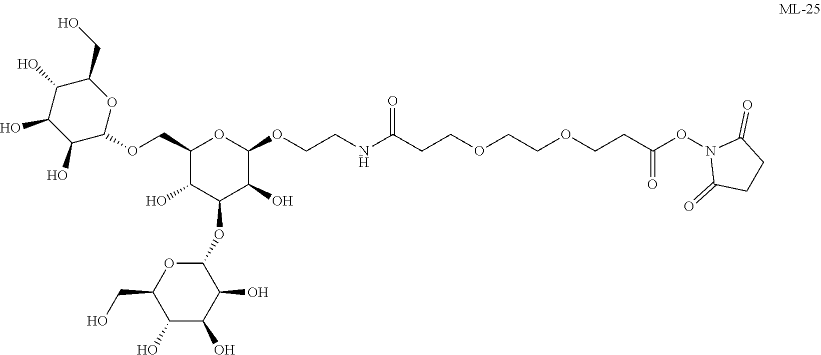

The title compound was prepared using procedures analogous to those described for ML-2 in Example 2, substituting 3,3′-(ethane-1,2-diylbis(oxy))dipropionic acid for octanedioic acid in Step 1, and substituting 2-aminoethyl α-D-mannopyranosyl-(1→3)-[α-D-mannopyranosyl-(1→6)]-β-D-mannopyranoside for 2-aminoethyl α-D-mannopyranosyl-(1→3)-[α-D-mannopyranosyl-(1→6)]-β-D-glucopyranoside in Step 2. UPLC Method B: calculated for C₃₂H₅₂N₂O₂₃ 832.30, observed m/e: 833.43 [M+1]; t_{R}=1.49 min.

### Example 26

### 12-[(2,5-Dioxopyrrolidin-1-yl)oxy]-N-(2-{[α-D-mannopyranosyl-(1→3)-[α-D-mannopyranosyl-(1→6)]-α-D-mannopyranosyl]oxy}ethyl)-12-oxo-dodecanamide (ML-26)

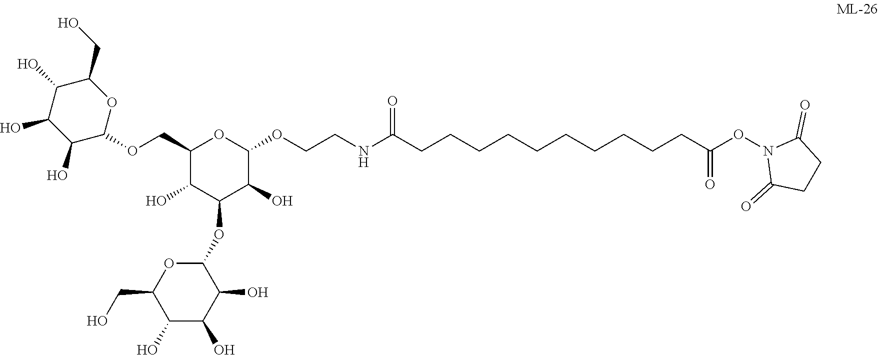

The title compound was prepared using procedures analogous to those described for ML-2 in Example 2, substituting dodecanedioic acid for octanedioic acid in Step 1, and substituting 2-aminoethyl α-D-mannopyranosyl-(1→3)-[α-D-mannopyranosyl-(1→6)]-α-D-mannopyranoside for 2-aminoethyl α-D-mannopyranosyl-(1→3)-[α-D-mannopyranosyl-(1→6)]-β-D-glucopyranoside in Step 2. UPLC Method B: calculated for C₃₆H₆₀N₂O₂₁ 856.37, observed m/e: 857.49 [M+1]; t_{R}=2.87 min.

### Example 27

### 12-[(2,5-Dioxopyrrolidin-1-yl)oxy]-N-(2-{[α-D-mannopyranosyl-(1→3)-[α-D-mannopyranosyl-(1→6)]-β-D-mannopyranosyl]oxy}ethyl)-12-oxo-dodecanamide (ML-27)

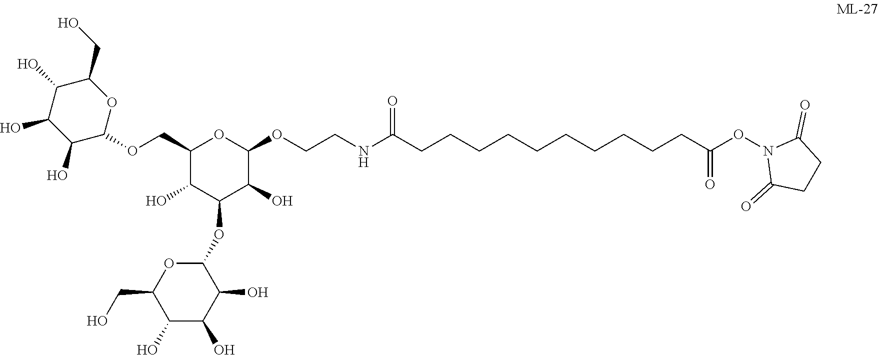

The title compound was prepared using procedures analogous to those described for ML-2 in Example 2, substituting dodecanedioic acid for octanedioic acid in Step 1, and substituting 2-aminoethyl α-D-mannopyranosyl-(1→3)-[α-D-mannopyranosyl-(1→6)]-β-D-mannopyranoside for 2-aminoethyl α-D-mannopyranosyl-(1→3)-[α-D-mannopyranosyl-(1→6)]-β-D-glucopyranoside in Step 2. UPLC Method B: calculated for C₃₆H₆₀N₂O₂₁ 856.37, observed m/e: 857.49 [M+1]; t_{R}=2.84 min.

### Example 28

### 14-[(2,5-Dioxopyrrolidin-1-yl)oxy]-N-(2-{[α-D-mannopyranosyl-(1→3)-[α-D-mannopyranosyl-(1→6)]-α-D-mannopyranosyl]oxy}ethyl)-14-oxo-tetradecanamide (ML-28)

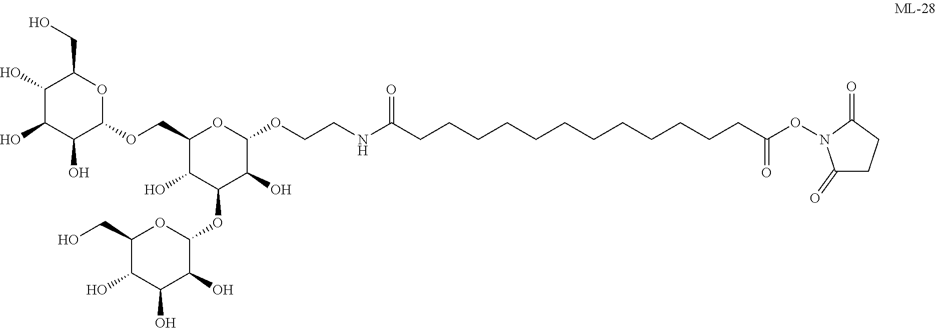 [SMILES: O=C(CCCCCCCCCCCCC(=O)ON1C(=O)CCC1=O)NCCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O]

The title compound was prepared using procedures analogous to those described for ML-2 in Example 2, substituting tetradecanedioic acid for octanedioic acid in Step 1, and substituting 2-aminoethyl α-D-mannopyranosyl-(1→3)-[α-D-mannopyranosyl-(1→6)]-α-D-mannopyranoside for 2-aminoethyl α-D-mannopyranosyl-(1→3)-[α-D-mannopyranosyl-(1→6)]-β-D-glucopyranoside in Step 2. UPLC Method B: calculated for C₃₈H₆₄N₂O₂₁ 884.40, observed m/e: 885.46 [M+1]; t_{R}=3.20 min.

### Example 29

### 14-[(2,5-Dioxopyrrolidin-1-yl)oxy]-N-(2-{[α-D-mannopyranosyl-(1→3)-[α-D-mannopyranosyl-(1→6)]-β-D-mannopyranosyl]oxy}ethyl)-14-oxo-tetradecanamide (ML-29)

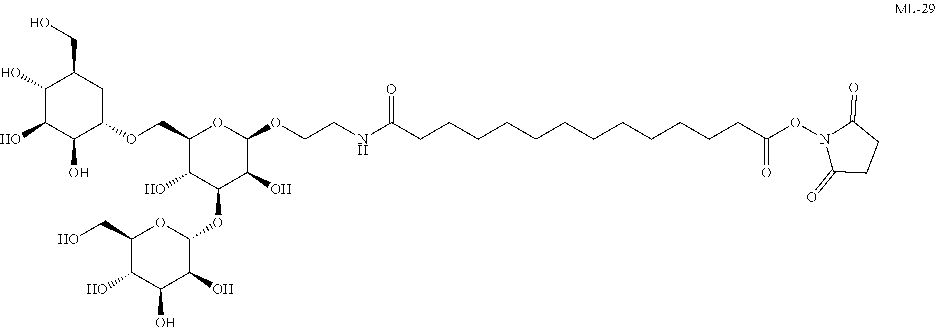 [SMILES: O=C(CCCCCCCCCCCCC(=O)ON1C(=O)CCC1=O)NCCO[C@@H]1O[C@H](CO[C@H]2C[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O]

The title compound was prepared using procedures analogous to those described for ML-2 in Example 2, substituting tetradecanedioic acid for octanedioic acid in Step 1, and substituting 2-aminoethyl α-D-mannopyranosyl-(1→3)-[α-D-mannopyranosyl-(1→6)]-β-D-mannopyranoside for 2-aminoethyl α-D-mannopyranosyl-(1→3)-[α-D-mannopyranosyl-(1→6)]-β-D-glucopyranoside in Step 2. UPLC Method B: calculated for C₃₈H₆₄N₂O₂₁ 884.40, observed m/e: 885.47 [M+1]; t_{R}=3.18 min.

### Example 30

### 16-[(2,5-Dioxopyrrolidin-1-yl)oxy]-N-(2-{[α-D-mannopyranosyl-(1→3)-[α-D-mannopyranosyl-(1→6)]-α-D-mannopyranosyl]oxy}ethyl)-16-oxo-hexadecanamide (ML-30)

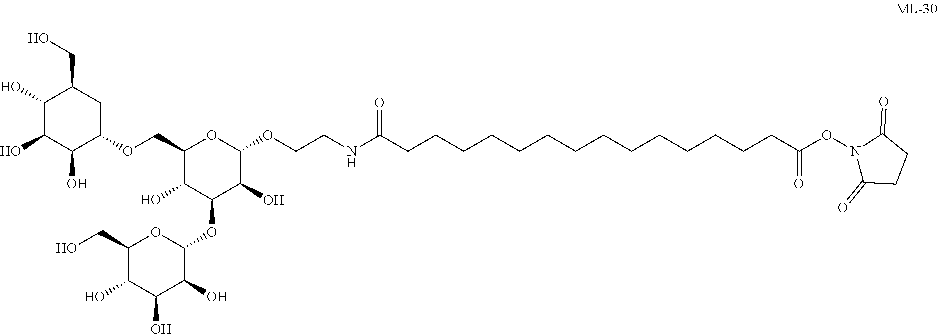 [SMILES: O=C(CCCCCCCCCCCCCCC(=O)ON1C(=O)CCC1=O)NCCO[C@H]1O[C@H](CO[C@H]2C[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O]

The title compound was prepared using procedures analogous to those described for ML-2 in Example 2, substituting hexadecanedioic acid for octanedioic acid in Step 1, and substituting 2-aminoethyl α-D-mannopyranosyl-(1→3)-[α-D-mannopyranosyl-(1→6)]-α-D-mannopyranoside for 2-aminoethyl α-D-mannopyranosyl-(1→3)-[α-D-mannopyranosyl-(1→6)]-β-D-glucopyranoside in Step 2. UPLC Method B: calculated for C₄₀H₆₈N₂O₂₁ 912.43, observed m/e: 913.50 [M+1]; t_{R}=3.64 min.

### Example 31

### 16-[(2,5-Dioxopyrrolidin-1-yl)oxy]-N-(2-{[α-D-mannopyranosyl-(1→3)-[α-D-mannopyranosyl-(1→6)]-β-D-mannopyranosyl]oxy}ethyl)-16-oxo-hexadecanamide (ML-31)

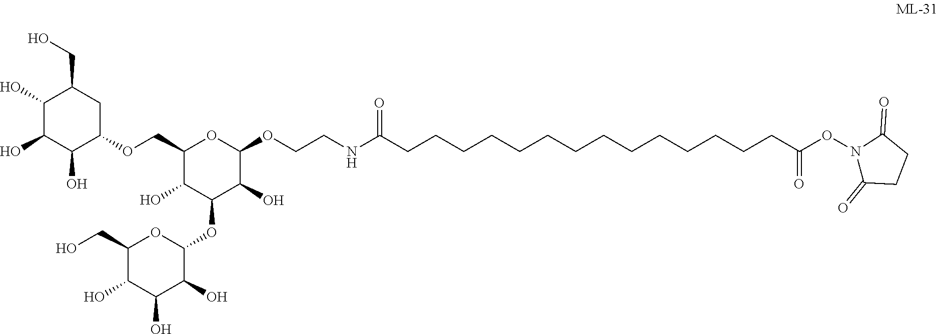 [SMILES: O=C(CCCCCCCCCCCCCCC(=O)ON1C(=O)CCC1=O)NCCO[C@@H]1O[C@H](CO[C@H]2C[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O]

The title compound was prepared using procedures analogous to those described for ML-2 in Example 2, substituting hexadecanedioic acid for octanedioic acid in Step 1, and substituting 2-aminoethyl α-D-mannopyranosyl-(1→3)-[α-D-mannopyranosyl-(1→6)]-β-D-mannopyranoside in for 2-aminoethyl α-D-mannopyranosyl-(1→3)-[α-D-mannopyranosyl-(1→6)]-β-D-glucopyranoside in Step 2. UPLC Method B: calculated for C₄₀H₆₈N₂O₂₁ 912.43, observed m/e: 913.50 [M+1]; t_{R}=3.57 min.

### Example 32

### 6-[(2,5-Dioxopyrrolidin-1-yl)oxy]-N-(3-[α-D-mannopyranosyl-(1→6)-α-D-mannopyranosyl-(1→3)-β-D-mannopyranose (1-O-β)benzyl 6-((3-propyl amino)-6-oxohexanoate (ML-32)

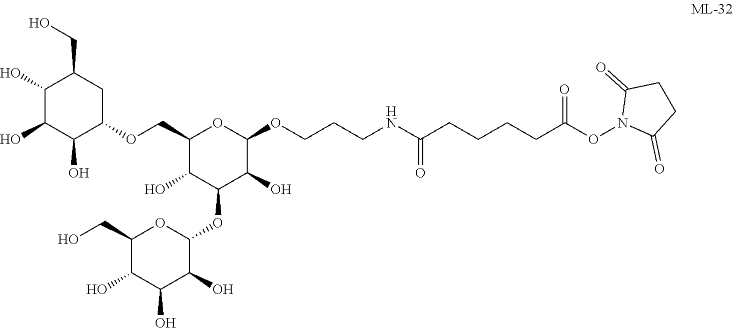 [SMILES: O=C(CCCCC(=O)ON1C(=O)CCC1=O)NCCCO[C@@H]1O[C@H](CO[C@H]2C[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O]

### Step 1: 2,3,4,6-Tetra-O-Benzoyl-D-mannose

In a 250 mL round bottom flask, 1,2,3,4,5-penta-O-benzoyl-D-mannopyranose (5 g, 7.14 mmol, 1.0 eq) was dissolved in ACN (25 mL). The solution was cooled to −20° C. To above solution was added dimethylamine (24.98 mL, 2.0N/THF, 50 mmol, 7.0 eq). The mixture was stirred at −20° C. for 5 h and then 25° C. for 2 h. The solvent was removed under reduced pressure. The crude was purified by chromatography on C18 reverse silica gel column, eluted with 0-100% EtOAc/hexanes in 16 CV. The fractions containing desired product were combined and concentrated to give the title compound. LC-MS 2 min: t_{R}=1.23 min (597.32 [M+H]⁺). ¹H NMR (in CDCl₃, 500 MHz): 7.2-8.2 (20H, Ph), 6.2 (t, 1H, H4), 6.0 (dd, 1H, H3), 5.8 (dd, 1H, H2), 5.5 (d, 1H, H1), 4.8 (dd, 1H, H6b), 4.7 (m, 1H, H5), 4.6 (br, 1H, —OH), 4. 1H NMR (in CDCl_{3, 500} MHz): 7.2-8.2 (20H, Ph), 6.2 (t, 1H, H4), 6.0 (dd, 1H, H2), 5.5 (d, 1H, H1), 4.8 (dd, 1H, H6b), 4.7 (m, 1H, H5), 4.6 (br, 1H, —OH), 4.5 (dd, 1H, H6a).

### Step 2: 2,3,4,6-Tetra-O-Benzoyl-α-D-mannopyranosyl trichloracetimidate

In a 40 mL vial, 2,3,4,6-tetra-O-benzoyl-D-mannose (500 mg, 0.838 mmol, 1.0 eq) was dissolved in anhydrous DCM (5.0 mL). The solution was cooled to 0° C. To above solution was added trichloroacetonitrile (363 mg, 2.51 mmol, 3.0 eq) followed by addition of DBU (0.139 mL, 0.922 mmol, 1.1 eq). The mixture was warmed to 25° C. and stirred for 1 h. The mixture was loaded on a 24 g silica gel column and eluted with 0-100% EtOAc/hexanes in 16 CV. The fractions containing desired product were combined and concentrated to give the title compound. ¹H NMR (in CDCl₃, 500 MHz): 8.9 (s, 1H, NH), 7.2-8.2 (20H, Ph), 6.5 (s, 1H, H1), 6.3(m, 2H), 5.6 (m, 2H), 4.8 (dd, 1H, H6b), 4.7 (m, 1H, H5), 4.5 (dd, 1H, H6a).

### Step 3: PerTrimethylsilane-D-mannose

In a 200 mL round bottom flask, D-mannose (20 g, 111 mmol, 1.0 eq) was dissolved in DMF (25 mL). To above solution was added TEA (80 mL, 577 mmol, 5.2 eq). The solution was cooled to 0° C. To above solution was added TMS-Cl (73.8 mL, 577 mmol, 5.2 eq) dropwise. The mixture was warmed to 25° C. and stirred at this temperature for 4 h. The mixture was poured into ice/hexanes (1/1, 100 mL), extracted with hexanes (50 ml×3), washed with water (20 mL×3). The organics were dried over MgSO₄, filtered and concentrated to give the title compound. ¹H NMR (in CDCl₃, 500 MHz): 4.89 (1H, H1, d, J=2.1 Hz), 3.5-3.9 (m, 6H, H2-H6), 0.1(m, 45H).

### Step 4: 3-Iodoproxy-α-D-mannopyranose and 3-Iodoproxy-β-D-mannopyranose

In a 250 ml round bottom flask, pertrimethylsilane-D-mannose (10 g, 18.5 mmol, 1.0 eq) was dissolved in DCM (20 mL). The solution was cooled to 0° C. To above solution was added iodotrimethylsilane (2.64 mL, 19.4 mmol, 1.05 eq). The mixture was warmed to 25° C. and stirred for 1 h. The mixture was cooled back to 0° C. To above solution was added oxetane (1.81 g, 27.7 mmol, 1.5 eq). The reaction was warmed to 25° C. and stirred for 6 h. The solvent was removed by rotary evaporation under reduced pressure. To the crude was added MeOH (20 mL) and ion exchange resin (DOWEX) H⁺ form (20 g, pre-washed with MeOH 10 mL×2). The mixture was stirred at 25° C. for 6 h. The resin was filtered. The filtrate was concentrated and purified by preparatory scale HPLC using a C8 10 μm, 100 ÅA, 50×250 mm column, eluted with 5-25% ACN/water containing 0.05% TFA in 20 min. The first eluted peak was 3-iodoproxy-β-D-mannopyranose, and the second eluted peak was 3-iodoproxy-α-D-mannopyranose. UPLC-MS C8 5 min: 3-iodoproxy-α-D-mannopyranose, 2.148 (371.00, [M+Na]⁺); 3-iodoproxy-β-D-mannopyranose, 2.148 (371.00, [M+Na]⁺). ¹H NMR (in CD₃OD, 500 MHz): 3-iodoproxy-α-D-mannopyranose, 2.10-2.05 (2H, m), 3.51 (1H, ddd, J=9.96, 6.27, 5.24 Hz), 3.57 (1H, ddd, J=9.47, 5.54, 2.36 Hz), 3.77-3.64 (3H, m), 3.87-3.81 (3H, m), 4.78 (1H, d, 1.75 Hz); 3-iodoproxy-β-D-mannopyranose, 2.15-2.09 (2H, m), 3.24 (1H, ddd, J=9.64, 5.79, 2.38 Hz), 3.47 (1H, dd, J=9.43, 3.24 Hz), 3.68-3.57 (2H, m), 3.74 (1H, dd, J=11.79, 5.79 Hz), 3.91-3.87 (2H, m), 3.98 (1H, dt, J=10.03, 5.72 Hz), 4.54 (1H, d, J=0.95 Hz).

### Step 5: 3-Azidoproxy-β-D-mannopyranose

In a 100 ml round bottom flask, 3-iodoproxy-β-D-mannopyranose (2 g, 5.74 mmol, 1.0 eq) was dissolved in DMF (10 ml). To above solution was added sodium azide (0.448 g, 6.89 mmol, 1.2 eq). The reaction was warmed to 60° C. and stirred for 12 h. DMF was removed under reduced pressure. The crude was redissolved in water, purified by C18 reverse phase chromatograph (130 g column, elute with 0-20% ACN/water in 16 CV). Fractions containing desired product were combined and lyophilized to give the title compound. LC-MS 2 min: t_{R}=0.23 min (264.16 [M+H]⁺). ¹H NMR (in CDCl_{3, 500} MHz): 1.89-1.83 (2H, m), 3.22-3.16 (1H, m), 3.44-3.41 (3H, m), 3.55 (1H, t, J=9.54 Hz), 3.70-3.59 (2H, m), 3.87-3.83 (2H, m), 3.98 (1H, dt, J=9.95, 5.98 Hz), 4.50 (1H, d, J=0.96 Hz).

### Step 6: 3,4-Dibenzoyl-3′-Azidoproxy-β-D-mannopyranose and 2,6-Dibenzoyl-3′-Azidoproxy-β-D-mannopyranose

To a suspension of 3-azidoproxy-β-D-mannopyranose (1030 mg, 3.91 mmol, 1.0 eq) in ACN (15 mL) was added triethyl orthobenzoate (2.352 mL, 10.17 mmol, 2.6 eq) followed by the addition of TFA (0.030 ml, 0.391 mmol) in ACN (0.5 mL). The mixture was allowed to stir at rt for 1 h. ACN was removed by rotary evaporation. To the above mixture, TFA (10% in water) (4.28 ml, 5.55 mmol) was added. The mixture was stirred at rt for 2 h. The residue was purified by silica gel column chromatography, eluting with 0-100% ether/CH₂Cl₂ in 16 CV, to give the above products 3,4-dibenzoyl-3′-azidoproxy-β-D-mannopyranose and 2,6-dibenzoyl-3′-azidoproxy-β-D-mannopyranose. LC-MS 2 min: t_{R}=1.11 min (472.36 [M+H]⁺). ¹H NMR (in CDCl₃, 500 MHz): 3,4-dibenzoyl-3′-azidoproxy-β-D-mannopyranose, 1.24-1.19 (1H, m), 1.94 (3H, s), 3.47-3.41 (2H, m), 3.66 (1H, ddd, J=9.84, 4.61, 2.32 Hz), 3.77-3.73 (2H, m), 3.83 (1H, dd, J=12.72, 2.35 Hz), 4.11-4.07 (1H, m), 4.36 (1H, d, J=3.04 Hz), 4.77 (1H, s), 5.30 (1H, s), 5.43 (1H, dd, J=10.01, 3.01 Hz), 5.76 (1H, t, J=9.91 Hz), 7.37 (4H, q, J=7.09 Hz), 7.51 (2H, q, J=8.31 Hz), 8.01-7.94 (4H, m); 2,6-dibenzoyl-3′-azidoproxy-β-D-mannopyranose, 1.92-1.85 (2H, m), 3.38 (2H, t, J=6.50 Hz), 3.70-3.66 (2H, m), 4.02-3.98 (1H, m), 4.19 (1H, t, J=9.67 Hz), 4.26 (1H, d, J=2.98 Hz), 4.66-4.63 (2H, m), 4.73 (1H, d, J=12.04 Hz), 5.09 (1H, dd, J=9.69, 3.04 Hz), 7.44 (4H, q, J=7.97 Hz), 7.57 (2H, q, J=6.92 Hz), 8.09 (4H, dd, J=16.83, 7.71 Hz).

### Step 7: 2,3,4,6-Tetra-O-Benzoyl-α-D-mannopyranosyl-(1→6)-2,3,4,6-Tetra-O-Benzoyl-α-D-mannopyranosyl-(1→3)-3-azidoproxy-2,4-dibenzoyl-β-D-mannopyranose

In a 100 mL round bottom flask, 2,6-dibenzoyl-3′-azidoproxy-β-D-mannopyranose (200 mg, 0.424 mmol, 1.0 eq), 2,3,4,6-tetra-O-benzoyl-α-D-mannopyranosyl trichloracetimidate (723 mg, 0.976 mmol, 2.3 eq) were dissolved in DCM (20 mL). To above solution was added 4 Å molecular sieves (200 mg). The mixture was cooled to −78° C. To above mixture was added TMSOTf (234, 0.127 mmol, 0.3 eq). The mixture was warmed slowly to 0° C. and stirred for 30 min. The reaction was then quenched with aq NaHCO₃, filtered through a pad of filter reagent diatomaceous earth (CELITE), diluted with DCM (20 mL), washed with brine and water. The organic was dried over MgSO₄, filtered and concentrated. The crude was purified by flash chromatography on a 220 g column, eluted with 0-60% EtOAc/hexanes in 15 CV. The fractions containing desired product were concentrated and dried over vacuum to give desired product. ¹H NMR (in CDCl₃, 500 MHz): 7.1-8.3 (m, 50H, Ph), 6.0-6.2 (m, 2H), 5.9-6.0 (m, 1H), 5.7-5.8 (m, 2H), 5.65 (dd, 1H, J=10.01, 3.31 Hz), 5.34 (1H, s), 5.30 (1H, s), 5.15 (1H, s), 4.2-5.0 (m, 9H), 4.1 (m, 1H), 3.2-4.0 (m, 5H), 1.9 (m, 2H).

### Step 8: α-D-mannopyranosyl-(1→6)-α-D-mannopyranosyl-(1→3)-3-azidoproxy-β-D-mannopyranose

In a 50 mL round bottom flask was added above product (2.3 g, 1.412 mmol, 1.0 eq) and MeOH (10 mL). To above solution was added sodium methoxide (30% in MeOH) dropwise until pH>10. The reaction was stirred at 25° C. for 18 h; LC-MS showed no starting material left. To above solution was added ion exchange resin (DOWEX) H⁺ form (50 W×8 -200) resin till pH ˜7. The mixture was filtered, concentrated, extracted with Et₂O. The aqueous portion was concentrated to give the title crude compound without purification. LC-MS 2 min: t_{R}=0.18 min (588.46, [M+H]⁺). ¹H NMR (in CD₃OD, 500 MHz): 1.88-1.82 (2H, m), 3.64-3.54 (6H, m), 3.87-3.67 (11H, m), 3.97-3.90 (3H, m), 4.09 (1H, d, J=3.12 Hz), 4.50 (1H, d, J=0.88 Hz), 4.82 (1H, S), 5.06 (1H, d, J=1.70 Hz).

### Step 9: α-D-mannopyranosyl-(1→6)-α-D-mannopyranosyl-(1→3)-3-aminoproxy-β-D-mannopyranose

In a 50 mL round bottom flask, above compound (760 mg, 1.294 mmol, 1.0 eq) was dissolved in water (10 mL). To above solution was added Pd(OH)₂ (20%, 91 mg, 0.129 mmol, 0.1 eq). The reaction was stirred at 25° C. under H₂ for 1 h. LC-MS showed no starting material left. The mixture was filtered through a pad of filter reagent diatomaceous earth (CELITE), concentrated to give the title compound. LC-MS 4 min: t_{R}=0.14 min (562.27, [M+H]⁺).

### Step 10: Benzyl 6-([3-α-D-mannopyranosyl-(1→6)-α-D-mannopyranosyl-(1→3)-β-D-mannopyranose (1-O-β)oxy]propyl}amino-6-oxohexanoate

In a 40 mL vial, α-D-mannopyranosyl-(1→6)-α-D-mannopyranosyl-(1→3)-3-aminoproxy-β-D-mannopyranose (700 mg, 1.247 mmol, 1.0 eq) was dissolved in DMF (3 ml). The solution was cooled to 0° C. To above solution was added benzyl (2,5-dioxopyrrolidin-1-yl) adipate (499 mg, 1.496 mmol, 1.2 eq) and TEA (0.226 mL, 1.621 mmol, 1.3 eq). The mixture was stirred at 0° C. for 2 h. UPLC indicated formation of desired product. The mixture was diluted with water (3 mL), concentrated and purified by C18 reverse phase chromatography (40 g, eluted with 0-40% ACN/water in 16 CV). The fractions containing desired product were combined and concentrated to give α-D-mannopyranosyl-(1→6)-α-D-mannopyranosyl-(1→3)-β-D-mannopyranose (1-043) benzyl 6-((3-propyl amino)-6-oxohexanoate. UPLC-MS C18 column 5 min: t_{R}=3.23 min (780.3703 [M+H]⁺).

### Step 11: 6-({3-[α-D-mannopyranosyl-(1→6)-α-D-mannopyranosyl-(1→3)-β-D-mannopyranose (1-O-β)oxy]propyl}amino-6-oxohexanoic acid

The above product was dissolved in water (5 ml), added Pd/C (10%, 66.3 mg). The mixture was stirred at 25° C. under H₂ for 18 h. LC-MS showed no starting material left. To mixture was filtered through a pad of filter reagent diatomaceous earth (CELITE), concentrated to give the titled compound. UPLC-MS C18 column 5 min: t_{R}=3.07 min (690.3533 [M+H]⁺).

### Step 12: 6-[(2,5-Dioxopyrrolidin-1-yl)oxy]-N-(3-[α-D-mannopyranosyl-(1→6)-α-D-mannopyranosyl-(1→3)-β-D-mannopyranose (1-O-β) benzyl 6-((3-propyl amino)-6-oxohexanoate

The title compound was prepared using procedures analogous to those described for ML-1, Example 1, Step 4, substituting 6-({3-1α-D-mannopyranosyl-(1→6)-α-D-mannopyranosyl-(1→3)-β-D-mannopyranose (1-O-(3) oxy]propyl}amino-6-oxohexanoic acid for 6-({2-[(α-D-mannopyranosyl-(1→3)-[α-D-mannopyranosyl-(1 →6)]-β-D-glucopyranosyl)oxy]ethyl}amino)-6-oxohexanoic acid. UPLC-MS C18 column 5 min: t_{R}=3.39 min (787.3816 [M+H]+).

### Example 33

### 6-[(2,5-Dioxopyrrolidin-1-yl)oxy]-N-β-[α-D-mannopyranosyl-(1→6)-α-D-mannopyranosyl-(1→2)-α-D-mannopyranose (1-O-β) benzyl 6-((3-propyl amino)-6-oxohexanoate (ML-33)

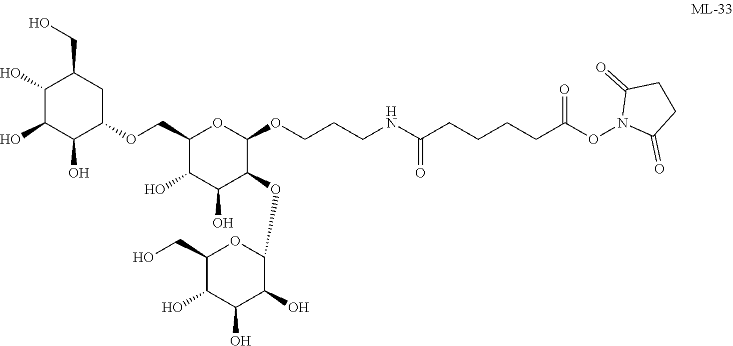 [SMILES: O=C(CCCCC(=O)ON1C(=O)CCC1=O)NCCCO[C@@H]1O[C@H](CO[C@H]2C[C@H](CO)[C@@H](O)[C@H](O)[C@@H]2O)[C@@H](O)[C@H](O)[C@@H]1O[C@H]1O[C@H](CO)[C@@H](O)[C@H](O)[C@@H]1O]

The title compound was prepared using procedures analogous to those described for ML-32 in Example 32, substituting α-D-mannopyranosyl-(1→6)-α-D-mannopyranosyl-(1→2)-3-aminoproxy-β-D-mannopyranose for α-D-mannopyranosyl-(1→4)-α-D-mannopyranosyl-(1→3)-3-aminoproxy-β-D-mannopyranose. UPLC-MS calculated for C₃₁H₅₀N₂O₂₁, 786.73, observed m/e: 787.40 (M+H)⁺, t_{R}=3.68 min.

### Example 34

### 6-[(2,5-Dioxopyrrolidin-1-yl)oxy]-N-(2-[α-D-mannopyranosyl-(1→4)-α-D-mannopyranosyl-(1→2)-β-D-mannopyranose (1-O-α) benzyl 6-((2-ethoxyl amino)-6-oxohexanoate (ML-34)

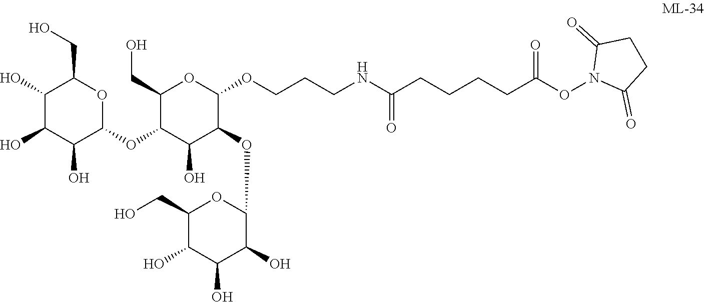 [SMILES: O=C(CCCCC(=O)ON1C(=O)CCC1=O)NCCCO[C@H]1O[C@H](CO)[C@@H](O[C@H]2O[C@H](CO)[C@@H](O)[C@H](O)[C@@H]2O)[C@H](O)[C@@H]1O[C@H]1O[C@H](CO)[C@@H](O)[C@H](O)[C@@H]1O]

### Step 1: 3,6-Dibenzoyl-2′-Azidoethoxy-α-D-mannopyranose and 2,6-Dibenzoyl-2′-Azidoethoxy-α-D-mannopyranose

The title compounds were prepared using procedures analogous to those described for Example 32 (ML-32), Step 6, substituting 2-azidoethoxy-α-D-mannopyranose for 3-azidoproxy-β-D-mannopyranose. UPLC-MS calculated for and 2,6-dibenzoyl-2′-azidoethoxy-α-D-mannopyranose C₂₂H₂₃N₃O₈, 457.43, observed m/e: 458.27 (M+H)⁺, t_{R}=1.08 min. ¹H NMR (in CDCl₃, 500 MHz) 3,6-dibenzoyl-2′-azidoethoxy-α-D-mannopyranose: 3.46 (2H, t, J=5.05 Hz), 3.73-3.67 (1H, m), 3.97-3.93 (1H, m), 4.14-4.03 (2H, m), 4.24 (1H, dd, J=3.23, 1.80 Hz), 4.61 (1H, dd, J=12.10, 2.21 Hz), 4.79 (1H, dd, J=12.10, 4.70 Hz), 4.97 (1H, d, J=1.77 Hz), 5.42 (1H, dd, J=9.57, 3.23 Hz), 7.46-7.43 (4H, m), 7.61-7.57 (2H, m), 8.10-8.07 (4H, m). 2,6-dibenzoyl-2′-azidoethoxy-α-D-mannopyranose: 3.47-3.35 (3H, m), 3.69-3.61 (2H, m), 3.96-3.84 (3H, m), 4.19 (1H, dt, J=8.22, 4.11 Hz), 4.55 (1H, dd, J=12.08, 1.69 Hz), 4.78 (1H, dd, J=12.07, 3.42 Hz), 4.97 (1H, d, J=1.75 Hz), 5.38 (1H, dd, J=3.33, 1.76 Hz), 7.0-8.1 (m, 10H). Regiochemistry was confirmed by 2D ¹H-¹H gCOSY and 1H-13C one-bond correlation (HSQC) and gHMBC experiments.

### Step 2: 2,3,4,6-Tetra-O-Benzoyl-α-D-mannopyranosyl-(1→4)-2,3,4,6-Tetra-O-Benzoyl-α-D-mannopyranosyl-(1→2)-2-azidoethoxy-3,6-dibenzoyl-α-D-mannopyranose

The title compound was prepared using procedures analogous to those described for Example 32 (ML-32), Step 7, substituting 2,6-dibenzoyl-3′-azidoproxy-β-D-mannopyranose for 3,6-dibenzoyl-2′-azidoproxy-β-D-mannopyranose. ¹H NMR (in CDCl₃, 500 MHz) 3.56-3.40 (3H, m), 3.85 (1H, ddd, J=10.44, 6.22, 3.98 Hz), 4.59-4.34 (7H, m), 4.71-4.63 (2H, m), 4.79 (1H, dd, J=12.22, 4.65 Hz), 4.88 (1H, dd, J=12.21, 2.03 Hz), 5.17 (2H, s), 5.56-5.52 (2H, m), 5.87-5.81 (3H, m), 5.93 (1H, dd, J=10.13, 3.24 Hz), 6.05 (2H, dt, J=18.55, 10.06 Hz), 7.15-8.20 (m, 15

### H).

### Step 3: α-D-mannopyranosyl-(1→4)-α-D-mannopyranosyl-(1→2)-2-aminoethoxy-α-D-mannopyranose

The title compounds was prepared using procedures analogous to those described for Example 32 (ML-32, Step 8, substituting 2,3,4,6-tetra-O-benzoyl-α-D-mannopyranosyl-(1→4)-2,3,4,6-tetra-O-benzoyl-α-D-mannopyranosyl-(1→2)-2-azidoethoxy-3,6-dibenzoyl-α-D-mannopyranose for 2,3,4,6-tetra-O-benzoyl-α-D-mannopyranosyl-(1→6)-2,3,4,6-tetra-O-benzoyl-α-D-mannopyranosyl-(1→3)-3-azidoproxy-2,4-dibenzoyl-O-D-mannopyranose. UPLC-MS calculated C₂₀H₃₇NO₁₆, 547.51, observed m/e: 548.28 [M+H]⁺, t_{R}=1.30 min.

### Step 4: 6-[(2,5-Dioxopyrrolidin-1-yl)oxy]-N-(2-[α-D-mannopyranosyl-(1→4)-α-D-mannopyranosyl-(1→2)-β-D-mannopyranose (1-O-α) benzyl 6-((2-ethoxyl amino)-6-oxohexanoate

The title compound was prepared using procedures analogous to those described for Example 2, Step 4 (ML-2) substituting α-D-mannopyranosyl-(1→4)-α-D-mannopyranosyl-(1→2)-2-aminoethoxy-α-D-mannopyranose for 8-({2-[(α-D-mannopyranosyl-(1→3)-[α-D-mannopyranosyl-(1→6)]-β-D-glucopyranosyl)oxy]ethyl}amino)-8-oxo-octanoic acid. UPLC-MS calculated for C₃₀H₄₈N₂O₂₁, 772.70, observed m/e: 773.36 [M+H]⁺, t_{R}=3.47 min.

### Example 35

### 6-1(2,5-Dioxopyrrolidin-1-yl)oxy]-N-(2-[α-D-mannopyranosyl-(1→4)-α-D-mannopyranosyl-(1→3)-β-D-mannopyranose (1-O-α) benzyl 6-((2-ethoxyl amino)-6-oxohexanoate (ML-35)

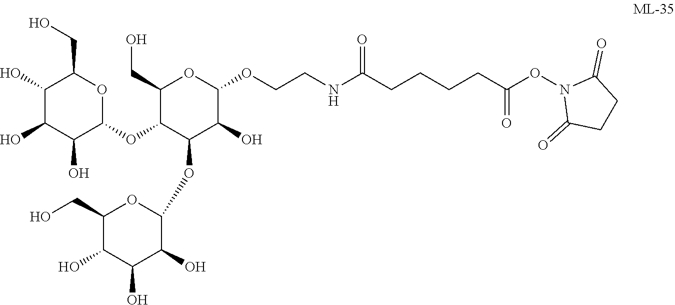 [SMILES: O=C(CCCCC(=O)ON1C(=O)CCC1=O)NCCO[C@H]1O[C@H](CO)[C@@H](O[C@H]2O[C@H](CO)[C@@H](O)[C@H](O)[C@@H]2O)[C@H](O[C@H]2O[C@H](CO)[C@@H](O)[C@H](O)[C@@H]2O)[C@@H]1O]

The title compound was prepared using procedures analogous to those described for ML-33 in Example 33, substituting 2,6-dibenzoyl-2′-azidoethoxy-α-D-mannopyranose for 3,4-dibenzoyl-3′-azidoproxy-β-D-mannopyranose. UPLC-MS calculated for C₃₀H₄₈N₂O₂₁, 772.70, observed m/e: 773.36 [M+H]⁺, t_{R}=3.91 min.

### Example 36

### 6-[(2,5-Dioxopyrrolidin-1-yl)oxy]-N-(2-[L-fucosyl-(1→6)-α-D-mannopyranosyl-(1→3)-α-D-mannopyranose (1-O-α) benzyl 6-((2-ethoxyl amino)-6-oxohexanoate (ML-36)

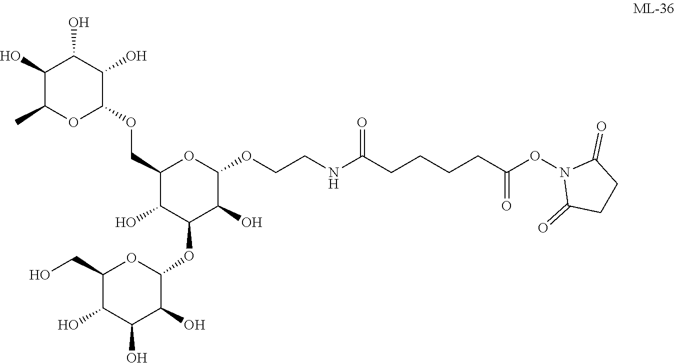 [SMILES: C[C@@H]1O[C@@H](OC[C@H]2O[C@H](OCCNC(=O)CCCCC(=O)ON3C(=O)CCC3=O)[C@@H](O)[C@@H](O[C@H]3O[C@H](CO)[C@@H](O)[C@H](O)[C@@H]3O)[C@@H]2O)[C@@H](O)[C@@H](O)[C@@H]1O]

### Step 1: 6-Trityl-2,4-di-O-benzoyl-2-azidoethoxy-α-D-mannopyranose

In a 250 ml round bottom flask, 2,4-di-O-benzoyl-2-azidoethoxy-α-D-mannopyranoside (1 g, 2.186 mmol, 1.0 eq) was dissolved in pyridine (50 mL). To above solution was added DMAP (13 mg, 0.109 mmol, 0.05 eq) followed by trityl chloride (0.762 g, 2.73 mmol, 1.25 eq). The reaction was heated to 80° C. and stirred for 18 h. Pyridine was removed under reduced pressure. The mixture was loaded on a 40 g silica gel column and purified by flash chromatography, eluted with 0-50% EtOAc/hexanes in 16 CV. The fractions containing desired product were combined and concentrated to give title compound. UPLC-MS Method B: 4.5 (722.2955, [M+Na]⁺). ¹H NMR (in CDCl₃, 500 MHz): 7.0-8.3 (m, 25H, Ph), 5.8 (t, 1H, H4), 5.5(m, 1H, H2), 5.2 (s, 1H, H1), 4.3 (m, 1H), 4.1 (m, 2H), 4.0 (m, 1H), 3.5 (m, 1H), 3.4 (m, 2H), 3.2 (dd, 1H), 2.7 (d, 1H).

### Step 2: 2,3,4,6-Tetra-O-Benzoyl-α-D-mannopyranosyl-(1→3)-6-trityl-2,4-di-O-benzoyl-2-azidoethoxy-α-D-mannopyranose

In a 100 mL round bottom flask was added 6-trityl-2,4-di-O-benzoyl-2-azidoethoxy-α-D-mannopyranose (400 mg, 0.572 mmol, 1.0 eq), 2,3,4,6-tetra-O-benzoyl-α-D-mannopyranosyl trichloracetimidate (508 mg, 0.686 mmol, 1.2 eq) and 4 Å molecular sieves (300 mg). To above mixture was added DCM (5 mL). The reaction was cooled to −78° C. To above mixture was added TMSOTf (10.33 μL, 0.057 mmol, 0.1 eq). The mixture was warmed slowly to 0° C. and stirred for 30 min. The reaction was then quenched with aq NaHCO₃, filtered through a pad of filter reagent diatomaceous earth (CELITE), diluted with DCM (20 mL), washed with brine and water. The organic was dried over MgSO₄, filtered and concentrated. The crude was purified by flash chromatography using a 80 g silica gel column, eluted with 0-100% EtOAc/hexanes in 33 min. The fractions containing the product were concentrated and dried over vacuum. UPLC-Method B: 3.14 (1278.80 [M+H]⁺). ¹H NMR (in CDCl₃, 500 MHz): 7.1-8.3 (m, 30H, Ph), 6.0 (t, 1H), 5.8 (t, 1H), 5.7 (m, 2H), 5.4 (s, 1H), 5.38 (m, 1H), 5.2 (s, 1H), 4.7 (dd, 1H), 4.6 (dd, 1H), 4.45 (m, 1H), 4.35 (dd, 1H), 3.9-4.0 (m, 2H), 3.8 (m, 2H), 3,7 (m, 1H), 3.4 (m, 2H).

### Step 3: Tetra-O-Benzoyl-α-D-mannopyranosyl-(1→3)-2,4-di-O-benzoyl-2-azidoethoxy-α-D-mannopyranose

In a 50 mL round bottom flask was added 2,3,4,6-tetra-O-benzoyl-α-D-mannopyranosyl-(1→3)-6-trityl-2,4-di-O-benzoyl-2-azidoethoxy-α-D-mannopyranose (450 mg, 0.352 mmol, 1.0 eq) and DCM (3 mL). To above solution was added TFA (3 mL, 38.9 mmol, 110 eq). The reaction was stirred at 25° C. for 1 h. The reaction was then diluted with DCM (10 mL), washed with water (15 mL×3) and brine (10 mL). The organic was dried over MgSO₄, filtered and concentrated. The crude was purified by flash chromatography, using a 40 g silica gel column, eluted with 0-100% EtOAc/hexanes in 19 min. The fractions containing the product were concentrated and dried over vacuum. UPLC-MS Method B: 1.79 (1036.31 [M+H]⁺). ¹H NMR (in CDCl₃, 500 MHz): 7.0-8.5 (m, 45H, Ph), 6.1 (t, 1H), 6.0 (t, 1H), 5.7 (m, 2H), 5.4 (s, 1H), 5.3 (s, 1H), 5.25 (s, 1H), 4.6 (m, 2H), 4.5 (m, 1H), 4.3 (m, 1H), 4.0-4.2 (m, 3h), 3.8 (m, 1H), 3.3-3.5 (m, 3H).

### Step 4: Per-TMS-L-Fucose

In a 250 mL round bottom flask, L-fucose (4 g, 24.37 mmol, 1.0 eq) was dissolved in DMF (25 mL). To above solution was added TEA (17.32 ml, 124 mmol, 5.1 eq). The mixture was cooled to 0° C. To above mixture at 0° C. was added TMS-Cl (15.88 ml, 124 mmol, 5.1 eq). The reaction was warmed to 25° C. and stirred for 4 h. The mixture was poured to ice and hexanes (100 mL, 1:1), extracted with hexanes, and the organics were dried over MgSO₄, filtered and concentrated to give the titled product. ¹H NMR (in CDCl₃, 500 MHz): 5.0 (s, 1H), 4.0 (m, 1H), 3.8 (m, 2H), 3.6 (s, 1H), 1.2 (m, 3H), 0-0.3 (m, 36H).

### Step 5: L-Fucosyl (1→6)-Tetra-O-Benzoyl-α-D-mannopyranosyl-(1→3)-2,4-di-O-benzoyl-2-azidoethoxy-α-D-mannopyranose

To a stirred solution of per-TMS L-fucose (1311 mg, 2.90 mmol) in dry CH₂Cl₂ (5 ml), TMS-I (394 μl, 2.90 mmol) was added at 25° C., and the reaction was stirred for 30 min. The mixture was then added to a solution of tetra-O-benzoyl-α-D-mannopyranosyl-(1→3)-2,4-di-O-benzoyl-2-azidoethoxy-α-D-mannopyranose (600 mg, 0.579 mmol) and 2,6-di-tert-butylpyridine (125 mg, 0.656 mmol) in dry DCM (2 mL) and stirred at 25° C. for 4 h. To above mixture was added MeOH (5 mL). The reaction was stirred at 25° C. for 30 min. The reaction mixture was neutralized using ion exchange resin (DOWEX) OH⁻ form, filtered, concentrated and purified by flash chromatography, using a 80 g silica gel column, eluted with 0-15% MeOH/DCM in 40 min, to afford the titled product. iH NMR (in CDCl₃, 500 MHz): 1.24 (3H, t, J=6.67 Hz), 3.47-3.35 (2H, m), 3.68-3.64 (3H, m), 3.81 (2H, s), 4.00-3.92 (3H, m), 4.16-4.06 (2H, m), 4.34 (1H, dd, J=12.38, 3.48 Hz), 4.45-4.42 (1H, m), 4.65-4.58 (2H, m), 4.87 (1H, t, J=3.81 Hz), 5.11 (1H, d, J=1.87 Hz), 5.32-5.27 (2H, m), 5.67-5.64 (2H, m), 6.10-5.99 (2H, m), 7.19-8.25 (m, 30H).

### Step 6: L-Fucosyl (1→6)-α-D-mannopyranosyl-(1→3)-2-aminoethoxy-α-D-mannopyranose

The title compound was prepared using procedures analogous to those described for Example 32, Steps 8 and 9 (ML-32), substituting L-fucosyl (1→6)-tetra-O-benzoyl-α-D-mannopyranosyl-(1→3)-2,4-di-O-benzoyl-2-azidoethoxy-α-D-mannopyranose for 2,3,4,6-tetra-O-benzoyl-α-D-mannopyranosyl-(1→6)-2,3,4,6-tetra-O-benzoyl-α-D-mannopyranosyl-(1→3)-3-azidoproxy-2,4-dibenzoyl-β-D-mannopyranose. UPLC-MS calculated C₂₀H₃₇NO₁₅, 531.51, observed m/e: 532.21[M+H]⁺, t_{R}=0.16 min.

### Step 7: 6-[(2,5-Dioxopyrrolidin-1-yl)oxy]-N-(2-[L-fucosyl-(1→6)-α-D-mannopyranosyl-(1→3)-α-D-mannopyranose (1-O-α) benzyl 6-((2-ethoxyl amino)-6-oxohexanoate

The title compound was prepared using procedures analogous to those described for Example 1, Step 4 (ML-1), substituting L-fucosyl (1→6)-α-D-mannopyranosyl-(1→3)-2-aminoethoxy-α-D-mannopyranose for 6-({2-[α-D-mannopyranosyl-(1→3)-[α-D-mannopyranosyl-(1→6)]-β-D-glucopyranosyl)oxy]ethyl}amino)-6-oxohexanoic acid. UPLC-MS calculated for C₃₀H₄₈N₂O₂₀, 756.70, observed m/e: 757.36 [M+H]⁺, t_{R}=3.53 min.

### Example 37

### 6-[(2,5-Dioxopyrrolidin-1-yl)oxy]-N-(2-[L-fucosyl-(1→6)-L-fucosyl-(1→3)-α-D-mannopyranose (1-O-α) benzyl 6-((2-ethoxyl amino)-6-oxohexanoate (ML-37)

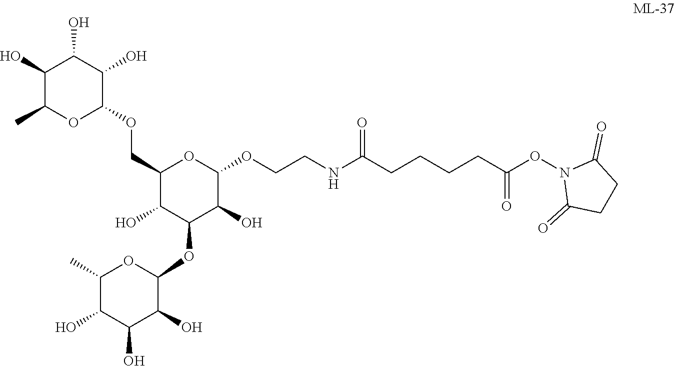 [SMILES: C[C@@H]1O[C@@H](OC[C@H]2O[C@H](OCCNC(=O)CCCCC(=O)ON3C(=O)CCC3=O)[C@@H](O)[C@@H](O[C@@H]3O[C@@H](C)[C@@H](O)[C@H](O)[C@@H]3O)[C@@H]2O)[C@@H](O)[C@@H](O)[C@@H]1O]

### Step 1: L-Fucosyl (1→6)-L-Fucose-(1→3)-2,4-di-O-benzoyl-2-azidoethoxy-α-D-mannopyranose

To a stirred solution of per-TMS L-fucose (5.2 g, 11.48 mmol, 3.5 eq) in dry CH₂Cl₂ (20 ml) was added TMS-I (1.56 ml, 11.48 mmol, 3.5 eq) at 25° C. The reaction was stirred for 30 min. The mixture was then added to a solution of 2,4-O-dibenzoyl-α-D-mannopyranosyl-2-azidoethoxy-α-D-mannopyranose (1.5 g, 3.28 mmol, 1.0 eq) premixed with 2,6-di-tert-butylpyridine (2.58 ml, 11.48 mmol) in dry DCM (10 ml). The reaction was stirred at 25° C. for 18 h. To above mixture was added MeOH (5 mL). The reaction was stirred at 25° C. for 60 min. The reaction mixture was neutralized using ion exchange resin (DOWEX) OH⁻ form, filtered, concentrated and purified by SFC (70% MeOH with 5% water/CO₂, 35° C., 100 bar, column 4.6) to afford the title product. ¹H NMR (in CDCl₃, 500 MHz): 0.8 (3H, d, J=6.58 Hz), 1.24 (3H, d, J=6.58 Hz), 3.21 (1H, s), 3.41-3.39 (2H, m), 3.5-3.8 (8H, m), 3.96 (3H, t, J=11.01 Hz), 4.04-4.02 (1H, m), 4.49-4.45 (2H, m), 5.03-5.00 (2H, m), 5.64 (1H, s), 5.89 (1H, t, J=9.90 Hz), 7.5-8.2 (10H, m).

### Step 2: L-Fucosyl (1→6)-L-Fucose-(1→3)-2-azidoethoxy-α-D-mannopyranose

The title compound was prepared using procedures analogous to those described for Example 2, Steps 8 and 9 (ML-2), substituting mannose for fucose. UPLC-MS calculated C₂₀H₃₇NO₁₄, 515.51, observed m/e: 516.17 [M+H]⁺, t_{R}=0.11 min.

### Step 3: 6-[(2,5-Dioxopyrrolichn-1-yl)oxy]-N-(2-[L-fucosyl-(1→6)-L-fucosyl-(1→3)-α-D-mannopyranose (1-O-α)benzyl 6-((2-ethoxyl amino)-6-oxohexanoate

The title compound was prepared using procedures analogous to those described for Example 1, Step 4 (ML-1), substituting L-fucosyl (1→6)-L-fucose-(1→3)-2-azidoethoxy-α-D-mannopyranose for 2-aminoethyl α-D-mannopyranosyl-(1→3)-[α-D-mannopyranosyl-(1→6)]-α-D-mannopyranoside in Step 2. UPLC-MS calculated for C₃₀H₄₈N₂O₁₉, 740.70, observed m/e: 741.21 [M+H]⁺, t_{R}=2.27 min.

### Example 38

### 6-[(2,5-Dioxopyrrolidin-1-yl)oxy]-N-(2-[α-D-mannopyranosyl-(1→3)-[α-D-mannopyranosyl-(1→6)]-α-D-mannopyranosyl]propyl)-6-oxohexanamide (ML-38)

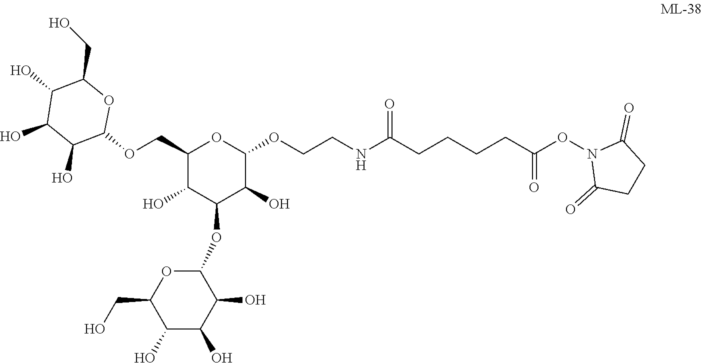

### Step 1: (9H-fluoren-9-yl)methyl (3-(α-D-mannopyranosyl)propyl)carbamate

To a solution of 3-azidopropyl-2,3,4,6-tetra-O-benzylα-D-mannopyranose

(Carbohydrate Research (1992), 223, 243-53) (7.7 g, 12.67 mmol) in a mixture of MeOH (60 ml) and water (20 ml) was added concentrated HCl (2 ml, 25.3 mmol) followed by Pd(OH)₂ (445 mg), and the resulting mixture stirred under H₂ overnight. The mixture was filtered through a pad of filter reagent diatomaceous earth (CELITE), and the filtrate evaporated. The residue was taken up into MeOH (60 ml) and water (20 ml) and concentrated HCl (2 ml, 25.3 mmol) added followed by 10% Pd/C (1.3 g), and the resulting mixture was hydrogenated on a Parr shaker at 50 psi overnight. The mixture was filtered through a pad of filter reagent diatomaceous earth (CELITE), and the filtrate was evaporated. The residue was diluted with water (100 ml) and lyophilized. The residue was dissolved in DMF (60 ml) and treated with Hünig's base (4.87 ml, 27.9 mmol) and Fmoc-OSU (4.27 g, 12.67 mmol), and the resulting mixture stirred at rt for 5 h. The mixture evaporated, and the residue purified by silica gel flash chromatography using a 275 g gold column, eluted with gradient 5-40% CH₃CN in water (10 CV) to afford the title compound. ¹H NMR δ (ppm)(DMSO-d₆): 1.44-1.41 (2H, m), 1.55-1.53 (2H, m), 3.01 (2H, d, J=6.89 Hz), 3.28 (1H, td, J=7.10, 3.19 Hz), 3.44-3.41 (1H, m), 3.52-3.47 (3H, m), 3.62 (2H, ddd, J=11.40, 6.18, 3.18 Hz), 4.22 (1H, t, J=6.92 Hz), 4.29 (2H, d, J=7.08 Hz), 4.41-4.37 (2H, m), 4.61 (1H, d, J=4.98 Hz), 4.71 (1H, d, J=5.15 Hz), 7.31 (1H, t, J=5.58 Hz), 7.34 (2H, t, J=7.47 Hz), 7.42 (2H, t, J=7.46 Hz), 7.70 (2H, d, J=7.49 Hz), 7.90 (2H, d, J=7.55 Hz).

### Step 2: (9H-fluoren-9-yl)methyl-β-(2,4-di-O-benzoyl-α-D-mannopyranosyl)propyl)carbamate

To a suspension of (9H-fluoren-9-yl)methyl (3-(α-D-mannopyranosyl)propyl) carbamate (3.3 g, 7.44 mmol) in ACN (50 ml) was added trimethyl orthobenzoate (2.3 ml, 13.39 mmol) followed by TFA (0.057 ml, 0.744 mmol), and the resulting mixture stirred at rt overnight. 10% aqueous TFA (1.9 ml, 2.46 mmol) was added, and the mixture stirred for a further 4 h. The mixture was evaporated, and the residue purified by silica gel flash chromatography using a 120 g gold column, eluted with gradient 0-100% EtOAc in hexanes (10 CV), to give the title compound. ¹H NMR δ (ppm)(CHCl₃-d): 1.70 (3H, br s), 1.88 (1H, d, J=11.32 Hz), 2.68 (1H, br s), 3.28 (2H, t, J=7.40 Hz), 3.80 (2H, s), 3.86-3.83 (1H, m), 4.23 (2H, t, J=6.77 Hz), 4.32 (1H, dd, J=9.05, 3.41 Hz), 4.44 (2H, d, J=6.81 Hz), 5.05 (1H, t, J=6.19 Hz), 5.36 (1H, s), 5.46 (1H, t, J=8.67 Hz), 7.33 (2H, td, J=7.48, 2.66 Hz), 7.41 (2H, t, J=7.54 Hz), 7.48 (4H, td, J=7.71, 3.01 Hz), 7.62-7.59 (4H, m), 7.78 (2H, d, J=7.56 Hz), 8.12-8.07 (4H, m).

### Step 3: (9H-fluoren-9-yl)methoxy)carbonyl)-3-amino-([2,3,4,6-penta-O-benzoyl-α-D-mannopyranosyl-(1→3)-[2,3,4,6-penta-O-benzoyl-α-D-mannopyranosyl-(1→6)]-2,4-di-O-benzoyl-α-D-mannopyranosyl]propane)

4 Å Molecular sieves (2 g) were weighed into a 250 ml round bottom flask. To this flask was added a solution of (9H-fluoren-9-yl)methyl-β-(2,4-di-O-benzoyl-α-D-mannopyranosyl) propyl)carbamate (1.2 g, 1.84 mmol) and 2,3,4,6-tetrabenzoyl 1-(2,2,2-tnchloroethaninnclate) α-d-mannopyranose (2.95 g, 4.05 mmol) in anhydrous DCM (40 ml). The mixture was cooled down to −30° C. and stirred for 10 min under N₂. At this temperature, trimethylsilyl-trifluoromethane sulfonate (0.033 ml, 0.184 mmol) was added, and the reaction mixture was stirred at −30° C. warming to 0° C. over 2 h. The reaction was quenched with TEA (0.128 ml, 0.921 mmol), filtered through a pad of filter reagent diatomaceous earth (CELITE), and the filtrate concentrated under vacuum. The residue was purified by silica gel flash chromatography using a 220 g gold column, eluted with gradient 0-75% EtOAc in hexanes (10 CV) to give the title compound. ¹H NMR δ (ppm) (CHCl₃-d): 1.72 (1H, br s), 1.90 (1H, br s), 2.01 (1H, d, J=13.59 Hz), 2.08 (1H, br s), 3.35 (1H, t, J=9.97 Hz), 3.51 (1H, br s), 3.88 (1H, d, J=10.12 Hz), 4.08(2H, s), 4.36-4.26 (4H, m), 4.42 (2H, s), 4.53-4.48 (3H, m), 4.61 (1H, dd, J=8.11, 3.57 Hz), 4.65 (1H, d, J=10.51 Hz), 4.76 (1H, d, J=12.24 Hz), 5.23 (1H, s), 5.45 (1H, s), 5.50 (1H, s), 5.60 (1H, t, J=3.62 Hz), 5.67 (1H, t, J=7.81 Hz), 5.79-5.77 (2H, m), 6.07-6.03 (3H, m), 6.23 (1H, t, J=10.13 Hz), 7.10 (2H, t, J=7.68 Hz), 7.15 (2H, dd, J=8.69, 6.58 Hz), 7.31-7.28 (2H, m), 7.40-7.33 (11H, m), 7.47-7.41 (8H, m), 7.54-7.50 (5H, m), 7.62-7.58 (5H, m), 7.71 (2H, dd, J=7.65, 3.48 Hz), 7.78-7.76 (2H, m), 7.81 (2H, dd, J=7.97, 1.44 Hz), 7.88 (2H, d, J=7.83 Hz), 7.92-7.90 (2H, m), 7.99-7.97 (2H, m), 8.05 (2H, d, J=7.81 Hz), 8.10 (2H, d, J=7.87 Hz), 8.14 (4H, dd, J=7.74, 4.00 Hz), 8.27-8.25 (2H, m).

### Step 4: 3-amino-([2,3,4,6-penta-O-benzoyl-α-D-mannopyranosyl-(1→3)-[2,3,4,6-penta-O-benzoyl-α-D-mannopyranosyl-(1→6)]-2,4-di-O-benzoyl-α-D-mannopyranosyl]propane)

To a solution of (9H-fluoren-9-yl)methoxy)carbonyl)-3-amino-([2,3,4,6-penta-O-benzoyl-α-D-mannopyranosyl-(1→3)-[2,3,4,6-penta-O-benzoyl-α-D-mannopyranosyl-(1→6)]-2,4-di-O-benzoyl-α-D-mannopyranosyl]propane) (1.4 g, 0.774 mmol) in DCM (15 ml) was added piperidine (1.53m1, 15.48 mmol), and the resulting mixture was stirred at rt for 2 h. The mixture was evaporated, and the residue purified by silica gel flash chromatography using a 80 g gold column, eluted with gradient 0-15% MeOH in DCM (8 CV) then hold 15% MeOH in DCM (6 CV) to give the title compound. ¹H NMR δ (ppm) (CHCl₃-d): 1.77-1.70 (2H, m), 1.95-1.87 (1H, m), 2.00 (1H, t, J=12.26 Hz), 2.91-2.81 (2H, m), 3.95 (1H, d, J=7.30 Hz), 4.24-4.21 (1H, m), 4.30 (2H, d, J=6.89 Hz), 4.42-4.35 (2H, m), 4.52-4.48 (2H, m), 4.63-4.60 (2H, m), 4.72 (1H, dd, J=12.20, 2.50 Hz), 5.23 (1H, s), 5.44 (1H, d, J=1.84 Hz), 5.51 (1H, s), 5.62 (1H, t, J=3.72 Hz), 5.75 (1H, t, J=7.14 Hz), 5.80-5.77 (2H, m), 6.01 (1H, dd, J=10.16, 3.26 Hz), 6.06 (1H, t, J=10.06 Hz), 6.19 (1H, t, J=10.12 Hz), 7.21 (2H, t, J=7.73 Hz), 7.34 (2H, d, J=7.54 Hz), 7.41-7.36 (8H, m), 7.44-7.41 (6H, m), 7.54-7.50 (5H, m), 7.58-7.55 (4H, m), 7.61-7.59 (2H, m), 7.77 (2H, dd, J=7.99, 1.44 Hz), 7.83 (4H, td, J=8.04, 1.41 Hz), 7.90 (2H, dd, J=7.98, 1.40 Hz), 8.02 (2H, dd, J=3.93, 1.54 Hz), 8.04 (2H, dd, J=3.97, 1.38 Hz), 8.09 (2H, dd, J=7.95, 1.42 Hz), 8.15-8.11 (4H, m), 8.28-8.26 (2H, m).

### Step 5: benzyl N-(3-[2,3,4,6-penta-O-benzoyl-α-D-mannopyranosyl-(1→3)-[2,3,4,6-penta-O-benzoyl-α-D-mannopyranosyl-(1→6)]-2,4-di-O-benzoyl-α-D-mannopyranosyl]propyl)-6-oxohexanoate

To a solution of 3-amino-([2,3,4,6-penta-O-benzoyl-α-D-mannopyranosyl-(1→3)-[2,3,4,6-penta-O-benzoyl-α-D-mannopyranosyl-(1→6)]-2,4-di-O-benzoyl-α-D-mannopyranosyl] propane) (800 mg, 0.504 mmol) and 6-(benzyloxy)-6-oxohexanoic acid (143 mg, 0.605 mmol) in DCM (5 ml) was added EDC (155 mg, 0.807 mmol) and HOBt (108 mg, 0.706 mmol), and the resulting mixture stirred at rt for 1 h. The mixture was diluted with further DCM (5 ml) and washed with water (10 ml); the organic layer was dried over Na₂SO₄, filtered and evaporated. The residue was purified by silica gel flash chromatography using a 40 g gold column) eluted with gradient 0-100% EtOAc in hexanes (8 CV) to give the title compound. ¹H NMR δ (ppm) (CHCl₃-d): 1.69 (2H, s), 1.92-1.84 (2H, m), 2.02-1.97 (3H, m), 2.15 (2H, t, J=7.28 Hz), 3.30-3.24 (1H, m), 3.57-3.51 (1H, m), 3.84 (1H, d, J=10.29 Hz), 4.26 (2H, d, J=10.37 Hz), 4.42-4.31 (3H, m), 4.48 (1H, dt, J=10.19, 3.19 Hz), 4.53 (1H, dd, J=12.25, 3.82 Hz), 4.60 (1H, dd, J=7.99, 3.54 Hz), 4.64 (1H, dt, J=10.16, 3.06 Hz), 4.77 (1H, dd, J=12.22, 2.50 Hz), 5.07 (2H, s), 5.20 (1H, s), 5.44 (1H, d, J=1.83 Hz), 5.49 (1H, s), 5.58 (1H, t, J=3.67 Hz), 5.62 (1H, t, J=7.84 Hz), 5.73 (1H, dd, J=3.32, 1.82 Hz), 5.77 (1H, dd, J=10.07, 3.28 Hz), 5.98 (1H, dd, J=10.15, 3.29 Hz), 6.03 (1H, t, J=10.05 Hz), 6.20 (1H, t, J=10.12 Hz), 6.54 (1H, t, J=5.60 Hz), 7.22 (2H, t, J=7.76 Hz), 7.39-7.33 (11H, m), 7.46-7.39 (8H, m), 7.55-7.50 (5H, m), 7.63-7.57 (4H, m), 7.78-7.76 (2H, m), 7.80-7.78 (2H, m), 7.84-7.82 (2H, m), 7.91-7.89 (2H, m), 7.97-7.95 (2H, m), 8.04 (2H, dd, J=7.97, 1.40 Hz), 8.10-8.08 (2H, m), 8.13 (2H, dd, J=4.64, 1.58 Hz), 8.15-8.14 (2H, m), 8.26-8.24 (2H, m).

### Step 6: Methyl N-(3-[α-D-mannopyranosyl-(1→3)-[α-D-mannopyranosyl-(1→6)]-α-D-mannopyranosyl]propyl)-6-oxohexanoate

To a solution of benzyl N-(3-[2,3,4,6-penta-O-benzoyl-α-D-mannopyranosyl-(1→3)-[2,3,4,6-penta-O-benzoyl-α-D-mannopyranosyl-(1→6)]-2,4-di-O-benzoyl-α-D-mannopyranosyl]propyl)-6-oxohexanoate (750 mg, 0.416 mmol) in a mixture of anhydrous MeOH (6 ml) and anhydrous DCM (3 ml) was added sodium methoxide (0.077 ml of a 30% wt solution in MeOH, 0.416 mmol), and the resulting mixture stirred at rt overnight. The mixture was neutralized by the addition of ion exchange resin (DOWEX) H⁺ form and filtered, and the filtrate evaporated to a volume ˜2 ml. This mixture was added dropwise to ACN (40 ml). The mixture was centrifuged at 3500 rpm for 20 min, the supernatant was decanted, and the pellet re-suspended in ACN (40 ml) and centrifuged at 3500 rpm for 20 min. The supernatant was decanted, and the pellet was dried under a stream of dry nitrogen to give the title compound. UPLC Method B: calculated for C₂₈H₄₉NO₁₈ 687.29, observed m/e=688.36 [M+1]; t_{R}=2.28 min.

### Step 7: N-(3-[α-D-mannopyranosyl-(1→3)-[α-D-mannopyranosyl-(1→6)]-α-D-mannopyranosyl]propyl)-6-oxohexanoic acid

To a solution of methyl N-(3-[α-D-mannopyranosyl-(1→3)-[α-D-mannopyranosyl-(1→6)]-α-D-mannopyranosyl]propyl)-6-oxohexanoate (260 mg, 0.378 mmol) in water (4 ml) was added 1N aq NaOH (0.416 ml, 0.416 mmol), and the resulting mixture was stirred at rt for 90 min. The mixture was neutralized by the addition of ion exchange resin (DOWEX) H⁺ form, filtered and lyophilized to give the title compound. UPLC Method B: calculated for C₂₇H₄₇NO₁₈ 673.28, observed m/e=674.33 [M+1]; t_{R}=2.04 min.

### Step 8: 6-[(2,5-Dioxopyrrolidin-1-yl)oxy]-N-(3-[α-D-mannopyranosyl-(1→3)-[α-D-mannopyranosyl-(1→6)]-α-D-mannopyranosyl]propyl)-6-oxohexanamide

The title compound was prepared using procedures analogous to those described for Example 1, Step 4 (ML-1) substituting 6-({2-[(α-D-mannopyranosyl-(1→3)-[α-D-mannopyranosyl-(1→6)]-β-D-glucopyranosyl)oxy]ethyl}amino)-6-oxohexanoic acid with N-(3-[α-D-mannopyranosyl-(1→3)-[α-D-mannopyranosyl-(1→6)]-α-D-mannopyranosyl]propyl)-6-oxohexanoic acid to give the title compound. UPLC Method B: calculated for C₃₁H₅₀N₂O₂₀ 770.30, observed m/e=771.35 [M+H]⁺; t_{R}=2.50 min.

### Example 39

### 2-{([α-D-mannopyranosyl-(1→3)-[α-D-mannopyranosyl-(1→6)]-α-D-mannopyranosyl]oxy)ethyl}{6-[(2,5-dioxopyrrolidin-1-yl)oxy]-6-oxohexyl}-N-methylamine (ML-39)

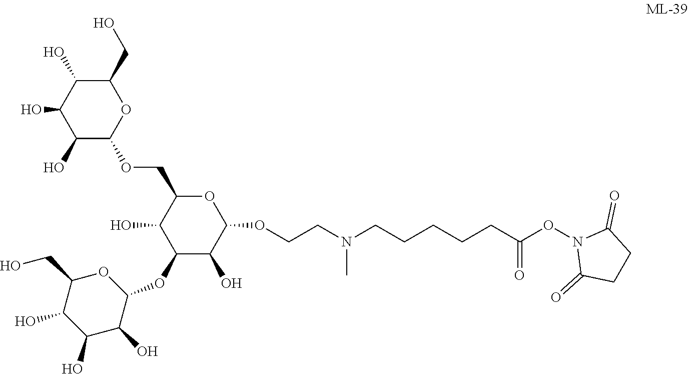 [SMILES: CN(CCCCCC(=O)ON1C(=O)CCC1=O)CCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O]

### Step 1: benzyl 6-({2,3,4,6-penta-O-benzoyl-α-D-mannopyranosyl-(1→3)-[2,3,4,6-penta-O-benzoyl-α-D-mannopyranosyl-(1→6)]-2,4-di-O-benzoyl-α-D-mannopyranosyl]-2-oxyethyl}-N-methylamino)hexanoate.

To a solution of benzyl 6-({2,3,4,6-penta-O-benzoyl-α-D-mannopyranosyl-(1→3)-[2,3,4,6-penta-O-benzoyl-α-D-mannopyranosyl-(1→6)]-2,4-di-O-benzoyl-α-D-mannopyranosyl]-2-oxyethyl}amino) hexanoate (WO 2015051052) (1.18 g, 0.658 mmol) and 37% aq formaldehyde (0.098 ml, 1.31 mmol) in DCM (5 ml) was added AcOH (0.038 ml, 0.658 mmol) followed by sodium triacetoxyborohydride (209 mg, 0.987 mmol), and the resulting mixture stirred at rt overnight. The mixture was evaporated, and the residue dissolved in EtOAc (30 ml) and washed with sat. NaHCO₃ (2×30 ml), sat. NaCl (20 ml), dried over Na₂SO₄, filtered and evaporated to give the title compound. UPLC Method C: calculated for C₁₀₄H₉₅NO₂₈ 1805.60, observed m/e=1806.69 [M+1]; t_{R}=4.29 min, ¹H NMR δ (ppm)(CHCl₃-d): 1.35 (1H, s), 1.52 (1H, s), 1.62 (3H, s), 1.67 (2H, br s), 2.34 (4H, t, J=7.11 Hz), 2.43 (2H, s), 2.71 (1H, d, J=7.07 Hz), 3.80 (1H, d, J=10.68 Hz), 4.35 (2H, br s), 4.51 (2H, s), 4.66-4.55 (2H, m), 5.08 (1H, s), 5.18 (1H, d, J=9.40 Hz), 5.38 (1H, s), 5.73 (1H, dd, J=10.08, 3.13 Hz), 5.76 (1H, s), 5.80 (1H, s), 6.00 (2H, br s), 6.06 (1H, dd, J=10.33,9.63 Hz), 6.15 (1H, t, J=10.10 Hz), 7.23 (2H, t, J=7.74 Hz), 7.34-7.30 (10H, m), 7.44-7.37 (10H, m), 7.54-7.48 (3H, m), 7.60-7.55 (6H, m), 7.74-7.72 (2H, m), 7.78 (2H, d, J=7.84 Hz), 7.88-7.85 (3H, m), 8.04 (3H, d, J=8.19 Hz), 8.09-8.07 (4H, m), 8.11 (2H, d, J=7.89 Hz), 8.15 (2H, d, J=7.88 Hz), 8.32 (2H, s).

### Step 2: 6-({-α-D-mannopyranosyl-(1→3)-[-α-D-mannopyranosyl-(1→6)]-α-D-mannopyranosyl]-2-oxyethyl}-N-methylamino) hexanoic acid

Benzyl 6-({2,3,4,6-penta-O-benzoyl-α-D-mannopyranosyl-(1→3)-[2,3,4,6-penta-O-benzoyl-α-D-mannopyranosyl-(1→6)]-2,4-di-O-benzoyl-α-D-mannopyranosyl]-2-oxy ethyl}-N-methylamino) hexanoate. (1.15 g, 0.636 mmol) was suspended in anhydrous MeOH (20 ml), 25wt % sodium methoxide in MeOH (0.292 ml, 1.272 mmol) was added, and the resulting mixture stirred at rt overnight. The mixture was evaporated, and the residue dissolved in water (10 ml) and extracted with EtOAc (2×15 ml). The organic layers were discarded, and the aqueous layers were treated with 5N aq NaOH until pH=12. The mixture stirred at rt overnight. The mixture was acidified by the addition of AcOH and lyophilized to give the title compound. UPLC Method B: calculated for C₂₇H₄₉NO₁₈ 675.29, observed m/e=676.37 [M+1]; t_{R}=1.20 min.

### Step 3: (2-{([α-D-mannopyranosyl-(1→3)-[α-D-mannopyranosyl-(1→0]-α-D-mannopyranosyl]oxy)ethyl}{6-[(2,5-dioxopyrrolidin-1-yl)oxy]-6-oxohexyl}-N-methylamine)

The title compound was prepared using procedures analogous to those described for Example 1, Step 4 (ML-1) substituting 6-({2-[(α-D-mannopyranosyl-(1→3)-[α-D-mannopyranosyl-(1→6)1-β-D-glucopyranosyl)oxy]ethyl}amino)-6-oxohexanoic acid with 6-({-α-D-mannopyranosyl-(1→3)-[α-D-mannopyranosyl-(1→6)]-α-D-mannopyranosyl]-2-oxyethyl}-N-methylamino) hexanoic acid to give the title compound. UPLC Method B: calculated for C₃₁H₅₂N₂O₂₀ 772.31, observed m/e=773.39 [M+H]⁺; t_{R}=1.53 min.

### Example 40

### (1R,4R)-4-[(2,5-Dioxopyrrolidin-1-yl)oxy]-N-(2-{[α-D-mannopyranosyl-(1→3)-[α-D-mannopyranosyl-(1→6)]-α-D-mannopyranosyl]oxy}ethylcarbamoyl)-cyclohexane-1-carboxamide (ML-40)

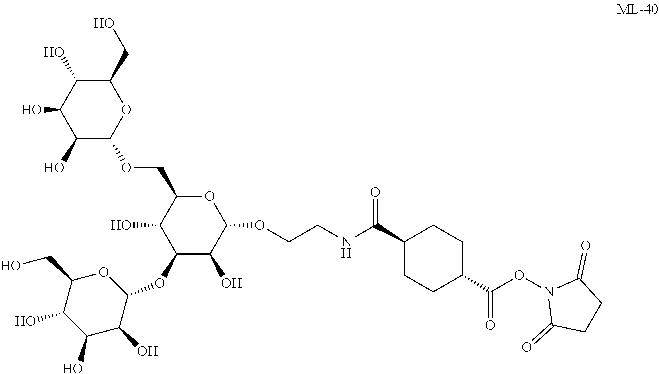 [SMILES: O=C1CCC(=O)N1OC(=O)[C@H]1CC[C@H](C(=O)NCCO[C@H]2O[C@H](CO[C@H]3O[C@H](CO)[C@@H](O)[C@H](O)[C@@H]3O)[C@@H](O)[C@H](O[C@H]3O[C@H](CO)[C@@H](O)[C@H](O)[C@@H]3O)[C@@H]2O)CC1]

### Step 1: methyl (1R,4R)-4-[(2,5-Dioxopyrrolidin-1-yl)oxy]-N-(2-{[α-D-mannopyranosyl-(1→3)-[α-D-mannopyranosyl-(1→6)]-α-D-mannopyranosyl]oxy}ethylcarbamoyl)-cyclohexan-1-oate

The title compound was prepared using procedures analogous to those described for Example 2, Step 2 (ML-2) substituting 8-(benzyloxy)-8-oxooctanoic acid with (1R,4R)-4-(methoxycarbonyl)cyclohexanecarboxylic acid to give the title compound. UPLC Method B: calculated for C₂₉H₄₉NO₁₉ 715.29, observed m/e=716.36 [M+1]; t_{R}=3.35 min.

### Step 2: (1R,4R)-4-[(2,5-Dioxopyrrolidin-1-yl)oxy]-N-(2-{[α-D-mannopyranosyl-(1→3)-[α-D-mannopyranosyl-(1→6)]-α-D-mannopyranosyl]oxy}ethylcarbamoyl)-cyclohexan-1-oic acid

The title compound was prepared using procedures analogous to those described for Example 38, Step 7 (ML-38) substituting methyl N-(3-[α-D-mannopyranosyl-(1→3)-[α-D-mannopyranosyl-(1→6)]-α-D-mannopyranosyl]propyl)-6-oxohexanoate with methyl (1R,4R)-4-[(2,5-dioxopyrrolidin-1-yl)oxy]-N-(2-{[α-D-mannopyranosyl-(1→3)-[α-D-mannopyranosyl-(1→6)]-α-D-mannopyranosyl]oxy}ethylcarbamoyl)-cyclohexan-1-oate to give the title compound. UPLC Method B: calculated for C₂₈H₄₇NO₁₉ 701.27, observed m/e=702.35 [M+H]⁺; t_{R}=4.2 min.

### Step 3: (1R,4R)-4-[(2,5-Dioxopyrrolidin-1-yl)oxy]-N-(2-{[α-D-mannopyranosyl-(1→3)-[α-D-mannopyranosyl-(1→6)]-α-D-mannopyranosyl]oxy}ethylcarbamoyl)-cyclohexane-1-carboxamide

The title compound was prepared using procedures analogous to those described for Example 1, Step 4 (ML-1) substituting 6-({2-[(α-D-mannopyranosyl-(1→3)-[α-D-mannopyranosyl-(1→6)]-β-D-glucopyranosyl)oxy]ethyl}amino)-6-oxohexanoic acid with (1R,4R)-4-[(2,5-dioxopyrrolidin-1-yl)oxy]-N-(2-{[α-D-mannopyranosyl-(1→3)-[α-D-mannopyranosyl-(1→6)1-α-D-mannopyranosyl]oxy}ethylcarbamoyl)-cyclohexan-1-oic acid to give the title compound. UPLC Method B: calculated for C₃₂H₅₀N₂O₂₁ 798.29, observed m/e=799.38 [M+1]; t_{R}=4.46 min.

### Example 41

### 2-[(2,5-Dioxopyrrolidin-1-yl)oxy]-N-(2-{[α-D-mannopyranosyl-(1→3)-[α-D-mannopyranosyl-(1→6)]-β-D-mannopyranosyl]oxy}ethyl)-1-(2-oxo-(piperidin-4-yl)ethane) (ML-41)

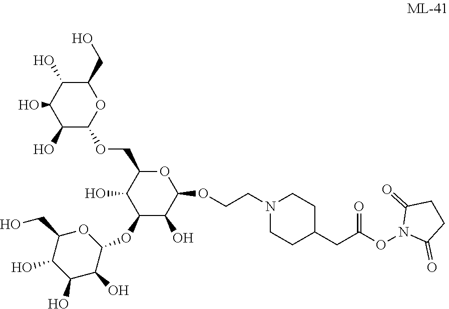 [SMILES: O=C(CC1CCN(CCO[C@@H]2O[C@H](CO[C@H]3O[C@H](CO)[C@@H](O)[C@H](O)[C@@H]3O)[C@@H](O)[C@H](O[C@H]3O[C@H](CO)[C@@H](O)[C@H](O)[C@@H]3O)[C@@H]2O)CC1)ON1C(=O)CCC1=O]

### Step 1: allyl 2,4,-di-O-benzoyl-β-D-mannopyranose

The title compound was prepared using procedures analogous to those described for Example 38, Step 2 (ML-38) substituting (9H-fluoren-9-yl)methyl (3-(α-D-mannopyranosyl) propyl)carbamate with allyl β-D-mannopyranose to give the title compound. ¹H NMR δ (ppm) (CHCl₃-d): 3.71 (1H, ddd, J=9.54, 5.05, 2.56 Hz), 3.82 (1H, dd, J=12.39, 5.08 Hz), 3.91 (1H, dd, J=12.38, 2.57 Hz), 4.21-4.17 (2H, m), 4.39 (1H, ddt, J=13.15, 4.83, 1.58 Hz), 4.86 (1H, d, J=1.14 Hz), 5.21 (1H, dq, J=10.45, 1.43 Hz), 5.30 (1H, dq, J=17.24, 1.64 Hz), 5.45 (1H, t, J=9.54 Hz), 5.75 (1H, dd, J=3.43, 1.12 Hz), 5.90 (1H, dddd, J=17.24, 10.44, 6.16, 4.84 Hz), 7.51-7.47 (4H, m), 7.62-7.59 (2H, m), 8.07-8.05 (2H, m), 8.16-8.14 (2H, m).

### Step 2: Allyl 2-O-acetyl-3,4,6-tri-O-benzoyl-α-D-mannopyranosyl-(1→3)-[2-O-acetyl-3,4,6-tri-O-benzoyl-α-D-mannopyranosyl-(1→6)]-2,4,-di-O-benzoyl-β-D-mannopyranoside

The title compound was prepared using procedures analogous to those described for Example 38, Step 3 (ML-38) substituting ((9H-fluoren-9-yl)methyl-β-(2,4-di-O-benzoyl-α-D-mannopyranosyl)propyl)carbamate with allyl 2,4,-di-O-benzoyl-β-D-mannopyranose and 2,3,4,6-tetra-benzoyl 1-(2,2,2-trichloroethanimidate) α-D-mannopyranose with 2-O-acetyl-3,4,6-tri-benzoyl 1-(2,2,2-trichloroethanimidate) α-D-mannopyranose to give the title compound. ¹H NMR δ (ppm) (CHCl₃-d): 2.07 (3H, s), 2.13 (3H, s), 3.80 (1H, dd, J=10.66, 2.63 Hz), 3.94 (1H, ddd, J=9.88, 6.90, 2.62 Hz), 4.20 (1H, dd, J=10.71, 6.93 Hz), 4.27-4.23 (1H, m), 4.34 (1H, dd, J=9.62, 3.44 Hz), 4.46-4.43 (2H, m), 4.57-4.48 (5H, m), 4.67-4.64 (1H, m), 4.71 (1H, dd, J=12.16, 2.60 Hz), 4.86 (1H, d, J=1.12 Hz), 5.01 (1H, d, J=1.77 Hz), 5.16-5.15 (2H, m), 5.25 (1H, dd, J=10.46, 1.62 Hz), 5.40 (1H, dq, J=17.23, 1.65 Hz), 5.51 (1H, dd, J=3.33, 1.78 Hz), 5.55 (1H, dd, J=9.63, 2.96 Hz), 5.74 (1H, t, J=9.72 Hz), 5.87-5.82 (3H, m), 5.95-5.88 (4H, m), 7.32-7.29 (3H, m), 7.40-7.36 (11H, m), 7.55-7.44 (15H, m), 7.61-7.56 (4H, m), 7.80-7.78 (2H, m), 7.91 (3H, dd, J=4.59, 1.54 Hz), 7.93 (3H, dd, J=4.72, 1.39 Hz), 7.99-7.97 (3H, m), 8.07-8.05 (3H, m), 8.09-8.07 (3H, m), 8.16-8.14 (2H, m), 8.26-8.24 (2H, m).

### Step 3: 2-{[2-O-acetyl-3,4,6-tri-O-benzoyl-α-D-mannopyranosyl-(1→3)-[2-O-acetyl-3,4,6-tri-O-benzoyl-α-D-mannopyranosyl-(1→6)]-2,4-di-O-benzoyl-β-D-mannopyranosyl]oxy}ethanal)

Allyl 2-O-acetyl-3,4,6-tri-O-benzoyl-α-D-mannopyranosyl-(1→3)-[2-O-acetyl-3,4,6-tri-O-benzoyl-α-D-mannopyranosyl-(1→6)]-2,4-di-O-benzoyl-p-D-mannopyranoside (6 g, 4.11 mmol) was dissolved in a mixture of water (11.5 ml) and acetone (45 ml). N-methyl morpholine-N-oxide (962 mg, 8.21 mmol) was added in one portion followed by OsO₄ (2.06 ml of a 2.5% solution in tert-butanol, 0.164 mmol), and the resulting mixture stirred at rt overnight. To this reaction mixture was added a solution of sodium metaperiodate (1.76 g, 8.21 mmol) in water (15 ml) and stirred for 4 h. The mixture was filtered through a pad of filter reagent diatomaceous earth (CELITE), and the precipitate washed with further acetone. The combined filtrates were evaporated, and the residue was dissolved in EtOAc (100 ml) and washed with sat. NaHCO₃ (100 ml); the aqueous portion was back-extracted with EtOAc (2×75 ml) and combined EtOAc layers washed with sat. NaCl (75 ml), dried over Na₂SO₄, filtered and evaporated. The residue purified by silica gel flash chromatography using a 120 g gold column, eluted with gradient 50-100% EtOAc in hexanes to give the title compound. ¹H NMR δ (ppm) (CHCl₃-d): 2.07 (3H, s), 2.13 (3H, s), 3.76 (1H, dd, J=10.90, 2.53 Hz), 3.92 (1H, ddd, J=9.89, 6.56, 2.51 Hz), 4.17 (1H, d, J=5.88 Hz), 4.32-4.30 (4H, m), 4.46-4.40 (3H, m), 4.57-4.53 (3H, m), 4.67-4.64 (1H, m), 4.72 (1H, dd, J=12.12, 2.58 Hz), 4.85 (1H, d, J=1.11 Hz), 4.98 (1H, d, J=1.78 Hz), 5.16 (3H, d, J=1.96 Hz), 5.52 (1H, dd, J=3.35, 1.78 Hz), 5.56 (1H, dd, J=9.64, 2.66 Hz), 5.76 (1H, t, J=9.73 Hz), 5.86-5.82 (3H, m), 5.93 (1H, t, J=9.88 Hz), 6.03 (1H, d, J=3.33 Hz), 7.32-7.29 (3H, m), 7.41-7.35 (11H, m), 7.48-7.44 (7H, m), 7.53-7.48 (8H, m), 7.57-7.55 (1H, m), 7.62-7.57 (3H, m), 7.80-7.78 (3H, m), 7.92 (3H, dd, J=2.66, 1.48 Hz), 7.94 (2H, dt, J=2.66, 1.25 Hz), 8.05-8.04 (4H, m), 8.06 (4H, dd, J=3.22, 1.32 Hz), 8.17-8.15 (2H, m), 8.26 (3H, dd, J=7.91, 1.45 Hz), 9.73 (1H, t, J=1.18 Hz).

### Step 4: methyl 2-{[2-O-acetyl-3,4,6-tri-O-benzoyl-α-D-mannopyranosyl-(1→3)-[2-O-acetyl-3,4,6-tri-O-benzoyl-α-D-mannopyranosyl-(1→6)]-2,4-di-O-benzoyl-β-D-mannopyranosyl]oxy}ethyl)-1-(piperidin-4-yl) acetate

To a suspension of 2-{[2-O-acetyl-3,4,6-tri-O-benzoyl-α-D-mannopyranosyl-(1→3)-[2-O-acetyl-3,4,6-tri-O-benzoyl-α-D-mannopyranosyl-(1→6)]-2,4-di-O-benzoyl-β-D-mannopyranosyl]oxy}ethanal) (1 g, 0.692 mmol) and methyl 2-(piperidin-4-yl)acetate hydrochloride (110 mg, 0.568 mmol) in DCM (5 ml) was added sodium triacetoxyborohydride (241 mg, 1.136 mmol), and the resulting mixture stirred at rt overnight. The mixture was evaporated, and the residue was partitioned between EtOAc (40 ml) and sat. NaHCO₃ (50 ml). The organic layer washed with sat. NaCl (20 ml), dried over Na₂SO₄, filtered and evaporated. The residue was purified by silica gel flash chromatography using a 40 g gold column, eluted with gradient 2-10% MeOH in DCM (7 CV) to give the title compound. ¹H NMR δ (ppm) (CHCl₃-d): 1.86 (3H, s), 2.13 (3H, s), 2.18 (3H, s), 3.64 (3H, s), 3.83 (1H, d, J=10.89 Hz), 3.91 (2H, d, J=8.93 Hz), 4.16-4.13 (1H, m), 4.30 (1H, dd, J=9.65, 3.37 Hz), 4.43 (5H, br s), 4.50-4.46 (3H, m), 4.58 (1H, br s), 4.71 (1H, d, J=12.03 Hz), 4.85 (1H, s), 5.02 (1H, s), 5.15-5.14 (2H, m), 5.56 (2H, dd, J=9.20, 3.15 Hz), 5.87-5.83 (3H, m), 5.95-5.91 (3H, m), 7.56-7.50 (9H, m), 7.59 (3H, t, J=7.71 Hz), 7.79-7.77 (3H, m), 7.89 (3H, d, J=7.90 Hz), 7.92 (3H, d, J=7.97 Hz), 8.04 (7H, d, J=8.48 Hz), 8.13 (3H, d, J=7.85 Hz), 8.25 (3H, d, J=7.65 Hz).

### Step 5: 2-{[α-D-mannopyranosyl-(1→3)-[α-D-mannopyranosyl-(1→6)]-β-D-mannopyranosyl]oxy}ethyl)-1-(piperidin-4-yl) acetic acid

To a solution of methyl 2-{[2-O-acetyl-3,4,6-tri-O-benzoyl-α-D-mannopyranosyl-(1→3)-[2-O-acetyl-3,4,6-tri-O-benzoyl-α-D-mannopyranosyl-(1→6)]-2,4,-di-O-benzoyl-β-D-mannopyranosyl]oxy}ethyl)-1-(piperidin-4-yl) acetate (322 mg, 0.201 mmol) in a mixture of DCM (3 ml) and MeOH (3 ml) was added sodium methoxide (0.037 ml of a 30% solution in MeOH), and the mixture was stirred at rt for 1 h. The DCM was removed by evaporation and further MeOH (3 ml) added and continued stirring overnight. The mixture was evaporated to a volume ˜3 ml and added dropwise to ACN (40 ml). The mixture was centrifuged at 3500 rpm for 15 min. The supernatant was decanted, and the solids were re-suspended in ACN (40 ml) and centrifuged at 3500 rpm for 15 min. The supernatant was decanted, and the remaining solids were dried under a stream of dry nitrogen. The solids were taken up into water (3 ml) and NaOH (0.401 ml of a 1M aq solution, 0.401 mmol) added, and the resulting mixture stirred at rt for 5 h. The mixture was lyophilized to give the title compound. UPLC Method B: calculated for C₂₇H₄₇NO₁₈ 673.28, observed m/e=674.33 [M+H]+; t_{R}=1.63 min.

### Step 6: 2-[(2,5-Dioxopyrrolidin-1-yl)oxy]-N-(2-{[α-D-mannopyranosyl-(1→3)-[α-D-mannopyranosyl-(1→6)]-β-D-mannopyranosyl]oxy}ethyl)-1-(2-oxo-(piperidin-4-yl)ethane)

The title compound was prepared using procedures analogous to those described for Example 1, Step 4 (ML-1) substituting 6-({2-[(α-D-mannopyranosyl-(1→3)-[α-D-mannopyranosyl-(1→6)]-β-D-glucopyranosyl)oxy]ethyl}amino)-6-oxohexanoic acid with 2-{[α-D-mannopyranosyl-(1→3)-[α-D-mannopyranosyl-(1→6)]-β-D-mannopyranosyl]oxy}ethyl)-1-(piperidin-4-yl) acetic acid to give the title compound. UPLC Method B: calculated for C₃₁H₅₀N₂O₂₀ 770.30, observed m/e=771.34 [M+H]⁺; t_{R}=1.57 min.

### Example 42

### 2-[(2,5-Dioxopyrrolidin-1-yl)oxy]-N-(2-{[α-D-mannopyranosyl-(1→3)-[α-D-mannopyranosyl-(1→6)]-β-D-mannopyranosyl]oxy}ethyl)-1-(4-oxo-(piperidin-4-yl)butane) (ML-42)

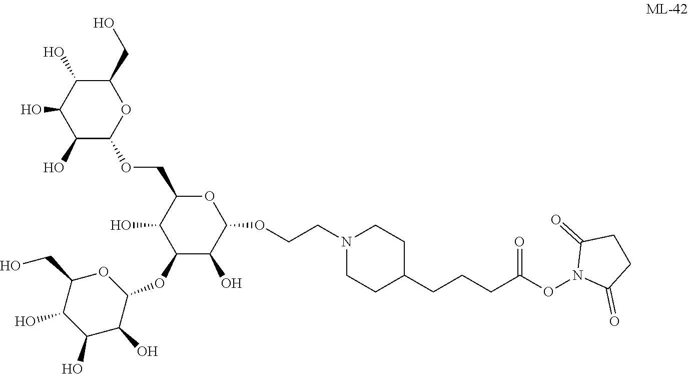 [SMILES: O=C(CCCC1CCN(CCO[C@H]2O[C@H](CO[C@H]3O[C@H](CO)[C@@H](O)[C@H](O)[C@@H]3O)[C@@H](O)[C@H](O[C@H]3O[C@H](CO)[C@@H](O)[C@H](O)[C@@H]3O)[C@@H]2O)CC1)ON1C(=O)CCC1=O]

The title compound was prepared using procedures analogous to those described for Example 41 (ML-41), substituting methyl 2-(piperidin-4-yl)acetate hydrochloride with ethyl 4-(piperidin-4-yl)butanoate hydrochloride in Step 4 to give the title compound. UPLC Method B: calculated for C₃₃H₅₄N₂O₂₀ 798.33, observed m/e=799.37 [M+H]⁺; t_{R}=2.96 min.

### Example 43

### [(2,5-Dioxopyrrolidin-1-yl)oxy]-N-(2-{[α-D-mannopyranosyl-(1→3)-[α-D-mannopyranosyl-(1→6)]-α-D-mannopyranosyl]oxy}ethyl)-(R)-1-(2-oxoethyl)pyrrolidine-3-carboxylate (ML-43)

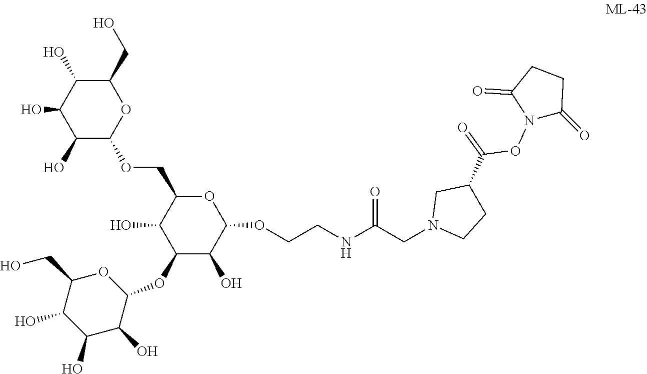 [SMILES: O=C(CN1CC[C@@H](C(=O)ON2C(=O)CCC2=O)C1)NCCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O]

The title compound was prepared using procedures analogous to those described for ML-40 in Example 40, substituting (1R,4R)-4-(methoxycarbonyl)cyclohexanecarboxylic acid with (R)-2-(3-(methoxycarbonyl)pyrrolidin-1-yl)acetic acid hydrochloride in Step 1 to give the title compound. UPLC Method B: calculated for C₃₁H₄₉N₃O₂₁ 799.29, observed m/e=800.35 [M+H]⁺; t_{R}=1.34 min.

### Example 44

### 5-azido-N-(2-(((2S,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-ypoxy)ethyl)pentanamide (ML-44)

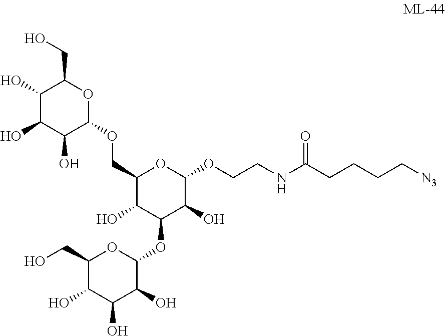 [SMILES: [N-]=[N+]=NCCCCC(=O)NCCO[C@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O]

To a solution of (2S,3S,4S,5S,6R)-2-(((2R,3R,4S,5S,6S)-6-(2-aminoethoxy)-3,5-dihydroxy-4-(((2R,3S,4S,5S,6R)-3,4,5-trihydroxy-6-(hydroxymethyl)tetrahydro-2H-pyran-2-yl)oxy)tetrahydro-2H-pyran-2-yl)methoxy)-6-(hydroxymethyl)tetrahydro-2H-pyran-3,4,5-triol (AETM) (100 mg, 0.183 mmol) in DMF (1.5 ml) was added 2,5-dioxopyrrolidin-1-yl 5-azidopentanoate (52.6 mg, 0.219 mmol) at rt, followed by Hünig's base (0.038 ml, 0.219 mmol), the mixture was stirred at rt for 4 h, the mixture was concentrated down by rotary evaporation, then was purified by silica gel chromatography, using a C18 120 g column, eluted with 0-40% ACN in water, combined fractions and lyophilized. UPLC Method A: m/e=673.316 [M+1]; t_{R}=3.82 min.

### Example 45

### 5-azido-N-(2-(((2R,3R,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)pentanamide (ML-45)

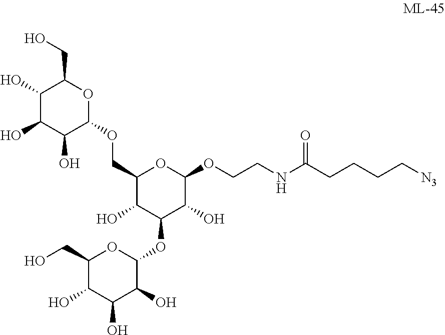 [SMILES: [N-]=[N+]=NCCCCC(=O)NCCO[C@@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@H]1O]

To a solution of (2S,3S,4S,5S,6R)-2-(((2R,3R,4S,5R,6R)-6-(2-aminoethoxy)-3,5-dihydroxy-4-(((2R,3S,4S,5S,6R)-3,4,5-trihydroxy-6-(hydroxymethyl)tetrahydro-2H-pyran-2-yl)oxy)tetrahydro-2H-pyran-2-yl)methoxy)-6-(hydroxymethyl)tetrahydro-2H-pyran-3,4,5-triol (100 mg, 0.183 mmol) in DMF (1.5 ml) was added 2,5-dioxopyrrolidin-1-yl 5-azidopentanoate (54.8 mg, 0.228 mmol) at rt, followed by Hilnig's base (0.040 ml, 0.228 mmol), the mixture was stirred at rt for 4 h. The mixture was concentrated down by rotary evaporator, then was purified by silica gel flash chromatography using a C18 120 g column, eluted with 0-40% ACN in water, combined fractions and lyophilized. UPLC Method A: m/e=673.388 [M+1]; t_{R}=2.64 min.

### Example 46

### 5-azido-N-(2-(((2R,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyptetrahydro-2H-pyran-2 yl)oxy)ethyl)pentanamide (ML-46)

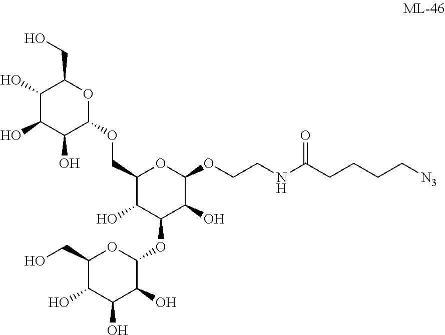 [SMILES: [N-]=[N+]=NCCCCC(=O)NCCO[C@@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O]

To solution of (2S,3S,4S,5S,6R)-2-(((2R,3R,4S,5S,6R)-6-(2-aminoethoxy)-3,5-dihydroxy-4-(((2R,3S,4S,5S,6R)-3,4,5-trihydroxy-6-(hydroxymethyl)tetrahydro-2H-pyran-2-yl)oxy)tetrahydro-2H-pyran-2-yl)methoxy)-6-(hydroxymethyl)tetrahydro-2H-pyran-3,4,5-triol β-AETM (380 mg, 0.694 mmol) in DMF (1.5 ml) was added 2,5-dioxopyrrolidin-1-yl 5-azidopentanoate (200 mg, 0.833 mmol) at rt, followed by Hünig's base (0.145 ml, 0.833 mmol), the mixture was stirred at rt for 4 h, the mixture was concentrated down by rotary evaporation, then was purified by silica gel flash chromatography using a C18 120 g column, eluted with 0-40% ACN in water, combined fractions and lyophilized). UPLC Method A: m/e=673.388 [M+1]; t_{R}=2.64 min.

### Example 47

### 2,5-dioxopyrrolidin-1-yl 6-((2-((2-(((2R,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-3,4-dioxocyclobut-1-en-1-yl)amino)hexanoate (ML-47)

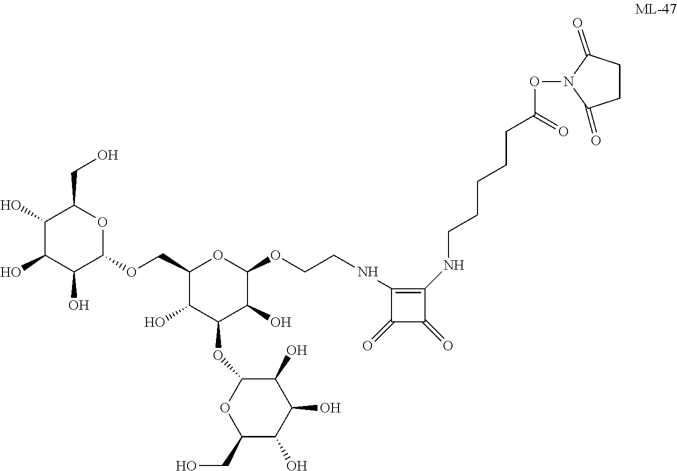 [SMILES: O=C(CCCCCNc1c(NCCO[C@@H]2O[C@H](CO[C@H]3O[C@H](CO)[C@@H](O)[C@H](O)[C@@H]3O)[C@@H](O)[C@H](O[C@H]3O[C@H](CO)[C@@H](O)[C@H](O)[C@@H]3O)[C@@H]2O)c(=O)c1=O)ON1C(=O)CCC1=O]

### Step 1: Benzyl 6-((2-isopropoxy-3,4-dioxocyclobut-1-en-1-yl)amino)hexanoate

A mixture of 6-amino-hexanoic acid benzyl ester, compound with toluene-4-sulfonic acid (500 mg, 1.271 mmol) and 3,4-diisopropoxy-3-cyclobutene-1,2-dione (252 mg, 1.271 mmol) in 12.7 ml of DMF was heated in the presence of Hünig's base (222 μl, 1.271 mmol) at 80° C. for a period of 24 h. The reaction mixture was concentrated and isolated the product by silica gel flash chromatography using a C18 column, gradient 0-100% of ACN-water-0.05% TFA. The title compound was isolated after lyophilization. UPLC-MS: calculated C₂₀H₂₅NO₅, 359.17 observed m/z: 359.0 (M+H), (t_{R}=1.39/2.00 min).

### Step 2: Benzyl 6-((2-((2-(((2R,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-3,4-dioxocyclobut-1-en-1-yl)amino)hexanoate

The mixture of 2-aminoethyl α-D-mannopyranosyl-(1→3)-1α-D-mannopyranosyl-(1→6)-β3-D-mannopyranoside (839 mg, 1.532 mmol), product of Step 1 (367 mg, 1.021 mmol), Hünig's base (0.535 ml, 3.06 mmol), was heated at 80° C. for 3 days using DMF (10 ml) as the solvent. The reaction mixture was concentrated and isolated the product by silica gel flash chromatography with a C18 120 g column, using gradient 0-100% of AcN-water-0.05% TFA. The product was lyophilized to give the title compound. UPLC-MS: calculated C₃₇H₅₄N₂O₂₀ 846.33, observed m/z: 847.4 (M+H) (t_{R}=3.40/5.00 min).

### Step 3: 6-((2-((2-(((2R,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-3,4-dioxocyclobut-1-en-1-yl)amino)hexanoic acid

The mixture of 100 mg (0.118 mmol) of the product of Step 2, 0.945 ml of THF, 0.236 ml of water, and 28.3 mg (1.81 mmol) of LiOH was stirred over 3 h. It was diluted with 10× volumes of water and adjusted pH to 7 using 1M HCl and lyophilized. The product was used in the next step without purification. UPLC-MS: calculated for C₃₀H₄₈N₂O₂₀ 756.28, observed m/z: 757.4 (M+H) (t_{R}=2.59/5.00 min).

### Step 4: 2,5-Dioxopyrrolidin-1-yl 6-((2-((2-(((2R,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-3,4-dioxocyclobut-1-en-1-yl)amino)hexanoate

The title compound was prepared using procedures analogous to those described in Example 1, Step 4 (ML-1), substituting 6-((2-((2-(((2R,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-3,4-dioxocyclobut-1-en-1-yl)amino)hexanoic acid for 6-({2-[(α-D-mannopyranosyl-(1→3)-[α-D-mannopyranosyl-(1→6)]-β-D-glucopyranosyl)oxy]ethyl} amino)-6-oxohexanoic acid. UPLC-MS: calculated for C₃₄H₅₁N₃O₂₂ 853.29, observed m/z: 854.1 (M+H) (t_{R}=3.26/5.00 min).

### Example 48

### 2,5-Dioxopyrrolidin-1-yl 5-β-(2-(((2R,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)ureido)pentanoate (ML-48)

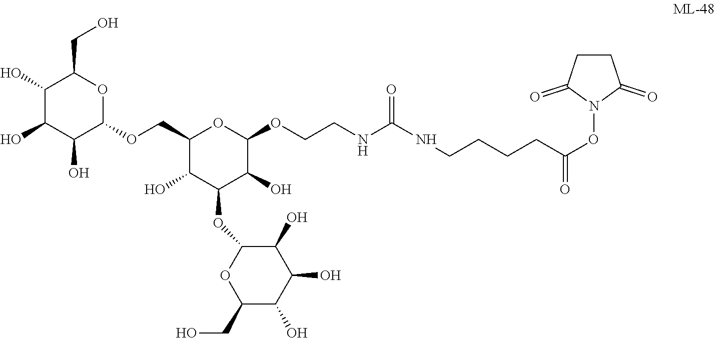 [SMILES: O=C(NCCCCC(=O)ON1C(=O)CCC1=O)NCCO[C@@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O]

### Step 1: Benzyl 5-(3-(2-(((2R,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)ureido)pentanoate

A mixture of diphenylphosphoryl azide (641 mg, 2.328 mmol), adipic acid monobenzyl ester (500 mg, 2.116 mmol), TEA (590 μl, 4.23 mmol) using chloroform (2.116 ml) as solvent is heated at 65° C. for 2 h, then removed heat and stirred overnight. The solvent was evaporated, and the crude was re-dissolved resulting crude benzyl 5-isocyanatopentanoate in 3.6 ml of DMF. 2-aminoethyl α-D-mannopyranosyl-(1→3)-[α-D-mannopyranosyl-(1→6)]-β-D-mannopyranoside (100 mg, 0.183 mmol), Hünig's base (63.8 μl, 0.365 mmol) was added, and the mixture was heated at 70° C., overnight. The reaction mixture was concentrated on a rotary evaporator and purified by silica gel column flash chromatography with a 13 g C18 column using gradient 0-40% of B in 30 min (flow 6 ml/min, Solvent A=water-0.05% TFA, solvent B=AcN-0.05% TFA). The title compound was isolated after lyophilization. UPLC-MS: calculated for C₃₃H₅₂N₂O₁₉ 780.31, observed m/z: 781.22 (M+H) (t_{R}=1.31/2.00 min).

### Step 2: 5-β-(2-(((2R,3S,4S,5R,6R)-3,5-Dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)ureido)pentanoic acid

A mixture of the product of Step 1 (100 mg, 0.128 mmol) in water (12.8 ml) was hydrogenated over Pearlman's catalyst (17.99 mg, 0.026 mmol) at 50 psi of H₂ over 3 h. The catalyst was filtered out, and the solution was lyophilized. UPLC-MS: calculated for: C₂₆H₄₆N₂O₁₉ 690.2695, observed 691.38 (M+H) (t_{R}=1.12/5.00 min).

### Step 3: 2,5-Dioxopyrrolidin-1-yl 5-β-(2-(((2R,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)ureido)pentanoate

The title compound was prepared using procedures analogous to those described for ML-1 in Example 1, substituting 5-(3-(2-(((2R,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyptetrahydro-2H-pyran-2-yl)oxy)methyptetrahydro-2H-pyran-2-yl)oxy)ethyl)ureido)pentanoic acid for 6-({2-[(α-D-mannopyranosyl-(1→3)-[α-D-mannopyranosyl-(1→6)]-β-D-glucopyranosyl)oxy]ethyl}amino)-6-oxohexanoic acid in Step 4. UPLC-MS: calculated for C₃₀H₄₉N₃O₂₁ 787.28, observed m/z: 787.47 (M+H) (t_{R}: 1.46/5.00 min).

### Example 49

### 2,5-Dioxopyrrolidin-1-yl 6-β-(2-(((2R,3S,4S,5R,6R)-3,5-dihydroxy-4-(((2R,3S,4S,5S,6R)-3,4,5-trihydroxy-6-(hydroxymethyl)tetrahydro-2H-pyran-2-yDoxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyDureido)hexanoate (ML-49)

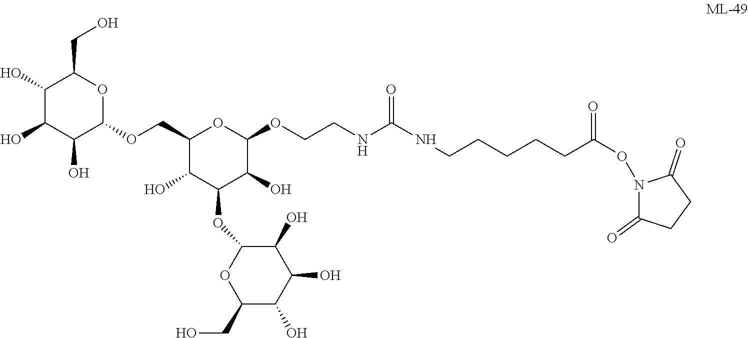 [SMILES: O=C(NCCCCCC(=O)ON1C(=O)CCC1=O)NCCO[C@@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O]

### Step 1: Ethyl 6-β-(2-(((2R,3S,4S,5R,6R)-3,5-dihydroxy-4-(((2R,3S,4S,5S,6R)-3,4,5-trihydroxy-6-(hydroxymethyl)tetrahydro- 2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)ureido)hexanoate

The mixture of ethyl 6-isocyanatohexanoate (33.8 mg, 0.183 mmol), 2-aminoethyl α-D-mannopyranosyl-(1→3)-[α-D-mannopyranosyl-(1→6)]-β-D-mannopyranoside (100 mg, 0.183 mmol), Hünig's base (63.8 1, 0.365 mmol) was heated using DMF (3653 μl) as the solvent, at 70° C. overnight. The mixture was concentrated using a rotary evaporator and purified by flash chromatography on a 13 g C18 column using gradient 0-40% of B in 30 min (flow 12 ml/min, Solvent A=water-0.05% TFA, Solvent B=AcN-0.05% TFA). The mixture was lyophilized to yield the title compound. UPLC-MS: calculated for: C₂₉H₅₂N₂O₁₉ 732.31, observed 733.23 (M+H) (t_{R}=1.10/2.00 min).

### Step 2: 6-β-(2-(((2R,3S,4S,5R,6R)-3,5-Dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)ureido)hexanoic acid

The product of Step 1 (95 mg, 0.130 mmol) was dissolved in water (1.3 ml), and NaOH (1M) (259 μl, 0.259 mmol) was added. The reaction was stirred for 2 h. The pH was adjusted to 7.0, and lyophilization produced the product. UPLC-MS: calculated for: C₂₇H₄₈N_{O}₁₉ 704.28, observed 705.17 (M+H) (t_{R}0.32/2.00 min).

### Step 3: 2,5-Dioxopyrrolidin-1-yl 6-(3-(2-(((2R,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)ureido)hexanoate

The title compound was prepared using procedures analogous to those described for ML-1 in Example 1, substituting 6-({2-[(α-D-mannopyranosyl-(1→3)-[α-D-mannopyranosyl-(1→6)]-β-D-glucopyranosyl)oxy]ethyl}amino)-6-oxohexanoic acid for 6-({2-[(α-D-mannopyranosyl-(1→3)-[α-D-mannopyranosyl-(1→6)]-β-d-glucopyranosyl)oxy]ethyl}amino)-6-oxohexanoic acid in Step 4. UPLC-MS: calculated for C₃₁H₅₁N₃O₂₁₁ 801.30, observed m/z: 802.4, (M+H) (t_{R} 1.727/5.00 min).

### Example 50

### 2,5-Dioxopyrrolidin-1-yl 6-((3-((2-(((2R,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-3-oxopropyl)sulfonamido) hexanoate (ML-50)

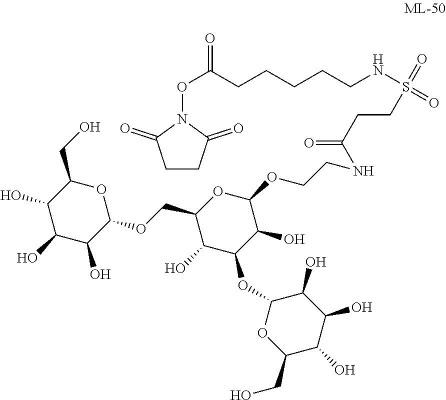 [SMILES: O=C(CCS(=O)(=O)NCCCCCC(=O)ON1C(=O)CCC1=O)NCCO[C@@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O]

### Step 1: benzyl 6-((3-methoxy-3-oxopropyl)sulfonamido)hexanoate

6-amino-hexanoic acid benzyl ester was dissolved with toluene-4-sulfonic acid (400 mg, 1.017 mmol) in pyridine (2.5 ml) and TEA (425 μl, 3.05 mmol) was added followed by methyl 3-(chlorosulfonyl)propanoate (379 mg, 2.033 mmol). The reaction mixture was stirred overnight and diluted with 50 ml of DCM, then washed with 30 ml of 1M HCl, 50 ml of NaHCO₃, and dried over Na₂SO₄. The product was purified by flash chromatography with a 12 g silica gel column, gradient 0-50% of EtOAc in hexanes in 20 min followed by 5 min hold with 50% EtOAc. UPLC-MS: calculated for C₁₇H₂₅NO₆S 371.14, observed m/z: 372.16, (M+H) (t_{R}=1.09/2.00 min).

### Step 2: 3-(N-(6-(benzyloxy)-6-oxohexyl)sulfamoyl)propanoic acid

The product of Step 1 (111 mg, 0.299 mmol) was dissolved in THF (1.12 ml) and a solution of LiOH (9.30 mg, 0.388 mmol) in water (374 μl) was added. The reaction mixture was stirred for 2 h, and the mixture was partitioned between 50 ml of EtOAc and 50 ml of 1M HCl. The organic phase was extracted 2×30 mL of EtOAc, and the combined organic phases were concentrated by rotary evaporation to obtain the title product. UPLC-MS: calculated for C₁₆H₂₃NO₆S 357.12, observed m/z: 358.11, (M+H) (t_{R}=0.97/2.00 min).

### Step 3: Benzyl 6-((3-((2-(((2R,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((25,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-3-oxopropyl)sulfonamido)hexanoate

To a mixture of the product of Step 2 (105 mg, 0.294 mmol) and 2-aminoethyl α-D-mannopyranosyl-(1→3)-[α-D-mannopyranosyl-(1→6)]-β-D-mannopyranoside (161 mg, 0.294 mmol) in DMF (2.9 ml) was added thinig's base (205 μl, 1.175 mmol), HOBT (90 mg, 0.588 mmol), and EDC (113 mg, 0.588 mmol). The mixture was stirred overnight, concentrated, and purified by silica gel flash chromatography using a 13 g C18 column, Solvent A=water-0.05% TFA, solvent B=AcN-0.05% TFA, gradient 0-30% in 30 min, flow 11 ml/min. The title compound was obtained after lyophilization. UPLC-MS: calculated for C₃₆H₅₈N₂O₂₁S 886.35, observed m/z: 887.29, (M+H) (t_{R}=1.43/2.00 min).

### Step 4: 6-((3-((2-(((2R,3S,4S,5R,6R)-3,5-Dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-3-oxopropyl)sulfonamido)hexanoic acid

The product of Step 3 (100 mg, 0.113 mmol) in water (11.3 ml) was hydrogenated over Pearlman's catalyst (15.83 mg, 0.023 mmol) using Parr shaker at 50 psi of H₂ overnight. The catalyst was removed by filtration, and the mixture was lyophilized to yield the title product. UPLC-MS: calculated for C₂₉H₅₂N₂O₂₁S 796.27, observed m/z: 797.4, (M+H) (t_{R}=1.48/5.00 min).

### Step 5: 2,5-Dioxopyrrolidin-1-yl 6-((3-((2-(((2R,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-3-oxopropyl)sulfonamido)hexanoate

The title compound was prepared using procedures analogous to those described for ML-1 in Example 1, substituting 6-((3-((2-(((2R,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)amino)-3-oxopropyl)sulfonamido)hexanoic acid for 6-({2-[(α-D-mannopyranosyl-(1→3)-[α-D-mannopyranosyl-(1→6)]-β-D-glucopyranosyl)oxy]ethyl}amino)-6-oxohexanoic acid in Step 4. UPLC-MS: calculated for C₃₃H₅₅N₃O₂₃S 893.29, observed m/z: 894.4 (M+H) (t_{R}=1.905/5.00 min).

### Example 51

### 2,5-Dioxopyrrolidin-1-yl 4-(((2-(((2R,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)carbamoyl)oxy)butanoate (ML-51)

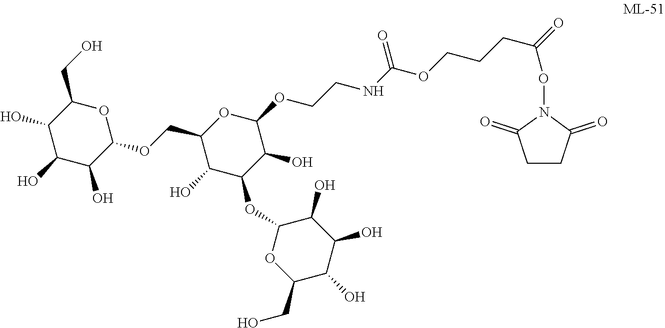 [SMILES: O=C(CCCOC(=O)NCCO[C@@H]1O[C@H](CO[C@H]2O[C@H](CO)[C@@H](O)[C@H](O)[C@@H]2O)[C@@H](O)[C@H](O[C@H]2O[C@H](CO)[C@@H](O)[C@H](O)[C@@H]2O)[C@@H]1O)ON1C(=O)CCC1=O]

### Step 1: methyl 4-(((4-nitrophenoxy)carbonyl)oxy)butanoate

To a solution of methyl 4-hydroxybutanoate (100 mg, 0.847 mmol) in DCM (4233 μl) was added pyridine (205 μl, 2.54 mmol) followed by 4-nitrophenyl chloroformate (171 mg, 0.847 mmol). The reaction mixture was overnight, diluted with 50 ml of DCM and washed with 50 ml of 1M HCl. The organic phase was dried over sodium sulfate. The product was purified on a SiO₂ column, gradient 0-30% EtOAc/hexanes in 25 min followed by ¹H NMR (500 MHz, CDCl₃) δ 2.16-2.11 (2H, m), 2.53 (2H, t, J=7.22 Hz), 3.74 (3H, s), 4.38 (2H, t, J=6.29 Hz), 7.42-7.40 (2H, m), 8.31-8.29 (2H, m).

### Step 2: Methyl 4-(((2-(((2R,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)carbamoyl)oxy)butanoate

A mixture of 2-aminoethyl α-D-mannopyranosyl-(1→3)-[α-D-mannopyranosyl-(1→6)]-β-D-mannopyranoside (100 mg, 0.183 mmol), the product of Step 1 (51.7 mg, 0.183 mmol), Hünig's base (159 μl, 0.913 mmol) in DMF (1.82 ml) was stirred overnight. The mixture was concentrated and purified by silica gel flash chromatography on a 13 g C18 column, solvent A=water/0.05% TFA, solvent B=ACN/0.05% TFA, flow 13 ml/min, gradient of B 0-30% in 20 min. The concentrated mixture was lyophilized to yield the title product. UPLC-MS: calculated for C₂₆H₄₅NO₂₀ 691.2535, observed m/z: 692.38 (M+H) (t_{R}=1.52/5.00 min).

### Step 3: 4-(((2-(((2R,3S,4S,5R,6R)-3,5-Dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)carbamoyl)oxy)butanoic acid

To a solution of Step 2 (74 mg, 0.107 mmol) in water (107 μl) was added sodium hydroxide (1.0 M) (214 μl, 0.214 mmol), and the reaction mixture was stirred for 4 h. The pH was adjusted to 7 with 1M HCl and removed solvent by lyophilization to obtain the product. UPLC-MS: calculated for C₂₅H₄₃NO₂₀ 677.2378, observed m/z: 678.36 (M+H) (t_{R}=1.11/5.00 min).

### Step 4: 2,5-dioxopyrrolidin-1-yl 4-(((2-(((2R,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)carbamoyl)oxy)butanoate

The title compound was prepared using procedures analogous to those described for ML-1 in Example 1, substituting 4-(((2-(((2R,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyptetrahydro-2H-pyran-2-yl)oxy)methyptetrahydro-2H-pyran-2-yl)oxy)ethyl)carbamoyl)oxy)butanoic acid for 6-({2-[(α-D-mannopyranosyl-(1→3)-1α-D-mannopyranosyl-(1→6)143-D-glucopyranosyl)oxy]ethyl}amino)-6-oxohexanoic acid in Step 4. UPLC-MS: calculated for C₂₉H₄₆N₂O₂₂ 774.25, observed m/z: 775.36 (M+H) (t_{R}=1.54/5.00 min).

### Example 52

### 2,5-Dioxopyrrolidin-1-yl 2-4-(1-(2-(((2R,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)-1H-1,2,3-triazol-4-yl)phenyl)acetate (ML-52)

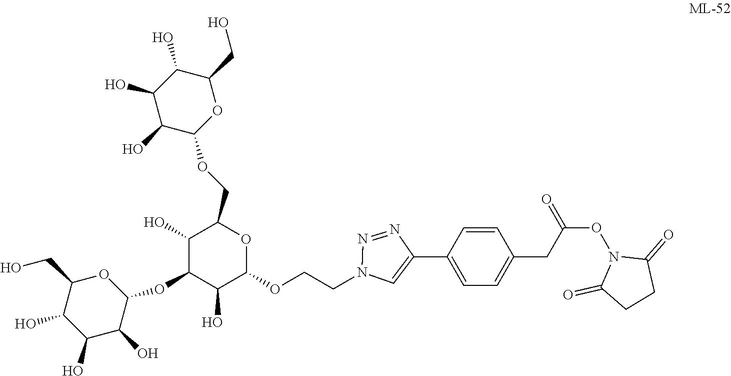 [SMILES: O=C(Cc1ccc(-c2cn(CCO[C@H]3O[C@H](CO[C@H]4O[C@H](CO)[C@@H](O)[C@H](O)[C@@H]4O)[C@@H](O)[C@H](O[C@H]4O[C@H](CO)[C@@H](O)[C@H](O)[C@@H]4O)[C@@H]3O)nn2)cc1)ON1C(=O)CCC1=O]

### Step 1: 2-4-(1-(2-(((2R,3S,4S,5R,6R)-3,5-dihydroxy-4-(((2R,3S,4S,5S,6R)-3,4,5-trihydroxy-6-(hydroxymethyl)tetrahydro- 2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)-1H-1,2,3-triazol-4-yl)phenyl)actic acid

In the glove box, to the mixture of 2-(4-ethynylphenyl) acetic acid (100 mg) and 2-azido manα(1-3)[manα(1-6)]man (430 mg) was added DMSO (3000 uL). To above solution was added CuBr-DMS solution (64.2 mg in 1000 uL). The mixture was stirred at rt for 4 h. The crude was loaded directly onto a C18 reverse phase column, eluted with 10% to 100% water in ACN over 30 min. The fractions containing desired product were combined and lyophilized. The lyophilized product was redissolved in small amount of water. The crude was by C18 reverse phase chromatography, using 0-20% ACN in water over 20 min, to give desired product. UPLC Method C: m/e=734.316, [M+H]; t_{R}=4.19.

### Step 2: 2,5-Dioxopyrrolidin-1-yl 2-4-(1-(2-(((2R,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)-1H-1,2,3-triazol-4-yl)phenyl)acetate

The title compound was prepared using procedures analogous to those described for ML-1 in Example 1, substituting 2-4-(1-(2-(((2R,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyptetrahydro-2H-pyran-2-yl)oxy)methyptetrahydro-2H-pyran-2-yl)oxy)ethyl)-1H-1,2,3-triazol-4-yl)phenyl)actic acid for 6-({2-[(α-D-mannopyranosyl-(1→3)-[α-D-mannopyranosyl-(1→6)]-β-D-glucopyranosyl)oxy]ethyl}amino)-6-oxohexanoic acid in Step 4. UPLC-MS Method B: calculated for C₃₄H₄₆N₄O₂₀ 830.744, observed m/z: 831.3506 (M+H) (t_{R}=3.91 min).

### Example 53

### 2,5-Dioxopyrrolidin-1-yl 3-(1-(2-(((2R,3S,4S,5R,6R)-3,5-dihydroxy-4-(((2R,3S,4S,5S,6R)-3,4,5-trihydroxy-6-(hydroxymethyl)tetrahydro-2H-pyran-2-yl)oxy)-6-((((2S,3S,4S,5S,6R)-3,4,5-trihydroxy-6-(hydroxymethyl)tetrahydro-2H-pyran-2-yl)oxy)methyl)tetrahydro-2H-pyran-2-yl)oxy)ethyl)-1H-1,2,3-triazol-4-yl)propanoate (ML-53)

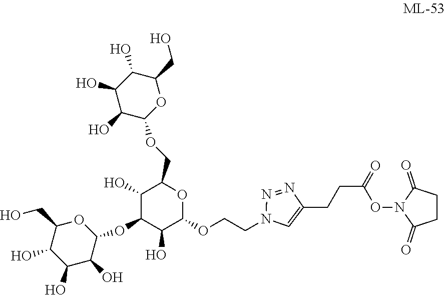 [SMILES: O=C(CCc1cn(CCO[C@H]2O[C@H](CO[C@H]3O[C@H](CO)[C@@H](O)[C@H](O)[C@@H]3O)[C@@H](O)[C@H](O[C@H]3O[C@H](CO)[C@@H](O)[C@H](O)[C@@H]3O)[C@@H]2O)nn1)ON1C(=O)CCC1=O]

The title compound was prepared using procedures analogous to those described for ML-52 in Example 52, substituting 2-(4-ethynylphenyl) acetic acid with pent-4-ynoic acid, and replacing 2-azido man α(1-3)[manα(1-6)]man with per-benzoyl 2-azido man α(1-3)[manα(1-6)]man in Step 1. The intermediate was deprotected using 30% NaOMe in MeOH. UPLC-MS Method B: observed m/z: 781.00 (M+H), t_{R}=0.30 min.

### Example 54

### 8-[(2,5-Dioxopyrrolidin-1-yl)oxy]-N-(2-{[α-D-mannopyranosyl-(1→3)-[α-D-mannopyranosyl-(1→6)]-β-D-mannopyranosyl]oxy}ethyl)-8-oxo-octanamide (ML-54)

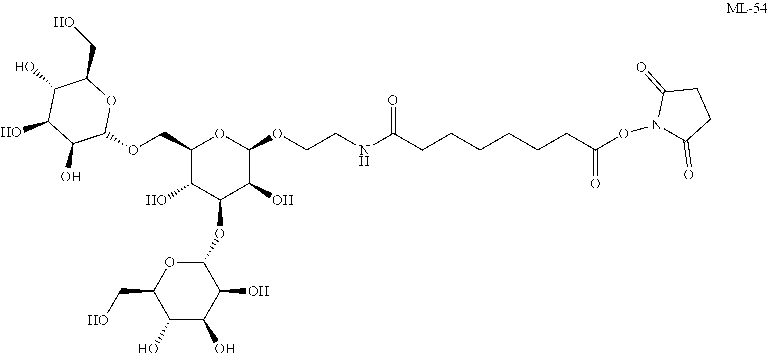

The title compound was prepared using procedures analogous to those described for ML-2 in Example 2, substituting 2-aminoethyl α-D-mannopyranosyl-(1→3)-[α-D-mannopyranosyl-(1→6)]-β-D-mannopyranoside 2-aminoethyl α-D-mannopyranosyl-(1→3)-[α-D-mannopyranosyl-(1→6)]-β-D-glucopyranoside for in Step 2. UPLC Method B: calculated for C₃₂H₅₂N₂O₂₁ 800.31, observed m/e: 801.45 [M+1]; t_{R}=1.900 min).

### Example 55

### 8-[(2,5-Dioxopyrrolidin-1-yl)oxy]-N-(2-{[α-D-mannopyranosyl-(1→3)-[α-D-mannopyranosyl-(1→6)]-α-D-mannopyranosyl]oxy}ethyl)-8-oxo-octanamide (ML-54)

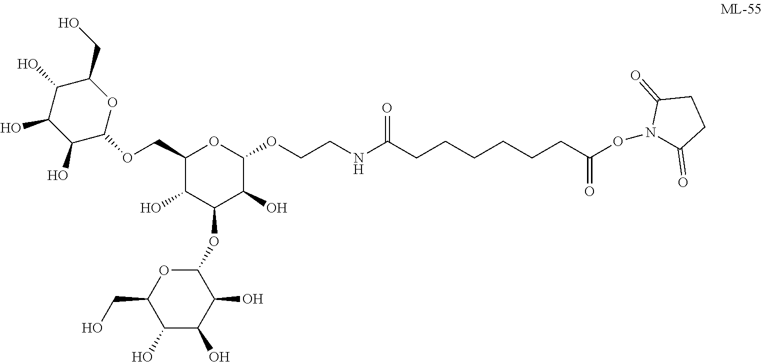

The title compound was prepared using procedures analogous to those described for ML-2 in Example 2, substituting 2-aminoethyl α-D-mannopyranosyl-(1→3)-[α-D-mannopyranosyl-(1→6)]-α-D-mannopyranoside for 2-aminoethyl α-D-mannopyranosyl-(1→3)-[α-D-mannopyranosyl-(1→6)]-β-D-glucopyranoside in Step 2. UPLC Method B: calculated for C₃₂H₅₂N₂O₂₁ 800.31, observed m/e: 801.45 [M+1]; t_{R}=1.89 min).

### Example 56

### 4-[(2,5-dioxopyrrolidin-1-yl)oxy]-N—((R)-1-((2-((2-{[α-D-mannopyranosyl-(1→3)-[α-D-mannopyranosyl-(1→6)]-α-D-mannopyranosyl]}oxy)ethyl)amino)-2-oxoethyl)amino)-1-oxopropan-2-yl)-4-oxobutanamide (ML-56)

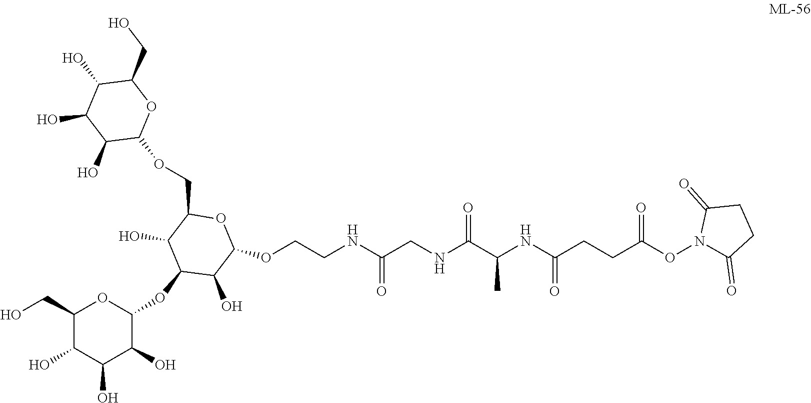

### Step 1: benzyl ((S)-1-((2-((2-{[α-D-mannopyranosyl-(1→3)-[α-D-mannopyranosyl-(1→6)]-α-D-mannopyranosyl]}oxy)ethyl)amino)-2-oxoethyl)amino)-1-oxopropan-2-yl)carbamate

To a solution of commercially available Z-ALA-GLY-OH (1000 mg, 3.57 mmol) in dry DMF (35.0 ml) was added EDC (1368 mg, 7.14 mmol) and HOBT (164 mg, 1.070 mmol) at 0° C. under N₂. The mixture was stirred at 0° C. for 30 min, and 2-aminoethyl α-D-mannopyranosyl-(1→3)-[α-D-mannopyranosyl-(1→6)]-α-D-mannopyranoside (2149 mg, 3.92 mmol) was added. The mixture was gradually warmed up to rt and stirred overnight. DMF was removed by rotary evaporation under reduced pressure at 37° C. The residue was purified by C18 reverse phase column, eluting with ACN/water (gradient from 0% to 50% in 22V). After lyophilizing overnight, the title compound was given. UPLC Method B: calculated for C₃₃H₅₁N₃O₂₀ 809.13, observed m/e=810.4096 [M+1]; t_{R}=3.46 min.

### Step 2: (S)-2-amino-N-((2-((2-{[α-D-mannopyranosyl-(1→3)-[α-D-mannopyranosyl-(1→6)]-α-D-mannopyranosyl]}oxy)ethyl)amino)-2-oxoethyl)propenamide

A solution of benzyl ((S)-1-((2-((2-{[α-D-mannopyranosyl-(1→3)-[α-D-mannopyranosyl-(1→6)]-α-D-mannopyranosyl]}oxy)ethyl)amino)-2-oxoethyl)amino)-1-oxopropan-2-yl) carbamate (2500 mg, 3.09 mmol) in water (80 ml) was added Pd/C (10% by weight) (329 mg, 0.309 mmol). The resulting solution was hydrogenated under H₂ at rt. The residue was filtered through a pad of filter reagent diatomaceous earth (CELITE) and washed with water and lyophilized to give the title compound. UPLC Method B: calculated for C₂₅H₄₅N₃O₁₈ 675.27, observed m/e=676.3251 [M+1]; t_{R}=1.12 min.

### Step 3: 4-(((S)-1-((2-((2{[α-D-mannopyranosyl-(1→3)-[-α-D-mannopyranosyl-(1→6)]-α-D-mannopyranosyl]}oxy)ethyl)amino)-2-oxoethyl)amino)-1-oxopropan-2-yl)amino)-4-oxobutanoic acid

To a solution of (S)-2-amino-N-((2-((2-{[α-D-mannopyranosyl-(1→3)-[α-D-mannopyranosyl-(1→6)]-α-D-mannopyranosyl]}oxy)ethyl)amino)-2-oxoethyl)propanamide (1013 mg, 1.499 mmol) in dry DMF (25 ml) under N₂ at 0° C., succinic anhydride (158 mg, 1.574 mmol) and TEA (0.219 ml, 1.57 mmol) were added. The mixture was stirred at 0° C. for 30 min, then resulting mixture was stirred and gradually warmed to rt overnight. DMF was removed by rotary evaporation under reduced pressure at 40° C. The residue was purified by C18 reverse phase column, eluting with ACN/water (gradient from 0% to 30% in 20V). After lyophlizing, the title compound was given (825 mg, 1.064 mmol, 70.9% yield). UPLC Method B: calculated for C₂₉H₄₉N₃O₂₁ 775.29, observed m/e=776.3879 [M+1]; t_{R}=1.13 min.

### Step 4: 4-[(2,5-dioxopyrrolidin-1-yl)oxy]-N—((S)-1-((2-((2-{[α-D-mannopyranosyl-(1→3)-[α-D-mannopyranosyl-(1→6)]-α-D-mannopyranosyl]}oxy)ethyl)amino)-2-oxoethyl)amino)-1-oxopropan-2-yl)-4-oxobutanamide

To a solution of 4-(((S)-1-((2-((2{[α-D-mannopyranosyl-(1→3)-[α-D-mannopyranosyl-(1→6)]-α-D-mannopyranosyl]}oxy)ethyl)amino)-2-oxoethyl)amino)-1-oxopropan-2-yl)amino)-4-oxobutanoic acid (825 mg, 1.064 mmol) in dry DMF (30 ml) at 0° C. was added TSTU (480 mg, 1.595 mmol) and Hünig's base (0.306 ml, 1.755 mmol). The mixture was stirred at 0° C. for 2 h, then DMF was removed by rotary evaporation under reduced pressure at 40° C. The residue was purified by C18 reverse phase column, eluting with ACN/water (gradient from 0% to 25%). After lyophlizing, the title compound was given. UPLC Method B: calculated for C₃₃H₅₂N₄O_{23 872.30,} observed m/e=873.3801 [M+1]; t_{R}=1.12 min.

### Example 57

### Synthesis of IOC-1

To a 20 mL scintillation vial containing human insulin (400 mg, 0.069 mmol) at rt was added DMSO (4.0 mL) and TEA (67.2 μL, 0.482 mmol). The mixture was stirred gently until the human insulin dissolved. In a separate vial, linker ML-1 (238 mg, 0.216 mmol) was dissolved in DMSO (2.0 mL) at rt. To the solution containing human insulin was added the solution of ML-1 in three equal portions in 20 to 30 min intervals. The reaction was quenched by adding 2-aminoethanol (125 μL, 2.066 mmol). After stirring at rt for 15 min, the resulting mixture was carefully diluted with cold H₂O (70 mL) at 0° C. The pH of the resulting mixture was adjusted to a final pH of 2.5 using 1N HCl (or 0.1N NaOH). The resulting solution was purified by preparatory scale HPLC using a C8 column, eluted with Buffer A: 0.05-0.1% TFA in deionized water; Buffer B: 0.05-0.1% TFA in ACN. The combined desired fractions were lyophilized. The solids were dissolved in water, and the pH was adjusted to 7 using 0.1N NaOH solution to provide a solution of IOC-1. UPLC-MS Method A: t_{R}=3.64 min; m/z=1946.61 (z=4).

EXAMPLES 58 through 75, Conjugates IOC-3 to IOC-5, IOC-10, IOC-11, IOC-13, IOC-16, IOC-19, IOC-26, IOC-31, IOC-46, IOC-48, IOC-50, IOC-53, IOC-84, IOC-86, IOC-88, IOC-91, and IOC-148, as listed in Table 1, were prepared according to procedures analogous to those described above for EXAMPLE 57, IOC-1, with the appropriate linkers.

TABLE 1

| EXAMPLE | Conjugate | Linker |
|---|---|---|
| 58 | IOC-3 | ML-8 |
| 59 | IOC-5 | ML-3 |
| 60 | IOC-10 | ML-17 |
| 61 | IOC-11 | ML-7 |
| 62 | IOC-13 | ML-9 |
| 63 | IOC-16 | ML-1 |
| 64 | IOC-19 | ML-18 |
| 65 | IOC-26 | ML-2 |
| 66 | IOC-31 | ML-19 |
| 67 | IOC-46 | ML-37 |
| 68 | IOC-48 | ML-32 |
| 69 | IOC-50 | ML-35 |
| 70 | IOC-53 | ML-36 |
| 71 | IOC-84 | ML-48 |
| 72 | IOC-86 | ML-49 |
| 73 | IOC-88 | ML-50 |
| 74 | IOC-91 | ML-51 |
| 75 | IOC-148 | ML-56 |

### Example 76

### Synthesis of IOC-66

Human insulin (800 mg, 0.138 mmol) was dissolved in aq. Na₂CO₃ (6.85 mL, 0.1M) and ACN (4.6 mL). The pH of the resulting solution was adjusted to 10.5, to which ML-8 (157 mg, 0.207 mmol) in DMSO (2.25 mL) in 4 portions over 80 min; the reaction mixture was quenched by adding 2-aminoethanol (41.7 μL, 0.689 mmol). After stirring at rt for 15 min, the reaction mixture was diluted with H₂O and pH was adjusted to about 2.5 using 1.0N HCl solution, concentrated. The resulting solution was purified by preparatory scale HPLC using a C4 50×250 mm column, gradient 24-28.5% ACN in H₂O with 0.1% TFA over 25 min, flow rate 85 mL/min. The combined desired fractions were lyophilized. The solids were dissolved in water, and the pH was adjusted to 7 using 0.1N NaOH solution to provide a solution of B29 mono conjugated intermediate. UPLC-MS Method A: t_{R}=3.75 min; m/z=1613.72 (z=4).

The title compound was prepared using procedures analogous to those described in Example 57, substituting B29 mono conjugated intermediate above for human insulin and ML-3 (2.0 eq) to give IOC-66. PLC-MS Method A: t_{R}=3.66 min; m/z=1941.66 (z=4).

EXAMPLES 77 and 78, Conjugates IOC-72 and IOC-74 as listed in Table 2, were prepared according to procedures analogous to those described above for EXAMPLE 76, IOC-66, with the appropriate linkers.

TABLE 2

| EXAMPLE | Conjugate | Linker |
|---|---|---|
| 77 | IOC-72 | ML-3, 12 |
| 78 | IOC-74 | ML-3, 12 |

### Example 79

### Synthesis of IOC-2

To a 20 mL scintillation vial containing human insulin (400 mg, 0.069 mmol) at rt was added DMSO (4.0 mL) and TEA (67.2 μL, 0.482 mmol). The mixture was stirred gently until the human insulin dissolved. In a separate vial, linker ML-6 (152 mg, 0.138 mmol) was dissolved in DMSO (2.0 mL) at rt. To the solution containing human insulin was added the solution of ML-6 in three equal portions in 20 to 30 min intervals. The reaction was quenched by adding 2-aminoethanol (125 μL, 2.066 mmol). After stirring at rt for 15 min, the resulting mixture was carefully diluted with cold H₂O (70 mL) at 0° C. The pH of the resulting mixture was adjusted to a final pH of 2.5 using 1N HCl (or 0.1N NaOH). The resulting solution was purified by p preparatory scale HPLC using a C8 10 μm, 100 Å, 50×250 mm column, eluted with Buffer A: 0.05-0.1% TFA in deionized water; Buffer B: 0.05-0.1% TFA in ACN. The combined desired fractions were lyophilized. The solids were dissolved in water, and the pH was adjusted to 7 using 0.1N NaOH solution to provide a solution of IOC-2. UPLC-MS Method A: t_{R}=3.76 min; m/z=1767.38 (z=4).

EXAMPLES 80 through 171, Conjugates IOC-4, IOC-6, IOC-9, IOC-12, IOC-14, IOC-15, IOC-18, IOC-20, IOC-22, IOC-24, IOC-25, IOC-27, IOC-28, IOC-30, IOC-32, IOC-33, IOC-41, IOC-42, IOC-43, IOC-44, IOC-45, IOC-47, IOC-49, IOC-51, IOC-52, IOC-54, IOC-55, IOC-56, IOC-57, IOC-58, IOC-59, IOC-61, IOC-62, IOC-63, IOC-75, IOC-76, IOC-77, IOC-78, IOC-80, IOC-81, IOC-85, IOC-87, IOC-89, IOC-90, IOC-92, IOC-94, IOC-95, IOC-96, IOC-97, IOC-98, IOC-99, IOC-100, IOC-101, IOC-107, IOC-108, IOC-109, IOC-110, IOC-111, IOC-112, IOC-113, IOC-114, IOC-115, IOC-116, IOC-117, IOC-118, IOC-119, IOC-120, IOC-121, IOC-122, IOC-123, IOC-124, IOC-125, IOC-126,I0C-127, IOC-128, IOC-129, IOC-131, IOC-132, IOC-133, IOC-134, IOC-135, IOC-136, IOC-137, IOC-138, IOC-139, IOC-140, IOC-141, IOC-142, IOC-143, IOC-144, IOC-145, and IOC-147, as listed in Table 3, were prepared according to procedures analogous to those described above for EXAMPLE 79, IOC-2, with the appropriate linkers.

TABLE 3

| EXAMPLE | Conjugate | Linker |
|---|---|---|
| 80 | IOC-2 | ML-6 |
| 81 | IOC-4 | ML-8 |
| 82 | IOC-6 | ML-3 |
| 83 | IOC-9 | ML-17 |
| 84 | IOC-12 | ML-7 |
| 85 | IOC-14 | ML-9 |
| 86 | IOC-15 | ML-1 |
| 87 | IOC-18 | ML-18 |
| 88 | IOC-20 | ML-13 |
| 89 | IOC-22 | ML-13 |
| 90 | IOC-24 | ML-1 |
| 91 | IOC-25 | ML-2 |
| 92 | IOC-27 | ML-15 |
| 93 | IOC-28 | ML-16 |
| 94 | IOC-30 | ML-19 |
| 95 | IOC-32 | ML-14 |
| 96 | IOC-33 | ML-10 |
| 97 | IOC-41 | ML-22 |
| 98 | IOC-42 | ML-23 |
| 99 | IOC-43 | ML-26 |
| 100 | IOC-44 | ML-27 |
| 101 | IOC-45 | ML-37 |
| 102 | IOC-47 | ML-32 |
| 103 | IOC-49 | ML-33 |
| 104 | IOC-51 | ML-35 |
| 105 | IOC-52 | ML-34 |
| 106 | IOC-54 | ML-36 |
| 107 | IOC-55 | ML-39 |
| 108 | IOC-56 | ML-43 |
| 109 | IOC-57 | ML-40 |
| 110 | IOC-58 | ML-41 |
| 111 | IOC-59 | ML-42 |
| 112 | IOC-61 | ML-52 |
| 113 | IOC-62 | ML-53 |
| 114 | IOC-63 | ML-3 |
| 115 | IOC-75 | ML-3 |
| 116 | IOC-76 | ML-3 |
| 117 | IOC-77 | ML-3 |
| 118 | IOC-80 | ML-24 |
| 119 | IOC-81 | ML-23 |
| 120 | IOC-85 | ML-48 |
| 121 | IOC-87 | ML-49 |
| 122 | IOC-89 | ML-50 |
| 123 | IOC-90 | ML-51 |
| 124 | IOC-92 | ML-3 |
| 125 | IOC-94 | ML-1 |
| 126 | IOC-95 | ML-1 |
| 127 | IOC-96 | ML-1 |
| 128 | IOC-97 | ML-55 |
| 129 | IOC-98 | ML-55 |
| 130 | IOC-99 | ML-20 |
| 131 | IOC-100 | ML-21 |
| 132 | IOC-101 | ML-4 |
| 133 | IOC-107 | ML-45 |
| 134 | IOC-108 | ML-46 |
| 135 | IOC-109 | ML-3 |
| 136 | IOC-110 | ML-3 |
| 137 | IOC-111 | ML-3 |
| 138 | IOC-112 | ML-3 |
| 139 | IOC-113 | ML-3 |
| 140 | IOC-114 | ML-3 |
| 141 | IOC-115 | ML-3 |
| 142 | IOC-116 | ML-3 |
| 143 | IOC-117 | ML-3 |
| 144 | IOC-118 | ML-3 |
| 145 | IOC-119 | ML-3 |
| 146 | IOC-120 | ML-3 |
| 147 | IOC-121 | ML-3 |
| 148 | IOC-122 | ML-3 |
| 149 | IOC-123 | ML-3 |
| 150 | IOC-124 | ML-3 |
| 151 | IOC-125 | ML-3 |
| 152 | IOC-126 | ML-3 |
| 153 | IOC-127 | ML-3 |
| 154 | IOC-128 | ML-3 |
| 155 | IOC-129 | ML-3 |
| 156 | IOC-131 | ML-20 |
| 157 | IOC-132 | ML-20 |
| 158 | IOC-133 | ML-55 |
| 159 | IOC-134 | ML-55 |
| 160 | IOC-135 | ML-21 |
| 161 | IOC-136 | ML-54 |
| 162 | IOC-137 | ML-21 |
| 163 | IOC-138 | ML-54 |
| 164 | IOC-139 | ML-2 |
| 165 | IOC-140 | ML-2 |
| 166 | IOC-141 | ML-10 |
| 167 | IOC-142 | ML-2 |
| 168 | IOC-143 | ML-3 |
| 169 | IOC-144 | ML-3 |
| 170 | IOC-145 | ML-3 |
| 171 | IOC-147 | ML-56 |

### Example 172

### Synthesis of IOC-60

In a 100 round bottom flask was charged with human insulin (600 mg, 0.103 mmol), to which was added DMF (3.0 mL), and TEA (0.144 mL, 1.03 mmol). To the resulting mixture was added 2,5-dioxopyrrolidin-1-yl pent-4-ynoate (46 mg, 0.236 mmol). After stirring at rt for 2 h, the mixture was diluted with 5 ml water and purified by preparatory scale HPLC using a C8 10 μm, 100 Å, 50×250 mm column, eluted 210 nm, flow rate at 85 ml/min, 0.05% TFA in ACN/H₂O, 27% ACN to 37% ACN in H₂O, 20 min ramp. Desired fractions were combined and freeze-dried to give N^{A11},N^{εB29}-Bis(pent-4-ynamide)Human Insulin (206 mg yield 33.4%). UPLC Method A: m/e=1492.652 [(M+4)/4]; t_{R}=4.23 min.

In a 20 ml vial, 50 mg N^{A1}N^{εB29}-Bis(pent-4-ynamide)Human Insulin was dissolved in a mixed solvent solution of 6 mL DMSO and 9 ml water. To the mixture was added pH=7.0 triethylammonium acetate buffer solution (2 ml, final concentration is 0.2 mM). In another 20 ml vial, ML-44 (15 mg) was dissolved in a mixed solvent solution of 6 mL DMSO and 9 ml water. The two solution were mixed and subjected to vortex. To the mixture was added 2 ml fresh ascorbic acid solution (5 mM, 10 mg ascorbic acid in 10 mL distilled water), and the mixture was subjected to vortex. The mixed solution was degassed by bubbling N₂ for lmin. To the degassed mixture was added lml Cu(II)-TBTA in 55% DMSO (10 mM), and the mixture was flushed with nitrogen. The reaction was allowed to stand at rt overnight.

The mixture was diluted with 100 ml mixed solvent of 20% ACN/80% H₂O (pH=3.0), then pH of the mixture was re-adjusted to 2.5 with 0.1N HCl. The mixture was concentrated to 8 ml by 10K membrane centrifuge tube (Amicon). The mixture was purified by preparatory scale HPLC using a C8 10 μm, 100 Å, 50×250 mm column at 210 nm, flow rate at 85 ml/min, 0.05% TFA in ACN/H₂O, 27% ACN to 32% ACN in H₂O, 20 min ramp. The desired fractions were combined and freeze-dried to give IOC-60. UPLC Method A: m/e=1829.054 [(M+4)/4]; t_{R}=3.74 min.

### Example 173

### Synthesis of IOC-65

### Step 1: Synthesis of Insulin B29 Mono-Conjugated Intermediate

Human insulin (800 mg, 0.138 mmol) was dissolved in aq Na₂CO₃ (6.85 mL, 0.1M) and ACN (4.6 mL). The pH of the resulting solution was adjusted to 10.5, to which ML-8 (157 mg, 0.207 mmol) in DMSO (2.25 mL) in 4 portions over 80 min. The reaction mixture was quenched by adding 2-aminoethanol (41.74, 0.689 mmol). After stirring at rt for 15 min, the reaction mixture was diluted with H₂O and pH was adjusted to about 2.5 using 1.0N HCl solution, concentrated. The resulting solution was purified by preparatory scale HPLC with a C4 50×250 mm column, using gradient 24-28.5% ACN in H₂O with 0.1% TFA over 25 min, flow rate 85 mL/min. The combined desired fractions were lyophilized. The solids were dissolved in water and the pH adjusted to 7 using 0.1N NaOH solution to provide a solution of B29 mono conjugated intermediate. UPLC-MS Method A: t_{R}=3.75 min; m/z=1613.72 (z=4).

### Step 2: A1 conjugation of B29 Mono-Conjugated Intermediate

The procedure is analogous to those described in Example 56, substituting B29 mono conjugated intermediate above for human insulin and ML-3 (1.0 eq) to give IOC-65. HPLC-MS Method A: t_{R}=3.66 min; m/z=1941.66 (z=4).

EXAMPLES 174 through 179, Conjugates IOC-67 to IOC-71, and IOC-73, as listed in Table 4, were prepared according to procedures analogous to those described above for EXAMPLE 173, IOC-65, with the appropriate linkers.

TABLE 4

| EXAMPLE | Conjugate | Linker |
|---|---|---|
| 174 | IOC-67 | ML-3, 55 |
| 175 | IOC-68 | ML-3, 15 |
| 176 | IOC-69 | ML-16, 3 |
| 177 | IOC-70 | ML-1, 3 |
| 178 | IOC-71 | ML-12, 3 |
| 179 | IOC-73 | ML-3, 12 |

### Example 180

### Synthesis of IOC-7

To a solution of ATAl-Trifluoroacetyl Human Insulin (100 mg, 0.017 mmol; prepared according to the procedures disclosed in WO2015/051052) in DMSO (2 mL) at rt was added TEA (24μL, 0.169 mmol) and a solution of ML-3 (31.7 mg, 0.041 mmol) in DMSO (750 μL). After stirring at rt for 2.5 h, the mixture was added to ACN (42 mL). The precipitate was collected through centrifugation and dissolved in water (5 mL, pH=3.00), and the mixture was cooled down to 0° C., to which a solution of NH₄OH (5 mL, 28% in water) was added. The mixture was stirred at 0° C. for 2 hr and then diluted with water (20 mL, pH=3.00). The volume of the resulting solution was concentrated and reduced to 5 mL, and was further diafiltrated with water (100 mL, pH=3.00) to final volume about 7.5 mL, which was purified by HPLC to give the IOC-7. UPLC Method A: t_{R}=3.74 49 min; m/z=1780.50609.155 (z=54).

EXAMPLES 181 through 197, Conjugates IOC-17, IOC-21, IOC-23, IOC-29, IOC-38, IOC-39, IOC-64, IOC-82, IOC-83, IOC-93, IOC-102 to 106, IOC-130, and IOC-146, as listed in Table 5, were prepared according to procedures analogous to those described above for EXAMPLE 180, IOC-7, with the appropriate linkers.

TABLE 5

| EXAMPLE | Conjugate | Linker |
|---|---|---|
| 181 | IOC-17 | ML-1 |
| 182 | IOC-21 | ML-13 |
| 183 | IOC-23 | ML-1 |
| 184 | IOC-29 | ML-16 |
| 185 | IOC-38 | ML-33 |
| 186 | IOC-39 | ML-27 |
| 187 | IOC-64 | ML-30 |
| 188 | IOC-82 | ML-24 |
| 189 | IOC-83 | ML-23 |
| 190 | IOC-93 | ML-31 |
| 191 | IOC-102 | ML-4 |
| 192 | IOC-103 | ML-20 |
| 193 | IOC-104 | ML-21 |
| 194 | IOC-105 | ML-4 |
| 195 | IOC-106 | ML-5 |
| 196 | IOC-130 | ML-29 |
| 197 | IOC-146 | ML-1 |

### Example 198

### Synthesis of IOC-8

To a solution of N^{B29}-Trifluoroacetyl Human Insulin (90 mg, 0.015 mmol; prepared according to the procedures disclosed in WO2015/051052 A2) in DMSO (1.5 mL) at rt was added TEA (21 μL, 0.152 mmol) and a solution of ML-3 (36 mg, 0.046 mmol) in DMSO (300 μL). After stirring at rt for 4 h, the mixture was added to ACN (42 mL). The precipitate was collected through centrifugation. The collected solids were dissolved in water (5 mL, pH=3.00), and the mixture was cooled down to 0° C., to which a solution of NH₄OH (5 mL, 28% in water) was added. The mixture was stirred at 0° C. for 2 hr and then diluted with water (20 mL, pH=3.00). The volume of the resulting solution was concentrated and reduced to 7.5 mL, and was further diafiltrated with water (100 mL, pH=3.00) to final volume about 7.5 mL, which was purified by HPLC to give the IOC-8. UPLC-MS Method A: t_{R}=3.68 min; m/z=1780.53 (z=4).

EXAMPLES 199 through 202, Conjugates IOC-34 to IOC-37, as listed in Table 6, were prepared according to procedures analogous to those described above for EXAMPLE 198, IOC-8, with the appropriate linkers.

TABLE 6

| EXAMPLE | Conjugate | Linker |
|---|---|---|
| 199 | IOC-34 | ML-9 |
| 200 | IOC-35 | ML-18 |
| 201 | IOC-36 | ML-10 |
| 202 | IOC-37 | ML-14 |

### Binding Assays

### Insulin Receptor Phosphorylation Assays

CHO cells stably expressing human IR(B) were in grown in in F12 cell media containing 10% FBS and antibiotics (G418, Penicillin/Strepavidin) for at least 8 h, and then serum starved by switching to F12 media containing 0.5% BSA (insulin-free) in place of FBS for overnight growth. Cells were harvested and frozen in aliquots for use in the MSD pIR assay. Briefly, the frozen cells were plated in either 96-well (40,000 cells/well, Methods A) or 384-well (10,000 cells/well, Method B) clear tissue culture plates and allowed to recover. IOC molecules at the appropriate concentrations were added and the cells incubated for 8 min at 37° C. The media was aspirated and chilled MSD cell lysis buffer was added as per MSD kit instructions. The cells were lysed on ice for 40 min, and the lysate then was mixed for 10 min at rt. The lysate was transferred to the MSD kit pIR detection plates. The remainder of the assay was carried out following the MSD kit recommended protocol.

### Insulin Receptor Binding Assays

Two competition binding assays were utilized to determine IOC affinity for the human insulin receptor type B (IR(B)) against the endogenous ligand, insulin, labeled with ¹²⁵[I].

Method C: IR binding assay was a whole cell binding method using CHO cells overexpressing human IR(B). The cells were grown in F12 media containing 10% FBS and antibiotics (G418, Penicillin/Strepavidin), plated at 40,000 cells/well in a 96-well tissue culture plate for at least 8 h. The cells were then serum starved by switching to DMEM media containing 1% BSA (insulin-free) overnight. The cells were washed twice with chilled DMEM media containing 1% BSA (insulin-free) followed by the addition of IOC molecules at appropriate concentration in 90 μL of the same media. The cells were incubated on ice for 60 min. The ¹²⁵[I]-insulin (10 μL) was added at 0.015 nm final concentration and incubated on ice for 4 h. The cells were gently washed three times with chilled media and lysed with 30 μL of Cell Signaling lysis buffer (cat #9803) with shaking for 10 min at rt. The lysate was added to scintillation liquid and counted to determine ¹²⁵[I]-insulin binding to IR and the titration effects of IOC molecules on this interaction.

Method D: IR binding assay was run in a scintillation proximity assay (SPA) in 384-well format using cell membranes prepared from CHO cells overexpressing human IR(B) grown in F12 media containing 10% FBS and antibiotics (G418, Penicillin/Strepavidin). Cell membranes were prepared in 50 mM Tris buffer, pH 7.8 containing 5 mM MgCl₂. The assay buffer contained 50 mM Tris buffer, pH 7.5, 150 mM NaCl, 1 mM CaCl₂, 5 mgCl₂, 0.1% BSA and protease inhibitors (Complete-Mini-Roche). Cell membranes were added to WGA PVT PEI SPA beads (5 mg/mL final concentration) followed by addition of IOC molecules at appropriate concentrations. After 5 to 15 min incubation at rt, ¹²⁵[i]-insulin was added at 0.015 nm final concentration for a final total volume of 50 μL. The mixture was incubated with shaking at rt for 1 to 12 h followed by scintillation counting to determine ¹²⁵[I]-insulin binding to IR and the titration effects of IOC molecules on this interaction.

### Human Macrophage Mannose Receptor 1 (NIRC1) Binding Assays

The competition binding assay for Human macrophage mannose receptor 1 (MRC1) utilized a ligand, mannosylated-BSA labeled with the DELFIA Eu-N1-ITC reagent, as reported in the literature. Assay was performed either in a 96-well plate with 100 μL well volume (Method E) or in a 384-well plate with 25 μL well volume (Method F). Anti-MRC1 antibody (2 ng/μl) in PBS containing 1% stabilizer BSA was added to a Protein G plate that had been washed three times with 100 μl of 50 mM Tris buffer, pH 7.5 containing 100 mM NaCl, 5 mM CaCl₂, 1 mM MgCl₂ and 0.1% Tween-20 (wash buffer). The antibody was incubated in the plate for 1 h at rt with shaking. The plate was washed with wash buffer 3 to 5 times followed by addition of MRC1 (2 ng/μl final concentration) in PBS containing 1% stabilizer BSA. The plate was incubated at rt with gentle shaking for 1 h. The plate was washed three times with wash buffer. The IOC molecules in 12.5 μL (or 50 μL depending on plate format) buffer at appropriate concentrations were added followed by 12.5 μL (or 50 μL) Eu-mannosylated-BSA (0.1 nm final concentration) in 50 mM Tris, pH 7.5 containing 100 mM NaCl, 5 mM CaCl₂, 1 mM MgCl₂ and 0.2% stabilizer BSA. The plate was incubated for 2 h at rt with shaking followed by washing three times with wash buffer. Perkin Elmer Eu-inducer reagent was added and incubated for 30 min at rt prior to detection of the Eu signal (Excitation=340 nm: Emission=615 nm).

The following table lists conjugates that were prepared using appropriate intermediates following one of the General Methods described above. These conjugates were characterized using UPLC Method E or UPLC Method G noted by an asterisk (*) or UPLC Method F noted by a dagger (†), exhibiting either four charged, i.e. [(M+4)/4], (or five charged, i.e. [(M+5)/5]) species of parent compound at certain retention time (t_{R}). The in vitro biological activities towards insulin receptor (IR) were measured by either ligand competition assays or functional phosphorylation assays, as described above, labeled as following: Method A: IR phosphorylation assay based on 96-well; Method B: IR phosphorylation assay based on 384-well with automated liquid dispense; Method C: cell-based IR binding assay; Method D: SPA IR binding assay method E; Method E: MRC1 assay was performed in a 96-well plate; Method F: MRC1 assay was performed in a 384-well plate. The results are shown in Table 7.

TABLE 7

| | UPLC-MS | | | | | | | |
|---|---|---|---|---|---|---|---|---|
| | | Mass | IR Activation | | IR Binding | | MRC1 Binding | |
| | T_{R} | [(m + 4)/4 or | IP† | | IP‡ | | IP‡ | |
| IOC # | (min) | (m + 5)/5] | (nM) | Method | (nM) | Method | (nM) | Method |
| IOC-1 | 3.67 | 1924.45 | 29.1 | A | 6.151 | D | 8.09 | F |
| IOC-2 | 3.76 | 1767.38 | 39.46 | A | 5.185 | E | 36.39 | H |
| IOC-3 | 3.72 | 1934.98 | 24.96 | A | 2.941 | D | NA | NA |
| IOC-4 | 3.74 | 1774.87 | 24.9 | A | 6.28 | D | 45.09 | H |
| IOC-5 | 3.67 | 1945.94 | 4.494 | A | 1.07 | D | 4.57 | H |
| IOC-6 | 3.7 | 1780.52 | 8.001 | A | 3.531 | D | 27.4 | H |
| IOC-7 | 3.74 | 1780.50 | 6.467 | A | 2.138 | D | 27.44 | H |
| IOC-8 | 3.68 | 1780.53 | 1.438 | A | 0.8112 | D | 37.54 | H |
| IOC-9 | 3.5 | 1787.54 | 2.491 | C | 4.258 | E | 24.26 | H |
| IOC-10 | 3.44 | 1955.83 | 2.966 | C | 6.213 | E | 14.13 | H |
| IOC-11 | 3.37 | 1924.93 | 5.758 | C | 5.026 | E | 2.592 | H |
| IOC-12 | 3.39 | 1767.23 | 6.168 | C | 4.31 | E | 5.263 | H |
| IOC-13 | 3.46 | 1934.99 | 4.722 | C | 4.585 | E | 2.872 | H |
| IOC-14 | 3.48 | 1774.71 | 4.177 | C | 3.442 | E | 8.246 | H |
| IOC-15 | 3.45 | 1781.06 | 3.767 | C | 4.845 | E | 3.77 | H |
| IOC-16 | 3.43 | 1945.79 | 8.68 | C | 17.39 | E | 4.14 | H |
| IOC-17 | 3.51 | 1780.85 | 1.637 | C | 0.9264 | E | 1.581 | H |
| IOC-18 | 3.48 | 1787.90 | 3.125 | C | 2.429 | E | 3.156 | H |
| IOC-19 | 3.44 | 1955.89 | 3.315 | C | 2.326 | E | 1.351 | H |
| IOC-20 | 4.03 | 1782.68 | 3.832 | C | 5.011 | E | 142.4 | H |
| IOC-21 | 4.05 | 1782.82 | 0.4837 | C | 28.65 | D | 38.67 | G |
| IOC-22 | 3.56 | 1756.63 | 7.15 | C | 8.08 | E | 76.97 | H |
| IOC-23 | 3.48 | 1780.55 | 0.3946 | C | 0.8014 | E | 5.80 | G |
| IOC-24 | 3.47 | 1780.54 | 2.236 | C | 4.196 | E | 14.68 | H |
| IOC-25 | 3.46 | 1795.53 | 4.794 | C | 4.492 | E | 12.8 | H |
| IOC-26 | 3.44 | 1966.81 | 5.396 | C | 9.485 | E | 3.957 | H |
| IOC-27 | 4.01 | 1782.47 | 4.694 | C | 5.66 | E | 64.31 | H |
| IOC-28 | 4.02 | 1782.65 | 5.097 | C | 3.236 | E | 25.4 | H |
| IOC-29 | 4.04 | 1782.56 | 0.5152 | C | 0.01505 | E | 8.52 | G |
| IOC-30 | 3.47 | 1796.35 | 3.378 | C | 5.035 | E | 14.73 | H |
| IOC-31 | 3.46 | 1968.28 | 11.35 | C | 16 | E | 9.368 | H |
| IOC-32 | 4.3 | 1781.06 | 5.336 | C | 7.578 | E | 91.54 | H |
| IOC-33 | 4.09 | 1774.86 | 7.144 | C | 8.65 | E | 129.5 | H |
| IOC-34 | 3.85 | 1781.28 | 7.517 | C | 6.554 | E | 5.869 | H |
| IOC-35 | 4.16 | 1788.13 | 1.831 | C | 1.691 | E | 1.947 | H |
| IOC-36 | 4.19 | 1774.33 | 1.302 | C | 1.686 | E | 63.64 | H |
| IOC-37 | 4.61 | 1781.73 | 1.491 | C | 2.301 | E | 50.83 | H |
| IOC-38 | 3.48 | 1809.72 | 0.8859 | C | 1.259 | E | 39.13 | H |
| IOC-39 | 3.49 | 1809.73 | 1.429 | C | 1.229 | E | 4.952 | H |
| IOC-41 | 3.46 | 1810.16 | 4.931 | C | 3.618 | E | 18.02 | H |
| IOC-42 | 3.42 | 1809.32 | 7.815 | C | 5.158 | E | 6.556 | H |
| IOC-43 | 3.47 | 1823.66 | 6.167 | C | 10.88 | E | 34.38 | H |
| IOC-44 | 3.53 | 1823.69 | 6.522 | C | 11.44 | E | 5.308 | H |
| IOC-45 | 3.8 | 1765.3134 | 3.071 | C | 4.543 | E | 1128 | H |
| IOC-46 | 3.8 | 1921.4338 | 3.557 | C | 1.57 | E | 1594 | H |
| IOC-47 | 3.42 | 1787.908 | 3.765 | C | 8.482 | E | 3.42 | H |
| IOC-48 | 3.44 | 1957.1193 | 3.921 | C | 10.9 | E | 5.53 | H |
| IOC-49 | 4.44 | 1788.4076 | 4.824 | C | 6.477 | E | 35.51 | H |
| IOC-50 | 3.76 | 1945.8639 | 5.639 | C | 7.97 | E | 1707 | H |
| IOC-51 | 3.77 | 1781.5822 | 2.49 | C | 5.801 | E | 2260 | H |
| IOC-52 | 4.2 | 1780.9196 | 3.307 | C | 4.271 | E | 581.6 | H |
| IOC-53 | 4.07 | 1773.8245 | 4.72 | C | 9.144 | E | 88.03 | H |
| IOC-54 | 4.03 | 1933.6387 | 3.024 | C | 4.005 | E | 165.8 | H |
| IOC-55 | 3.4 | 1781.725 | 2.754 | C | 4.005 | E | 20.21 | H |
| IOC-56 | 4.27 | 1795.165 | 11.44 | C | 21.66 | E | 138.8 | H |
| IOC-57 | 3.58 | 1794.44 | 2.492 | C | 6.356 | E | 180.1 | H |
| IOC-58 | 3.06 | 1780.518 | 4.376 | C | 13.73 | E | 19.1 | H |
| IOC-59 | 4.32 | 1794.395 | 2.809 | C | 6.305 | E | 13.78 | H |
| IOC-60 | 3.74 | 1829.054 | 2.738 | C | 3.725 | E | 21.36 | H |
| IOC-61 | 3.95 | 1810.96 | 3.677 | C | 4.38 | E | 154.1 | H |
| IOC-62 | 3.90 | 1779.43 | 4.161 | C | 7.72 | E | 181.3 | H |
| IOC-63 | 3.53 | 1742.19 | 4.331 | C | 3.847 | E | 58.76 | H |
| IOC-64 | 4.16 | 1851.18 | 18.22 | C | 9.99 | E | 138.1 | H |
| IOC-65 | 3.90 | 1779.43 | 6.184 | C | 7.504 | E | 14.85 | H |
| IOC-66 | 3.7 | 1777.13 | 18.03 | C | 1.532 | E | 6.41 | H |
| IOC-67 | 3.66 | 1941.46 | 3.316 | C | 3.426 | E | 24.53 | H |
| IOC-68 | 3.54 | 1788.30 | 2.894 | C | 2.181 | E | 67.21 | H |
| IOC-69 | 3.51 | 1782.46 | 3.304 | C | 5.395 | E | 22.8 | H |
| IOC-70 | 3.49 | 1781.73 | 3.989 | C | 5.769 | E | 30.05 | H |
| IOC-71 | 3.49 | 1781.33 | 3.672 | C | 2.525 | E | 14.23 | H |
| IOC-72 | 3.37 | 1781.20 | 2.788 | C | 2.462 | E | 2.511 | H |
| IOC-73 | 3.34 | 1946.01 | 2.691 | C | 2.709 | E | 14.7 | H |
| IOC-74 | 3.33 | 1781.81 | 5.364 | C | 4.343 | E | 8.692 | H |
| IOC-75 | 3.61 | 1781.23 | 15.24 | C | 2.704 | E | 44.08 | H |
| IOC-76 | 4.05 | 1786.36 | 1.276 | C | 3.731 | E | 74.05 | H |
| IOC-77 | 4.01 | 1845.59 | 1.175 | C | 2.307 | E | 12.71 | H |
| IOC-79 | 3.63 | 1966.80 | 24.19 | C | 4.094 | E | 7.06 | H |
| IOC-80 | 3.39 | 1811.53 | 2.132 | C | 5.604 | E | 64.4 | H |
| IOC-81 | 3.38 | 1811.53 | 3.194 | C | 2.083 | E | 7.775 | H |
| IOC-82 | 3.36 | 1811.51 | 0.9426 | C | 0.5528 | E | 47.75 | H |
| IOC-83 | 3.36 | 1811.56 | 0.836 | C | 0.1612 | E | 2.752 | H |
| IOC-84 | 3.21 | 1957.37 | 4.609 | C | 9.728 | E | 19.85 | H |
| IOC-85 | 3.39 | 1788.89 | 2.165 | C | 2.584 | E | 2.253 | H |
| IOC-86 | 3.31 | 1967.84 | 3.526 | C | 3.086 | E | 1.559 | H |
| IOC-87 | 3.38 | 1795.32 | 2.244 | C | 2.27 | E | 4.925 | H |
| IOC-88 | 3.37 | 1629.2 | 2.825 | C | 2.589 | E | 0.7267 | H |
| IOC-89 | 3.33 | 1842.61 | 4.959 | C | 2.618 | E | 3.906 | H |
| IOC-90 | 3.25 | 1947.5 | 17.11 | C | 16.58 | E | 39.3 | H |
| IOC-91 | 3.28 | 1782.6 | 4.848 | C | 4.688 | E | 6.023 | H |
| IOC-92 | 3.4 | 1788.63 | 8.014 | C | 10.18 | E | 65.59 | H |
| IOC-93 | 4.07 | 1851.12 | 30.93 | C | 12.1 | E | 17.79 | H |
| IOC-94 | 3.36 | 1781.49 | 3.766 | C | 5.738 | E | 34.42 | H |
| | | (M + 4)/4 | | | | | | |
| IOC-95 | 3.39 | 1845.47 | 4.719 | C | 2.452 | E | 18.04 | H |
| IOC-96 | 3.73 | 1837.20 | 2.647 | C | 3.967 | E | 23.75 | H |
| IOC-97 | 3.39 | 1795.68 | 3.554 | C | 3.162 | E | 52.91 | H |
| IOC-98 | 3.35 | 1859.42 | 4.806 | C | 4.282 | E | 36.18 | H |
| IOC-99 | 3.39 | 1797.71 | 3.338 | C | 11.38 | E | 51.85 | H |
| IOC-101 | 3.38 | 1797.71 | 7.724 | C | 3.837 | E | 15.09 | H |
| IOC-102 | 3.38 | 1775.65 | 4.361 | C | 4.079 | E | 85.22 | H |
| IOC-103 | 3.4 | 1775.59 | 7.492 | C | 6.792 | E | 12.5 | H |
| IOC-104 | 3.41 | 1797.71 | 1.025 | C | 0.1994 | E | 36.32 | H |
| IOC-105 | 3.39 | 1797.77 | 0.9116 | C | 0.9455 | E | 7.035 | H |
| IOC-106 | 3.38 | 1775.64 | 0.6341 | C | 0.2511 | E | 61.47 | H |
| IOC-107 | 3.41 | 1775.61 | 1.078 | C | 0.7975 | E | 9.55 | H |
| IOC-108 | 3.38 | 1829.502 | 6.025 | C | 6.876 | E | 13.88 | H |
| IOC-109 | 3.39 | 1829.502 | 5.389 | C | 4.088 | E | 4.139 | H |
| IOC-110 | 3.44 | 1788.43 | 0.7285 | C | 0.7912 | E | 27.19 | H |
| IOC-111 | 4.11 | 1788.41 | 1.131 | C | 1.389 | E | 22.33 | H |
| IOC-112 | 3.53 | 1795.64 | 1.206 | C | 1.96 | E | 40.84 | H |
| IOC-113 | 4.04 | 1760.27 | 1.236 | C | 4.301 | E | 36.8 | H |
| IOC-114 | 4.06 | 1760.20 | 0.4731 | C | 1.248 | E | 12.41 | H |
| IOC-115 | 4.08 | 1759.79 | 2.572 | C | 6.093 | E | 86.25 | H |
| IOC-116 | 4.23 | 1766.17 | 1.456 | C | 2.457 | E | 55.96 | H |
| IOC-117 | 4.19 | 1766.68 | 1.789 | C | 2.988 | E | 20.15 | H |
| IOC-118 | 3.98 | 1765.91 | 1.422 | C | 7.128 | E | 38.26 | H |
| IOC-119 | 4.02 | 1766.63 | 0.8655 | C | 2.039 | E | 24.05 | H |
| IOC-120 | 4.04 | 1766.64 | 1.464 | C | 4.651 | E | 34.77 | H |
| IOC-121 | 3.5 | 1773.72 | 1.348 | C | 2.157 | E | 53.56 | H |
| IOC-122 | 4.12 | 1760.06 | 1.77 | C | 4.926 | E | 33.37 | H |
| IOC-123 | 4.06 | 1767.1189 | 4.706 | C | 6.42 | E | 7.046 | H |
| IOC-124 | 3.97 | 1766.9792 | 0.8071 | C | 1.834 | E | 6.753 | H |
| IOC-125 | 3.86 | 1795.4971 | 138 | C | 183.3 | E | 11.08 | H |
| IOC-126 | 3.59 | 1741.7902 | 6.857 | C | 7.722 | E | 35.14 | H |
| IOC-127 | 4.1 | 1760.05 | 0.9272 | C | 1.544 | E | 13.18 | H |
| IOC-128 | 4.04 | 1759.93 | 0.8071 | C | 2.148 | E | 16.95 | H |
| IOC-129 | 4.2 | 1767.58 | 0.6278 | C | 1.769 | E | 12.77 | H |
| IOC-130 | 3.52 | 1767.40 | 4.452 | C | 6.601 | E | 32.36 | H |
| IOC-131 | 3.82 | 1837.51 | 4.84 | C | 6.89 | E | 97.19 | H |
| IOC-132 | 3.4 | 1797.30 | 25.2 | C | 25.2 | E | 42.81 | H |
| IOC-133 | 3.4 | 1797.30 | 10.4 | C | 13.3 | E | 14.78 | H |
| IOC-134 | 3.43 | 1770.18 | 16.8 | C | 7.1 | E | 25.34 | H |
| IOC-135 | 3.39 | 1795.34 | 15.9 | C | 15.8 | E | 6.448 | H |
| IOC-136 | 3.4 | 1797.39 | 11.9 | C | 5.5 | E | 4.399 | H |
| IOC-137 | 3.37 | 1859.16 | 9.6 | C | 12.2 | E | 1.936 | H |
| IOC-138 | 3.39 | 1861.44 | 15.4 | C | 36.5 | E | 1.197 | H |
| IOC-139 | 3.41 | 1770.13 | 36.4 | C | 5.9 | E | 5.721 | H |
| IOC-140 | 3.4 | 1795.33 | 17.7 | C | 16.7 | E | 8.716 | H |
| IOC-141 | 3.37 | 1859.59 | 32 | C | 6.2 | E | 6.562 | H |
| IOC-142 | 3.39 | 1756.06 | 17.7 | C | 10.6 | E | 7.591 | H |
| IOC-143 | 3.46 | 1770.14 | 23.8 | C | 7.8 | E | 13.91 | H |
| IOC-144 | 4.04 | 1767.17 | 1.986 | C | 7.157 | E | 67.4 | H |
| IOC-145 | 4.02 | 1772.23 | 1.452 | C | 5.628 | E | 75.74 | H |
| IOC-146 | 4.03 | 1758.55 | 1000 | C | 2500 | E | 35.26 | H |
| IOC-147 | 3.08 | 1832.01 | 17.29 | C | 19.67 | E | 19.77 | H |
| IOC-148 | 3.06 | 1616.7 | 17.71 | C | 7.207 | E | 8.754 | H |

The effect of methyl α-d-mannopyranoside (αMM) on PK and PD of IOCs in Non-Diabetic minipigs was evaluated.

Male Yucatan miniature pigs, non-diabetic, instrumented with two Jugular vein vascular access ports (VAP), were used in these studies. Animals are fasted overnight prior to the study. On the day of the study, animals are restrained in slings, and VAPs accessed for infusion and sampling. At t=−60 min, a constant infusion of PBS (n=3) or 21.2% α-methyl mannose (αMM) (n=3) is started, at a rate of 2.67 mL/kg/h. This infusion was maintained for the duration of the study. At t=0 min, and after collecting a baseline blood sample for plasma glucose measurement, animals were administered IOC as a single bolus IV. Sampling continued for 90 min, with final readouts of plasma glucose and compound levels.

IOCs were formulated at 17-69 nmol/mL in NaCl (87 mM), phenol (21 mM), dibasic sodium phosphate (26.5 mM), Osmolality=275 mOsm, pH=7.4; QS with Water for Injection.

Time points for sample collection: −60 min, 0 min, 1 min, 2 min, 4 min, 6 min, 8 min, 10 min, 15 min, 20 min, 25 min, 30 min, 35 min, 45 min, 60 min, and 90 min.

Blood was collected in K3-EDTA tubes, supplemented with 10 μg/ml aprotinin, and kept on an ice bath until processing, within 30 min of collection. After centrifugation at 3000 rpm, 4° C., for 8 min, plasma was collected and aliquoted for glucose measurement using a Beckman Coulter AU480 Chemistry analyzer and for compound levels measurement by LC-MS.

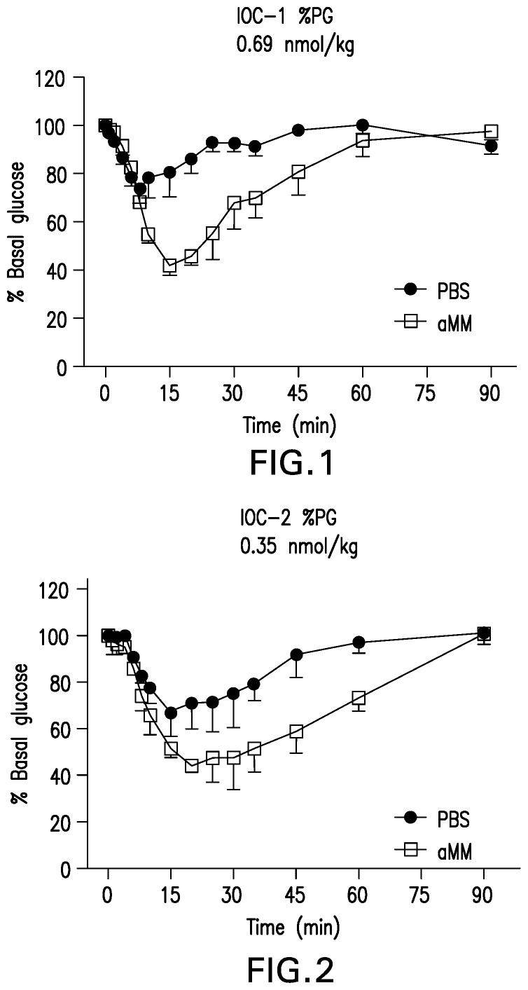

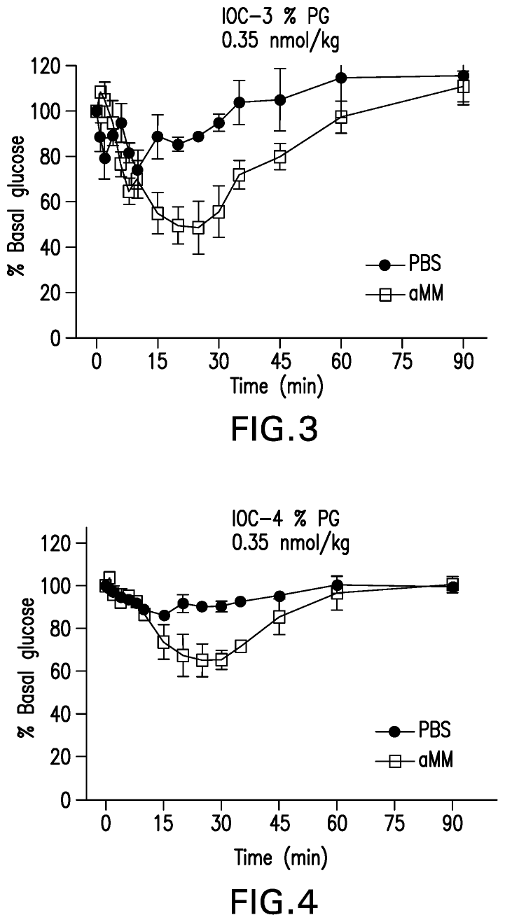

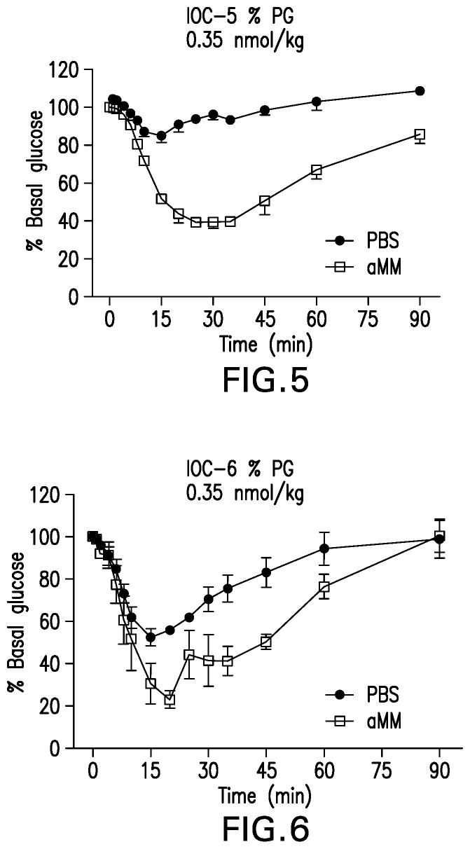

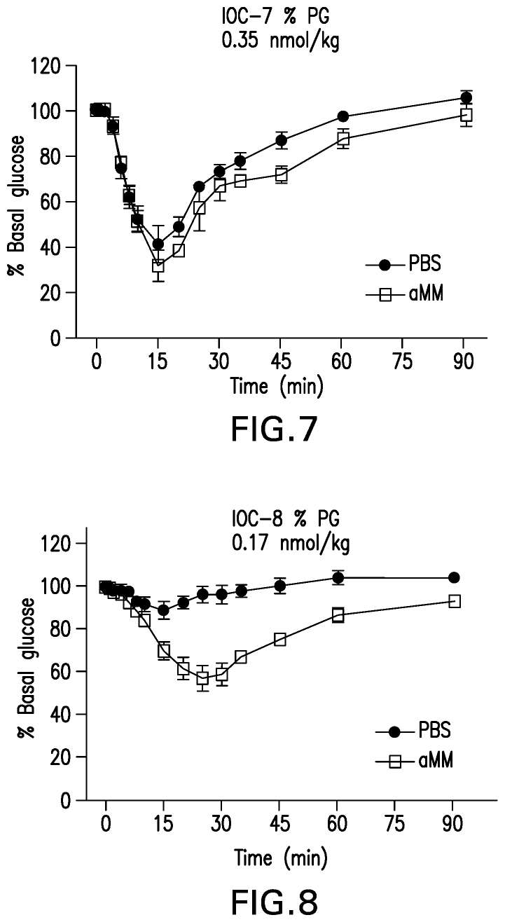

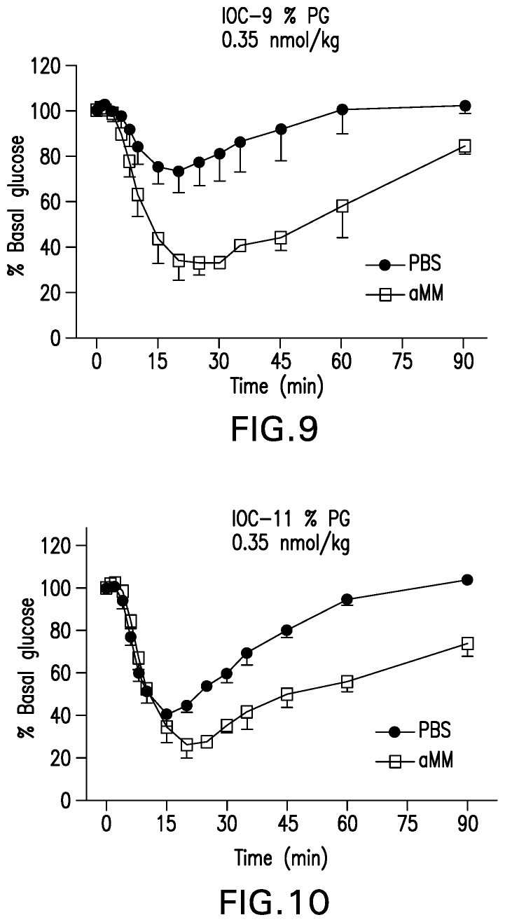

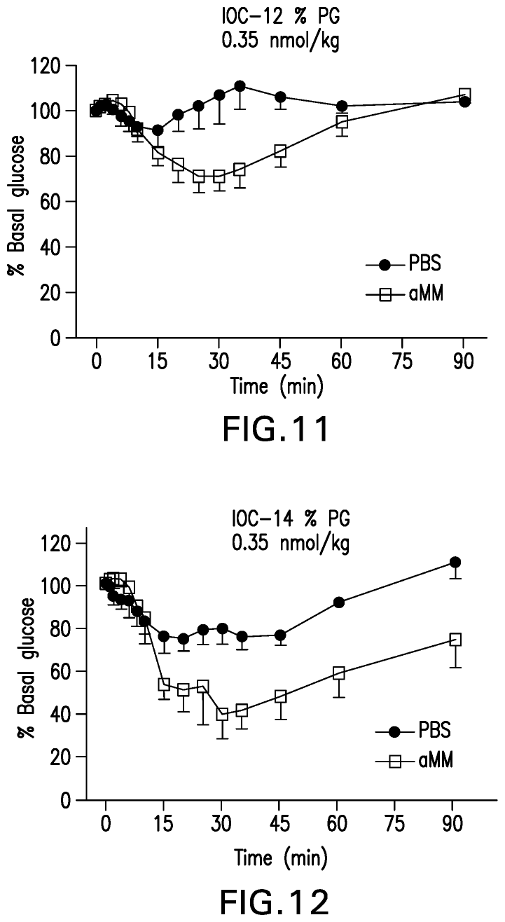

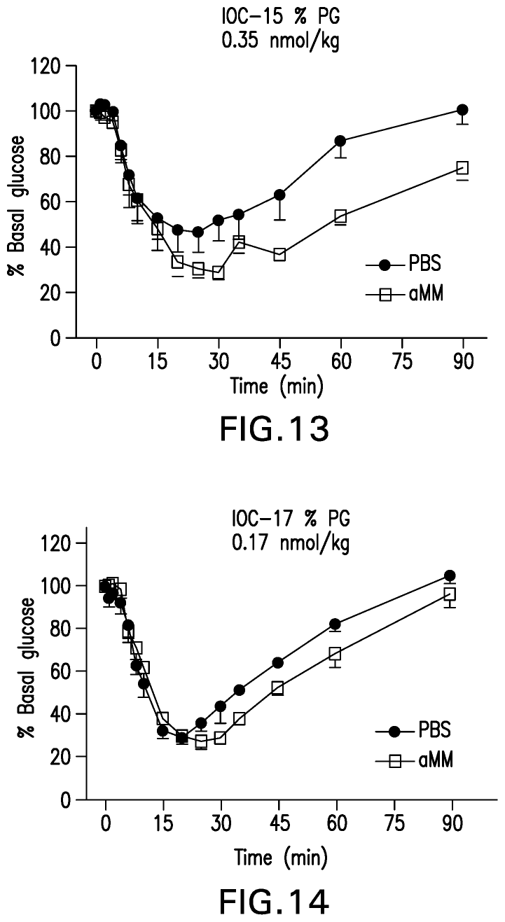

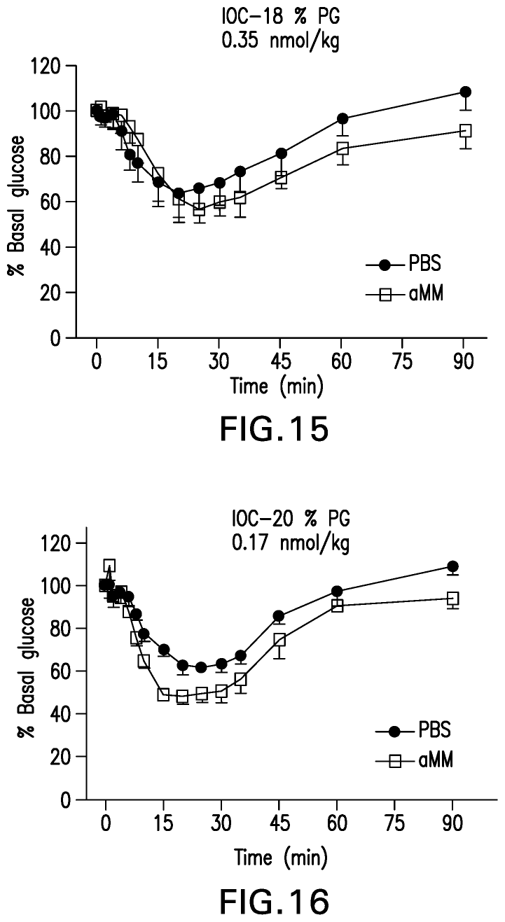

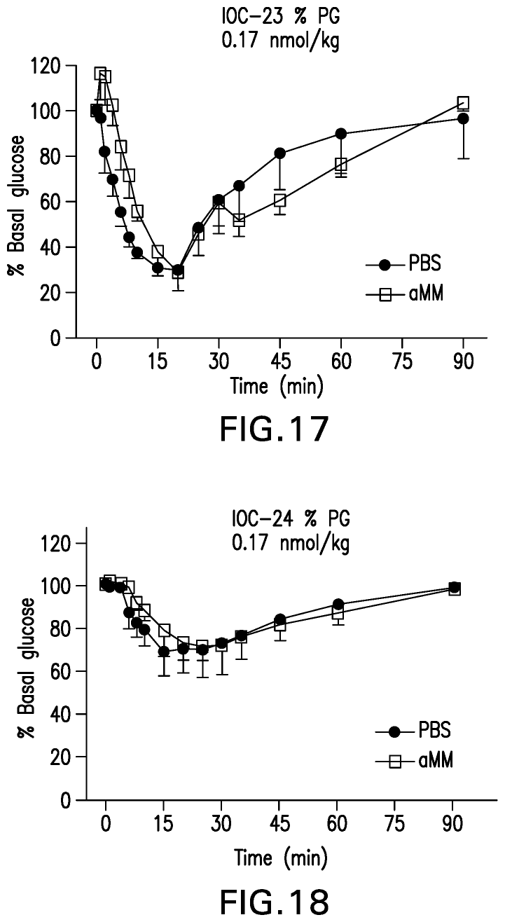

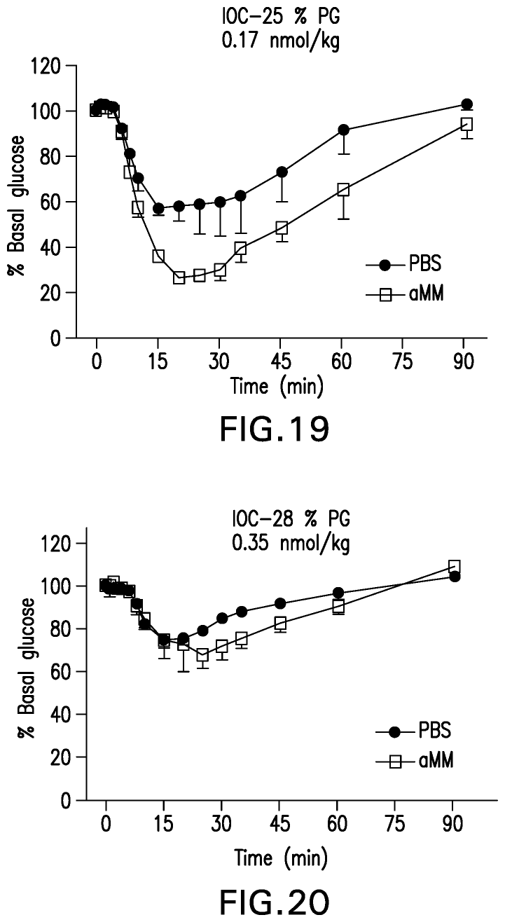

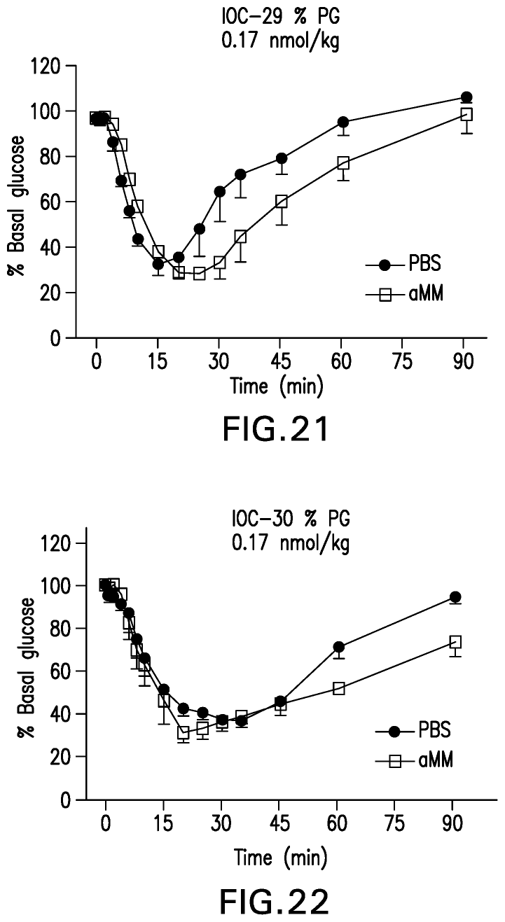

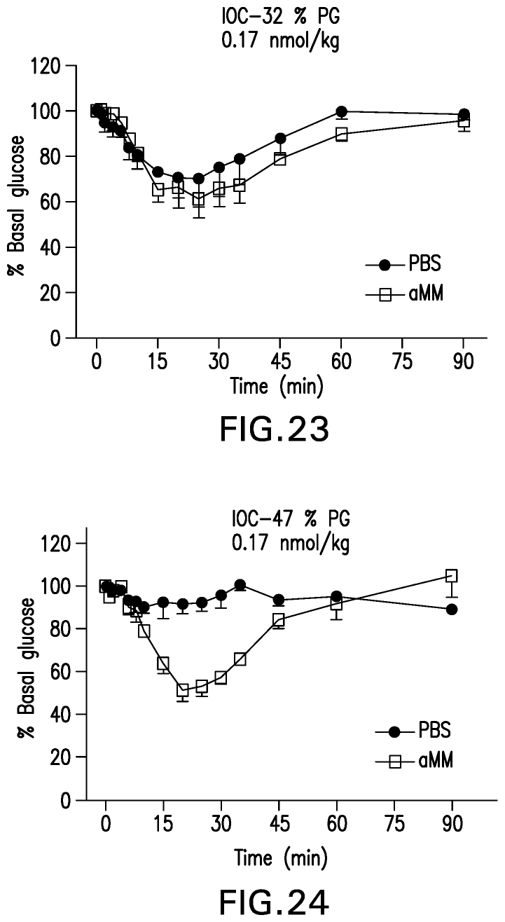

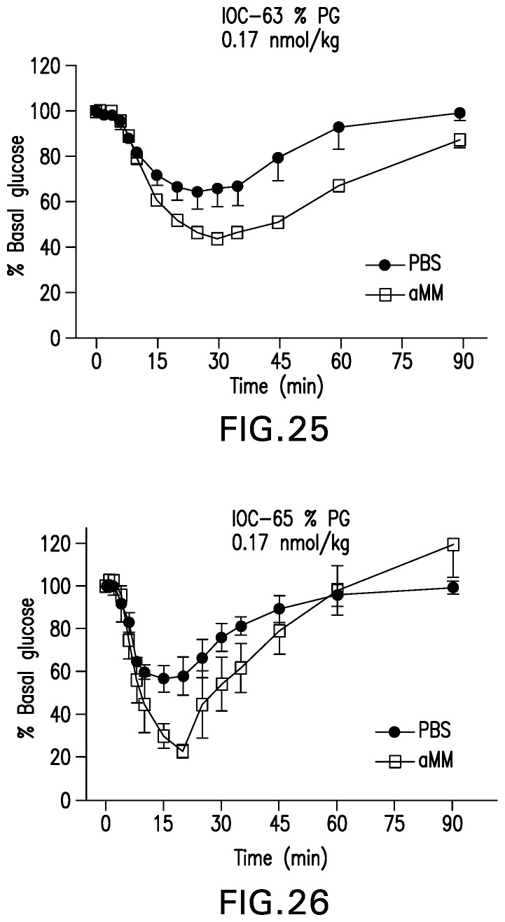

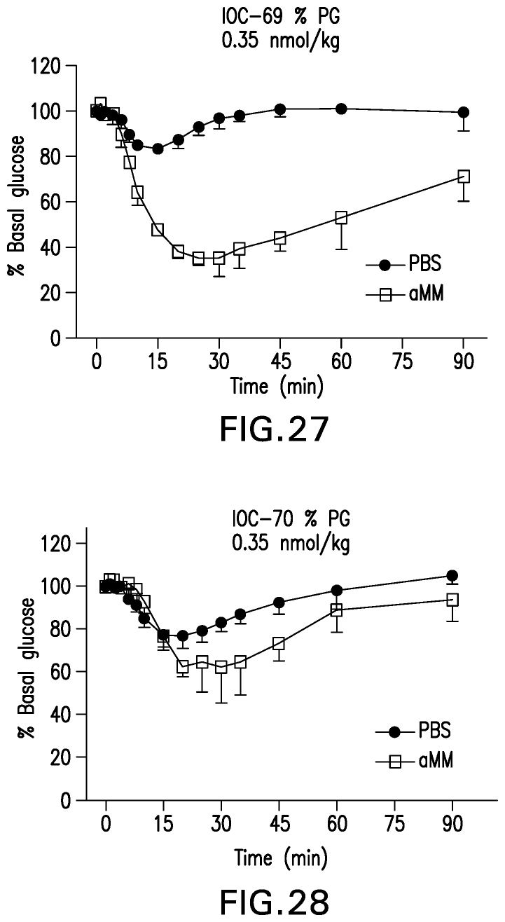

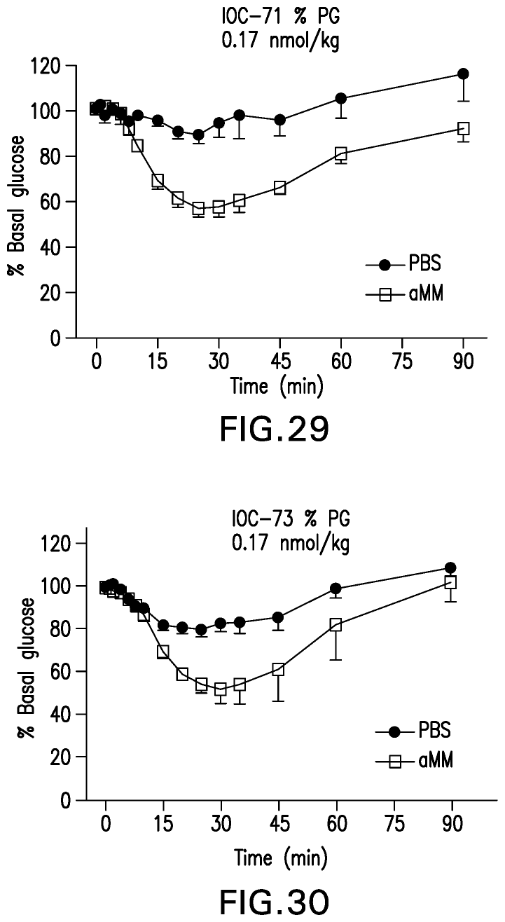

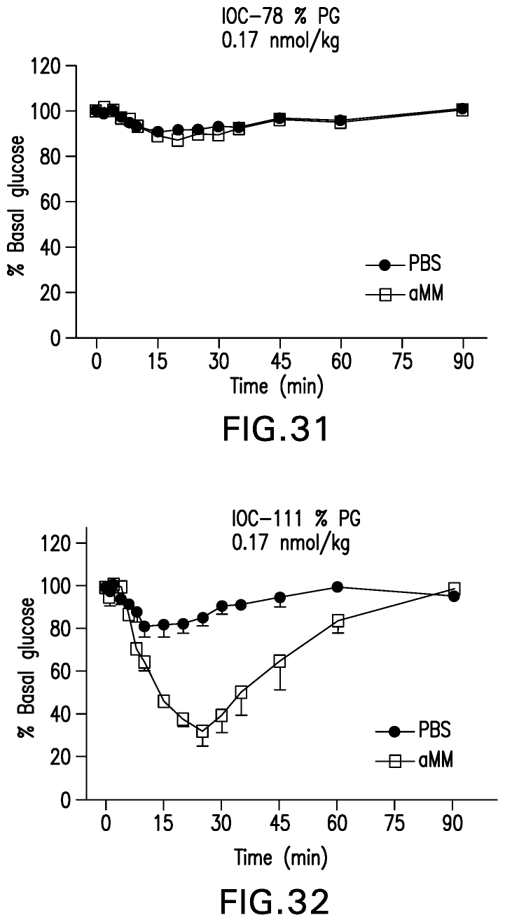

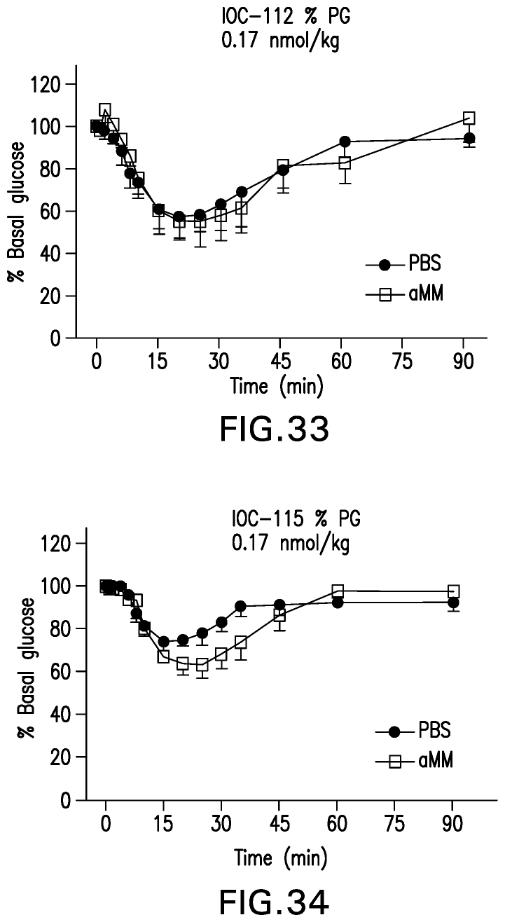

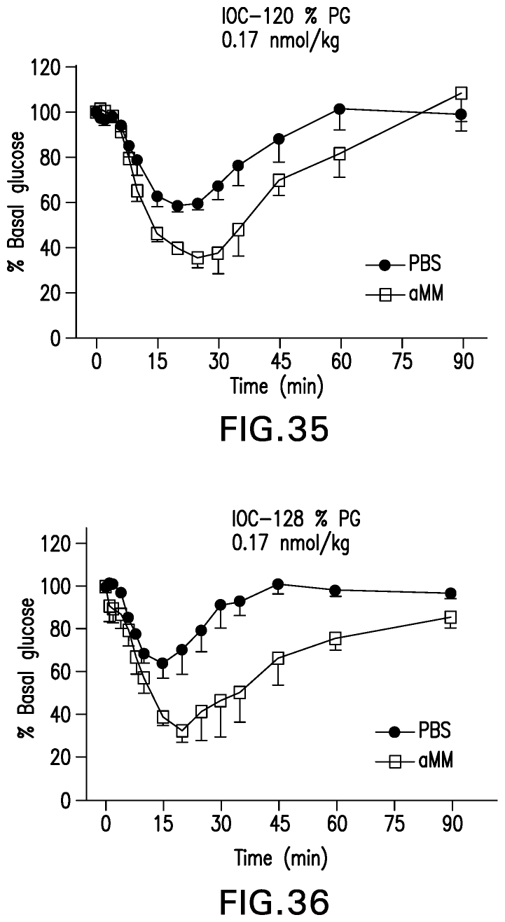

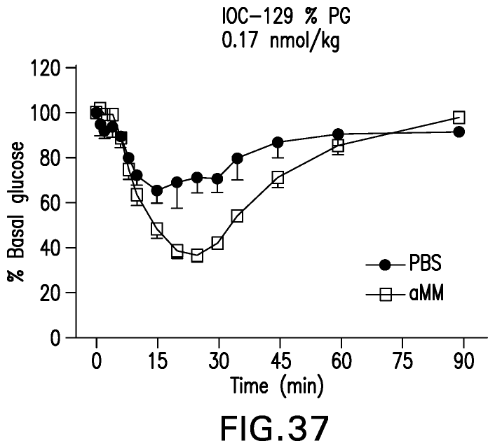

Glucose results were expressed as % changes over baseline values at t=0 min and are shown for IOC-1, IOC-2, IOC-3, IOC-4, IOC-5, IOC-6, IOC-7, IOC-8, IOC-9, IOC-11, IOC-12, IOC-14, IOC-15, IOC-17, IOC-18, IOC-20, IOC-23, IOC-24, IOC-25, IOC-28, IOC-29, IOC-30, IOC-32, IOC-47, IOC-63, IOC-65, IOC-69, IOC-70, IOC-71, IOC-73, IOC-75, IOC-78, IOC-111, IOC-112, IOC-115, IOC-120, IOC-18 and IOC-129 in FIGS. 1-37, respectively.

It will be appreciated that various of the above-discussed and other features and functions, or alternatives thereof, may be desirably combined into many other different systems or applications. It will also be appreciated that various presently unforeseen or unanticipated alternatives, modifications, variations or improvements therein may be subsequently made by those skilled in the art, which are also intended to be encompassed by the following claims.

1. A conjugate comprising an insulin or insulin analog molecule conjugated at its N-terminal amino groups of A-chain or B-chain or ε-amino group of a side chain of a Lys residue, to at least one trisaccharide cluster of sugar moieties via a linker.

2. The conjugate according to claim 1, wherein the conjugate comprises a first conjugation site, a second conjugation site, and a third conjugation site.

3. The conjugate according to claim 2, wherein the first conjugation site is conjugated to a first trisaccharide linker, the second conjugation site is conjugated to a second trisaccharide linker and the third conjugation site is conjugated to a third trisaccharide linker.

4. The conjugate according to claim 2, wherein the first two conjugation sites are conjugated to a trisaccharide and the third conjugation site is unconjugated or conjugated to a trisaccharide.

5. A conjugate having the general formula (I): 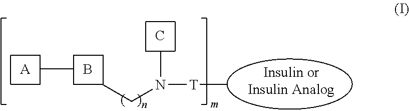
wherein
(a) the insulin or insulin analog is selected from human insulin, porcine insulin, insulin lispro, insulin aspart, insulin glulisine, insulin glargine, insulin detemir, GlyA21 human insulin, GlyA3 human insulin, LysA22 human insulin, LysB3 human insulin, HisA8 human insulin, GlyA21 ArgA22 human insulin, DesB30 human insulin, LysA9 DesB30 human insulin, GlyA21 DesB30 human insulin, LysA22 DesB30 human insulin, LysB3 DesB30 human insulin, LysA1 ArgB29 DesB30 human insulin, LysA5 ArgB29 DesB30 human insulin, LysA9 ArgB29 DesB30 human insulin, LysA10 ArgB29 DesB30 human insulin, LysA13 ArgB29 DesB30 human insulin, LysA14 ArgB29 DesB30 human insulin, LysA15 ArgB29 DesB30 human insulin, LysA18 ArgB29 DesB30 human insulin, LysA22 ArgB29 DesB30 human insulin, LysA1 GlyA21 ArgB29 DesB30 human insulin, GlyA21 ArgB29 DesB30 human insulin, LysB1 ArgB29 DesB30 human insulin, LysB3 ArgB29 DesB30 human insulin, LysB4 ArgB29 DesB30 human insulin, LysB16 ArgB29 DesB30 human insulin, LysB17 ArgB29 DesB30 human insulin, LysB25 ArgB29 DesB30 human insulin, GlyA21 ArgB31 ProB32 ArgB33 ProB34 ArgB35 human insulin, GlyA21 ArgA22 ArgB31 ProB32 ArgB33 human insulin, and insulin analogs that comprise
(i) an A chain polypeptide sequence comprising a sequence of X₁I X₂E X₃CCX₄ X₅ X₆CS X₇ X₈ X₉LE X₁₀YC X₁₁X₁₂ (SEQ ID NO: 3) and
(ii) a B chain polypeptide sequence comprising a sequence of
(SEQ ID NO: 4)
X₁₃VX₁₄X₁₅HLCGSHLVEALX₁₆X₁₇VCGERGFX₁₈YTX₁₉X₂₀X₂₁X₂₂
X₂₃X₂₄X₂₅X₂₆
wherein:
X₁ is glycine (G) or lysine (K);
X₂ is valine (V), glycine (G), or lysine (K);
X₃ is glutamine (Q) or lysine (K);
X₄ is threonine (T), histidine (H), or lysine (K);
X₅ is serine (S) or lysine (K);
X₆ is isoleucine (I) or lysine (K);
X₇ is leucine (L) or lysine (K);
X₈ is tyrosine (Y) or lysine (K);
X₉ is glutamine (Q) or lysine (K);
X₁₀ is asparagine (N) or lysine (K);
X₁₁ is asparagine (N), glycine (G), or lysine (K);
X₁₂ is arginine (R), lysine (K), or absent;
X₁₃ is phenylalanine (F) or lysine (K);
X₁₄ is asparagine (N) or lysine (K);
X₁₅ is glutamine (Q) or lysine (K);
X₁₆ is tyrosine (Y) or lysine (K);
X₁₇ is leucine (L) or lysine (K);
X₁₈ is phenylalanine (F) or lysine (K);
X₁₉ is proline (P) or lysine (K):
X₂₀ is lysine (K), proline (P), arginine (R), or is absent;
X₂₁ is threonine (T) or absent;
X₂₂ is arginine (R) if X₂₁ is threonine (T), or absent;
X₂₃ is proline (P) if X₂₂ is arginine (R), or absent;
X₂₄ is arginine (R) if X₂₃ is proline (P), or absent;
X₂₅ is proline (P) if X₂₄ is arginine (R), or absent; and
X₂₆ is arginine (R) if X₂s is proline (P), or absent,
with the proviso that at least one of X₁, X₃, X₅, X₆, X₇, X₈, X₉, X₁₀, X₁₂, X₁₃, X₁₄, X₁₅, X₁₆, X₁₇, X₁₈, and X₁₉ is a lysine (K) and when X₁₉ is lysine (K) then X₂₀ is absent or if X₂₀ is present then at least one of X₁, X₃, X₄, X₅, X₆, X₇, X₈, X₉, X₁₀, X₁₁, X₁₂, X₁₃, X₁₄, X₁₅, X₁₆, and X₁₇ is lysine (K), or X₄ is histidine (H), or X₁₁ is glycine (G); or at least one of X₁₂ or X₂₁ is present;
(b) the linker is covalently linked to the amino group at position A1 of the insulin or insulin analog molecule; position B1 of the insulin or insulin analog molecule; position B29 of the insulin or insulin analog molecule; or other lysine residue of the insulin or insulin analog molecule;
(c) each occurrence of [symbol] is an independently selected trisaccharide;
(d) each [symbol] is selected independently from CH₂ and O;
(e) each [symbol] is selected independently from H and CH₃;
(f) m is the number of individual, independently selected monomeric units 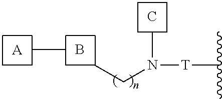
that are conjugated to the insulin or insulin analog, and is selected from 1, 2, or 3;
(g) n is the number of methylene units, and is selected from 1, 2, or 3.

6. The conjugate according to claim 5, wherein each T is independently selected from the group consisting of

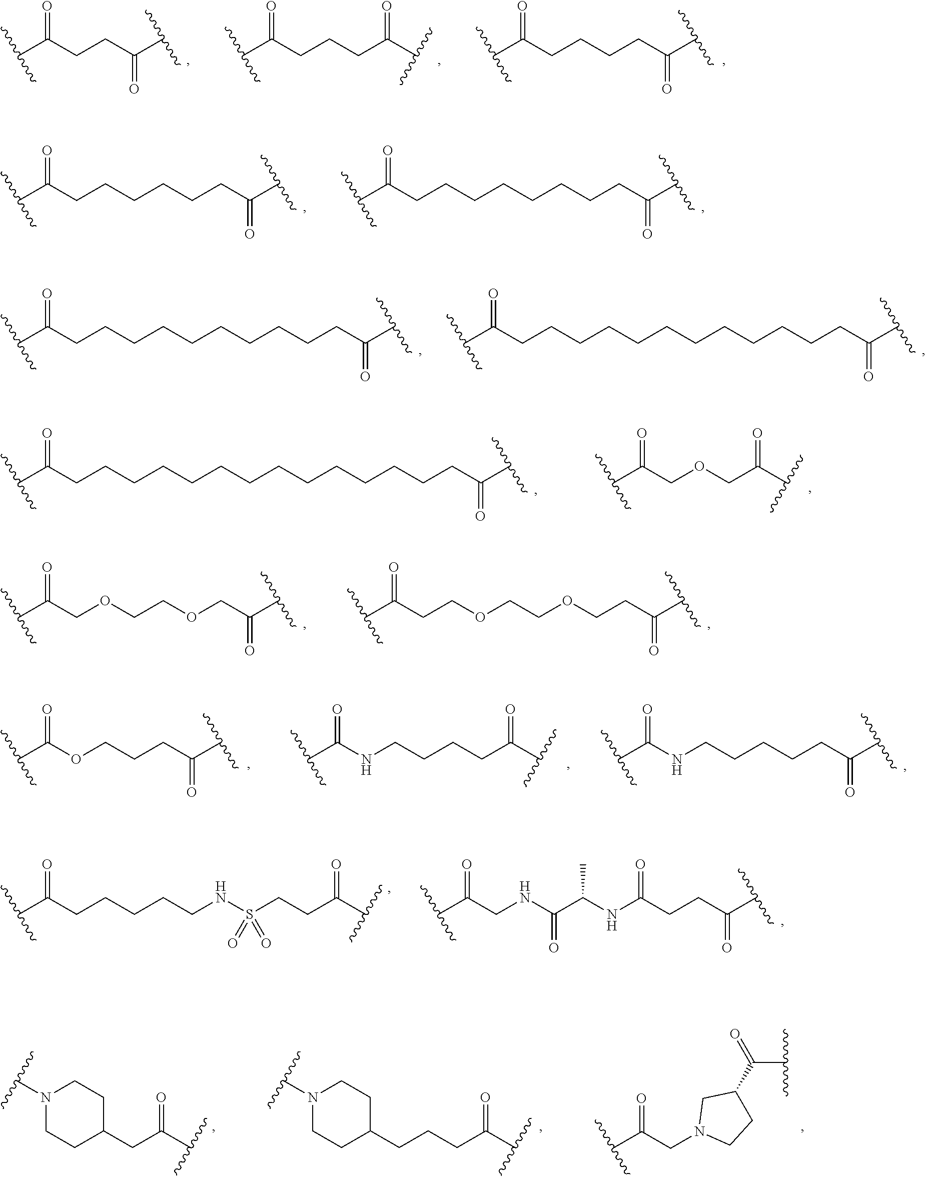

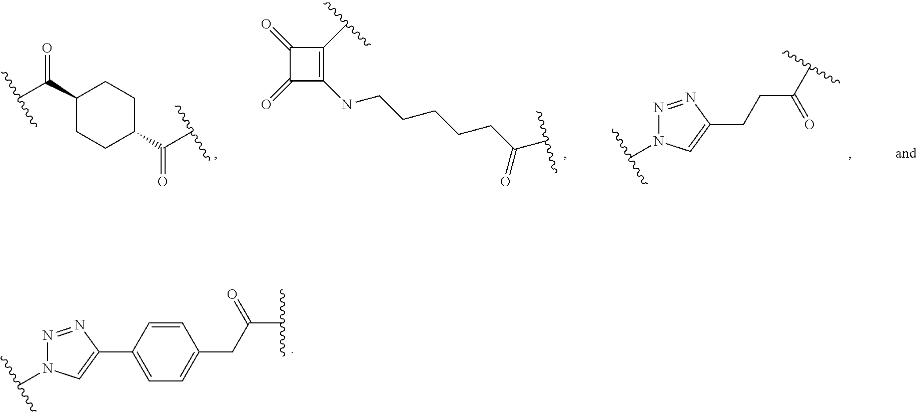 [SMILES: *C(=O)[C@H]1CC[C@H](C(*)=O)CC1 ; *C(=O)CCCCCNc1c(*)c(=O)c1=O ; *C(=O)CCc1cn(*)nn1 ; *C(=O)Cc1ccc(-c2cn(*)nn2)cc1]

7. The conjugate according to any claim 5, wherein the conjugate prepared using a reagent having a formula selected from the group consisting of ML-1, ML-2, ML-3, ML-4, ML-5, ML-6, ML-7, ML-8, ML-9, ML-10, ML-11, ML-12, ML-13, ML-14, ML-15, ML-16, ML-17, ML-18, ML-19, ML-20, ML-21, ML-22, ML-23, ML-24, ML-25, ML-26, ML-27, ML-28, ML-29, ML-30, ML-31, ML-32, ML-33, ML-34, ML-35, ML-36, ML-37, ML-38, ML-39, ML-40, ML-41, ML-42, ML-43, ML-44, ML-45, ML-46, ML-47, ML-48, ML-49, ML-50, ML-51, ML-52, ML-53, ML-54, ML-55, and ML-56.

8. The conjugate according to claim 5, wherein the conjugate has a formula selected from the group consisting of IOC-1, 10C-2, IOC-3, IOC-4, IOC-5, IOC-6, IOC-7, IOC-8, IOC-9, IOC-10, IOC-11, IOC-12, IOC-13, IOC-14, IOC-15, IOC-16, IOC-17, IOC-18, IOC-19, IOC-20, IOC-21, IOC-22, IOC-23, IOC-24, IOC-25, IOC-26, IOC-27, IOC-28, IOC-29, IOC-30, IOC-31, IOC-32, IOC-33, IOC-34, IOC-35, IOC-36, IOC-37, IOC-38, IOC-39, IOC-41, IOC-42, IOC-43, IOC-44, IOC-45, IOC-46, IOC-47, IOC-48, IOC-49, IOC-50, IOC-51, IOC-52, IOC-53, IOC-54, IOC-55, IOC-56, IOC-57, IOC-58, IOC-59, IOC-60, IOC-61, 10C-62, IOC-63, IOC-64, IOC-65, IOC-66, IOC-67, IOC-68, IOC-69, IOC-70, IOC-71, IOC-72, IOC-73, IOC-74, IOC-75, IOC-76, IOC-77, IOC-79, IOC-80, IOC-81, IOC-82, IOC-83, IOC-84, IOC-85, IOC-86, IOC-87, IOC-88, IOC-89, IOC-90, IOC-91, IOC-92, IOC-93, IOC-94, IOC-95, IOC-96, IOC-97, IOC-98, IOC-99, IOC-100, IOC-101, 10C-102, IOC-103, IOC-104, IOC-105, IOC-106, IOC-107, IOC-108, IOC-109, IOC-110, IOC-111, IOC-112, IOC-113, IOC-114, IOC-115, IOC-116, IOC-117, IOC-118, IOC-119, IOC-120, IOC-121, IOC-122, IOC-123, IOC-124, IOC-125, IOC-126, IOC-127, IOC-128, IOC-129, IOC-130, IOC-131, IOC-132, IOC-133, IOC-134, IOC-135, IOC-136, IOC-137, IOC-138, IOC-139, IOC-140, IOC-141, IOC-142, IOC-143, IOC-144, IOC-145, IOC-146, IOC-147, and IOC-148.

9. The conjugate according to claim 5, wherein each trisaccharide is independently selected from the group consisting of α-aminoethyl glucopyranoside, α-aminoethyl mannopyranoside, α-(1-3, 1-6) dimannopyranosyl-α-aminoethylglucopyranoside, α-(1-3, 1-6) dimannopyranosyl-β-aminoethylglucopyranoside, α-(1-3, 1-6) dimannopyranosyl-α-aminoethyl mannopyranoside, α-(1-3, 1-6) dimannopyranosyl-β-aminoethyl mannopyranoside, α-(1-3, 1-4) dimannopyranosyl-α-aminoethyl mannopyranoside, α-(1-3, 1-6) dimannopyranosyl α-aminoethyl (2-deoxy-2-fluoro-mannopyranoside, α-(1-3, 1-6) dimannopyranosyl α-aminoethyl (2-deoxy-2-fluoro-glucopyranoside, α-(1-3, 1-6) dimannopyranosyl (3-aminoethyl (2-deoxy-2-fluoro-glucopyranoside, α-(1-2, 1-4) dimannopyranosyl α-aminopropyl mannopyranoside, α-(1-2, 1-6) dimannopyranosyl β-aminopropyl mannopyranoside, α-(1-2) mannosyl α-(1-6) fucosyl α-aminopropyl mannopyranoside, α-(1-3, 1-6) difucosyl α-aminoethyl mannopyranoside, α-(1-3, 1-6) dimannopyranosyl-α-aminoethyl-C-mannopyranoside, α-(1-3, 1-6) dimannopyranosyl-α-N-methyl aminoethyl mannopyranoside, and α-(1-3, 1-6) dimannopyranosyl-β-aminoethyl-N-acetylglucosamine.

10. The conjugate according to claim 5, wherein each trisaccharide is independently selected from the group consisting of 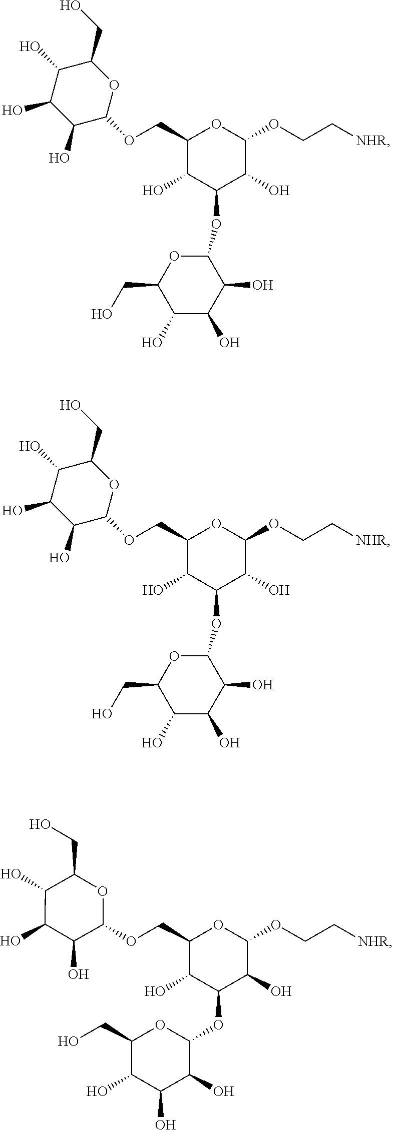 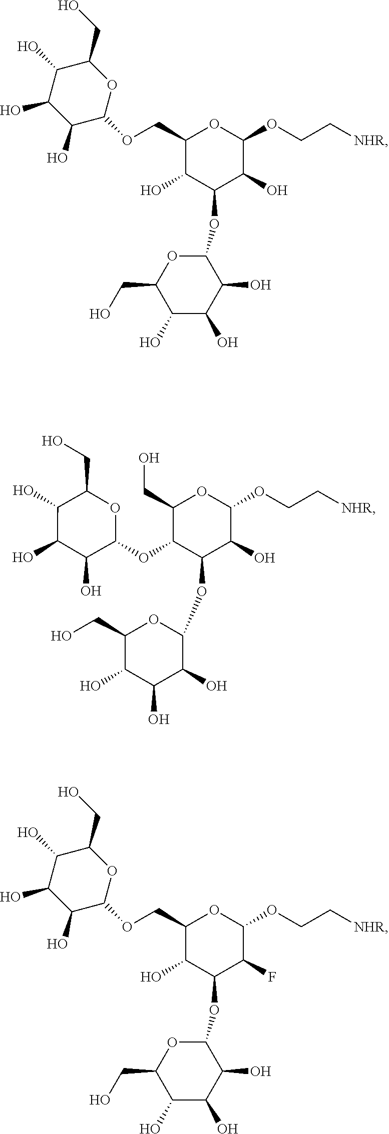 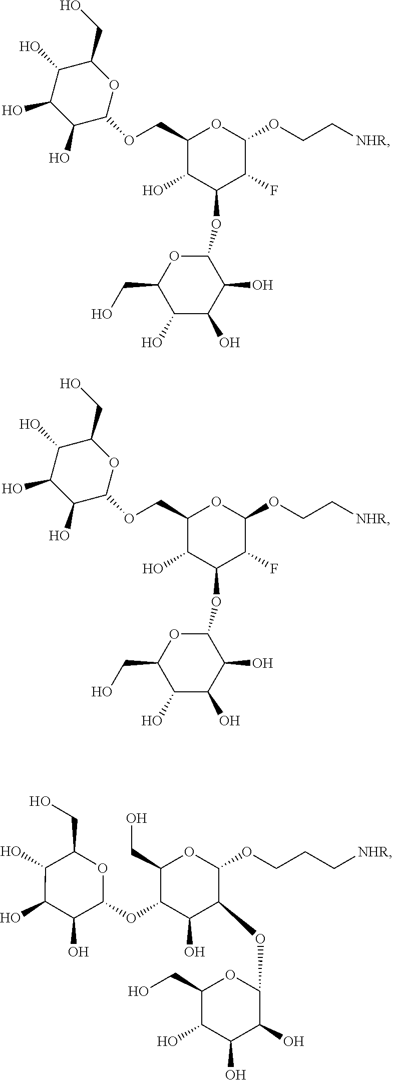 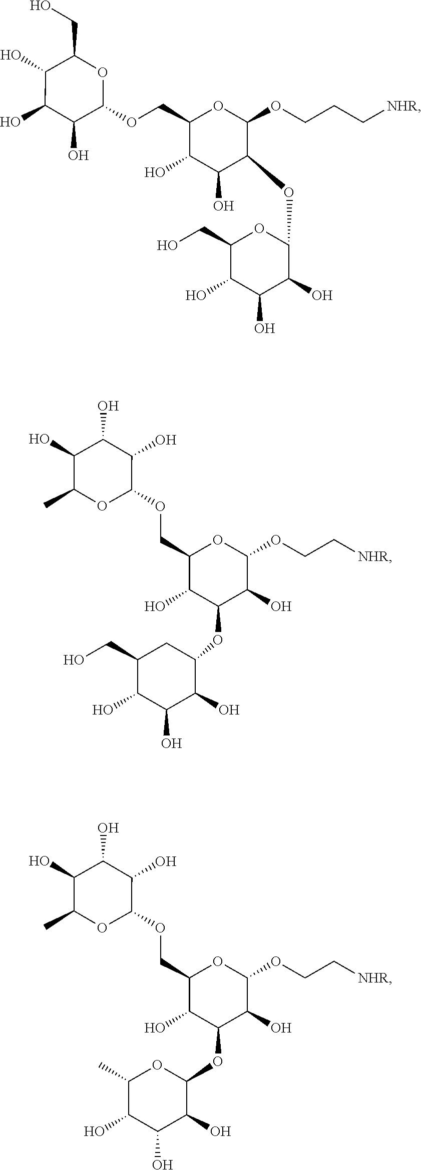 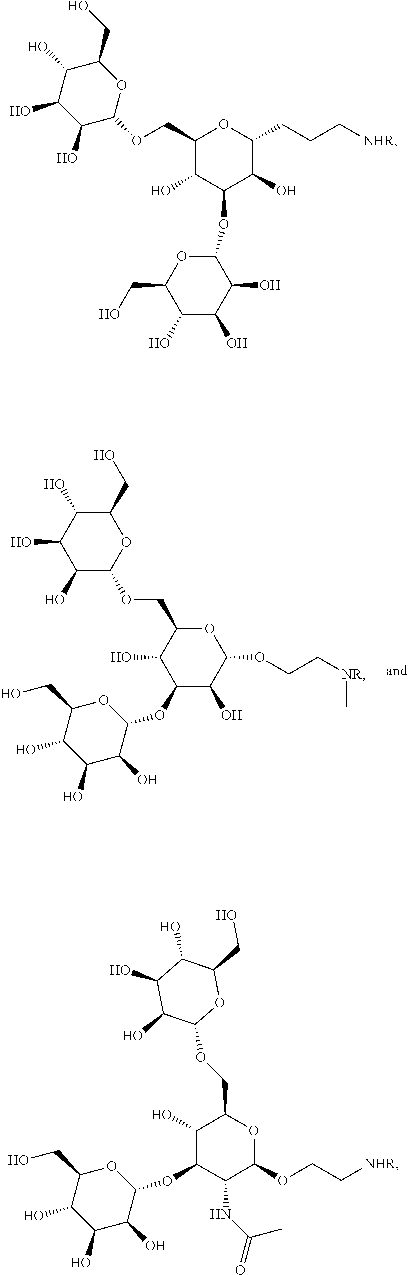
wherein R may be hydrogen or a carbonyl group of the linker.

11-12. (canceled)

13. A composition comprising the conjugate according to claim 5 and a pharmaceutically acceptable carrier.

14-15. (canceled)

16. A method for treating diabetes in a subject in need thereof, comprising:
administering to the subject an effective amount of the composition according to claim 13 for treating the diabetes.

17. The method according to claim 16, wherein the diabetes is Type I diabetes, Type II diabetes, or gestational diabetes.

18. A composition comprising the insulin analog conjugate according to claim 5, wherein the conjugate is characterized as having a ratio of EC50 or IP as determined by a functional insulin receptor phosphorylation assay to the IC50 or IP as determined by a competition binding assay at the macrophage mannose receptor that is about 0.5:1 to about 1:100; about 1:1 to about 1:50; about 1:1 to about 1:20; or about 1:1 to about 1:10; and a pharmaceutically acceptable carrier.

19. A method for treating diabetes in a subject in need thereof, comprising:
administering to the subject the composition of claim 18, wherein the administering treats the diabetes.

20. The method of claim 19, wherein the diabetes is Type I diabetes, Type II diabetes, or gestational diabetes.

21. A trisaccharide selected from the group consisting of α-aminoethyl glucopyranoside, α-aminoethyl mannopyranoside, α-(1-3, 1-6) dimannopyranosyl-α-aminoethylglucopyranoside, α-(1-3, 1-6) dimannopyranosyl-β-aminoethylglucopyranoside, α-(1-3, 1-6) dimannopyranosyl-α-aminoethyl mannopyranoside, α-(1-3, 1-6) dimannopyranosyl-β-aminoethyl mannopyranoside, α-(1-3, 1-4) dimannopyranosyl-α-aminoethyl mannopyranoside, α-(1-3, 1-6) dimannopyranosyl α-aminoethyl (2-deoxy-2-fluoro-mannopyranoside, α-(1-3, 1-6) dimannopyranosyl α-aminoethyl (2-deoxy-2-fluoro-glucopyranoside, α-(1-3, 1-6) dimannopyranosyl β-aminoethyl (2-deoxy-2-fluoro-glucopyranoside, α-(1-2, 1-4) dimannopyranosyl α-aminopropyl mannopyranoside, α-(1-2, 1-6) dimannopyranosyl β-aminopropyl mannopyranoside, α-(1-2) mannosyl α-(1-6) fucosyl α-aminopropyl mannopyranoside, α-(1-3, 1-6) difucosyl α-aminoethyl mannopyranoside, α-(1-3, 1-6) dimannopyranosyl-α-aminoethyl-C-mannopyranoside, α-(1-3, 1-6) dimannopyranosyl-α-N-methyl aminoethyl mannopyranoside, and α-(1-3, 1-6) dimannopyranosyl-β-aminoethyl-N-acetylglucosamine.

22. A trisaccharide selected from the group consisting of 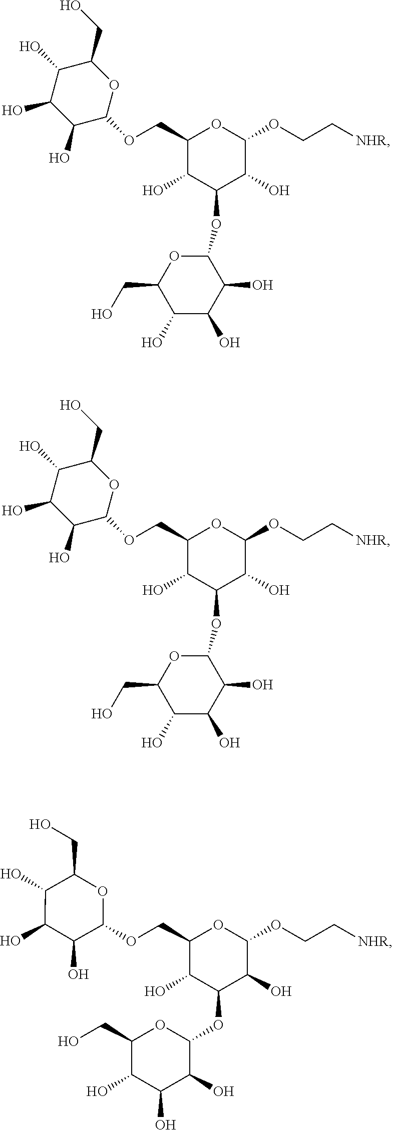 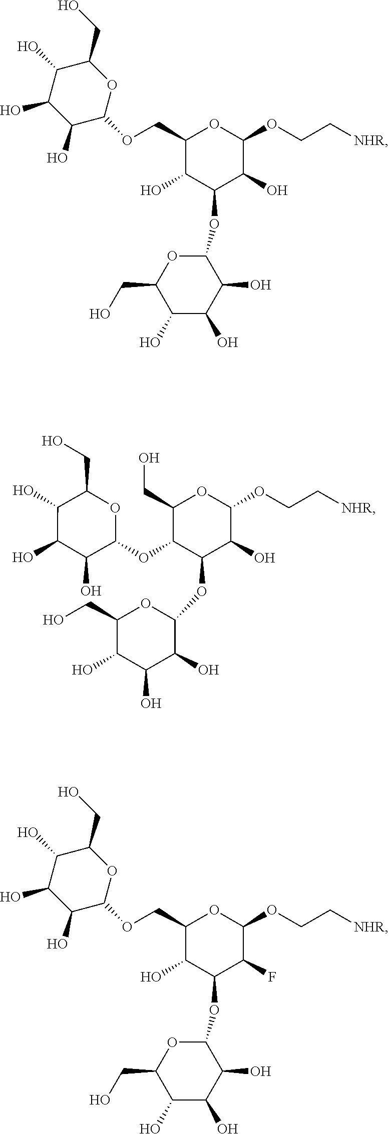 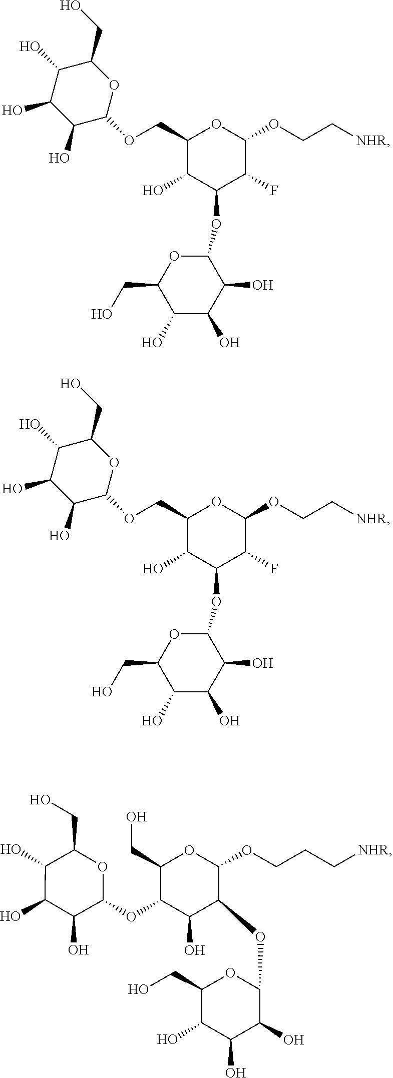 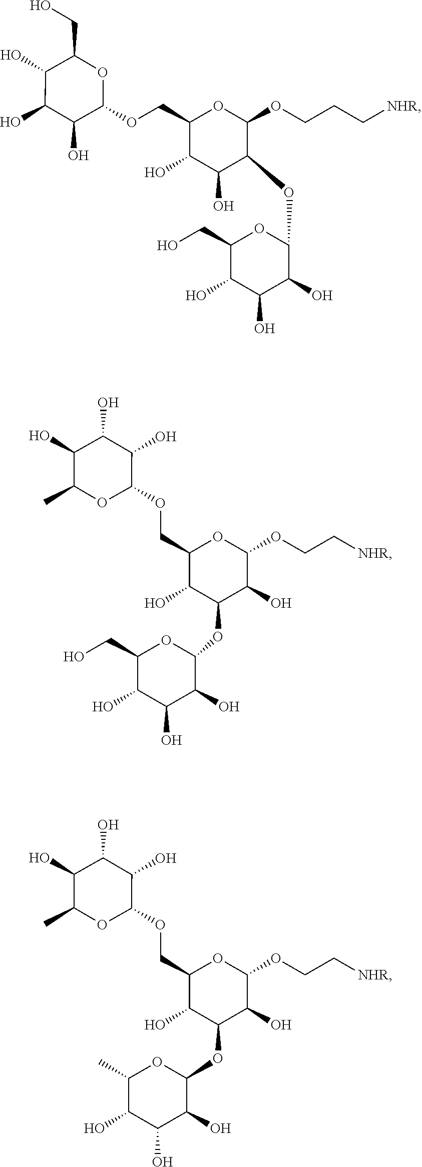 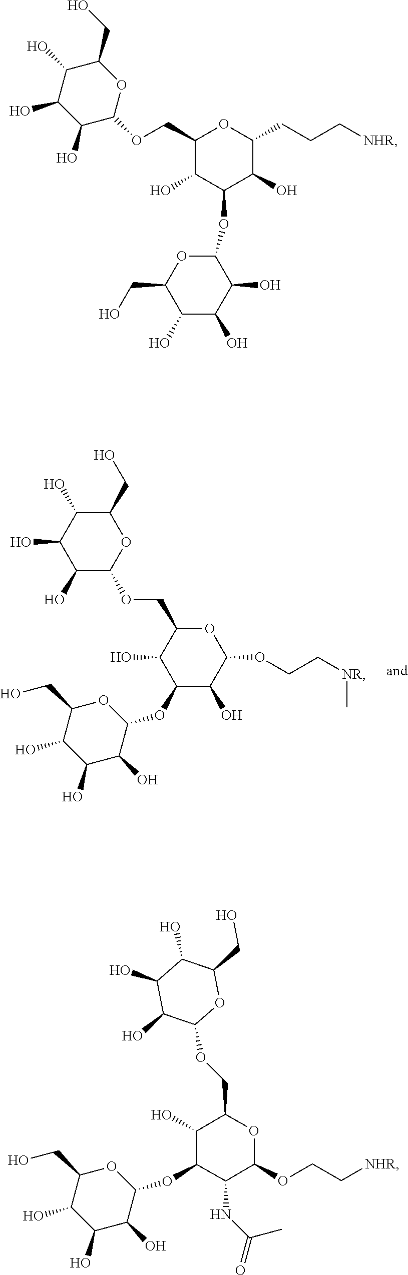
wherein R may be hydrogen or a carbonyl group of a linker.
